(19) United States
(12) Patent Application Publication
(10) Pub. No.: US 2021/0047649 A1
(43) Pub. Date: Feb. 18, 2021

(54) CRISPR/CAS ALL-IN-TWO VECTOR SYSTEMS FOR TREATMENT OF DMD

(71) Applicant: Vertex Pharmaceuticals Incorporated, Boston, MA (US)

(72) Inventors: Robert NG, Cambridge, MA (US); Seshidhar Reddy POLICE, Cambridge, MA (US); Yanfei YANG, Cambridge, MA (US)

(73) Assignee: Vertex Pharmaceuticals Incorporated, Boston, MA (US)

(21) Appl. No.: 16/870,478

(22) Filed: May 8, 2020

Related U.S. Application Data
(60) Provisional application No. 62/845,197, filed on May 8, 2019.

(51) Int. Cl. C12N 15/66; C12N 9/22; C12N 15/86

(52) U.S. Cl. CPC C12N 15/66; C12N 9/22; C12N 2710/10011; C12N 2310/20; C12N 15/86

(57) ABSTRACT

The present disclosure provides materials and methods for treating a patient with Duchenne Muscular Dystrophy (DMD), e.g., through ex vivo and in vivo methods of genome editing. The present disclosure also relates to methods and compositions for use of self-inactivating/self-targeting CRISPR/Cas or CRISPR/Cpf1 systems to genetically modify cells, e.g., to modulate the expression, function, and/or activity of the dystrophin gene.

### RELATED APPLICATIONS

This application claims the benefit of U.S. Provisional Patent Application Ser. No. 62/845,197, filed May 8, 2019. The entire contents of which is incorporated herein by reference.

### SEQUENCE LISTING

The instant application contains a Sequence Listing which has been submitted electronically in ASCII format and is hereby incorporated by reference in its entirety. Said ASCII copy, created on Oct. 28, 2020, is named 2020-10-28_01245-0026-00US_ST25.txt and is 145,603 bytes in size.

### BACKGROUND

Multiple studies suggest that genome engineering would be an attractive strategy for treating DMD. Duchenne Muscular Dystrophy (DMD) is a severe X-linked recessive neuromuscular disorder effecting approximately 1 in 4,000 live male births. Patients are generally diagnosed by the age of 4, and wheel chair bound by the age of 10. Most patients do not live past the age of 25 due to cardiac and/or respiratory failure. Existing treatments are palliative at best. The most common treatment for DMD is steroids, which are used to slow the loss of muscle strength. However, because most DMD patients start receiving steroids early in life, the treatment delays puberty and further contributes to the patient's diminished quality of life.

DMD is caused by mutations in the dystrophin gene (Chromosome X: 31, 1 17,228-33,344,609 (Genome Reference Consortium—GRCh38/hg38)). With a genomic region of over 2.2 megabases in length, dystrophin is the second largest human gene. The dystrophin gene contains 79 exons that are processed into an 11,000 base pair mRNA that is translated into a 427 kDa protein. Functionally, dystrophin acts as a linker between the actin filaments and the extracellular matrix within muscle fibers. The N-terminus of dystrophin is an actin-binding domain, while the C-terminus interacts with a transmembrane scaffold that anchors the muscle fiber to the extracellular matrix. Upon muscle contraction, dystrophin provides structural support that allows the muscle tissue to withstand mechanical force. DMD is caused by a wide variety of mutations within the dystrophin gene that result in premature stop codons and therefore a truncated dystrophin protein. Truncated dystrophin proteins do not contain the C-terminus, and therefore cannot provide the structural support necessary to withstand the stress of muscle contraction. As a result, the muscle fibers pull themselves apart, which leads to muscle wasting.

There is a need in the field for a technology that allows for controlling gene expression with minimal off-target effects, for example, for developing safe and effective treatments for DMD, which is among the most prevalent and debilitating genetic disorders.

### SUMMARY

The present disclosure presents an approach to address the genetic basis of DMD. By using genome engineering tools (e.g., CRISPR/Cas systems) to create changes to the genome that can restore the dystrophin reading frame and restore the dystrophin protein activity by correcting the underlying genetic defect causing the disease.

Provided herein are cellular, ex vivo and in vivo methods for creating changes to the genome by deleting, inserting, or replacing (deleting and inserting) one or more exons in the dystrophin gene by genome editing and restoring the dystrophin reading frame and restoring the dystrophin protein activity, which can be used to treat Duchenne Muscular Dystrophy (DMD).

In one aspect, provided herein is a CRISPR/Cas two vector system comprising (a) a first vector comprising a nucleic acid encoding (i) a first guide RNA (gRNA) comprising a DNA targeting sequence that is complementary to a first portion of the human DMD gene, wherein the DNA targeting sequence is 19-24 nucleotides in length and comprises a nucleotide sequence selected from the group consisting of SEQ ID NOs: 1-17; and (ii) a second gRNA comprising a DNA targeting sequence that is complementary to a second portion of the human DMD gene, wherein the DNA targeting sequence is 19-24 nucleotides in length and comprises a nucleotide sequence selected from the group consisting of SEQ ID NOs: 18-31; and (b) a second vector comprising a nucleic encoding a site-directed Cas9 polypeptide or variant thereof, wherein the nucleic encoding the site-directed Cas9 polypeptide comprises (i) a first gRNA target sequence which binds the first gRNA; and (ii) a second gRNA target sequence which binds the second gRNA, wherein binding of the first and second gRNAs to the nucleic acid encoding the site-directed Cas9 polypeptide inhibits expression of the Cas9 polypeptide.

In some embodiments, the targeting sequence of the first gRNA comprises a nucleotide sequence selected from the group consisting of SEQ ID NOs: 1-17, and the DNA targeting sequence of the second gRNA comprises the nucleotide sequence set forth in SEQ ID NO: 25. In some embodiments, the first gRNA comprises the nucleotide sequence set forth in SEQ ID NO: 13, and the targeting sequence of the second gRNA comprises a nucleotide sequence selected from the group consisting of SEQ ID NOs: 18-31. In some embodiments, the first gRNA comprises the nucleotide sequence set forth in SEQ ID NO: 14, and the targeting sequence of the second gRNA comprises a nucleotide sequence selected from the group consisting of SEQ ID NOs: 18-31. In one embodiment, the first gRNA comprises the nucleotide sequence set forth in SEQ ID NO: 13, and the targeting sequence of the second gRNA comprises the nucleotide sequence set forth in SEQ ID NO: 25. In another embodiment, the first gRNA comprises the nucleotide sequence set forth in SEQ ID NO: 14, and the targeting sequence of the second gRNA comprises the nucleotide sequence set forth in SEQ ID NO: 25.

In some embodiments, the first gRNA comprises the nucleotide sequence set forth in SEQ ID NO: 32. In some embodiments, the first gRNA comprises the nucleotide sequence set forth in SEQ ID NO: 33. In some embodiments, the second gRNA comprises the nucleotide sequence set forth in SEQ ID NO: 34. In some embodiments, the first gRNA comprises the nucleotide sequence set forth in SEQ ID NO: 32 and the second gRNA comprises the nucleotide sequence set forth in SEQ ID NO: 34. In some embodiments, the first gRNA comprises the nucleotide sequence set forth in SEQ ID NO: 33 and the second gRNA comprises the nucleotide sequence set forth in SEQ ID NO: 34.

In some embodiments, the first gRNA that is complementary to a portion of the DMD is a single RNA molecule. In some embodiments, the second gRNA that is complementary to a portion of the DMD is a single RNA molecule. In some embodiments, the first and second gRNAs are single RNA molecules.

In other embodiments, the first gRNA that is complementary to a portion of the DMD gene is a two-molecule guide RNA. In other embodiments, the second gRNA that is complementary to a portion of the DMD gene is a two-molecule guide RNA. In other embodiments, the first and second gRNAs are two-molecule guide RNAs. In some embodiments, the two-molecule guide RNA comprises a CRISPR RNA (crRNA-like) molecule and a trans-activating CRISPR RNA (tracrRNA-like) molecule.

In some embodiments, the first vector comprises a nucleic acid encoding from 5′ to 3′ (i) a first inverted terminal repeat (ITR); (ii) a first promoter; (iii) the first gRNA; (iv) a detectable polypeptide; (v) a second promoter; (vi) the second gRNA; and (vii) a second ITR.

In some embodiments, the 5′ ITR in the first vector comprises the nucleotide sequence set forth in SEQ ID NO: 41. In some embodiments, the first promoter is a U6 promoter comprising the sequence set forth in SEQ ID NO: 42. In some embodiments, the first and second promoter are the same. In some embodiments, the 3′ ITR comprises the nucleotide sequence set forth in SEQ ID NO: 43. In some embodiments, the detectable polypeptide is an albumin polypeptide. In some embodiments, the albumin polypeptide is encoded by the nucleotide sequence set forth in SEQ ID NO: 44. In some embodiments, the detectable polypeptide is HPRT. In some embodiments, the HPRT polypeptide is encoded by the nucleotide sequence set forth in SEQ ID NO: 45.

In some embodiments, the second vector comprises a nucleic acid encoding from 5′ to 3′, (i) a first inverted terminal repeat (ITR); (ii) a promoter; (iii) the site directed Cas9 polypeptide or variant thereof comprising the first and second gRNA target sequences; and (iv) a second ITR.

In some embodiments, the first and second gRNA target sequences are in the same orientation in the vector sequence. In some embodiments, the first and second gRNA target sequences are in the opposite orientation in the vector sequence. In some embodiments, the second vector comprises a first gRNA target sequence selected from SEQ ID NO: 38 or SEQ ID NO: 39. In some embodiments, the second vector comprises a second gRNA target sequence comprising the nucleotide sequence set forth in SEQ ID NO: 40.

In some embodiments, the first ITR in the second vector comprises the nucleotide sequence set forth in SEQ ID NO: 41. In some embodiments, the second ITR comprises the nucleotide sequence set forth in SEQ ID NO: 43. In some embodiments the promoter in the second vector is a CMV promoter. In some embodiments, the CMV promoter comprises the nucleotide sequence set forth in SEQ ID NO: 51.

In some embodiments, the second vector comprises a nucleotide sequence that encodes Staphylococcus aureus Cas9 (SaCas9) or a variant thereof. In some embodiments, the second vector encodes a SaCas9 comprising the amino acid sequence set forth in SEQ ID NO: 46. In some embodiments, the second vector encodes a SaCas9 variant comprising the amino acid sequence set forth in SEQ ID NO: 47. In other embodiments, the second vector comprises a SaCas9 variant comprising the amino acid sequence set forth in SEQ ID NO: 48. In other embodiments, the second vector comprises a SaCas9 variant comprising the amino acid sequence set forth in SEQ ID NO: 49.

In some embodiments, the nucleotide sequence encoding the SaCas9 comprises the nucleotide set forth in SEQ ID NO: 52, or a codon optimized variant thereof. In some embodiment, the nucleotide sequence encoding the SaCas9 or variant thereof, comprises an intron inserted into the open reading frame. In some embodiments, the intron comprises a nucleotide sequence selected from SEQ ID NOs: 53-56. In one embodiment, the intron inserted into the SaCas9 open reading frame comprises SEQ ID NO: 53.

In some embodiments, the first gRNA target sequences in the second vector is located at the 5′ end of the open reading frame of the SaCas9 or variant thereof. In some embodiments, the second gRNA target sequence is located within the open reading frame. In some embodiments the second gRNA target sequence is located within an intron located within the open reading frame of the SaCas9 or variant thereof.

In some embodiments, the first vector further comprises a polyA sequence. In some embodiments, the polyA sequence in the first vector is located 5′ of the second promoter sequence. In some embodiments, the second vector further comprises a polyA sequence. In some embodiments, the polyA sequence in the second vector is located 5′ of the second ITR.

In related embodiments, the first vector of the CRISPR/Cas two vector system is an adeno-associated virus (AAV) vector. In other embodiments, the second vector is an adeno-associated virus (AAV) vector.

In some embodiments, the first vector of the CRISPR/Cas two vector system comprises the nucleotide sequence set forth in SEQ ID NO: 68. In some embodiments, the first vector of the CRISPR/Cas two vector system comprises the nucleotide sequence set forth in SEQ ID NO: 71.

In some embodiments, the second vector of the CRISPR/Cas two vector system comprises the nucleotide sequence set forth in SEQ ID NO: 67. In some embodiments, the second vector comprises the nucleotide sequence set forth in SEQ ID NO: 70.

In one embodiment, the first vector of the CRISPR/Cas two vector system comprises the nucleotide sequence set forth in SEQ ID NO: 68, and the second vector comprises the nucleotide sequence set forth in SEQ ID NO: 67. In one embodiment, the first vector of the CRISPR/Cas two vector system comprises the nucleotide sequence set forth in SEQ ID NO: 71, and the second vector comprises the nucleotide sequence set forth in SEQ ID NO: 70.

Also provided herein are cells comprising any of the CRISPR/Cas systems provided herein. In some embodiments, the cell is a genetically modified cell. In some embodiments, the genetically modified cell is selected from the group consisting of a somatic cell, a stem cell and a mammalian cell. In some embodiments, the genetically modified cell is a stem cell selected from the group consisting of an embryonic stem (ES) cell, and an induced pluripotent stem (iPS) cell. In one embodiment, the cell is a muscle cell.

Also provided herein is a method of correcting a mutation in the human DMD gene in a cell, the method comprising contacting the cell with any of the CRISPR/Cas two vector systems provided herein, wherein the correction of the mutant dystrophin gene comprises deletion of exon 51 of the human DMD gene. In some embodiments, the cell is a myoblast cell. In some embodiments, the cell is from a subject with Duchenne muscular dystrophy.

Also provided herein is a method of treating a subject having a mutation in the human DMD gene, comprising administering to the subject the any of the CRISPR/Cas two vector systems provided herein. In some embodiments, the method comprises ex vivo administration of the CRISPR/Cas two vector system. In some embodiments, the CRISPR/Cas two vector system is administered intramuscularly, for example, the muscle is skeletal muscle or cardiac muscle. In other embodiments, the CRISPR/Cas two vector system is administered intravenously.

Also provided herein is a pharmaceutical composition and kits comprising any of the CRISPR-Cas systems provided herein, or any of the genetically modified cells provided herein.

It is understood that the inventions described in this specification are not limited to the examples summarized in this Summary. Various other aspects are described and exemplified herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Various aspects of self-inactivating CRISPR/Cas/Cpf1 systems and uses thereof disclosed and described in this specification can be better understood by reference to the accompanying figures, in which:

FIG. 1 is a schematic representation of a target specific CRISPR/Cas9 two vector system utilized in Example 1.

FIG. 2 depicts the nucleotide sequence of vector CTX-212 in which the elements are annotated.

FIG. 3 depicts the nucleotide sequence of vector CTX-214 in which the elements are annotated.

FIG. 4 depicts the nucleotide sequence of vector CTX-217 in which the elements are annotated.

FIG. 5A depicts Cas9 expression in mice over a 48 hour period.

FIG. 5B is a graph depicting the excision efficiency of exon 51 of the dystrophin gene at day 2 and day 4 after injection of the CRISPR/Cas9 vector system.

FIG. 6A is a graph depicting SaCas9 protein levels in liver lysate at 2, 4 and 12 weeks post-injection of CRISPR/Cas9 SIN vectors and CRISPR/Cas9 non-SIN vectors.

FIG. 6B is a graph depicting SaCas9 protein levels in heart lysate at 2, 4 and 12 weeks post-injection of CRISPR/Cas9 SIN vectors and CRISPR/Cas9 non-SIN vectors.

FIG. 6C is a graph depicting exon 23 excision efficiency at 2, 4 and 12 weeks post-injection of CRISPR/Cas9 Universal SIN vectors and CRISPR/Cas9 non-SIN vectors.

FIG. 6D is a graph depicting exon 23 excision efficiency at 2, 4 and 12 weeks post-injection of CRISPR/Cas9 Target-Specific SIN vectors and CRISPR/Cas9 non-SIN vectors.

FIG. 7A is a graph depicting SaCas9 mRNA levels after injection of CRISPR/Cas9 Universal SIN vectors, CRISPR/Cas9 Target-Specific SIN vectors and CRISPR/Cas9 non-SIN vectors as a control.

FIG. 7B is a graph depicting SaCas9 protein levels in retinal lysate after injection of CRISPR/Cas9 Universal SIN vectors, CRISPR/Cas9 Target-Specific SIN vectors and CRISPR/Cas9 non-SIN vectors as a control.

FIG. 7C is a graph depicting exon 23 excision efficiency after injection of CRISPR/Cas9 Universal SIN vectors, CRISPR/Cas9 Target-Specific SIN vectors and CRISPR/Cas9 non-SIN vectors as a control.

FIG. 8 is a schematic of the CRISPR/Cas9 Universal SIN two vector system for excision of exon 51 of the human DMD gene.

FIG. 9 is a schematic of the CRISPR/Cas9 Target-Specific SIN two vector system for excision of exon 51 of the human DMD gene.

FIG. 10 depicts the nucleotide sequence of vector CTX-506 in which the elements are annotated.

FIG. 11 depicts the nucleotide sequence of vector CTX-507 in which the elements are annotated.

FIG. 12 depicts the nucleotide sequence of vector CTX-603 in which the elements are annotated.

FIG. 13 depicts the nucleotide sequence of vector CTX-1074 in which the elements are annotated.

FIG. 14 depicts the nucleotide sequence of vector CTX-769 in which the elements are annotated.

FIG. 15 depicts the nucleotide sequence of vector CTX-1047 in which the elements are annotated.

FIG. 16 depicts the nucleotide sequence of vector CTX-1070 in which the elements are annotated.

FIG. 17 depicts the nucleotide sequence of vector CTX-525 in which the elements are annotated.

FIG. 18 depicts the nucleotide sequence of vector CTX-1048 in which the elements are annotated.

FIG. 19 depicts the nucleotide sequence of vector CTX-1075 in which the elements are annotated.

FIG. 20 is a graph depicting SaCas9 protein levels at days 1, 3 and 6 after transduction of HEK293 cells with the CRISPR/Cas9 Universal SIN two vector system and the CRISPR/Cas9 Target-Specific SIN two vector system.

FIG. 21 is a graph depicting exon 51 excision efficiency at days 1, 3 and 6 after transduction of HEK293T cells with the CRISPR/Cas9 Universal SIN two vector system and the CRISPR/Cas9 Target-Specific SIN two vector system.

FIG. 22A depicts SaCas9 protein levels over time utilizing the CRISPR/Cas9 Universal SIN two vector system.

FIG. 22B depicts SaCas9 protein levels over time utilizing the CRISPR/Cas9 Target-Specific SIN two vector system.

FIG. 23 depicts exon 51 excision efficiency over time after transduction of the CRISPR/Cas9 Universal SIN two vector system and the CRISPR/Cas9 Target-Specific SIN two vector system.

### DETAILED DESCRIPTION

The CRISPR/Cas/Cpf1 system is a powerful tool for development of next generation medicines to treat/cure intractable, inherited and acquired diseases; however, sustained CRISPR/Cas9 or CRISPR/Cpf1 expression in a cell is no longer necessary once all copies of a gene in the genome of a cell of interest have been edited. Chronic and constitutive endonuclease activity of Cas9 or Cpf1 can increase the number of off-target mutations and/or can generate anti-Cas9 or anti-Cpf1 immune responses resulting in elimination of the gene edited cells. Thus, temporal- and/or spatial-limited expression of Cas9 or Cpf1 is desirable to reduce or eliminate unwanted off-target effects of the endonuclease activity of Cas9 or Cpf1. The spatiotemporal control of Cas9 or Cpf1 expression can be also executed to lower/eliminate immune responses to Cas9 or Cpf1 resulting in enhanced safety and efficacy of gene editing.

### I. Terminology

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and can be isolated from genes, or chemically synthesized by methods known in the art. The terms “polynucleotide” and “nucleic acid” should be understood to include, as applicable to the aspects being described, single-stranded (such as sense or antisense) and double-stranded polynucleotides.

“Genomic DNA” refers to the DNA of a genome of an organism including, but not limited to, the DNA of the genome of a bacterium, fungus, archea, plant or animal.

“Manipulating” DNA encompasses binding, nicking one strand, or cleaving (i.e., cutting) both strands of the DNA, or encompasses modifying the DNA or a polypeptide associated with the DNA. Manipulating DNA can silence, activate, or modulate (either increase or decrease) the expression of an RNA or polypeptide encoded by the DNA.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem can include one or more base mismatches. Alternatively, the base-pairing can be exact, i.e. not include any mismatches.

By “hybridizable” or “complementary” or “substantially complementary” it is meant that a nucleic acid (e.g. RNA) comprises a sequence of nucleotides that enables it to non-covalently bind, e.g.: form Watson-Crick base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA].

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides, through “seed sequences”. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration can be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide can hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides can be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction can be sequence-specific. Binding interactions are generally characterized by a dissociation constant (K_{d}) of less than 10⁻⁶ M, less than 10⁻⁷ M, less than 10⁻⁸M, less than 10⁻⁹M, less than 10⁻¹⁰ M, less than 10⁻¹¹M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, or mafft.cbrc.jp/alignment/software/. See, e.g., Altschul et al. (1990), J. Mol. Bio. 215:403-10. Sequence alignments standard in the art are used according to the invention to determine amino acid residues in a Cas9 ortholog that “correspond to” amino acid residues in another Cas9 ortholog. The amino acid residues of Cas9 orthologs that correspond to amino acid residues of other Cas9 orthologs appear at the same position in alignments of the sequences.

A DNA sequence that “encodes” a particular RNA is a DNA nucleic acid sequence that is transcribed into RNA. A DNA polynucleotide can encode an RNA (mRNA) that is translated into protein, or a DNA polynucleotide can encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, can be used to drive the various vectors of the present invention.

A promoter can be a constitutively active promoter (i.e., a promoter that is constitutively in an active/“ON” state), it can be an inducible promoter (i.e., a promoter whose state, active/“ON” or inactive/“OFF”, is controlled by an external stimulus, e.g., the presence of a particular temperature, compound, or protein.), it can be a spatially restricted promoter (i.e., transcriptional control element, enhancer, etc.)(e.g., tissue specific promoter, cell type specific promoter, etc.), and it can be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., site-directed modifying polypeptide, or Cas9 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9 protein), the chimeric polypeptide includes amino acid sequences that are derived from different polypeptides. A chimeric polypeptide can comprise either modified or naturally-occurring polypeptide sequences (e.g., a first amino acid sequence from a modified or unmodified Cas9 protein; and a second amino acid sequence other than the Cas9 protein). Similarly, “chimeric” in the context of a polynucleotide encoding a chimeric polypeptide includes nucleotide sequences derived from different coding regions (e.g., a first nucleotide sequence encoding a modified or unmodified Cas9 protein; and a second nucleotide sequence encoding a polypeptide other than a Cas9 protein).

The term “chimeric polypeptide” refers to a polypeptide which is not naturally occurring, e.g., is made by the artificial combination (i.e., “fusion”) of two otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric Cas9 protein, the RNA-binding domain of a naturally-occurring bacterial Cas9 polypeptide (or a variant thereof) can be fused to a heterologous polypeptide sequence (i.e. a polypeptide sequence from a protein other than Cas9 or a polypeptide sequence from another organism). The heterologous polypeptide can exhibit an activity (e.g., enzymatic activity) that will also be exhibited by the chimeric Cas9 protein (e.g., methyltransferase activity, acetyltransferase activity, kinase activity, ubiquitinating activity, etc.). A heterologous nucleic acid can be linked to a naturally-occurring nucleic acid (or a variant thereof) (e.g., by genetic engineering) to generate a chimeric polynucleotide encoding a chimeric polypeptide. As another example, in a fusion variant Cas9 site-directed polypeptide, a variant Cas9 site-directed polypeptide can be fused to a heterologous polypeptide (i.e. a polypeptide other than Cas9), which exhibits an activity that will also be exhibited by the fusion variant Cas9 site-directed polypeptide. A heterologous nucleic acid can be linked to a variant Cas9 site-directed polypeptide (e.g., by genetic engineering) to generate a polynucleotide encoding a fusion variant Cas9 site-directed polypeptide. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA can be present 5′ or 3′ from the open reading frame, where such sequences do not interfere with manipulation or expression of the coding regions, and can indeed act to modulate production of a desired product by various mechanisms (see “DNA regulatory sequences”, below). Alternatively, DNA sequences encoding RNA (e.g., guide RNA) that is not translated can also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but can be a naturally occurring amino acid sequence.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) can or cannot be operably linked to a promoter sequence and can or cannot be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA can or cannot be integrated (covalently linked) into the genome of the cell.

In prokaryotes, yeast, and mammalian cells for example, the transforming DNA can be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell can not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (e.g., spacer or spacer sequence) of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The target DNA can be a double-stranded DNA. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds gRNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain aspects, a complex comprising a guide RNA and a site-directed modifying polypeptide is used for targeted double-stranded DNA cleavage.

A “self-inactivating site” or “SIN site” as used herein is a site within a self-inactivating vector that comprises a protospacer sequence and neighboring protospacer adjacent motif (PAM). For example, a SIN site can comprise 5′-N₁₇₋₂₁NRG-3′ or 5′-N₁₉₋₂₄NNGRRT-3′ wherein N₁₇₋₂₁ or N₁₉₋₂₄ represent protospacer sequence and NRG or NNGRRT represent PAMs for SpCas9 or SaCas9, respectively. The DNA targeting segment (e.g., spacer) of a DNA targeting nucleic acid (e.g., gRNA) hybridizes to the complementary strand of the protospacer sequence of the SIN site.

In certain aspects, the DNA targeting segment of the DNA targeting nucleic acid can be completely complementary to, and hybridize with the SIN site. In certain aspects, the SIN site can be substantially complementary, for example, having 1 or more mismatches, to the DNA targeting segment of the DNA targeting nucleic acid to modulate timing of self-inactivation.

In some aspects, the SIN site can comprise a PAM sequence for S. aureus Cas9, S. pyogenes Cas9, T. denticola Cas9, N. menginitidis Cas9, Cpf1, C. jejuni Cas9, S. thermophilus Cas9 or other orthologs described herein. In certain aspects the PAM sequence may be: NNGRRT, NRG, NAAAAN, NAAAAC, NNNNGHTT, YTN, NNNNACA, NNNACAC, NNVRYAC, NNNVRYM, NNAAAAW, or NNAGAAW.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain can consist of more than one isolated stretch of amino acids within a given polypeptide.

By “site-directed polypeptide” or “RNA-binding site-directed polypeptide” or “RNA-binding site-directed modifying polypeptide” it is meant a polypeptide that binds RNA and is targeted to a specific DNA sequence. A site-directed polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “protein-binding segment.” By “segment” it is meant a segment/section/region of a molecule, e.g., a contiguous stretch of nucleotides in an RNA. A segment can also mean a region/section of a complex such that a segment can comprise regions of more than one molecule. For example, in some cases the protein-binding segment (described below) of a guide RNA is one RNA molecule and the protein-binding segment therefore comprises a region of that RNA molecule. In other cases, the protein-binding segment (described below) of a guide RNA comprises two separate molecules that are hybridized along a region of complementarity. As an illustrative, non-limiting example, a protein-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can or cannot include regions with complementarity to other molecules.

The DNA-targeting segment (or “DNA-targeting sequence”) comprises a nucleotide sequence that is complementary to a specific sequence within a target DNA (the complementary strand of the target DNA) designated the “protospacer-like” sequence herein. The DNA-targeting segment of a gRNA is also referred to as the spacer or spacer sequence herein. The protein-binding segment (or “protein-binding sequence”) interacts with a site-directed modifying polypeptide. When the site-directed modifying polypeptide is a Cas9, Cas9 related polypeptide, Cpf1, or Cpf1 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA.

The protein-binding segment of a guide RNA comprises, in part, two complementary stretches of nucleotides that hybridize to one another to form a double stranded RNA duplex (dsRNA duplex).

In some examples, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some examples, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

In some examples, a guide RNA comprises two separate RNA molecules (RNA polynucleotides: an “activator-RNA” and a “targeter-RNA”, see below) and is referred to herein as a “double-molecule guide RNA” or a “two-molecule guide RNA.” In other examples, the guide RNA is a single RNA molecule (single RNA polynucleotide) and is referred to herein as a “single-molecule guide RNA,” a “single-guide RNA,” or an “sgRNA.” The term “guide RNA” or “gRNA” is inclusive, referring both to double-molecule guide RNAs (also called a “split guide”) and to single-molecule guide RNAs (i.e., sgRNAs).

A two-molecule guide RNA comprises two separate RNA molecules (a “targeter-RNA” and an “activator-RNA”). Each of the two RNA molecules of a two-molecule guide RNA comprises a stretch of nucleotides that are complementary to one another such that the complementary nucleotides of the two RNA molecules hybridize to form the double stranded RNA duplex of the protein-binding segment.

An exemplary two-molecule guide RNA comprises a crRNA-like (“CRISPR RNA” or “targeter-RNA”) molecule (which includes a CRISPR repeat or CRISPR repeat-like sequence) and a corresponding tracrRNA-like (“trans-activating CRISPR RNA” or “activator-RNA” or “tracrRNA”) molecule. A crRNA-like molecule (targeter-RNA) comprises both the DNA-targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator-RNA) comprises a stretch of nucleotides (duplex-forming segment) that forms the other half of the dsRNA duplex of the protein-binding segment of the guide RNA. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each crRNA-like molecule can be said to have a corresponding tracrRNA-like molecule. The crRNA-like molecule additionally provides the single stranded DNA-targeting segment. Thus, a crRNA-like and a tracrRNA-like molecule (as a corresponding pair) hybridize to form a guide RNA. A double-molecule guide RNA can comprise any corresponding crRNA and tracrRNA pair.

A two-molecule guide RNA can be designed to allow for controlled (i.e., conditional) binding of a targeter-RNA with an activator-RNA. Because a two-molecule guide RNA is not functional unless both the activator-RNA and the targeter-RNA are bound in a functional complex with Cas9, a two-molecule guide RNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

A single-molecule guide RNA comprises two stretches of nucleotides (a targeter-RNA and an activator-RNA) that are complementary to one another, are covalently linked (directly, or by intervening nucleotides), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The term “activator-RNA” is used herein to mean a tracrRNA-like molecule of a double-molecule guide RNA. The term “targeter-RNA” is used herein to mean a crRNA-like molecule of a double-molecule guide RNA. The term “duplex-forming segment” is used herein to mean the stretch of nucleotides of an activator-RNA or a targeter-RNA that contributes to the formation of the dsRNA duplex by hybridizing to a stretch of nucleotides of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the guide RNA. Therefore, a double-molecule guide RNA can be comprised of any corresponding activator-RNA and targeter-RNA pair.

RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer cannot be able to bind to the cognate targeter-RNA unless the aptamer is bound by the appropriate drug; (ii) a targeter-RNA with an aptamer cannot be able to bind to the cognate activator-RNA unless the aptamer is bound by the appropriate drug; and (iii) a targeter-RNA and an activator-RNA, each comprising a different aptamer that binds a different drug, cannot be able to bind to each other unless both drugs are present. As illustrated by these examples, a two-molecule guide RNA can be designed to be inducible.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell. PSCs can be target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs can be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells can be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which can be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This quiescent state can be temporary, i.e. reversible, or it can be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair can result in an alteration of the sequence of the target molecule (e.g., insertion, deletion, mutation), if the donor polynucleotide differs from the target molecule and part or all of the sequence of the donor polynucleotide is incorporated into the target DNA. In some examples, the donor polynucleotide, a portion of the donor polynucleotide, a copy of the donor polynucleotide, or a portion of a copy of the donor polynucleotide integrates into the target DNA.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, “treating” and the like are used herein to generally mean obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which can be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent can be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The term “comprising” or “comprises” is used in reference to compositions, methods, and respective component(s) thereof, that are essential to the present disclosure, yet open to the inclusion of unspecified elements, whether essential or not.

The term “consisting essentially of” refers to those elements required for a given aspect. The term permits the presence of additional elements that do not materially affect the basic and novel or functional characteristic(s) of that aspect of the present disclosure.

The term “consisting of” refers to compositions, methods, and respective components thereof as described herein, which are exclusive of any element not recited in that description of the aspect.

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “2.4 to 7.6” is not expressly recited in the text of the specification. Accordingly, the Applicant reserves the right to amend this specification, including the claims, to expressly recite any sub-range of the same numerical precision subsumed within the ranges expressly recited in this specification. All such ranges are inherently described in this specification such that amending to expressly recite any such sub-ranges will comply with written description, sufficiency of description, and added matter requirements, including the requirements under 35 U.S.C. § 112(a) and Article 123(2) EPC. Also, unless expressly specified or otherwise required by context, all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameter.

It is appreciated that certain features of the invention, which are, for clarity, described in the context of separate examples, can also be provided in combination in a single example. Conversely, various features of the invention, which are, for brevity, described in the context of a single example, can also be provided separately or in any suitable sub-combination. All combinations of the examples pertaining to the disclosure are specifically embraced by the present invention and are disclosed herein just as if each and every combination was individually and explicitly disclosed. In addition, all sub-combinations of the various examples and elements thereof are also specifically embraced by the present invention and are disclosed herein just as if each and every such sub-combination was individually and explicitly disclosed herein.

Genome editing generally refers to the process of editing or changing the nucleotide sequence of a genome, preferably in a precise, desirable and/or pre-determined manner. Examples of compositions, systems, and methods of genome editing described herein use of site-directed nucleases to cut or cleave DNA at precise target locations in the genome, thereby creating a double-strand break (DSB) in the DNA. Such breaks can be repaired by endogenous DNA repair pathways, such as homology directed repair (HDR) and/or non-homologous end-joining (NHEJ) repair (see e.g., Cox et al., (2015) Nature Medicine 21 (2):121-31). One of the major obstacles to efficient genome editing in non-dividing cells is lack of homology directed repair (HDR). Without HDR, non-dividing cells rely on non-homologous end joining (NHEJ) to repair double-strand breaks (DSB) that occur in the genome. The results of NHEJ-mediated DNA repair of DSBs can include correct repair of the DSB, or deletion or insertion of one or more nucleotides or polynucleotides.

### II. Donor Polynucleotides

The disclosure provides donor polynucleotides that, upon insertion into a DSB, correct or induce a mutation in a target nucleic acid (e.g., a genomic DNA). In some embodiments, the donor polynucleotides provided by the disclosure are recognized and used by the HDR machinery of a cell to repair a double strand break (DSB) introduced into a target nucleic acid by a site-directed nuclease, wherein repair of the DSB results in the insertion of the donor polynucleotide into the target nucleic acid. Alternatively, a donor polynucleotide may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter at the integration site, namely the promoter that drives expression of the endogenous gene into which the donor is inserted. However, in some embodiments, the donor template comprises an exogenous promoter and/or enhancer, for example a constitutive promoter, an inducible promoter, or tissue-specific promoter.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, the donor polynucleotides comprise a nucleotide sequence which corrects or induces a mutation in a genomic DNA (gDNA) molecule in a cell, wherein when the donor polynucleotide is introduced into the cell in combination with a site-directed nuclease, a HDR DNA repair pathway inserts the donor polynucleotide into a double-stranded DNA break (DSB) introduced into the gDNA by the site-directed nuclease at a location proximal to the mutation, thereby correcting the mutation.

In some embodiments, the donor polynucleotide comprises a nucleotide sequence which corrects or induces a mutation, wherein the nucleotide sequence that corrects or induces a mutation comprises a single nucleotide. In some embodiments, the nucleotide sequence which corrects or induces a mutation comprises two or more nucleotides. In some embodiments, the nucleotide sequence which corrects or induces a mutation comprises a codon. In some embodiments, the nucleotide sequence which corrects or induces a mutation is comprises one or more codons. In some embodiments, the nucleotide sequence which corrects or induces a mutation comprises an exonic sequence. In some embodiments, the donor polynucleotide comprises a nucleotide sequence which corrects or induces a mutation, wherein the nucleotide sequence which corrects or induces a mutation comprises an intronic sequence.

In some embodiments, the donor polynucleotide sequence is identical to or substantially identical to (having at least one nucleotide difference) an endogenous sequence of a target nucleic acid. In some embodiments, the endogenous sequence comprises a genomic sequence of the cell. In some embodiments, the endogenous sequence comprises a chromosomal or extrachromosomal sequence. In some embodiments, the donor polynucleotide sequence comprises a sequence that is substantially identical (comprises at least one nucleotide difference/change) to a portion of the endogenous sequence in a cell at or near the DSB. In some embodiments, repair of the target nucleic acid molecule with the donor polynucleotide results in an insertion, deletion, or substitution of one or more nucleotides of the target nucleic acid molecule. In some embodiments, the insertion, deletion, or substitution of one or more nucleotides results in one or more amino acid changes in a protein expressed from a gene comprising the target sequence. In some embodiments, the insertion, deletion, or substitution of one or more nucleotides results in one or more nucleotide changes in an RNA expressed from the target gene. In some embodiments, the insertion, deletion, or substitution of one or more nucleotides alters the expression level of the target gene. In some embodiments, the insertion, deletion, or substitution of one or more nucleotides results in increased or decreased expression of the target gene. In some embodiments, the insertion, deletion, or substitution of one or more nucleotides results in gene knockdown. In some embodiments, the insertion, deletion, or substitution of one or more nucleotides results in gene knockout. In some embodiments, the repair of the target nucleic acid molecule with the donor polynucleotide results in replacement of an exon sequence, an intron sequence, a transcriptional control sequence, a translational control sequence, a sequence comprising a splicing signal, or a non-coding sequence of the target gene.

The donor polynucleotide is of a suitable length to correct or induce a mutation in a gDNA. In some embodiments, the donor polynucleotide comprises 10, 15, 20, 25, 50, 75, 100 or more nucleotides in length. In some embodiments (for example those described herein where a donor polynucleotide is incorporated into the cleaved nucleic acid as an insertion mediated by non-homologous end joining) the donor polynucleotide has no homology arms. In some embodiments, the donor polynucleotide is about 10-100, about 20-80, about 30-70, or about 40-60 nucleotides in length. In some embodiments, the donor polynucleotide is about 10-100 nucleotides in length. In some embodiments, the donor polynucleotide is about 20-80 nucleotides in length. In some embodiments, the donor polynucleotide is about 30-70 nucleotides in length. In some embodiments, the donor polynucleotide is about 40-60 nucleotides in length. In some embodiments, the donor polynucleotide is 40, 41, 42, 43, 44, 45, 46, 46, 48, 49, 50, 51, 52, 53, 54, 55, 56, 57, 58, 59 or 60 nucleotides in length. In some embodiments, the donor polynucleotide is 40 nucleotides in length. In some embodiments, the donor polynucleotide is 41 nucleotides in length. In some embodiments, the donor polynucleotide is 42 nucleotides in length. In some embodiments, the donor polynucleotide is 43 nucleotides in length. In some embodiments, the donor polynucleotide is 44 nucleotides in length. In some embodiments, the donor polynucleotide is 45 nucleotides in length. In some embodiments, the donor polynucleotide is 46 nucleotides in length. In some embodiments, the donor polynucleotide is 47 nucleotides in length. In some embodiments, the donor polynucleotide is 48 nucleotides in length. In some embodiments, the donor polynucleotide is 49 nucleotides in length. In some embodiments, the donor polynucleotide is 50 nucleotides in length. In some embodiments, the donor polynucleotide is 51 nucleotides in length. In some embodiments, the donor polynucleotide is 52 nucleotides in length. In some embodiments, the donor polynucleotide is 53 nucleotides in length. In some embodiments, the donor polynucleotide is 54 nucleotides in length. In some embodiments, the donor polynucleotide is 55 nucleotides in length. In some embodiments, the donor polynucleotide is 56 nucleotides in length. In some embodiments, the donor polynucleotide is 57 nucleotides in length. In some embodiments, the donor polynucleotide is 58 nucleotides in length. In some embodiments, the donor polynucleotide is 59 nucleotides in length. In some embodiments, the donor polynucleotide is 60 nucleotides in length.

In some embodiments, a donor polynucleotide provided by the disclosure comprises an intronic sequence. In some embodiments, the donor polynucleotide comprises an intronic sequence which corrects or induces a mutation in a gDNA. In some embodiments, the donor polynucleotide comprises an exonic sequence. In some embodiments, the donor polynucleotide comprises an exonic sequence which corrects or induces a mutation in a gDNA.

The donor polynucleotides provided by the disclosure are produced by suitable DNA synthesis method or means known in the art. DNA synthesis is the natural or artificial creation of deoxyribonucleic acid (DNA) molecules. The term DNA synthesis refers to DNA replication, DNA biosynthesis (e.g., in vivo DNA amplification), enzymatic DNA synthesis (e.g., polymerase chain reaction (PCR); in vitro DNA amplification) or chemical DNA synthesis.

In some embodiments, each strand of the donor polynucleotide is produced by oligonucleotide synthesis. Oligonucleotide synthesis is the chemical synthesis of relatively short fragments or strands of single-stranded nucleic acids with a defined chemical structure (sequence). Methods of oligonucleotide synthesis are known in the art (see e.g., Reese (2005) Organic & Biomolecular Chemistry 3(21):3851). The two strands can then be annealed together or duplexed to form a donor polynucleotide.

In some aspects, the insertion of a donor polynucleotide into a DSB is determined by a suitable method known in the art. For example, after the insertional event, the nucleotide sequence of PCR amplicons generated using PCR primer that flank the DSB site is analyzed for the presence of the nucleotide sequence comprising the donor polynucleotide. Next-generation sequencing (NGS) techniques are used to determine the extent of donor polynucleotide insertion into a DSB analyzing PCR amplicons for the presence or absence of the donor polynucleotide sequence. Further, since each donor polynucleotide is a linear, dsDNA molecule, which can insert in either of two orientations, NGS analysis can be used to determine the extent of insertion of the donor polynucleotide in either direction.

In some aspects, the insertion of the donor polynucleotide and its ability to correct a mutation is determined by nucleotide sequence analysis of mRNA transcribed from the gDNA into which the donor polynucleotide is inserted. An mRNA transcribed from gDNA containing an inserted donor polynucleotide is analyzed by a suitable method known in the art. For example, conversion of mRNA extracted from cells treated or contacted with a donor polynucleotide or system provided by the disclosure is enzymatically converted into cDNA, which is further by analyzed by NGS analysis to determine the extent of mRNA molecule comprising the corrected mutation.

In other aspects, the insertion of a donor polynucleotide and its ability to correct a mutation is determined by protein sequence analysis of a polypeptide translated from an mRNA transcribed from the gDNA into which the donor polynucleotide is inserted. In some embodiments, a donor polynucleotide corrects or induces a mutation by the incorporation of a codon into an exon that makes an amino acid change in a gene comprising a gDNA molecule, wherein translation of an mRNA from the gene containing the inserted donor polynucleotide generates a polypeptide comprising the amino acid change. The amino acid change in the polypeptide is determined by protein sequence analysis using techniques including, but not limited to, Sanger sequencing, mass spectrometry, functional assays that measure an enzymatic activity of the polypeptide, or immunoblotting using an antibody reactive to the amino acid change.

In some embodiments, a donor polynucleotide provided by the disclosure is used to correct or induce a mutation in a gDNA in a cell by insertion of the donor polynucleotide into a target nucleic acid (e.g., gDNA) at a cleavage site (e.g, a DSB) induced by a site-directed nuclease, such as those described herein. In some embodiments, HDR DNA repair mechanisms of the cell repair the DSB using the donor polynucleotide, thereby inserting the donor polynucleotide into the DSB and adding the nucleotide sequence of the donor polynucleotide to the gDNA. In some embodiments, the donor polynucleotide comprises a nucleotide sequence which corrects a disease-causing mutation in a gDNA in a cell. In some embodiments, the donor polynucleotide is inserted at a location proximal to the mutation, thereby correcting the mutation. In some embodiments, the mutation is a substitution, missense, nonsense, insertion, deletion or frameshift mutation. In some embodiments the mutation is in an exon. In some embodiments, the mutation is a substitution, insertion or deletion and is located in an intron. In some embodiments, the mutation is proximal to a cleavage site in a gDNA. In some embodiments, the mutation is a protein-coding mutation. In some embodiments, the mutation is associated with or causes a disease.

In some embodiments, the donor polynucleotide is inserted into the DSB by HDR DNA repair. In some embodiments, the donor polynucleotide, a portion of the donor polynucleotide is inserted into the target nucleic acid cleavage site by HDR DNA repair. In certain aspects, insertion of a donor polynucleotide into the target nucleic acid via HDR repair can result in, for example, mutations, deletions, alterations, integrations, gene correction, gene replacement, gene tagging, transgene insertion, nucleotide deletion, gene disruption, translocations and/or gene mutation of the endogenous gene sequence.

In some embodiments, the disclosure provides donor polynucleotides used to repair a DSB introduced into a target nucleic acid molecule (e.g., gDNA) by a site-directed nuclease (e.g., Cas9) in a cell. In some embodiments, the donor polynucleotide is used by the HDR repair pathway of the cell to repair the DSB in the target nucleic acid molecule. In some embodiments, the site-directed nuclease is a Cas nuclease. In some embodiments, the Cas nuclease is Cas9. The site-directed nucleases described herein can introduce DSB in target nucleic acids (e.g., genomic DNA) in a cell. The introduction of a DSB in the genomic DNA of a cell, induced by a site-directed nuclease, will stimulate the endogenous DNA repair pathways, such as those described herein. The HDR pathway can be used to insert a polynucleotide (e.g., a donor polynucleotide) into the DSB during repair.

Accordingly, in some embodiments, a single donor polynucleotide or multiple copies of the same donor polynucleotide are provided. In other embodiments, two or more donor polynucleotides are provided such that repair may occur at two or more target sites. For example, different donor polynucleotides are provided to repair a single gene in a cell, or two different genes in a cell. In some embodiments, the different donor polynucleotides are provided in independent copy numbers.

In some embodiments, the donor polynucleotide are incorporated into the target nucleic acid as an insertion mediated by HDR. In some embodiments, the donor polynucleotide sequence has no similarity to the nucleic acid sequence near the cleavage site. In some embodiments, a single donor polynucleotide or multiple copies of the same donor polynucleotide are provided. In other embodiments, two or more donor polynucleotides having different sequences are inserted at two or more sites by non-homologous end joining. In some embodiments, the different donor polynucleotides are provided in independent copy numbers.

### III. CRISPR/Cas Nuclease Systems

Naturally-occurring CRISPR/Cas systems are genetic defense systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et al., (2017) Curr Opin Microbiol 37:67-78).

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g.: Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g. genes).

A. Cas Nuclease

In some embodiments, the disclosure provides compositions and systems (e.g. an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9, and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein are from Streptococcus thermophilus (StCas9). In some embodiments, the Cas9 protein are from Neisseria meningitides (NmCas9). In some embodiments, the Cas9 protein are from Staphylococcus aureus (SaCas9). In some embodiments, the Cas9 protein are from Campylobacter jejuni (CjCas9).

In some embodiments, a Cas nuclease may comprise more than one nuclease domain. For example, a Cas9 nuclease may comprise at least one RuvC-like nuclease domain (e.g. Cpf1) and at least one HNH-like nuclease domain (e.g. Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In alternative embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

B. Modified Nucleases

In some embodiments, the nuclease is optionally modified from its wild-type counterpart. The site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acids Res, 39(21): 9275-9282 (2011), or Cas9 from S.aureus, WO2015/071474 Sequence ID No. 244], and various other site-directed polypeptides.

In some embodiments, the site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes or S. aureus, supra).

In some embodiments, The site-directed polypeptide can comprise at least 70, 75, 80, 85, 90, 95, 97, 99, or 100% identity to a wild-type site-directed polypeptide (e.g., Cas9 from S. pyogenes or S. aureus, supra) over 10 contiguous amino acids. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids. The site-directed polypeptide can comprise at least: 70, 75, 80, 85, 90, 95, 97, 99, or 100% identity to a wild-type site-directed polypeptide (e.g., Cas9 from S. pyogenes or S. aureus, supra) over 10 contiguous amino acids in a HNH nuclease domain of the site-directed polypeptide. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids in a HNH nuclease domain of the site-directed polypeptide. The site-directed polypeptide can comprise at least: 70, 75, 80, 85, 90, 95, 97, 99, or 100% identity to a wild-type site-directed polypeptide (e.g., Cas9 from S. pyogenes or S. aureus, supra) over 10 contiguous amino acids in a RuvC nuclease domain of the site-directed polypeptide. The site-directed polypeptide can comprise at most: 70, 75, 80, 85, 90, 95, 97, 99, or 100% identity to a wild-type site-directed polypeptide (e.g., Cas9 from S. pyogenes or S. aureus, supra) over 10 contiguous amino acids in a RuvC nuclease domain of the site-directed polypeptide.

In some embodiments, the modified form of the wild-type exemplary site-directed polypeptide can comprise a mutation that reduces the nucleic acid-cleaving activity of the site-directed polypeptide. The modified form of the wild-type exemplary site-directed polypeptide can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes or S. aureus, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide can comprise a mutation such that it can induce a single-strand break (SSB) on a target nucleic acid (e.g., by cutting only one of the sugar-phosphate backbones of a double-strand target nucleic acid). The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes or S. aureus, supra). The mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). The residues to be mutated can correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. Additional examples of mutations can include N497A, R661A, N692A, M694A, Q695A, H698A, E762A, K810A, K848A, K855A, N863A, Q926A, D986A, K1003A and R1060A. One skilled in the art will recognize that mutations other than alanine substitutions can be suitable.

A D10A mutation can be combined with one or more of H840A, N854A, or N856A mutations to produce a site-directed polypeptide substantially lacking DNA cleavage activity. A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, residues in the wild-type exemplary S.aureus Cas9 polypeptide, such as Asp10 or Asn580 are mutated to inactivate one or more of the plurality of nucleic acid-cleaving domains (e.g., nuclease domains). Non-limiting examples of mutations include D10A and N580A. A D10A mutation can be combined with one or more mutations, including N580A to produce a site-directed polypeptide substantially lacking DNA cleavage activity.

Site-directed polypeptides that comprise one substantially inactive nuclease domain are referred to as “nickases”. Nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes.

Mutations contemplated can include substitutions, additions, and deletions, or any combination thereof. The mutation converts the mutated amino acid to alanine. The mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagines, glutamine, histidine, lysine, or arginine). The mutation converts the mutated amino acid to a non-natural amino acid (e.g., selenomethionine). The mutation converts the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

The site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target DNA. The site-directed polypeptide (e.g. variant, mutated, enzymatically inactive and/or conditionally enzymatically inactive endoribonuclease) can target RNA.

The site-directed polypeptide can comprise an amino acid sequence comprising at least 15% amino acid identity to a Cas9 from a bacterium (e.g., S. pyogenes or S. aureus), a nucleic acid binding domain, and two nucleic acid cleaving domains (i.e., a HNH domain and a RuvC domain).

The site-directed polypeptide can comprise an amino acid sequence comprising at least 15% amino acid identity to a Cas9 from a bacterium (e.g., S. pyogenes or S. aureus), and two nucleic acid cleaving domains (i.e., a HNH domain and a RuvC domain).

The site-directed polypeptide can comprise an amino acid sequence comprising at least 15% amino acid identity to a Cas9 from a bacterium (e.g., S. pyogenes or S. aureus), and two nucleic acid cleaving domains, wherein one or both of the nucleic acid cleaving domains comprise at least 50% amino acid identity to a nuclease domain from Cas9 from a bacterium (e.g., S. pyogenes).

The site-directed polypeptide can comprise an amino acid sequence comprising at least 15% amino acid identity to a Cas9 from a bacterium (e.g., S. pyogenes or S. aureus), two nucleic acid cleaving domains (i.e., a HNH domain and a RuvC domain), and non-native sequence (for example, a nuclear localization signal) or a linker linking the site-directed polypeptide to a non-native sequence.

The site-directed polypeptide can comprise an amino acid sequence comprising at least 15% amino acid identity to a Cas9 from a bacterium (e.g., S. pyogenes or S. aureus), two nucleic acid cleaving domains (i.e., a HNH domain and a RuvC domain), wherein the site-directed polypeptide comprises a mutation in one or both of the nucleic acid cleaving domains that reduces the cleaving activity of the nuclease domains by at least 50%.

The site-directed polypeptide can comprise an amino acid sequence comprising at least 15% amino acid identity to a Cas9 from a bacterium (e.g., S. pyogenes or S. aureus), and two nucleic acid cleaving domains (i.e., a HNH domain and a RuvC domain), wherein one of the nuclease domains comprises mutation of aspartic acid 10, and/or wherein one of the nuclease domains can comprise a mutation of histidine 840, and/or wherein one of the nuclease domains can comprise a mutation of Asparagine 580 and wherein the mutation reduces the cleaving activity of the nuclease domain(s) by at least 50%.

The one or more site-directed polypeptides, e.g. DNA endonucleases, can comprise two nickases that together effect one double-strand break at a specific locus in the genome, or four nickases that together effect or cause two double-strand breaks at specific loci in the genome. Alternatively, one site-directed polypeptide, e.g. DNA endonuclease, can effect or cause one double-strand break at a specific locus in the genome.

In some embodiments, the site-directed polypeptide can comprise one or more non-native sequences (e.g., the site-directed polypeptide is a fusion protein). In some embodiments, the nuclease is fused with at least one heterologous protein domain. At least one protein domain is located at the N-terminus, the C-terminus, or in an internal location of the nuclease. In some embodiments, two or more heterologous protein domains are at one or more locations on the nuclease.

In some embodiments, the protein domain may facilitate transport of the nuclease into the nucleus of a cell. For example, the protein domain is a nuclear localization signal (NLS). In some embodiments, the nuclease is fused with 1-10 NLS(s). In some embodiments, the nuclease is fused with 1-5 NLS(s). In some embodiments, the nuclease is fused with one NLS. In other embodiments, the nuclease is fused with more than one NLS. In some embodiments, the nuclease is fused with 2, 3, 4, or 5 NLSs. In some embodiments, the nuclease is fused with 2 NLSs. In some embodiments, the nuclease is fused with 3 NLSs. In some embodiments, the nuclease is fused with no NLS. In some embodiments, the NLS may be a monopartite sequence, such as, e.g., the SV40 NLS, PKKKRKV (SEQ ID NO: 72) or PKKKRRV (SEQ ID NO: 73). In some embodiments, the NLS is a bipartite sequence, such as, e.g., the NLS of nucleoplasmin, KRPAATKKAGQAKKKK (SEQ ID NO: 74). In some embodiments, the NLS is genetically modified from its wild-type counterpart.

In some embodiments, the protein domain is capable of modifying the intracellular half-life of the nuclease. In some embodiments, the half-life of the nuclease may be increased. In some embodiments, the half-life of the nuclease is reduced. In some embodiments, the entity is capable of increasing the stability of the nuclease. In some embodiments, the entity is capable of reducing the stability of the nuclease. In some embodiments, the protein domain act as a signal peptide for protein degradation. In some embodiments, the protein degradation is mediated by proteolytic enzymes, such as, e.g., proteasomes, lysosomal proteases, or calpain proteases. In some embodiments, the protein domain comprises a PEST sequence. In some embodiments, the nuclease is modified by addition of ubiquitin or a polyubiquitin chain. In some embodiments, the ubiquitin is a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub 1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the protein domain is a marker domain. Non-limiting examples of marker domains include fluorescent proteins, purification tags, epitope tags, and reporter gene sequences. In some embodiments, the marker domain is a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain is a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, S1, T7, V5, VSV-G, 6xHis, biotin carboxyl carrier protein (BCCP), and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the protein domain may target the nuclease to a specific organelle, cell type, tissue, or organ.

In further embodiments, the protein domain is an effector domain. When the nuclease is directed to its target nucleic acid, e.g., when a Cas9 protein is directed to a target nucleic acid by a guide RNA, the effector domain may modify or affect the target nucleic acid. In some embodiments, the effector domain is chosen from a nucleic acid binding domain, a nuclease domain, an epigenetic modification domain, a transcriptional activation domain, or a transcriptional repressor domain.

Certain embodiments of the invention also provide nucleic acids encoding the nucleases (e.g., a Cas9 protein) described herein provided on a vector. In some embodiments, the nucleic acid is a DNA molecule. In other embodiments, the nucleic acid is an RNA molecule. In some embodiments, the nucleic acid encoding the nuclease is an mRNA molecule. In certain embodiments, the nucleic acid is an mRNA encoding a Cas9 protein.

In some embodiments, the nucleic acid encoding the nuclease is codon optimized for efficient expression in one or more eukaryotic cell types. In some embodiments, the nucleic acid encoding the nuclease is codon optimized for efficient expression in one or more mammalian cells. In some embodiments, the nucleic acid encoding the nuclease is codon optimized for efficient expression in human cells. Methods of codon optimization including codon usage tables and codon optimization algorithms are available in the art.

### IV. Guide RNAs (gRNAs)

Engineered CRISPR/Cas systems comprise at least two components: 1) a guide RNA (gRNA) molecule and 2) a Cas nuclease, which interact to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g. a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA:tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA provided by the disclosure comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can comprise a 20 nucleotide spacer sequence at the 5′ end of the sgRNA sequence. The sgRNA can comprise a less than 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence.

The sgRNA can comprise no uracil at the 3′ end of the sgRNA sequence. The sgRNA can comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

By way of illustration, guide RNAs used in the CRISPR/Cas system, or other smaller RNAs can be readily synthesized by chemical means, as illustrated herein and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

A. Spacer Sequences

In some embodiments, the gRNAs provided by the disclosure comprise a spacer sequence. A spacer sequence is a sequence that defines the target site of a target nucleic acid (e.g.: DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

The spacer sequence hybridizes to a sequence in a target nucleic acid of interest. The spacer of a DNA-targeting nucleic acid can interact with a target nucleic acid in a sequence-specific manner via hybridization (i.e., base pairing). The nucleotide sequence of the spacer can vary depending on the sequence of the target nucleic acid of interest. The spacer sequence is also referred to as the DNA-targeting segment.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the target sequence and the PAM comprises the formula 5′ N19-21-N-R-G-3′, wherein N is any nucleotide, and wherein R is a nucleotide comprising the nucleobase adenine (A) or guanine (G), and wherein the three 3′ terminal nucleic acids, N-R-G represent the S. pyogenes PAM. In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

The spacer sequence that hybridizes to the target nucleic acid can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which can be thought of as a bulge or bulges.

In some embodiments, the spacer comprise at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

B. CRISPR Repeat Sequences

A minimum CRISPR repeat sequence can be a sequence with at least about 30%, about 40%, about 50%, about 60%, about 65%, about 70%, about 75%, about 80%, about 85%, about 90%, about 95%, or 100% sequence identity to a reference CRISPR repeat sequence (e.g., crRNA from S. pyogenes or S. aureus).

A minimum CRISPR repeat sequence can comprise nucleotides that can hybridize to a minimum tracrRNA sequence in a cell. The minimum CRISPR repeat sequence and a minimum tracrRNA sequence can form a duplex, i.e. a base-paired double-stranded structure. Together, the minimum CRISPR repeat sequence and the minimum tracrRNA sequence can bind to the site-directed polypeptide. At least a part of the minimum CRISPR repeat sequence can hybridize to the minimum tracrRNA sequence. At least a part of the minimum CRISPR repeat sequence can comprise at least about 30%, about 40%, about 50%, about 60%, about 65%, about 70%, about 75%, about 80%, about 85%, about 90%, about 95%, or 100% complementary to the minimum tracrRNA sequence. At least a part of the minimum CRISPR repeat sequence can comprise at most about 30%, about 40%, about 50%, about 60%, about 65%, about 70%, about 75%, about 80%, about 85%, about 90%, about 95%, or 100% complementary to the minimum tracrRNA sequence.

The minimum CRISPR repeat sequence can have a length from about 7 nucleotides to about 100 nucleotides. For example, the length of the minimum CRISPR repeat sequence is from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The minimum CRISPR repeat sequence can be approximately 9 nucleotides in length. The minimum CRISPR repeat sequence can be approximately 12 nucleotides in length.

The minimum CRISPR repeat sequence can be at least about 60% identical to a reference minimum CRISPR repeat sequence (e.g., wild-type crRNA from S. pyogenes or S. aureus) over a stretch of at least 6, 7, or 8 contiguous nucleotides. For example, the minimum CRISPR repeat sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides. The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides.

The duplex can comprise a mismatch (i.e., the two strands of the duplex are not 100% complementary). The duplex can comprise at least about 1, 2, 3, 4, or 5 or mismatches. In some examples, the duplex comprises at most about 1, 2, 3, 4, or 5 or mismatches. The duplex can comprise no more than 2 mismatches.

C. Bulges

In some cases, there can be a “bulge” in the duplex between the minimum CRISPR RNA and the minimum tracrRNA. A bulge is an unpaired region of nucleotides within the duplex. A bulge can contribute to the binding of the duplex to the site-directed polypeptide. The number of unpaired nucleotides on the two sides of the duplex can be different.

In one example, a bulge can be modelled on tracrRNA sequence strand. In other examples, bulges or the unpaired nucleotides can be on the crRNA. Other examples can include multiple bulges on one or more strands. These may occur with or without unpaired nucleotides or changes in the sequence.

A bulge on the minimum CRISPR repeat side of the duplex can comprise at least 1, 2, 3, 4, or 5 or more unpaired nucleotides. The number of bulges in the minimum crRNA sequence side of the duplex can be 1, 2, 3, 4, 5 or more.

A bulge on the minimum tracrRNA sequence side of the duplex can comprise at least 1, 2, 3, 4, 5, 6, 7, 8, 9, or 10 or more unpaired nucleotides. The number of bulges in the minimum tracrRNA sequence side of the duplex can be 1, 2, 3, 4, 5 or more.

A bulge can include wobble pairing or nucleotides not thought to bind.

The sequence of the crRNA and tracrRNA sequence can be modified to have base swaps or have additions or deletions. These changes can be introduced with and without added bulges.

D. Hairpins

In various examples, one or more hairpins can be located 3′ to the minimum tracrRNA in the 3′ tracrRNA sequence.

The hairpin can start at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. The hairpin can start at most about 1, 2, 3, 4, 5, 6, 7, 8, 9 or 10 or more nucleotides 3′ of the last paired nucleotide in the minimum CRISPR repeat and minimum tracrRNA sequence duplex.

The hairpin can comprise at least about 1, 2, 3, 4, 5, 6, 7, 8, 9, 10, 15, or 20 or more consecutive nucleotides. The hairpin can comprise at most about 1, 2, 3, 4, 5, 6, 7, 8, 9, 10, 15, or more consecutive nucleotides.

The hairpin can comprise a CC dinucleotide (i.e., two consecutive cytosine nucleotides).

The hairpin can comprise duplexed nucleotides (e.g., nucleotides in a hairpin, hybridized together). For example, a hairpin can comprise a CC dinucleotide that is hybridized to a GG dinucleotide in a hairpin duplex of the 3′ tracrRNA sequence.

One or more of the hairpins can interact with guide RNA-interacting regions of a site-directed polypeptide.

In some examples, there are two or more hairpins, and in some other examples there are three or more hairpins.

E. 3′ tracrRNA Sequence

A 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes or S. aureus).

The 3′ tracrRNA sequence can have a length from about 6 nucleotides to about 100 nucleotides. For example, the 3′ tracrRNA sequence can have a length from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The 3′ tracrRNA sequence can have a length of approximately 14 nucleotides.

The 3′ tracrRNA sequence can be at least about 60% identical to a reference 3′ tracrRNA sequence (e.g., wild type 3′ tracrRNA sequence from S. pyogenes or S. aureus) over a stretch of at least 6, 7, or 8 contiguous nucleotides. For example, the 3′ tracrRNA sequence can be at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes or S. aureus) over a stretch of at least 6, 7, or 8 contiguous nucleotides.

The 3′ tracrRNA sequence can comprise more than one duplexed region (e.g., hairpin, hybridized region). The 3′ tracrRNA sequence can comprise two duplexed regions.

The 3′ tracrRNA sequence can comprise a stem loop structure. The stem loop structure in the 3′ tracrRNA can comprise at least 1, 2, 3, 4, 5, 6, 7, 8, 9, 10, 15 or 20 or more nucleotides. The stem loop structure in the 3′ tracrRNA can comprise at most 1, 2, 3, 4, 5, 6, 7, 8, 9 or 10 or more nucleotides. The stem loop structure can comprise a functional moiety. For example, the stem loop structure can comprise an aptamer, a ribozyme, a protein-interacting hairpin, a CRISPR array, an intron, or an exon. The stem loop structure can comprise at least about 1, 2, 3, 4, or 5 or more functional moieties. The stem loop structure can comprise at most about 1, 2, 3, 4, or 5 or more functional moieties.

The hairpin in the 3′ tracrRNA sequence can comprise a P-domain. The P-domain can comprise a double-stranded region in the hairpin.

F. tracrRNA Extension Sequences

A tracrRNA extension sequence can be provided whether the tracrRNA is in the context of single-molecule guides or double-molecule guides. The tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. The tracrRNA extension sequence can have a length from about 20 to about 5000 or more nucleotides. The tracrRNA extension sequence can have a length of more than 1000 nucleotides. The tracrRNA extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence can have a length of less than 1000 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. The functional moiety can function in a eukaryotic cell. The functional moiety can function in a prokaryotic cell. The functional moiety can function in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like). The tracrRNA extension sequence can comprise a primer binding site or a molecular index (e.g., barcode sequence). The tracrRNA extension sequence can comprise one or more affinity tags.

G. Single-Molecule Guide Linker Sequences

The linker sequence of a single-molecule guide nucleic acid can have a length from about 3 nucleotides to about 100 nucleotides. In Jinek et al., supra, for example, a simple 4 nucleotide “tetraloop” (-GAAA-) was used, Science, 337(6096):816-821 (2012). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence -GAAA- was used, Science, 337(6096):816-821 (2012), but numerous other sequences, including longer sequences can likewise be used.

The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

H. Methods of Making gRNAs

The gRNAs of the present disclosure is produced by a suitable means available in the art, including but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some aspects, non-natural modified nucleobases are introduced into polynucleotides, e.g., gRNA, during synthesis or post-synthesis. In certain embodiments, modifications are on internucleoside linkages, purine or pyrimidine bases, or sugar. In particular embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some aspects, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

Certain embodiments of the invention also provide nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, the gRNAs provided by the disclosure are synthesized by enzymatic methods (e.g., in vitro transcription, IVT).

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In certain embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g.:spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR/Cas9 system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

### V. Target Sites

In some embodiments, the site-directed nucleases described herein are directed to and cleave (e.g., introduce a DSB) a target nucleic acid molecule. In some embodiments, a Cas nuclease is directed by a guide RNA to a target site of a target nucleic acid molecule (gDNA), where the guide RNA hybridizes with the complementary strand of the target sequence and the Cas nuclease cleaves the target nucleic acid at the target site. In some embodiments, the complementary strand of the target sequence is complementary to the targeting sequence (e.g.: spacer sequence) of the guide RNA. In some embodiments, the degree of complementarity between a targeting sequence of a guide RNA and its corresponding complementary strand of the target sequence is about 50%, 55%, 60%, 65%, 70%, 75%, 80%, 85%, 90%, 95%, 97%, 98%, 99%, or 100%. In some embodiments, the complementary strand of the target sequence and the targeting sequence of the guide RNA is 100% complementary. In other embodiments, the complementary strand of the target sequence and the targeting sequence of the guide RNA contains at least one mismatch. For example, the complementary strand of the target sequence and the targeting sequence of the guide RNA contain 1, 2, 3, 4, 5, 6, 7, 8, 9, or 10 mismatches. In some embodiments, the complementary strand of the target sequence and the targeting sequence of the guide RNA contain 1-6 mismatches. In some embodiments, the complementary strand of the target sequence and the targeting sequence of the guide RNA contain 5 or 6 mismatches.

The length of the target sequence may depend on the nuclease system used. For example, the target sequence for a CRISPR/Cas system comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the target sequence comprise 18-24 nucleotides in length. In some embodiments, the target sequence comprise 19-21 nucleotides in length. In some embodiments, the target sequence comprise 20 nucleotides in length.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In other embodiments, the cell is a eukaryotic cell. In some embodiments, the eukaryotic cell is a mammalian cell. In some embodiments, the eukaryotic cell may be a rodent cell. In some embodiments, the eukaryotic cell may be a human cell. In further embodiments, the target sequence may be a viral sequence. In yet other embodiments, the target sequence may be a synthesized sequence. In some embodiments, the target sequence may be on a eukaryotic chromosome, such as a human chromosome.

In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

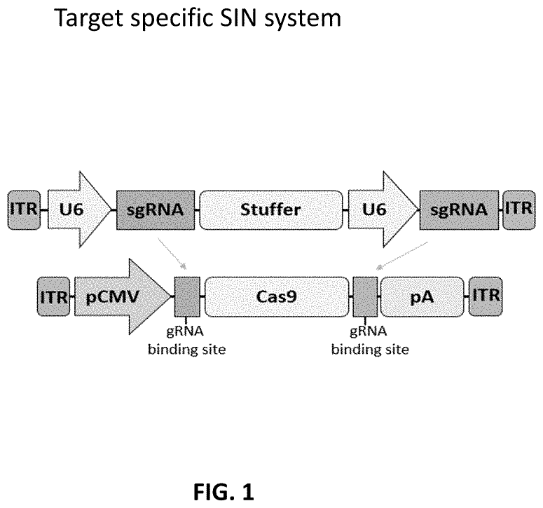

In some embodiments, the target sequence may be adjacent to a protospacer adjacent motif (PAM), a short sequence recognized by a CRISPR/Cas9 complex. In some embodiments, the PAM may be adjacent to or within 1, 2, 3, or 4, nucleotides of the 3′ end of the target sequence. The length and the sequence of the PAM may depend on the Cas9 protein used. For example, the PAM may be selected from a consensus or a particular PAM sequence for a specific Cas9 nuclease or Cas9 ortholog, including those disclosed in FIG. 1 of Ran et al., (2015) Nature, 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (St1Cas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), and NNNNGATT (NmCas9) (see e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLoS ONE 7:e40913; Esvelt et al., (2013) Nat Methods 10(11):1116-1121 (wherein N is defined as any nucleotide, W is defined as either A or T, R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

A. Modified Donor Polynucleotides

In some embodiments, donor polynucleotides are provided with chemistries suitable for delivery and stability within cells. Furthermore, in some embodiments, chemistries are provided that are useful for controlling the pharmacokinetics, biodistribution, bioavailability and/or efficacy of the donor polynucleotides described herein. Accordingly, in some embodiments donor polynucleotides described herein may be modified, e.g., comprise a modified sugar moiety, a modified internucleoside linkage, a modified nucleoside, a modified nucleotide and/or combinations thereof. In addition, the modified donor polynucleotides may exhibit one or more of the following properties: are not immune stimulatory; are nuclease resistant; have improved cell uptake compared to unmodified donor polynucleotides; and/or are not toxic to cells or mammals.

Nucleotide and nucleoside modifications have been shown to make a polynucleotide (e.g., a donor polynucleotide) into which they are incorporated more resistant to nuclease digestion than the native polynucleotide and these modified polynucleotides have been shown to survive intact for a longer time than unmodified polynucleotides. Specific examples of modified oligonucleotides include those comprising modified backbones (i.e. modified internucleoside linkage), for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see e.g., De Mesmaeker et al., Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the polynucleotide is replaced with a polyamide backbone, the nucleotides being bound directly or indirectly to the aza nitrogen atoms of the polyamide backbone, see Nielsen et al., Science 1991, 254, 1497). Phosphorus-containing modified linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321, 131; 5,399,676; 5,405,939; 5,453,496; 5,455, 233; 5,466,677; 5031272.1 5,476,925; 5,519,126; 5,536,821; 5,541,306; 5,550, 111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010).

Cyclohexenyl nucleic acid oligonucleotide mimetics are described in Wang et al., J. Am. Chem. Soc, 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5, 166,315; 5,185,444; 5,214,134; 5,216, 141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

In some embodiments, the donor polynucleotides of the disclosure are stabilized against nucleolytic degradation such as by the incorporation of a modification (e.g., a nucleotide modification). In some embodiments, donor polynucleotides of the disclosure include a phosphorothioate at least the first, second, and/or third internucleotide linkage at the 5′ and/or 3′ end of the nucleotide sequence. In some embodiments, donor polynucleotides of the disclosure include one or more 2′-modified nucleotides, e.g., 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In some embodiments, donor polynucleotides of the disclosure include a phosphorothioate and a 2′-modified nucleotide as described herein.

Any of the modified chemistries described herein can be combined with each other, and that one, two, three, four, five, or more different types of modifications can be included within the same molecule. In some embodiments, the donor polynucleotide comprises 1, 2, 3, 4, 5, 6, 7, 8, 9, 10 or modifications.

### VI. mRNA Components

In some embodiments, the systems provided by the disclosure comprise an engineered nuclease encoded by an mRNA. In some embodiments, the compositions provided by the disclosure comprise a nuclease system, wherein the nuclease comprising the nuclease system is encoded by an mRNA. In some embodiments, the mRNA may be a naturally or non-naturally occurring mRNA. In some embodiments, the mRNA may include one or more modified nucleobases, nucleosides, or nucleotides, as described below, in which case it may be referred to as a “modified mRNA”. In some embodiments, the mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. In some embodiments, an mRNA as described herein may include a 5′ cap structure, a chain terminating nucleotide, optionally a Kozak or Kozak-like sequence (also known as a Kozak consensus sequence), a stem-loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GpppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

A. Modified RNA

In some embodiments, an RNA of the disclosure (e.g.: gRNA or mRNA) comprises one or more modified nucleobases, nucleosides, nucleotides or internucleoside linkages. In some embodiments, modified mRNAs and/or gRNAs may have useful properties, including enhanced stability, intracellular retention, enhanced translation, and/or the lack of a substantial induction of the innate immune response of a cell into which the mRNA and/or gRNA is introduced, as compared to a reference unmodified mRNA and/or gRNA. Therefore, use of modified mRNAs and/or gRNAs may enhance the efficiency of protein production, intracellular retention of nucleic acids, as well as possess reduced immunogenicity.

In some embodiments, an mRNA and/or gRNA includes one or more (e.g., 1, 2, 3 or 4) different modified nucleobases, nucleosides, nucleotides or internucleoside linkages. In some embodiments, an mRNA and/or gRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified gRNA may have reduced degradation in a cell into which the gRNA is introduced, relative to a corresponding unmodified gRNA. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mchm⁵s²U), 5-aminomethyl-2-thio-uridine (nm⁵s²U), 5-methylaminomethyl-uridine (mnm⁵U), 5-methylaminomethyl-2-thio-uridine (nmm⁵s²U), 5-methylaminomethyl-2-seleno-uridine (mnm⁵se²U), 5-carbamoylmethyl-uridine (τcm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m⁵U, i.e., having the nucleobase deoxythymine), 1-methyl-pseudouridine 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ w), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-0-trimethyl-cytidine (m⁴₂ Cm), 1-thio-cytidine, 2′-F-ara-cytidine, 2′-F-cytidine, and 2′-0H-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹ A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine2-methylthio-N6-threonylcarbamoyl-(m⁶t⁶A), adenosine (ms²g⁶A), N6,N6-dimethyl-adenosine (m⁶2 A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2 Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-0H-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine N2-methyl-guanosine (m²G), N2,N2-dimethyl-guanosine (m²2 G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2 Gm), 1-methyl-2′-O-methyl-guanosine (m¹⁻Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA and/or gRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (w), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m¹ψ) and the mRNA of the disclosure is fully modified with N1-methylpseudouridine (m¹ψ). In some embodiments, N1-methylpseudouridine (m¹ψ) represents from 75-100% of the uracils in the mRNA. In some embodiments, N1-methylpseudouridine (m¹ψ) represents 100% of the uracils in the mRNA.

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m¹ψ), 5-methoxy-uridine (mo⁵U), 5-methyl-cytidine (m⁵C), pseudouridine (w), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In certain embodiments, an mRNA and/or a gRNA of the disclosure is uniformly modified (i.e., fully modified, modified through-out the entire sequence) for a particular modification. For example, an mRNA can be uniformly modified with N1-methylpseudouridine (m¹ψ) or 5-methyl-cytidine (m⁵C), meaning that all uridines or all cytosine nucleosides in the mRNA sequence are replaced with N1-methylpseudouridine (m¹ψ) or 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

### VII. Ribonucleoproteins

In certain aspects, the site-directed polypeptide (e.g.: Cas nuclease) and genome-targeting nucleic acid (e.g.: gRNA or sgRNA) may each be administered separately to a cell or a subject. In certain aspects, the site-directed polypeptide may be pre-complexed with one or more guide RNAs, or one or more sgRNAs. Such pre-complexed material is known as a ribonucleoprotein particle (RNP). In some embodiments, the nuclease system comprises a ribonucleoprotein (RNP).

The site-directed polypeptide in the RNP can be, for example, a Cas9 endonuclease or a Cpf1 endonuclease. The site-directed polypeptide can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The weight ratio of DNA-targeting nucleic acid to site-directed polypeptide in the RNP can be 1:1. For example, the weight ratio of sgRNA to Cas9 endonuclease in the RNP can be 1:1. In some embodiments, a purified Cas9 protein and a purified gRNA is pre-complexed to form an RNP. Cas9 protein can be expressed and purified by any means known in the art. Ribonucleoproteins are assembled in vitro and can be delivered directly to cells using standard electroporation or transfection techniques known in the art.

In some embodiments, the nuclease system comprises a Cas9 RNP comprising a purified Cas9 protein in complex with a gRNA. Cas9 protein can be expressed and purified by any means known in the art. Ribonucleoproteins are assembled in vitro and can be delivered directly to cells using standard electroporation or transfection techniques known in the art.

### VIII. Vectors

In some embodiments, the site-directed nuclease (e.g., Cas nuclease) and the donor polynucleotide may be provided by one or more vectors. The term “vector” refers to a nucleic acid molecule capable of transporting another nucleic acid to which it has been linked. A vector can be an expression vector. An “expression vector” is a replicon, such as plasmid, phage, virus, or cosmid, to which another DNA segment, i.e. an “insert”, can be attached so as to bring about the replication of the attached segment in a cell.

In some embodiments, the vector may be a DNA vector. In some embodiments, the vector may be circular. In other embodiments, the vector may be linear. Non-limiting exemplary vectors include plasmids, phagemids, cosmids, artificial chromosomes, minichromosomes, transposons, viral vectors, and expression vectors.

In some examples, vectors can be capable of directing the expression of nucleic acids to which they are operatively linked. Such vectors are referred to herein as “recombinant expression vectors”, or “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

In some embodiments, the vector may be a viral vector. In some embodiments, the viral vector may be genetically modified from its wild-type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (Ψ) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas9 protein, while a second AAV vector may contain one or more guide sequences and one or more copies of donor polynucleotide.

In certain embodiments, a viral vector may be modified to target a particular tissue or cell type. For example, viral surface proteins may be altered to decrease or eliminate viral protein binding to its natural cell surface receptor(s). The surface proteins may also be engineered to interact with a receptor specific to a desired cell type. Viral vectors may have altered host tropism, including limited or redirected tropism. Certain engineered viral vectors are described, for example, in WO2011130749 [HSV], WO2015009952 [HSV], U.S. Pat. No. 5,817,491 [retrovirus], WO2014135998 [T4], and WO2011125054 [T4]. In some embodiments, the vector may be capable of driving expression of one or more coding sequences in a cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding the nuclease described herein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter. In some embodiments, the nucleotide sequence encoding the nuclease may be operably linked to at least one transcriptional or translational control sequence.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1α) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1α promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

Spatially restricted promoters can also be referred to as enhancers, transcriptional control elements, control sequences, etc. Any convenient spatially restricted promoter can be used and the choice of suitable promoter (e.g., a liver-specific promoter, a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, muscle-specific promoters, liver-specific promoters, neuron-specific promoters, adipocyte-specific promoters, cardiomyocyte-specific promoters, smooth muscle-specific promoters, photoreceptor-specific promoters, etc.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyilrek et al. (2000) Mol. Med. 6:983; and U.S. Pat. No. 7,169,874); a smoothelin promoter (see, e.g., WO 2001/018048); a-smooth muscle actin promoter; a Cke8 promoter (see, e.g., WO 2018/107003 and WO 2018/1292960); the SPc5-12 promoter (see, e.g., US 2004/0175727 and WO 2009/045813), and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

In some embodiments, the nuclease encoded by the vector may be a Cas protein, such as a Cas9 protein or Cpf1 protein. The vector system may further comprise a vector comprising a nucleotide sequence encoding the guide RNA described herein. In some embodiments, the vector system may comprise one copy of the guide RNA. In other embodiments, the vector system may comprise more than one copy of the guide RNA. In embodiments with more than one guide RNA, the guide RNAs may be non-identical such that they target different target sequences, or have other different properties, such as activity or stability within the Cas9 RNP complex. In some embodiments, the nucleotide sequence encoding the guide RNA may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, H1 and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA. In other embodiments, the crRNA and the tracr RNA may be driven by their corresponding promoters on the same vector. In yet other embodiments, the crRNA and the tracr RNA may be encoded by different vectors.

In some embodiments, the nucleotide sequence encoding the guide RNA may be located on the same vector comprising the nucleotide sequence encoding a Cas9 protein. In some embodiments, expression of the guide RNA and of the Cas9 protein may be driven by different promoters. In some embodiments, expression of the guide RNA may be driven by the same promoter that drives expression of the Cas9 protein. In some embodiments, the guide RNA and the Cas9 protein transcript may be contained within a single transcript. For example, the guide RNA may be within an untranslated region (UTR) of the Cas9 protein transcript. In some embodiments, the guide RNA may be within the 5′ UTR of the Cas9 protein transcript. In other embodiments, the guide RNA may be within the 3′ UTR of the Cas9 protein transcript. In some embodiments, the intracellular half-life of the Cas9 protein transcript may be reduced by containing the guide RNA within its 3′ UTR and thereby shortening the length of its 3′ UTR. In additional embodiments, the guide RNA may be within an intron of the Cas9 protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

In some embodiments, the vector system may further comprise a vector comprising the donor polynucleotide described herein. In some embodiments, the vector system may comprise one copy of the donor polynucleotide. In other embodiments, the vector system may comprise more than one copy of the donor polynucleotide. In some embodiments, the vector system may comprise 2, 3, 4, 5, 6, 7, 8, 9, 10, or more copies of the donor polynucleotide. The multiple copies of the donor polynucleotide may be located on the same or different vectors. The multiple copies of the donor polynucleotide may also be adjacent to one another, or separated by other nucleotide sequences or vector elements.

A vector system may comprise 1-3 vectors. In some embodiments, the vector system may comprise one single vector. In other embodiments, the vector system may comprise two vectors. In additional embodiments, the vector system may comprise three vectors. When different guide RNAs or donor polynucleotides are used for multiplexing, or when multiple copies of the guide RNA or the donor polynucleotide are used, the vector system may comprise more than three vectors.

In some embodiments, the nucleotide sequence encoding a Cas9 protein, a nucleotide sequence encoding the guide RNA, and a donor polynucleotide may be located on the same or separate vectors. In some embodiments, all of the sequences may be located on the same vector. In some embodiments, two or more sequences may be located on the same vector. The sequences may be oriented in the same or different directions and in any order on the vector. In some embodiments, the nucleotide sequence encoding the Cas9 protein and the nucleotide sequence encoding the guide RNA may be located on the same vector. In some embodiments, the nucleotide sequence encoding the Cas9 protein and the donor polynucleotide may be located on the same vector. In some embodiments, the nucleotide sequence encoding the guide RNA and the donor polynucleotide may be located on the same vector. In a some embodiments, the vector system may comprise a first vector comprising the nucleotide sequence encoding the Cas9 protein, and a second vector comprising the nucleotide sequence encoding the guide RNA and the donor polynucleotide.

Methods of introducing a nucleic acid into a host cell are known in the art, and any known method can be used to introduce a nucleic acid (e.g., an expression construct) into a cell. Nucleotides encoding a guide RNA (introduced either as DNA or RNA) and/or a site-directed modifying polypeptide (introduced as DNA or RNA) and/or a donor polynucleotide can be provided to the cells using well-developed transfection techniques; see, e.g. Angel and Yanik (2010) PLoS ONE 5(7): e 11756, and the commercially available TransMessenger® reagents from Qiagen, Stemfect™ RNA Transfection Kit from Stemgent, and TranslT®-mRNA Transfection Kit from Mims Bio LLC (See, also Beumer et al. (2008) Efficient gene targeting in Drosophila by direct embryo injection with zinc-finger nucleases. PNAS 105(50):19821-19826). Alternatively, nucleic acids encoding a guide RNA and/or a site-directed modifying polypeptide and/or a chimeric site-directed modifying polypeptide and/or a donor polynucleotide can be provided on DNA vectors. Many vectors, e.g. plasmids, cosmids, minicircles, phage, viruses, etc., useful for transferring nucleic acids into target cells are available. The vectors comprising the nucleic acid(s) can be maintained episomally, e.g. as plasmids, minicircle DNAs, viruses such cytomegalovirus, adenovirus, etc., or they can be integrated into the target cell genome, through homologous recombination or random integration, e.g. retrovirus-derived vectors such as MMLV, HIV-1, ALV, etc.

Vectors can be provided directly to the cells. In other words, the cells are contacted with vectors comprising the nucleic acid encoding guide RNA and/or a site-directed modifying polypeptide and/or a chimeric site-directed modifying polypeptide and/or a donor polynucleotide such that the vectors are taken up by the cells. Methods for contacting cells with nucleic acid vectors that are plasmids, including electroporation, calcium chloride transfection, microinjection, and lipofection are well known in the art. For viral vector delivery, the cells can be contacted with viral particles comprising the nucleic acid encoding a guide RNA and/or a site-directed modifying polypeptide and/or a chimeric site-directed modifying polypeptide and/or a donor polynucleotide. Retroviruses, for example, lentiviruses, are suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid can be packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line can be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also be introduced by direct micro-injection (e.g., injection of RNA into a zebrafish embryo).

Vectors used for providing the nucleic acids encoding guide RNA and/or a site-directed modifying polypeptide and/or a chimeric site-directed modifying polypeptide and/or a donor polynucleotide to the cells can typically comprise suitable promoters for driving the expression, that is, transcriptional activation, of the nucleic acid of interest. In other words, the nucleic acid of interest will be operably linked to a promoter. This can include ubiquitously acting promoters, for example, the CMV-13-actin promoter, or inducible promoters, such as promoters that are active in particular cell populations or that respond to the presence of drugs such as tetracycline. By transcriptional activation, it can be intended that transcription will be increased above basal levels in the target cell by at least about 10 fold, by at least about 100 fold, more usually by at least about 1000 fold. In addition, vectors used for providing a guide RNA and/or a site-directed modifying polypeptide and/or a chimeric site-directed modifying polypeptide and/or a donor polynucleotide to the cells can include nucleic acid sequences that encode for selectable markers in the target cells, so as to identify cells that have taken up the guide RNA and/or a site-directed modifying polypeptide and/or a chimeric site-directed modifying polypeptide and/or a donor polynucleotide.

The nucleic acid encoding a DNA-targeting nucleic acid of the disclosure and/or a site-directed polypeptide can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### IX. Self-Targeting/Self-Inactivating CRISPR/Cas Systems

Another aspect of the disclosure is a self-targeting CRISPR/Cas or CRISPR/Cpf1 system that utilizes a non-coding targeting sequence within the CRISPR vector itself that is substantially complementary to the target gene in the vector. In some examples, the self-targeting CRISPR/Cas or CRISPR/Cpf1 system targets, but does not inactivate the system. Such self-targeting CRISPR/Cas or CRISPR/Cpf1 systems would allow for tracking of edited loci, for example.

In some embodiments, the self-targeting CRISPR/Cas or CRISPR/Cpf1 system can inactivate expression of the site-directed polypeptide (i.e., Cas9 or Cpf1). In this regard, after expression begins, the CRISPR system will lead to its own destruction, but before destruction is complete it will have time to edit one or more genomic copies of the target gene. The self-inactivating CRISPR/Cas or CRISPR/Cpf1 system can include self-inactivating (SIN) sites that target the coding sequence for the site-directed polypeptide itself, or that targets one or more non-coding sequences in the site-directed polypeptide expression vector (e.g., SIN sites).

In some embodiments, the self-targeting/self-inactivating CRISPR/Cas or CRISPR/Cpf1 system can be engineered to have altered sequences downstream of a target site to have a canonical or non-canonical PAM, such as NRG or variants thereof (e.g.: NGG, NAG or NGA). In some examples, the self-targeting/self-inactivating CRISPR/Cas or CRISPR/Cpf1 system can be engineered to have altered sequences downstream of a target site to have a canonical or non-canonical PAM, such as NNGRRN, or any variants thereof. In some examples, the self-targeting/self-inactivating CRISPR/Cas or CRISPR/Cpf1 system can be engineered to have altered sequences downstream of a target site to have a canonical or non-canonical PAM, such as NNGRRT or any variants thereof (e.g.: CTGAAT, GAGAGT, ATGAGT, CAGAGT, TTGAGT or TGGAAT).

In some embodiments, the self-inactivating CRISPR/Cas or CRISPR/Cpf1 system can be an “all-in-two” vector system. The dual vector system can allow for delivery of Homology Directed Repair (HDR) templates, site-directed polypeptide, and more than one guide RNA (gRNA). Expression of more than one gRNA allows for the introduction of double-stranded breaks in the target gene and also a mutation in the coding sequence and/or a decrease or termination of Cas9 or Cpf1 expression as well as temporal control over termination of Cas9 or Cpf1 expression.

In some embodiments, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., guide RNA); and one or more third segments (e.g., SIN site) comprising a nucleotide sequence that is substantially complementary to the second segment (e.g., gRNA).

In another aspect, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., gRNA or sgRNA); and one or more third segments comprising a nucleotide sequence that is substantially complementary to the nucleotide sequence of the DNA-targeting nucleic acid (e.g., SIN sites).

In another aspect, described herein is a self-inactivating CRISPR/Cas or CRISPR/Cpf1 system comprising a first segment comprising a nucleotide sequence that encodes a site-directed polypeptide (e.g., a CRISPR enzyme); a second segment comprising a nucleotide sequence that encodes a DNA-targeting nucleic acid (e.g., gRNA or sgRNA); and one or more third segments (e.g., SIN sites) comprising a nucleotide sequence that is substantially complementary to the nucleotide sequence of the DNA-targeting nucleic acid, wherein the sequence of the first segment comprises the sequence of the third segment. For example, the nucleotide sequence that encodes a site-directed polypeptide comprises a SIN site nucleotide sequence.

In some embodiments, the first segment comprising a nucleotide sequence that encodes a site-directed polypeptide, can further comprise a start codon, a stop codon, and a poly(A) termination site. In other examples, the first segment comprising a nucleotide sequence that encodes a site-directed polypeptide, can further comprise one or more naturally occurring or chimeric introns inserted into, upstream, and/or downstream of a Cas9 open reading frame (ORF). The chimeric intron can comprise a 5′-donor site from the first intron of the human β-globin gene and the branch and a 3″-acceptor site from the intron of an immunoglobulin gene heavy chain variable region. The chimeric intron introduced into Cas9 ORF can be used to insert one or more gRNA binding sites utilized for self-inactivation (e.g.: SIN site). Introns and/or their splicing can enhance almost every step of gene expression, from transcription to translation. For example, intron-containing transgenes in mice are transcribed up to 100-fold more efficiently than the same genes lacking introns. The enhancing effects of introns on the posttranscriptional stages of gene expression are commonly attributed to proteins recruited to the mRNA during splicing. Intron enhanced expression of Cas9 may also allow use of less AAV vector doses for in vivo gene editing. In addition, introns allow the use of PAM sites recognized by different Cas9 orthologues, as well as protospacer-like sequences recognized by different DNA-targeting nucleic acids, making SIN vector systems readily adaptable for use with Cas9 orthologues. In certain aspects, introns that can be used in the expression constructs described herein include, but are not limited to, SEQ ID NOs: 53-56. SIN sites may be inserted into these introns at various locations, which may or may not include deletion of one or more nucleotides in the intronic sequence.

In some embodiments, a nucleic acid sequence encoding a promoter can be operably linked to the first segment.

In some embodiments, the site-directed polypeptide can be Cas9, Cpf1, or any variants thereof. In other examples, the site directed polypeptide can be Streptococcus pyogenes Cas9 (SpCas9) or any variants thereof. In other examples, the site directed polypeptide can be Campylobacter jejuni Cas9 (CjCas9) or any variants thereof. In other examples, the site directed polypeptide can be Staphylococcus aureus Cas9 (SaCas9) or any variants thereof. The SaCas9 variant can comprise a D10A mutation in the amino acid sequence set forth in SEQ ID NO: 47. The Cas9 variant can comprise an N580A mutation in the amino acid sequence set forth in SEQ ID NO: 48. The SaCas9 variant can comprise both a D10A mutation and an N580A mutation in the amino acid sequence set forth in SEQ ID NO: 49. SaCas9 can comprise a nucleotide sequence as set forth in SEQ ID NO: 52, or codon optimized variants thereof.

In some embodiments, the DNA-targeting nucleic acid can be a guide RNA (gRNA) or single-molecule guide RNA (sgRNA). The gRNA or sgRNA can be synthesized inside the cells or be delivered from outside the cells as synthetic sgRNA or synthetic dual gRNAs. The gRNA or sgRNA can also be partly synthesized and partly delivered from outside of the cell.

In some embodiments, one or more third segments can comprise a SIN site. In some examples, one or more third segments can comprise a protospacer adjacent motif (PAM). In other examples, the PAM can be NNGRRN or any variants thereof (e.g.: NNGRRT, NNGRRV). In other examples, the PAM can be NNGRYT or NNGYRT, or any variants thereof (Friedland et al., 2015, Genome Biology, 16(257):1-10). In some examples, one or more third segments can comprise a DNA-target.

In some embodiments, one or more third segments can be located at any one or more of: a 5′ end of the first segment, upstream of the start codon and/or downstream of the transcriptional start site; within one or more naturally occurring or chimeric inserted introns; or a 3′ end of the first segment between the stop codon and poly(A) termination site.

In some embodiments, the third segment is not fully complementary to the second segment in at least one, two, three, four, five or more locations along the length of the third segment.

In some embodiments, the third segment is not fully complementary to the second segment. In some examples, the third segment is not fully complementary to the second segment and (1) differs in sequence at one, two, three or more bases and (2) differs in length with one or more bulges from extra bases in the guide or target DNA sequences.

In some embodiments, the third segment is not fully complementary to the nucleotide sequence of the DNA-targeting nucleic acid in at least one location. In other examples, the third segment is not fully complementary to the nucleotide sequence of the DNA-targeting nucleic acid in at least two locations. In other examples, the third segment is not fully complementary to the nucleotide sequence of the DNA-targeting nucleic acid in at least three, four, five or more locations.

In some embodiments, the third segment has a canonical protospacer adjacent motif (PAM), such as NGG, or has an alternative PAM. An example of an alternative PAM for the SpCas9 is NAG. In some examples, the third segment has a PAM proceeded by a bulge, such as NNGG (N can be any nucleotide, including wild-type).

In some embodiments, the third segment has a canonical protospacer adjacent motif (PAM) for one or more orthologue Cas9, such as NNGRRT, or has an alternative PAM, such as NNGRRN, NNGRYT, NNGYRT, NNGRRV.

In some embodiments, the third segment has a canonical protospacer adjacent motif (PAM) for one or more orthologue Cas9, such as, NNNNACA or has an alternative PAM, such as NNNACAC, NNVRYAC, or NNNVRYM.

In some embodiments, the site-directed polypeptide can be S. pyogenes (Sp) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some embodiments, the site-directed polypeptide can be SpCas9 and the DNA-targeting nucleic acid can be a gRNA or sgRNA that targets the one or more third segments, wherein the one or more third segments is located within one or more naturally occurring or chimeric inserted introns.

In some embodiments, the site-directed polypeptide can be SpCas9 and the DNA-targeting nucleic acid can be a gRNA or sgRNA that targets the one or more third segments, wherein the one or more third segments is located at the 3′ end of the first segment between the stop codon and poly(A) termination site.

In some embodiments, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some embodiments, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some embodiments, the site-directed polypeptide can be SpCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some embodiments, the site-directed polypeptide can be SpCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some embodiments, the site-directed polypeptide can be C. jejuni (Cj) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some embodiments, the site-directed polypeptide can be CjCas9 and the DNA-targeting nucleic acid can be a gRNA or sgRNA that targets the one or more third segments, wherein the one or more third segments is located within one or more naturally occurring or chimeric inserted introns.

In some embodiments, the site-directed polypeptide can be CjCas9 and the DNA-targeting nucleic acid can be a gRNA or sgRNA that targets the one or more third segments, wherein the one or more third segments is located at the 3′ end of the first segment between the stop codon and poly(A) termination site.

In some embodiments, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some embodiments, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some embodiments, the site-directed polypeptide can be CjCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some embodiments, the site-directed polypeptide can be CjCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some embodiments, the site-directed polypeptide can be S. aureus (Sa) Cas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site.

In some embodiments, the site-directed polypeptide can be SaCas9 and the DNA-targeting nucleic acid can be a gRNA or sgRNA that targets the one or more third segments, wherein the one or more third segments is located within one or more naturally occurring or chimeric inserted introns.

In some embodiments, the site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site.

In some embodiments, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and at the 3′ end of the first segment between the stop codon and poly(A) termination site.

In some embodiments, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; and within one or more naturally occurring or chimeric inserted introns.

In some embodiments, the site-directed polypeptide can be SaCas9 and the DNA-targeting nucleic acid can be a gRNA or sgRNA that targets the one or more third segments, wherein the one or more third segments is located at the 3′ end of the first segment between the stop codon and poly(A) termination site; and within one or more naturally occurring or chimeric inserted introns.

In some embodiments, the site-directed polypeptide can be SaCas9 and the DNA-targeting nucleic acid can be a gRNA or sgRNA that targets the one or more third segments, wherein the one or more third segments is located at the 5′ end of the first segment, upstream of the start codon and/or downstream of the transcriptional start site; at the 3′ end of the first segment between the stop codon and poly(A) termination site; and within one or more naturally occurring or chimeric inserted introns.

In some embodiments, the third segment of the self-inactivating CRISPR/Cas or CRISPR/Cpf1 system comprises a nucleotide sequence that is less than 100 nucleotides in length (e.g., less than 75, less than 50, less than 25 nucleotides in length; or ranging from about 20-50, 20-75, 25-100, 75-100, or 50-75 nucleotides in length). In some examples, the third segment comprises a nucleotide sequence that is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length.

The first embodiments, the second segment, and the third segment of the self-inactivating CRISPR/Cas or CRISPR/Cpf1 system, can be delivered via one or more vectors. For example, the first segment, the second segment, and the third segment of the self-inactivating CRISPR/Cas or CRISPR/Cpf1 system can be delivered via the same vector. In another example, the first segment and the third segment can be provided together in a first vector and the second segment can be provided in a second vector. The third segment can be present in the vector at a location 5′ of the first segment. The third segment can be present in the vector at a location 3′ of the first segment. The one or more third segments can be present in the vector at the 5′ and 3′ ends of the first segment. The one or more third segments can be present in the vector within the first segment, for example, within introns of the first segment.

The vector can be one or more adeno-associated virus (AAV) vectors. The adeno-associated virus (AAV) vector can be AAV2. The adeno-associated virus (AAV) vector can be AAV1-AAV9, or any variants thereof.

When provided by a separate vector, the second segment can be administered sequentially or simultaneously with the vector encoding the first segment and the third segment. When administered sequentially, the vector encoding the second segment is delivered after the vector encoding the first segment and the third segment to allow for the intended gene editing or gene engineering to occur. This period can be a period of minutes (e.g. 5 minutes, 10 minutes, 20 minutes, 30 minutes, 45 minutes, 60 minutes), hours (e.g. 2 hours, 4 hours, 6 hours, 8 hours, 12 hours, 24 hours), days (e.g. 2 days, 3 days, 4 days, 7 days), weeks (e.g. 2 weeks, 3 weeks, 4 weeks), months (e.g. 2 months, 4 months, 8 months, 12 months) or years (2 years, 3 years, 4 years). In this regard, the site-directed polypeptide can associate with a first gRNA/sgRNA capable of hybridizing to a target gene sequence, such as a genomic locus or loci of interest and undertakes the function(s) desired of the CRISPR/Cas or CRISPR/Cpf1 system (e.g., gene engineering); and subsequently the site-directed polypeptide can then associate with the third segment capable of hybridizing to the sequence comprising a nucleotide sequence that encodes at least part of the site-directed polypeptide or guide RNA targeting the target DNA. Where the third segment targets the nucleotide sequence encoding expression of the site-directed polypeptide, the enzyme becomes impeded and the system becomes self-inactivating. In various example, CRISPR RNA that targets site-directed polypeptide expression applied via, for example liposome, lipofection, nanoparticles, microvesicles as explained herein, can be administered sequentially or simultaneously.

In some aspects, a third segment comprising a SIN site can be provided that is located downstream of a site-directed polypeptide start codon. A gRNA is capable of hybridizing to the SIN site whereby after a period of time there is a mutation in the coding sequence of the site-directed polypeptide and/or loss of the site-directed polypeptide expression. In some aspects, one or more SIN site(s) are provided that are located 5′ and 3′ of site-directed polypeptide ORF. A gRNA is capable of hybridizing to the one or more SIN sites, whereby after a period of time there is an inactivation of the site-directed polypeptide.

### X. Delivery

The delivery systems can be viral vectors, lipid nonaparticles (LNPs) or synthetic polymers. Timing of delivery of AAV vectors and LNPs can be varied (delivered at the same time or sequentially) to further achive spatiotemporal control of Cas9 expression and the self-inactivation.

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

Polynucleotides, such as guide RNA, sgRNA, and mRNA or DNA encoding an endonuclease, can be delivered to a cell or a patient by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs can also be comprised of hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

The lipids can be combined in any number of molar ratios to produce a LNP. In addition, the polynucleotide(s) can be combined with lipid(s) in a wide range of molar ratios to produce a LNP.

As stated previously, the site-directed polypeptide and DNA-targeting nucleic acid can each be administered separately to a cell or a patient. On the other hand, the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

RNA is capable of forming specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

The endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio. Alternatively, the endonuclease, crRNA and tracrRNA can be generally combined in a 1:1:1 molar ratio. However, a wide range of molar ratios can be used to produce a RNP.

A recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692. See Table 1

TABLE 1

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

A method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. See Table 2.

TABLE 2

| Tissue/Cell Type | Serotype |
|---|---|
| Liver | AAV3, AAV5, AAV8, AAV9 |
| Skeletal muscle | AAV1, AAV7, AAV6, AAV8, AAV9 |
| Central nervous system | AAV5, AAV1, AAV4, AAV8, AAV9 |
| RPE | AAV5, AAV4, AAV2, AAV8, AAV9, |
| | AAVrh8R |
| Photoreceptor cells | AAV5 , AAV8, AAV9, AAVrh8R |
| Lung | AAV9, AAV5 |
| Heart | AAV8 |
| Pancreas | AAV8 |
| Kidney | AAV2, AAV8 |

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

In some cases, Cas9 mRNA, sgRNA targeting one or two loci in target genes, and donor DNA are each separately formulated into lipid nanoparticles, or are all co-formulated into one lipid nanoparticle.

In some examples, Cas9 mRNA is formulated in a lipid nanoparticle, while sgRNA and donor DNA are delivered in an AAV vector.

Options are available to deliver the Cas9 nuclease as a DNA plasmid, as mRNA or as a protein. The guide RNA can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nano-particles can be used to deliver the protein and guide RNA, while AAV can be used to deliver a donor DNA.

### XI. Genetically Modified Cells

The term “genetically modified cell” refers to a cell that comprises at least one genetic modification introduced by genome editing (e.g., using the CRISPR/Cas9/Cpf1 system). A genetically modified cell comprising an exogenous DNA-targeting nucleic acid and/or an exogenous nucleic acid encoding a DNA-targeting nucleic acid is contemplated herein.

In some examples, a genetically modified cell can comprise any of the self-inactivating CRISPR/Cas or CRISPR/Cpf1 systems disclosed herein.

In some examples, the cell can be selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

The term “isolated cell” refers to a cell that has been removed from an organism in which it was originally found, or a descendant of such a cell. Optionally, the cell can be cultured in vitro, e.g., under defined conditions or in the presence of other cells. Optionally, the cell can be later introduced into a second organism or re-introduced into the organism from which it (or the cell from which it is descended) was isolated.

The term “isolated population” with respect to an isolated population of cells refers to a population of cells that has been removed and separated from a mixed or heterogeneous population of cells. In some cases, the isolated population can be a substantially pure population of cells, as compared to the heterogeneous population from which the cells were isolated or enriched. In some cases, the isolated population can be an isolated population of human progenitor cells, e.g., a substantially pure population of human progenitor cells, as compared to a heterogeneous population of cells comprising human progenitor cells and cells from which the human progenitor cells were derived.

In some of the above applications, the methods can be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual). Because the guide RNA provide specificity by hybridizing to target DNA, a mitotic and/or post-mitotic cell of interest in the disclosed methods can include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.). Suitable host cells include naturally-occurring cells; genetically modified cells (e.g., cells genetically modified in a laboratory, e.g., by the “hand of man”); and cells manipulated in vitro in any way. In some cases, a host cell can be isolated.

Any type of cell can be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells can be from established cell lines or they can be primary cells, where “primary cells”, “primary cell lines”, and “primary cultures” are used interchangeably herein to refer to cells and cells cultures that have been derived from a and allowed to grow in vitro for a limited number of passages, i.e. splittings, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. Target cells can be in many examples unicellular organisms, or can be grown in culture.

If the cells are primary cells, such cells can be harvested from an individual by any convenient method. For example, leukocytes can be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution can be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

Following the methods described above, a DNA region of interest can be cleaved and modified, i.e. “genetically modified”, ex vivo. In some examples, as when a selectable marker has been inserted into the DNA region of interest, the population of cells can be enriched for those comprising the genetic modification by separating the genetically modified cells from the remaining population. Prior to enriching, the “genetically modified” cells can make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells can be achieved by any convenient separation technique appropriate for the selectable marker used. For example, if a fluorescent marker has been inserted, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells can be selected against dead cells by employing dyes associated with dead cells (e.g. propidium iodide). Any technique can be employed which is not unduly detrimental to the viability of the genetically modified cells. Cell compositions that are highly enriched for cells comprising modified DNA can be achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition can be a substantially pure composition of genetically modified cells.

Genetically modified cells produced by the methods described herein can be used immediately. Alternatively, the cells can be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells can be cultured in vitro under various culture conditions. The cells can be expanded in culture, i.e. grown under conditions that promote their proliferation. Culture medium can be liquid or semi-solid, e.g. containing agar, methylcellulose, etc. The cell population can be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture can contain growth factors to which the regulatory T cells are responsive. Growth factors, as defined herein, can be molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way can be transplanted to a subject for purposes such as gene therapy, e.g. to treat a disease or as an antiviral, antipathogenic, or anticancer therapeutic, for the production of genetically modified organisms in agriculture, or for biological research. The subject can be a neonate, a juvenile, or an adult. Of particular interest are mammalian subjects. Mammalian species that can be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) can be used for experimental investigations.

Cells can be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells can be introduced to the subject via any of the following routes: parenteral, subcutaneous, intravenous, intracranial, intraspinal, intraocular, or into spinal fluid. The cells can be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint; by continuous infusion, e.g. by cannulation, e.g. with convection (see e.g. US Application No. 20070254842, incorporated herein by reference); or by implanting a device upon which the cells have been reversably affixed (see e.g. US Application Nos. 20080081064 and 20090196903, incorporated herein by reference). Cells can also be introduced into an embryo (e.g., a blastocyst) for the purpose of generating a transgenic animal (e.g., a transgenic mouse).

The number of administrations of treatment to a subject can vary. Introducing the genetically modified cells into the subject can be a one-time event; but in certain situations, such treatment can elicit improvement for a limited period of time and require an on-going series of repeated treatments. In other situations, multiple administrations of the genetically modified cells can be required before an effect is observed. The exact protocols depend upon the disease or condition, the stage of the disease and parameters of the individual subject being treated.

### XII. Pharmaceutical Compositions

The present disclosure includes pharmaceutical compositions comprising a donor polynucleotide, a gRNA, and a Cas9 protein, in combination with one or more pharmaceutically acceptable excipient, carrier or diluent.

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, and range from a pH of about 3 to a pH of about 11, about pH 3 to about pH 7, depending on the formulation and route of administration. In alternative examples, the pH can be adjusted to a range from about pH 5.0 to about pH 8. In some examples, the compositions comprise a therapeutically effective amount of at least one compound as described herein, together with one or more pharmaceutically acceptable excipients.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Pharmaceutical compositions can be formulated into preparations in solid, semi¬solid, liquid or gaseous forms, such as tablets, capsules, powders, granules, ointments, solutions, suppositories, injections, inhalants, gels, microspheres, and aerosols. As such, administration of a guide RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent can be systemic after administration or can be localized using regional administration, intramural administration, or use of an implant that acts to retain the active dose at the site of implantation. The active agent can be formulated for immediate activity or it can be formulated for sustained release.

In some cases, the components of the composition are individually pure, e.g., each of the components is at least about 75%, at least about 80%, at least about 90%, at least about 95%, at least about 98%, at least about 99%, or at least 99%, pure. In some cases, the individual components of a composition are pure before being added to the composition.

In some embodiments, the donor polynucleotideis encapsulated in a nanoparticle, e.g., a lipid nanoparticle. In some embodiments, the gRNA is encapsulated in a nanoparticle. In some embodiments, a Cas nuclease (e.g. SpCas9) is encapsulated in a nanoparticle. In particular embodiments, an mRNA encoding a Cas nuclease or nanoparticle encapsulating a Cas nuclease is present in a pharmaceutical composition. In various embodiments, the one or more mRNA present in the pharmaceutical composition is encapsulated in a nanoparticle, e.g., a lipid nanoparticle. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ratio of the first mRNA to the second mRNA is greater than

In some embodiments, the ratio between the lipid composition and the donor polynucleotide can be about 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide is about 20:1 or about 15:1.

In one embodiment, the lipid nanoparticles described herein can comprise polynucleotides (e.g., donor polynucleotide) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

Typically, an effective amount of a self-inactivating CRISPR/Cas or CRISPR/Cpf1 system comprising a guide RNA and/or site-directed modifying polypeptide and/or donor polynucleotide can be provided. The amount of recombination can be measured by any convenient method, e.g. as described above and known in the art. The calculation of the effective amount or effective dose of a self-inactivating CRISPR/Cas or CRISPR/Cpf1 system comprising a guide RNA and/or site-directed modifying polypeptide and/or donor polynucleotide to be administered is within the skill of one of ordinary skill in the art, and can be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a self-inactivating CRISPR/Cas or CRISPR/Cpf1 system comprising a guide RNA and/or site-directed modifying polypeptide and/or donor polynucleotide can be obtained from a suitable commercial source. As a general proposition, the total pharmaceutically effective amount of a guide RNA and/or site-directed modifying polypeptide and/or donor polynucleotide administered parenterally per dose will be in a range that can be measured by a dose response curve.

Therapies based on a self-inactivating CRISPR/Cas or CRISPR/Cpf1 system comprising a guide RNA and/or site-directed modifying polypeptide and/or donor polynucleotides, i.e. preparations of a guide RNA and/or site-directed modifying polypeptide and/or donor polynucleotide to be used for therapeutic administration, must be sterile. Sterility is readily accomplished by filtration through sterile filtration membranes (e.g., 0.2 μm membranes). Therapeutic compositions can be generally placed into a container having a sterile access port, for example, an intravenous solution bag or vial having a stopper pierceable by a hypodermic injection needle. The therapies based on a self-inactivating CRISPR/Cas or CRISPR/Cpf1 system comprising a guide RNA and/or site-directed modifying polypeptide and/or donor polynucleotide can be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution of compound, and the resulting mixture is lyophilized. The infusion solution can be prepared by reconstituting the lyophilized compound using bacteriostatic Water-for-Injection.

### XIII. Kits

The present disclosure provides kits for carrying out the methods described herein. A kit can include one or more of a DNA-targeting nucleic acid, a polynucleotide encoding a DNA-targeting nucleic acid, a site-directed polypeptide, a polynucleotide encoding a site-directed polypeptide, and/or any nucleic acid or proteinaceous molecule necessary to carry out the aspects of the methods described herein, or any combination thereof. Components of a kit can be in separate containers, or combined in a single container.

Any kit described above can further comprise one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

### XIV. Methods of Editing the Dystrophin Gene

Provided herein are cellular, ex vivo and in vivo methods for using the Crispr/Cas systems and vectors provided herein to create permanent changes to the genome that can restore the dystrophin reading frame and restore dystrophin protein activity. Such methods use endonucleases, such as Crispr/Cas nucleases, to permanently delete (excise), insert, or replace (delete and insert) exons (i.e., exon 51) in the genomic locus of the dystrophin gene. Use of the CRISPR/cas systems and vectors provided herein restores the reading frame with as few as a single treatment (rather than delivering exon skipping oligos for the lifetime of the patient).

Provided herein are methods for treating a patient with DMD using the Crispr/Cas systems and vectors provided herein. An example of such method is an ex vivo cell based therapy. For example, a DMD patient specific iPS cell line is created. Then, the chromosomal DNA of these iPS cells is corrected using the materials and methods described herein. Next, the corrected iPSCs are differentiated into Pax7+ muscle progenitor cells. Finally, the progenitor cells are implanted into the patient. There are many advantages to this ex vivo approach.

One advantage of an ex vivo cell therapy approach is the ability to conduct a comprehensive analysis of the therapeutic prior to administration. All nuclease based therapeutics have some level of off-target effects. Performing gene correction ex vivo allows one to fully characterize the corrected cell population prior to implantation.

In some embodiments, the methods provided herein include sequencing the entire genome of the corrected cells to ensure that the off-target cuts, if any, are in genomic locations associated with minimal risk to the patient. Furthermore, clonal populations of cells can be isolated prior to implantation.

Another advantage of ex vivo cell therapy relates to genetic correction in iPSCs compared to other primary cell sources. iPSCs are prolific, making it easy to obtain the large number of cells that will be required for a cell based therapy.

Furthermore, iPSCs are an ideal cell type for performing clonal isolations. This allows screening for the correct genomic correction, without risking a decrease in viability. In contrast, other potential cell types, such as primary myoblasts, are viable for only a few passages and difficult to clonally expand. Also, patient specific DMD myoblasts will be unhealthy due to the lack of dystrophin protein. On the other hand, patient derived DMD iPSCs will not display a diseased phenotype, as they do not express dystrophin in this differentiation state. Therefore, manipulation of DMD iPSCs will be much easier, and will shorten the amount of time needed to make the desired genetic correction.

A further advantage of ex vivo cell therapy relates to the implantation of myogenic Pax7+ progenitors versus myoblasts. Pax7+ cells are accepted as myogenic satellite cells. Pax7+ progenitors are mono-nuclear cells that sit on the periphery of the multi-nucleated muscle fibers. In response to injury, the progenitors divide and fuse to the existing fibers. In contrast, myoblasts fuse directly to the muscle fiber upon implantation and have minimal proliferative capacity in vivo. Therefore, myoblasts cannot aid in healing following repeated injury, while Pax7+ progenitors can function as a reservoir and help heal the muscle for the lifetime of the patient.

In other embodiments, the Crispr/Cas systems and vectors provided herein can be used in method which is an in vivo based therapy. In this method, the chromosomal DNA of the cells in the patient is corrected using the materials and methods described herein.

The advantage of in vivo gene therapy is the ease of therapeutic production and administration. The same therapeutic cocktail will have the potential to reach a subset of the DMD patient population (n>1). In contrast, the ex vivo cell therapy proposed requires a custom therapeutic to be developed for each patient (n=1). Ex vivo cell therapy development requires time, which certain advanced DMD patients may not have.

Also provided herein is a cellular method for editing the dystrophin gene in a human cell by administering the Crispr/Cas systems and vectors provided herein. For example, a cell is isolated from a patient or animal. Then, the chromosomal DNA of the cell is corrected using the materials and methods described herein.

A. Human Cells

For ameliorating DMD, as described and illustrated herein, the principal targets for gene editing are human cells. For example, in the ex vivo methods, the human cells can be somatic cells, which after being modified using the techniques as described, can give rise to Pax7+ muscle progenitor cells. For example, in the in vivo methods, the human cells can be muscle cells or muscle precursor cells.

By performing gene editing in autologous cells that are derived from and therefore already completely matched with the patient in need, it is possible to generate cells that can be safely re-introduced into the patient, and effectively give rise to a population of cells that can be effective in ameliorating one or more clinical conditions associated with the patient's disease.

Progenitor cells (also referred to as stem cells herein) are capable of both proliferation and giving rise to more progenitor cells, these in turn having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or may be induced artificially upon treatment with various factors. In many biological instances, stem cells can be also “multipotent” because they can produce progeny of more than one distinct cell type, but this is not required for “stem-ness.”

Self-renewal can be another important aspect of the stem cell. In theory, self-renewal can occur by either of two major mechanisms. Stem cells can divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated,” or “differentiating” is a relative term. A “differentiated cell” is a cell that has progressed further down the developmental pathway than the cell to which it is being compared. Thus, stem cells can differentiate into lineage-restricted precursor cells (such as a myocyte progenitor cell), which in turn can differentiate into other types of precursor cells further down the pathway (such as a myocyte precursor), and then to an end-stage differentiated cell, such as a myocyte, which plays a characteristic role in a certain tissue type, and may or may not retain the capacity to proliferate further.

B. Induced Pluripotent Stem Cells

In some examples, the genetically engineered human cells described herein can be induced pluripotent stem cells (iPSCs). An advantage of using iPSCs is that the cells can be derived from the same subject to which the progenitor cells are to be administered. That is, a somatic cell can be obtained from a subject, reprogrammed to an induced pluripotent stem cell, and then re-differentiated into a progenitor cell to be administered to the subject (e.g., autologous cells). Because the progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Although differentiation is generally irreversible under physiological contexts, several methods have been recently developed to reprogram somatic cells to iPSCs. Exemplary methods are known to those of skill in the art and are described briefly herein below.

The term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Reprogramming encompasses complete reversion of the differentiation state of a differentiated cell (e.g., a somatic cell) to a pluripotent state or a multipotent state. Reprogramming can encompass complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain examples described herein, reprogramming of a differentiated cell (e.g., a somatic cell) can cause the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a myogenic stem cell). Reprogramming is also distinct from promoting the self-renewal or proliferation of cells that are already pluripotent or multipotent, although the compositions and methods described herein can also be of use for such purposes, in some examples.

Many methods are known in the art that can be used to generate pluripotent stem cells from somatic cells. Any such method that reprograms a somatic cell to the pluripotent phenotype would be appropriate for use in the methods described herein.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, Cell 126(4): 663-76 (2006). iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission [see, e.g., Maherali and Hochedlinger, Cell Stem Cell. 3(6):595-605 (2008)], and tetraploid complementation.

Human iPSCs can be obtained using similar transduction methods, and the transcription factor trio, OCT4, SOX2, and NANOG, has been established as the core set of transcription factors that govern pluripotency; see, e.g., Budniatzky and Gepstein, Stem Cells Transl Med. 3(4):448-57 (2014); Barrett et al., Stem Cells Trans Med 3: 1-6 sctm.2014-0121 (2014); Focosi et al., Blood Cancer Journal 4: e21 1 (2014); and references cited therein. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 7(5):618-30 (2010). Reprogramming can be achieved by introducing a combination of nucleic acids encoding stem cell-associated genes, including, for example, Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not effected by a method that alters the genome. Thus, in such examples, reprogramming can be achieved, e.g., without the use of viral or plasm id vectors.

The efficiency of reprogramming (i.e., the number of reprogrammed cells) derived from a population of starting cells can be enhanced by the addition of various agents, e.g., small molecules, as shown by Shi et al., Cell-Stem Cell 2:525-528 (2008); Huangfu et al., Nature Biotechnology 26(7):795-797 (2008) and Marson et al., Cell-Stem Cell 3: 132-135 (2008). Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., C1-994 (e.g., N-acetyl dinaline) and MS-27-275), MGCD0103, NVP-LAQ-824, CBHA (m-carboxycinnaminic acid bishydroxamic acid), JNJ16241 199, Tubacin, A-161906, proxamide, oxamflatin, 3-CI-UCHA (e.g., 6-(3-chlorophenylureido)caproic hydroxamic acid), AOE (2-amino-8-oxo-9, 10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rexl, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve, not only RT-PCR, but can also include detection of protein markers. Intracellular markers can be best identified via RT-PCR, or protein detection methods such as immunocytochemistry, while cell surface markers are readily identified, e.g., by immunocytochemistry.

The pluripotent stem cell character of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

C. DMD Patient Specific iPSCs

One step of the ex vivo methods of the present disclosure can involve creating a DMD patient specific iPS cell, DMD patient specific iPS cells, or a DMD patient specific iPS cell line. There are many established methods in the art for creating patient specific iPS cells, as described in Takahashi and Yamanaka 2006; Takahashi, Tanabe et al. 2007. In addition, differentiation of pluripotent cells toward the muscle lineage can be accomplished by technology developed by Anagenesis Biotechnologies, as described in International patent application publication numbers WO2013/030243 and WO2012/101 1 14. For example, the creating step can comprise: a) isolating a somatic cell, such as a skin cell or fibroblast from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX2, KLF4, Lin28, NANOG, and cMYC.

A step of the ex vivo methods of the present disclosure involves editing/correcting the DMD patient specific iPS cells using genome engineering. Likewise, a step of the in vivo methods of the present disclosure involves editing/correcting the muscle cells in a DMD patient using genome engineering. Similarly, a step in the cellular methods of the present disclosure involves editing/correcting the dystrophin gene in a human cell by genome engineering.

The methods provide gRNA pairs that delete exon 51 by cutting the gene twice, one gRNA cutting at the 5′ end of exon 51 and the other gRNA cutting at the 3′ end of exon 51.

Alternatively, the methods provide one gRNA or a pair of gRNAs that can be used to facilitate incorporation of a new sequence from a polynucleotide donor template to insert or replace a sequence in exon 51.

Alternatively, some methods provide one gRNA from the preceding paragraph to make one double-strand cut that facilitates insertion of a new sequence from a polynucleotide donor template to replace a sequence in exon 51.

D. Differentiation of Corrected iPSCs into Pax7+ Muscle Progenitor Cells

Another step of the ex vivo methods of the present disclosure involves differentiating the corrected iPSCs into Pax7+ muscle progenitor cells. The differentiating step can be performed according to any method known in the art. For example, the differentiating step can comprise contacting the genome-edited iPSC with specific media formulations, including small molecule drugs, to differentiate it into a Pax7+ muscle progenitor cell, as shown in Chal, Oginuma et al. 2015. Alternatively, iPSCs, myogenic progenitors, and cells of other lineages can be differentiated into muscle using any one of a number of established methods that involve transgene over expression, serum withdrawal, and/or small molecule drugs, as shown in the methods of Tapscott, Davis et al. 1988, Langen, Schols et al. 2003, Fujita, Endo et al. 2010, Xu, Tabebordbar et al. 2013, Shoji, Woltj en et al. 2015.

E. Implanting Pax7+ Muscle Progenitor Cells into Patients

Another step of the ex vivo methods of the invention involves implanting the Pax7+ muscle progenitor cells into patients. This implanting step can be accomplished using any method of implantation known in the art. For example, the genetically modified cells can be injected directly in the patient's muscle.

F. Administration & Efficacy

The terms “administering,” “introducing” and “transplanting” are used interchangeably in the context of the placement of cells, e.g., progenitor cells, into a subject, by a method or route that results in at least partial localization of the introduced cells at a desired site, such as a site of injury or repair, such that a desired effect(s) is produced. The cells e.g., progenitor cells, or their differentiated progeny, can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment. For example, in some aspects described herein, an effective amount of myogenic progenitor cells is administered via a systemic route of administration, such as an intraperitoneal or intravenous route.

The terms “individual”, “subject,” “host” and “patient” are used interchangeably herein and refer to any subject for whom diagnosis, treatment or therapy is desired. In some aspects, the subject is a mammal. In some aspects, the subject is a human being.

When provided prophylactically, progenitor cells described herein can be administered to a subject in advance of any symptom of DMD, e.g., prior to the development of muscle wasting. Accordingly, the prophylactic administration of a muscle progenitor cell population can serve to prevent DMD.

When provided therapeutically, muscle progenitor cells can be provided at (or after) the onset of a symptom or indication of DMD, e.g., upon the onset of muscle wasting.

The muscle progenitor cell population being administered according to the methods described herein can comprise allogeneic muscle progenitor cells obtained from one or more donors. “Allogeneic” refers to a muscle progenitor cell or biological samples comprising muscle progenitor cells obtained from one or more different donors of the same species, where the genes at one or more loci are not identical. For example, a muscle progenitor cell population being administered to a subject can be derived from one more unrelated donor subjects, or from one or more non-identical siblings. In some cases, syngeneic muscle progenitor cell populations can be used, such as those obtained from genetically identical animals, or from identical twins. The muscle progenitor cells can be autologous cells; that is, the muscle progenitor cells are obtained or isolated from a subject and administered to the same subject, i.e., the donor and recipient are the same.

The term “effective amount” refers to the amount of a population of progenitor cells or their progeny needed to prevent or alleviate at least one or more signs or symptoms of DMD, and relates to a sufficient amount of a composition to provide the desired effect, e.g., to treat a subject having DMD. The term “therapeutically effective amount” therefore refers to an amount of progenitor cells or a composition comprising progenitor cells that is sufficient to promote a particular effect when administered to a typical subject, such as one who has or is at risk for DMD. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

For use in the various aspects described herein, an effective amount of progenitor cells comprises at least 10² progenitor cells, at least 5×10² progenitor cells, at least 10³ progenitor cells, at least 5×10³ progenitor cells, at least 10⁴ progenitor cells, at least 5×10⁴ progenitor cells, at least 10⁵ progenitor cells, at least 2×10⁵ progenitor cells, at least 3×10⁵ progenitor cells, at least 4×10⁵ progenitor cells, at least 5×10⁵ progenitor cells, at least 6×10⁵ progenitor cells, at least 7×10⁵ progenitor cells, at least 8×10⁵ progenitor cells, at least 9×10⁵ progenitor cells, at least 1×10⁶ progenitor cells, at least 2×10⁶ progenitor cells, at least 3×10⁶ progenitor cells, at least 4×10⁶ progenitor cells, at least 5×10⁶ progenitor cells, at least 6×10⁶ progenitor cells, at least 7×10⁶ progenitor cells, at least 8×10⁶ progenitor cells, at least 9×10⁶ progenitor cells, or multiples thereof. The progenitor cells can be derived from one or more donors, or can be obtained from an autologous source. In some examples described herein, the progenitor cells can be expanded in culture prior to administration to a subject in need thereof.

Modest and incremental increases in the levels of functional dystrophin expressed in cells of patients having DMD can be beneficial for ameliorating one or more symptoms of the disease, for increasing long-term survival, and/or for reducing side effects associated with other treatments. Upon administration of such cells to human patients, the presence of muscle progenitors that are producing increased levels of functional dystrophin is beneficial. In some cases, effective treatment of a subject gives rise to at least about 3%, 5%, or 7% functional dystrophin relative to total dystrophin in the treated subject. In some examples, functional dystrophin will be at least about 10% of total dystrophin. In some examples, functional dystrophin will be at least about 20% to 30% of total dystrophin. Similarly, the introduction of even relatively limited subpopulations of cells having significantly elevated levels of functional dystrophin can be beneficial in various patients because in some situations normalized cells will have a selective advantage relative to diseased cells. However, even modest levels of muscle progenitors with elevated levels of functional dystrophin can be beneficial for ameliorating one or more aspects of DMD in patients. In some examples, about 10%, about 20%, about 30%, about 40%, about 50%, about 60%, about 70%, about 80%, about 90% or more of the muscle progenitors in patients to whom such cells are administered are producing increased levels of functional dystrophin.

“Administered” refers to the delivery of a progenitor cell composition into a subject by a method or route that results in at least partial localization of the cell composition at a desired site. A cell composition can be administered by any appropriate route that results in effective treatment in the subject, i.e. administration results in delivery to a desired location in the subject where at least a portion of the composition delivered, i.e. at least 1×104 cells are delivered to the desired site for a period of time. Modes of administration include injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. For the delivery of cells, administration by injection or infusion can be made.

The cells are administered systemically. The phrases “systemic administration,” “administered systemically”, “peripheral administration” and “administered peripherally” refer to the administration of a population of progenitor cells other than directly into a target site, tissue, or organ, such that it enters, instead, the subject's circulatory system and, thus, is subject to metabolism and other like processes.

The efficacy of a treatment comprising a composition for the treatment of DMD can be determined by the skilled clinician. However, a treatment is considered “effective treatment,” if any one or all of the signs or symptoms of, as but one example, levels of functional dystrophin are altered in a beneficial manner (e.g., increased by at least 10%), or other clinically accepted symptoms or markers of disease are improved or ameliorated. Efficacy can also be measured by failure of an individual to worsen as assessed by hospitalization or need for medical interventions {e.g., reduced muscle wasting, or progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

The treatment according to the present disclosure can ameliorate one or more symptoms associated with DMD by increasing the amount of functional dystrophin in the individual. Early signs typically associated with DMD, include for example, delayed walking, enlarged calf muscle (due to scar tissue), and falling frequently. As the disease progresses, children become wheel chair bound due to muscle wasting and pain. The disease becomes life threatening due to heart and/or respiratory complications.

### EXAMPLES

The invention will be more fully understood by reference to the following examples, which provide illustrative non-limiting aspects of the invention.

### Example 1: All-in-Two Mouse Target Specific SIN Vector System for Excision of DMD Gene

AAV vector plasmid constructs used in this Example were built using standard cloning procedures and Gibson High-Fidelity assembly reactions based on manufacture's recommendations (New England Biolabs, Ipswich, Mass.). In this example, pairs of gRNAs were selected to flank the exon 51 acceptor site of the DMD gene. Seven SaCas9-SIN constructs were screened in plasmid format (FIG. 1). To examine the functionality of SIN sites in cleaving the SaCas9 constructs, linearized plasmids were incubated with ribonucleoprotein complexes (RNP) containing purified SaCas9 protein and gRNA (where the gRNA spacer is complementary to a portion of the gRNA binding site).

Purified plasmids were linearized with Psil enzyme (New England Biolabs) and purified using ZymoClean DNA gel extraction kit (Zymo Research, Irvine, Calif.). Purified SaCas9 protein was purchased (Aldevron, Madison, Wis.). sgRNAs were expressed and purified using manufacture's recommended protocols (GeneArt Precision gRNA synthesis Kit, Life Technologies, Grand Island, N.Y.). For DNA digestion assay, SaCas9, sgRNA, and plasmid substrates were mixed in ratio of 10:10:1 and incubated for 2 hours at 37° C. DNA digestion patterns were analyzed using Flash-gel electrophoresis. The resulting products were analyzed by agarose gel electrophoresis.

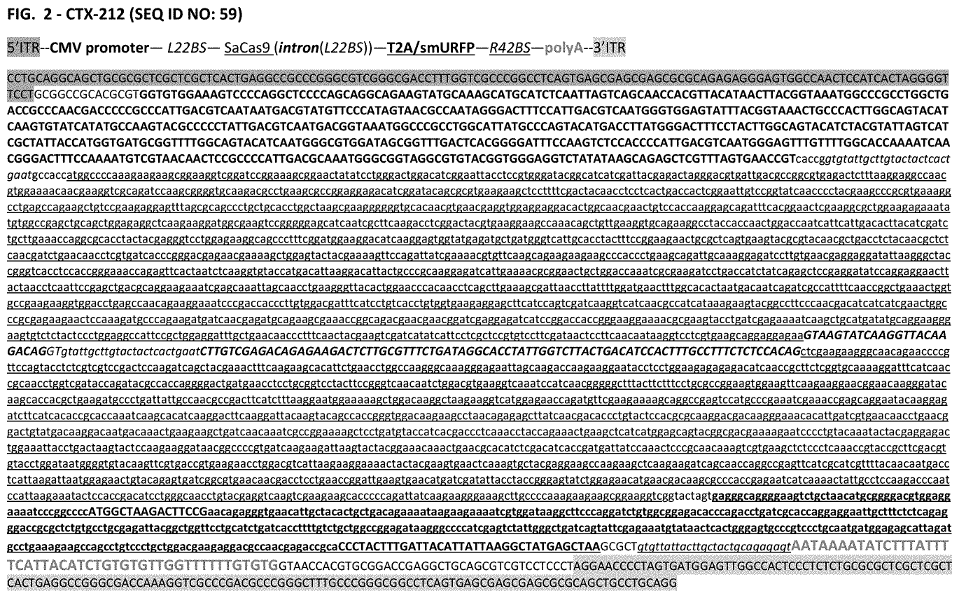

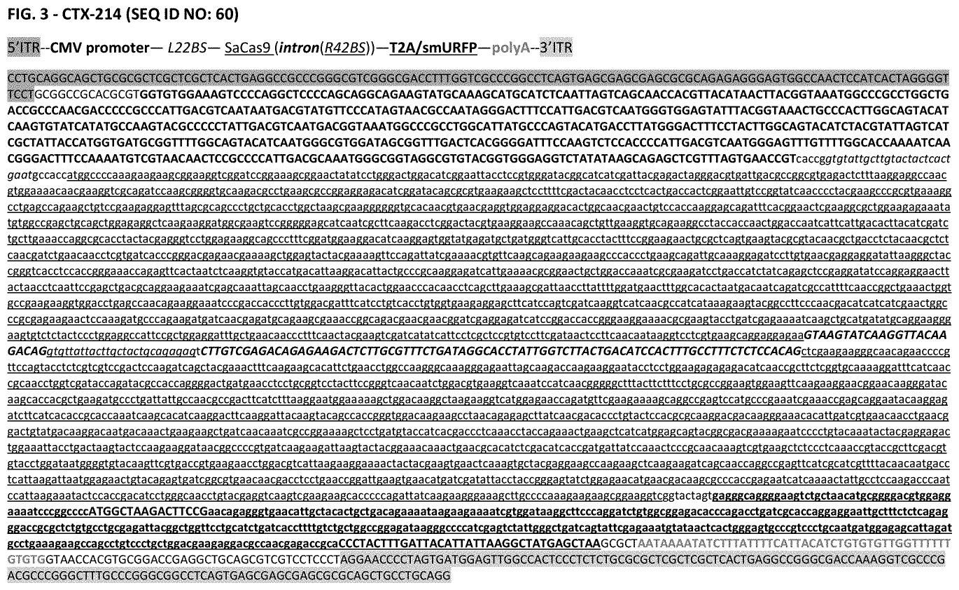

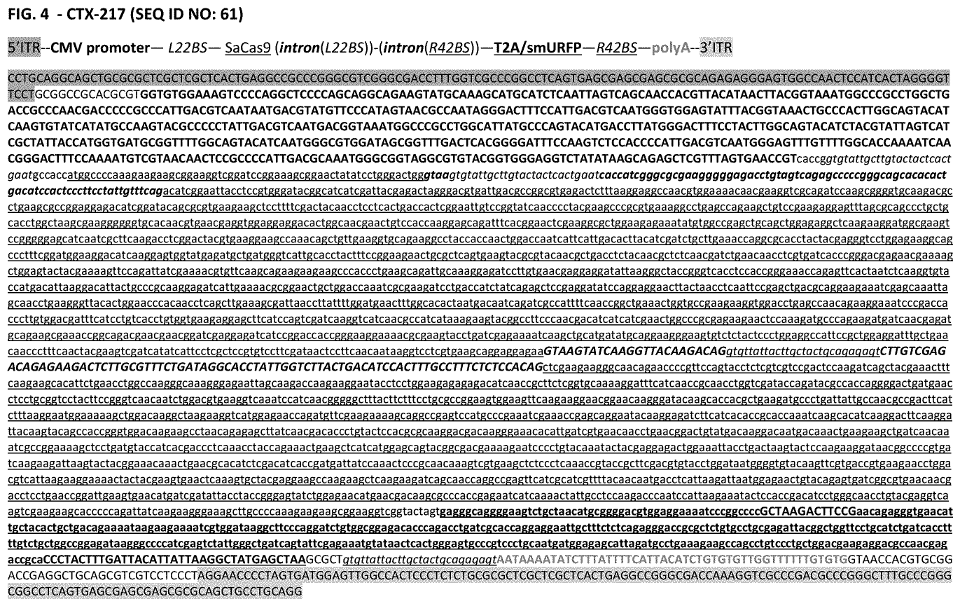

Three of the plasmid vectors were selected for further evaluation in AAV format. The nucleotide sequences are depicted in FIGS. 2-4. Each contains the following gRNA binding sites:

L22BS:
(SEQ ID NO: 75)
GTGTATTGCTTGTACTACTCACTGAAT
R42BS:
(SEQ ID NO: 50)
GTGTTATTACTTGCTACTGCAGAGAGT

The SIN-AAV vectors were injected into mice to study self-inactivation kinetics and also assess the impact of self-inactivation on editing efficiencies. For intravenous administration, six to eight week old C57BL/6 male mice were injected via the tail vein with 1e12 vg each vector/mouse of the AAV9 vector pairs for one week, two weeks, four weeks and twelve weeks. For intramuscular administration, Six to eight week old C57BL/6 male mice were injected via the tibialis anterior with 5e10 vg each vector/muscle of the AAV1 vector pairs for one week, two weeks, four weeks and twelve weeks. For subretinal injection, six to eight week old C57BL/6 male mice were injected with le10 vg/eye, for four weeks.

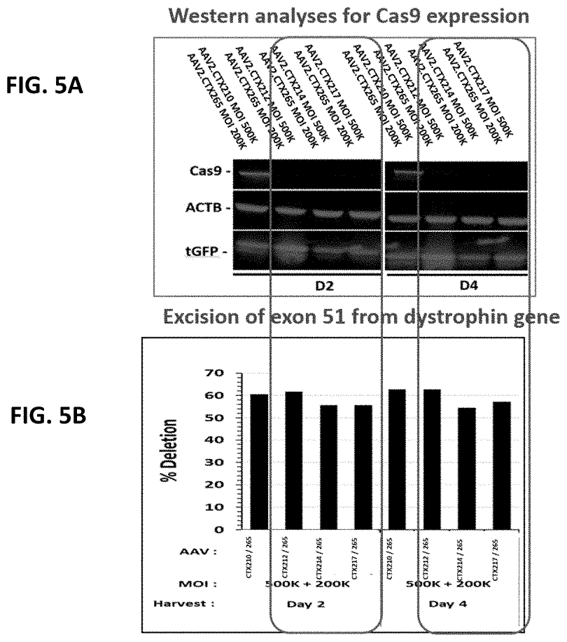

As shown in FIGS. 5A-5B, Both non- and SIN-AAV vectors mediated similar levels of editing (deletion of >55% of alleles). Similar data were obtained with two different pairs of sgRNAs specific to the mouse dystrophin gene (L64/R32 and mLT2/mRT2) with the following nucleotide sequences:

L64:
(SEQ ID NO: 32)
CTTAGAGGTCTTCTACATACAGTTTAAGTACTCTGTGCTGGAAACAGCAC
AGAATCTACTTAAACAAGGCAAAATGCCGTGTTTATCTCGTCAACTTGTT
GGCGAGATTTTTTT
R32:
(SEQ ID NO: 34)
CTATTCTGAGTACAGAGCATAGTTTAAGTACTCTGTGCTGGAAACAGCAC
AGAATCTACTTAAACAAGGCAAAATGCCGTGTTTATCTCGTCAACTTGTT
GGCGAGATTTTTTT
mLT2:
(SEQ ID NO: 35)
ACTATGATTAAATGCTTGATAGTTTAAGTACTCTGTGCTGGAAACAGCAC
AGAATCTACTTAAACAAGGCAAAATGCCGTGTTTATCTCGTCAACTTGTT
GGCGAGATTTTTTT
mRT2:
(SEQ ID NO: 36)
CTTAAAGGCTTCATATAAGGGGTTTAAGTACTCTGTGCTGGAAACAGCAC
AGAATCTACTTAAACAAGGCAAAATGCCGTGTTTATCTCGTCAACTTGTT
GGCGAGATTTTTTT

### Example 2: Study of all-in-Two Mouse DMD Specific SIN Vector Systems in Liver and Muscle

All-in-two CRISPR/Cas9 vector systems containing target specific gRNAs and SIN sites were prepared for intravenous (i.v.) injection using AAV9 serotype viral vectors containing mouse DMD specific dual guides as follows:
Target specific constructs: CTX525 (SEQ ID NO: 69)+CTX603 (SEQ ID NO: 64)
   CTX214 (SEQ ID NO: 60)+CTX603 (SEQ ID NO: 64)
Universal constructs: CTX604+CTX1001
   CTX604+CTX1004

Eighty-three six to eight week old C57BL/6 male mice (5 mice/group) were injected via the tail vein with 1e12 vg of each vector/mouse of the vector pairs Primary tissue samples from liver, heart, quadriceps, tibialis anterior (TA), and gastrocnemius were collected, pulverized and cryo-embedded at one week, two weeks, four weeks and twelve weeks. Analysis of the primary samples included LR-PCR/TapeStation, ddPCR for on-target activity, qPCR, Western, Mesco Scale Discovery (MSD) and/or IHC for SaCas9 expression levels.

Secondary samples of serum were also collected for analysis with Cas9 specific antibodies by ELISA.

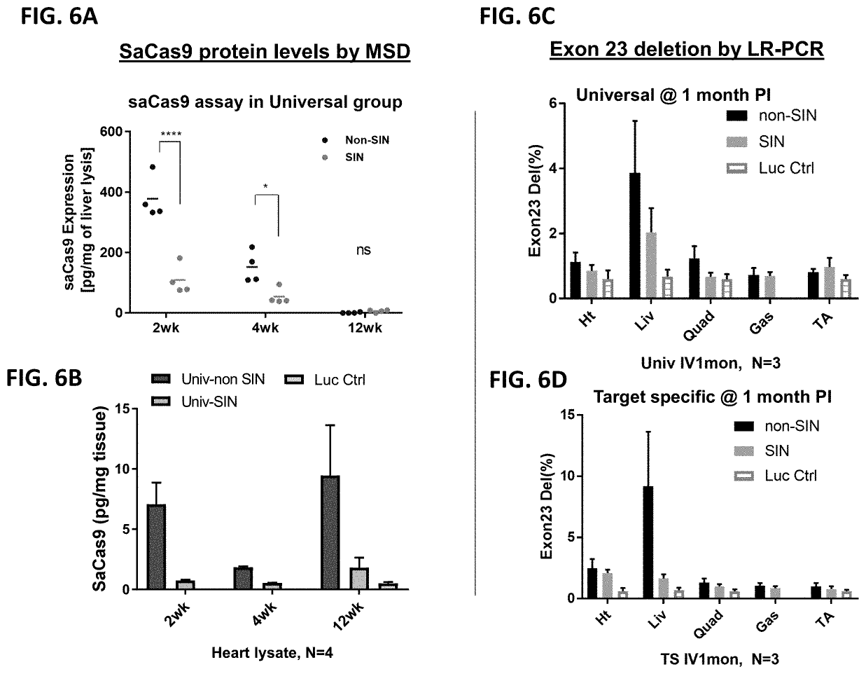

As shown in FIGS. 6A-6D, lower protein levels of SaCas9 were detected by MSD with SIN vector systems than non-SIN vector systems, and SaCas9 protein levels were reduced over time in the liver, while no reduction was observed in the heart. In addition, SaCas9 protein levels were detectable by IHC at one-month post injection, with protein levels lower in SIN vector groups than in non-SIN vector groups. Exon 23 excision efficiency, as measured by LR-PCR was approximately 2% in cardiac muscles and approximately 10% in liver.

Overall, these results indicated that the all-in-two DMD specific SIN vector systems mediated low editing in cardiac and skeletal muscles and liver.

### Example 3: Study of all-in-Two Mouse DMD Specific SIN Vector Systems in Mouse Retina

The expression and editing efficiency of the two vector systems used in Example 2, also were studied in the mouse retina. Thirty six to eight week old C57BL/6 male mice were injected with 1e10 vg/eye, and SaCas9 expression and gene editing was determined at one-month post injection.

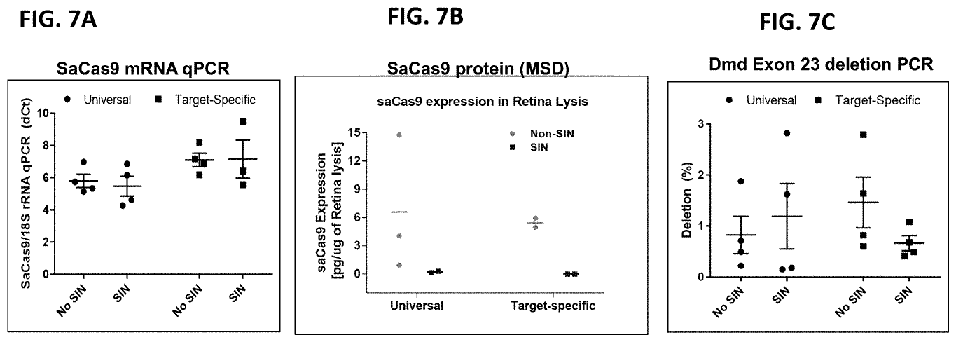

Similar levels of SaCas9 mRNA were detected with non-SIN and SIN vectors, but SaCas9 protein levels were reduced by up to 95% in mice treated with the SIN vectors. The excision efficiency of both SIN and non-SIN vectors was approximately 2.5%. Notably, editing efficiency was not impacted by the universal-SIN system, but was impacted with target-specific SIN vector systems. (FIGS. 7A-7C)

### Example 4: All-in-Two SIN Vector System for Excision of Exon 51 of Human DMD Gene

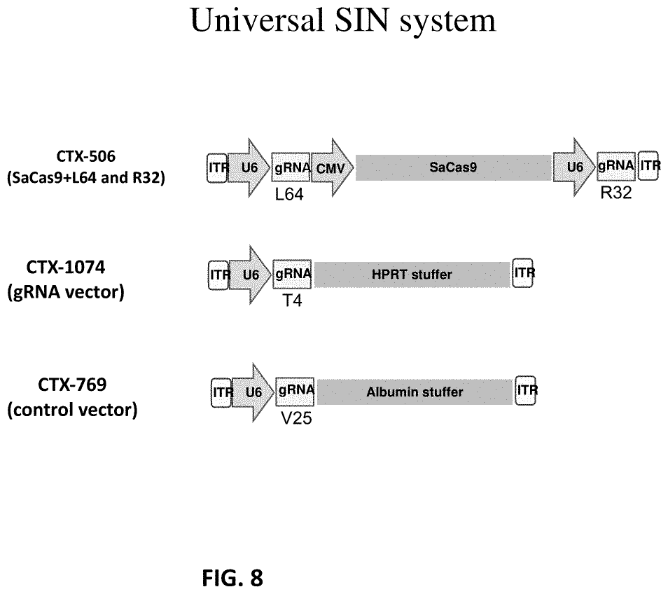

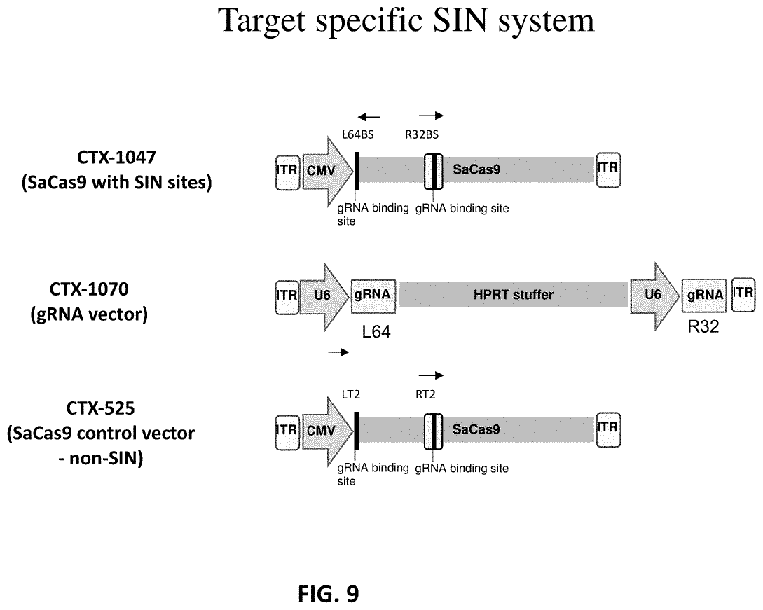

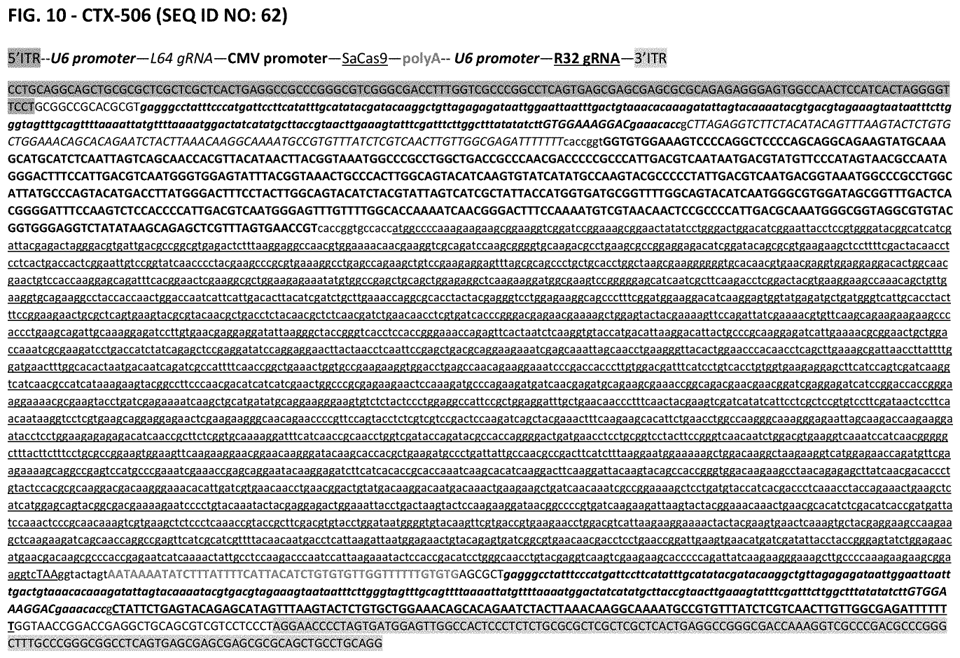

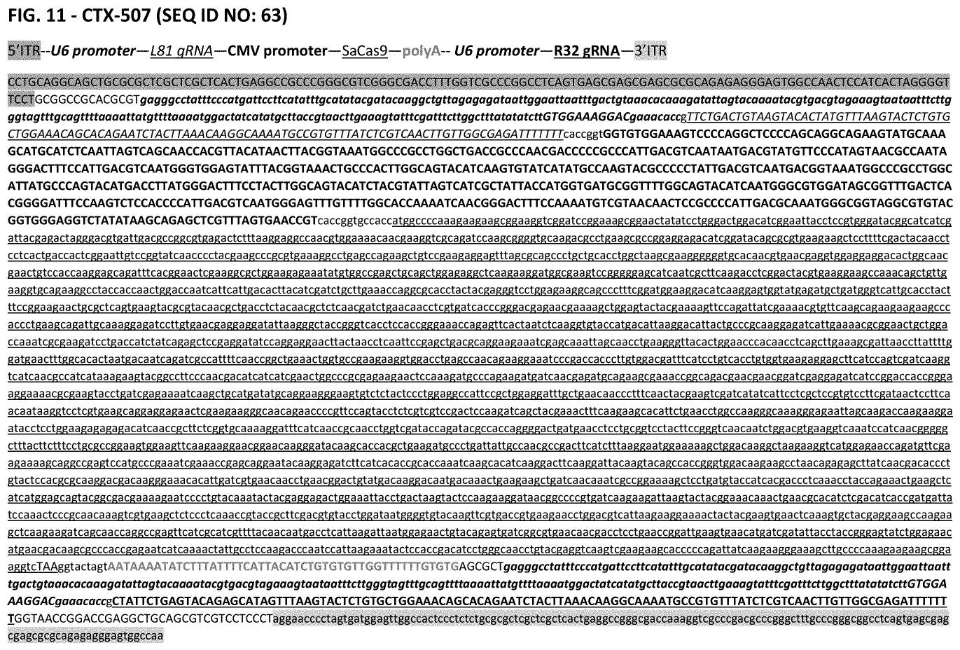

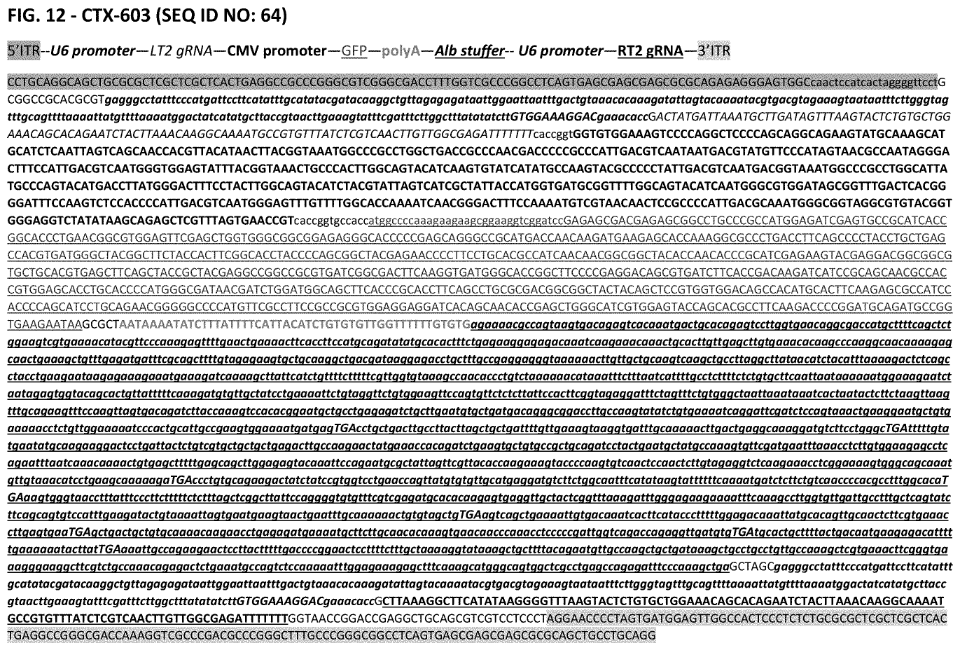

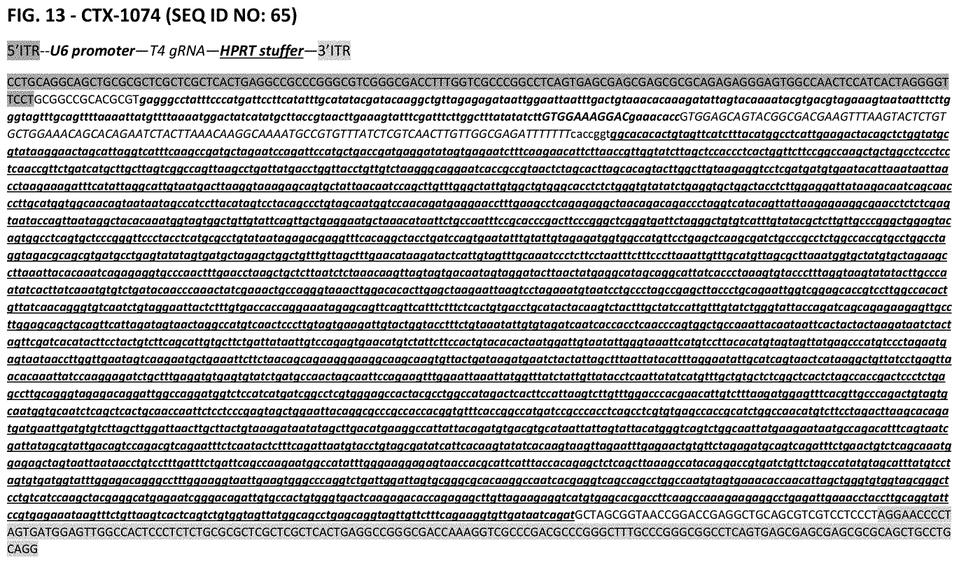

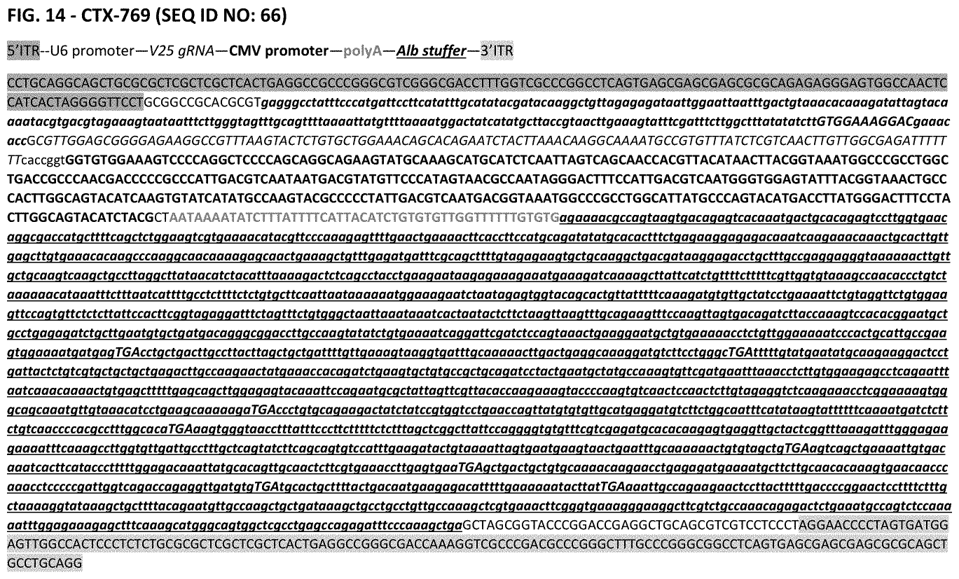

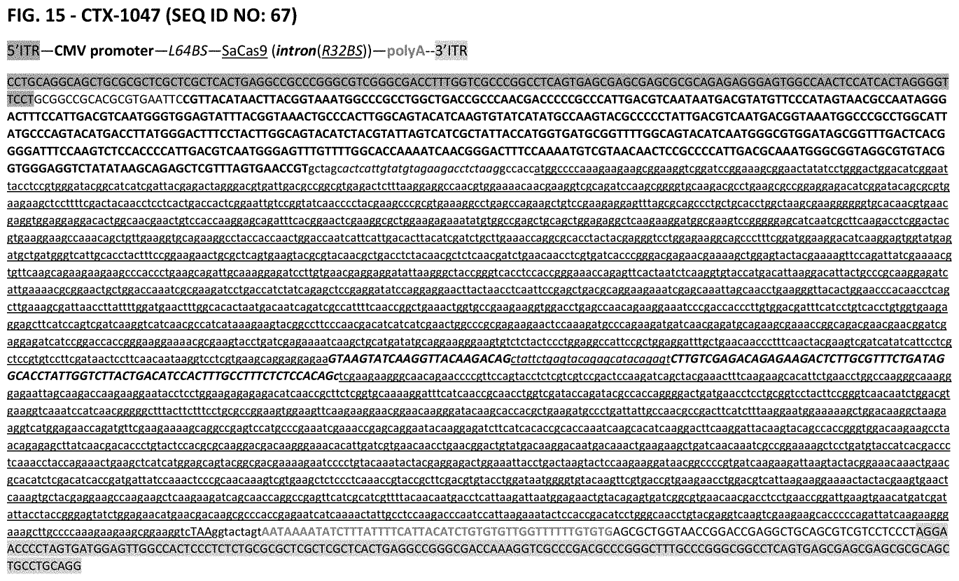

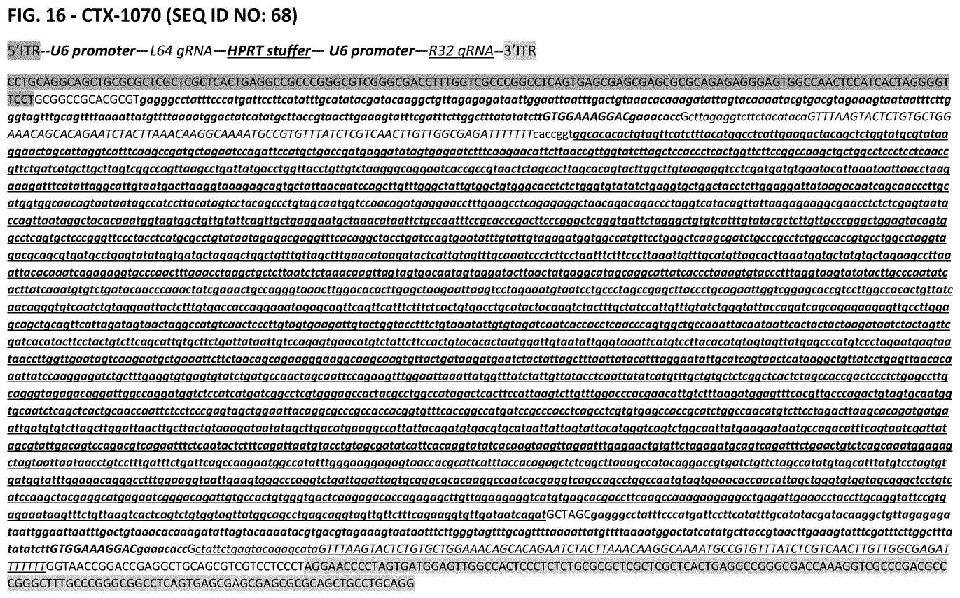

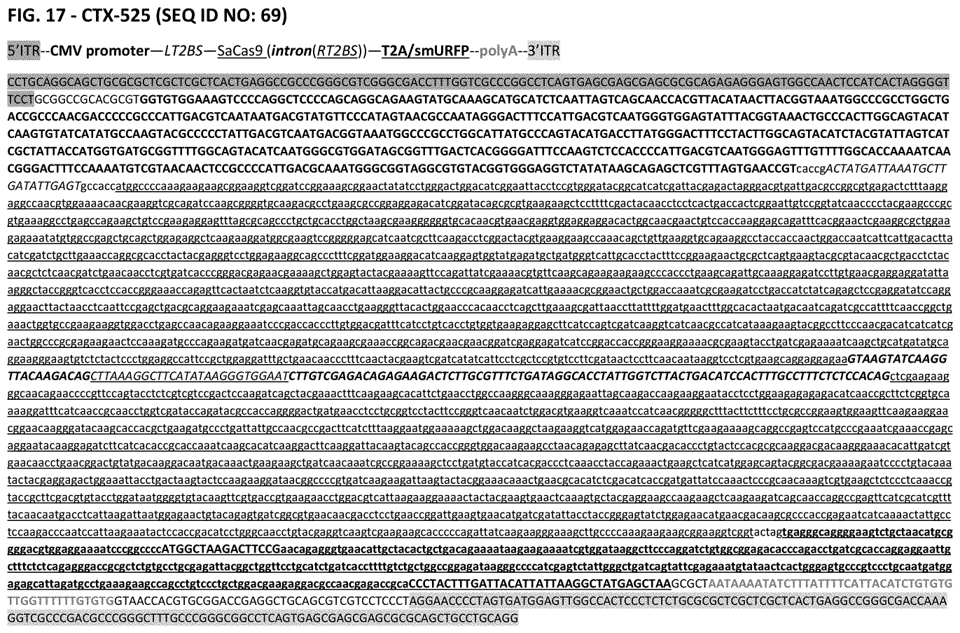

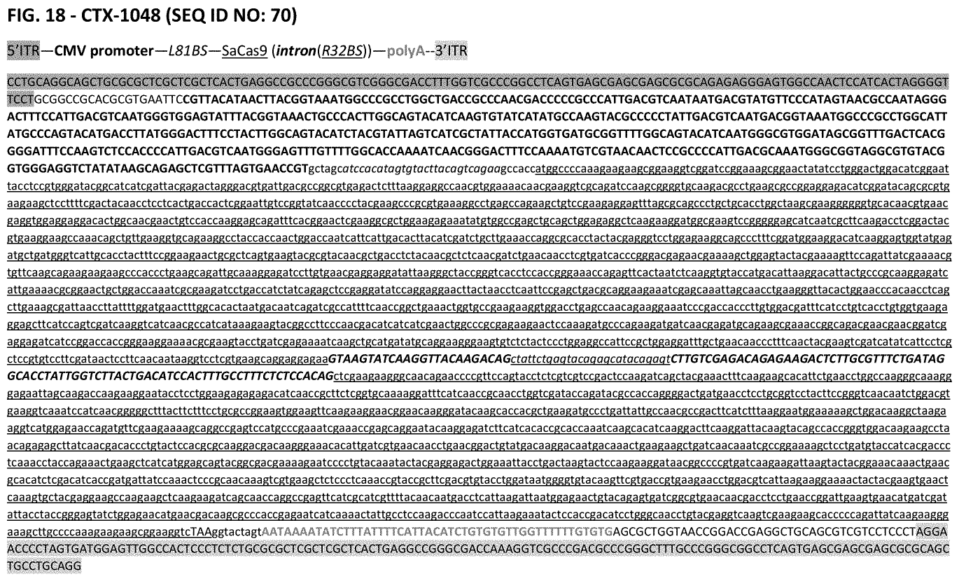

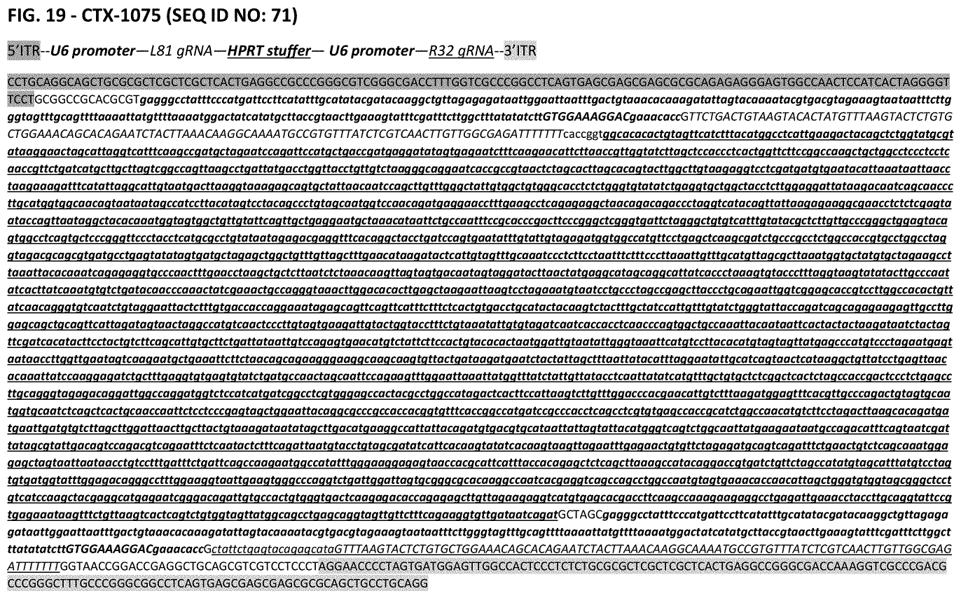

Design and generation of plasmid/vectors. AAV vector plasmid constructs used in these Examples were built using standard cloning procedures and Gibson High-Fidelity assembly reactions based on manufacture's recommendations (New England Biolabs, Ipswich, Mass.). The structures of the vectors are depicted in FIG. 8 and FIG. 9. The component sequences shown in Table 3.

Cell Transduction. Human Embryonic Kidney (HEK293T) cells (from ATCC, Manassas, Va.) and myoblasts (Cook Myosite, Pittsburgh, Pa.) were cultured and maintained at a low passage number as per the manufacture's recommendation. In preparation for transfection, HEK293T cells were added to 96-well or 12-well plates at 400,000 cells/ml and transfected 12-24 hours later using Jetprime reagent kit (VWR, Radnor, Pa.). For electroporation of myogenic cells, 200,000 cells were mixed with 5 μg of plasmids in Solution P1 and electroporated into cells using 4D Nucleofector DS150 Program. Prior to cell harvest, protein expression was analyzed using Evos fluorescence microscope.

Cas9 Protein Expression To determine Cas9 protein expression, cell pellets were treated with chilled RIPA buffer (Fisher Scientific, Waltham, Mass.) containing Protease Inhibitors (Sigma Aldrich, St. Louis, Mo.) and incubated at 4° C. for 30 minutes. Cell debris was cleared using high-speed spin at 10,000×g for 10 mins at 4° C. Protein samples were loaded onto Wes 12-230 kD capillary system (Protein Simple, San Jose, Calif.). SaCas9 (EPR19799) and (3-actin (RM112) protein antibodies were purchased (Abcam, Cambridge, Mass.). TurboGFP protein antibody was purchased (Fisher Scientific, Waltham, Mass.).

Exon 51 Excision Efficiency Genomic DNA was extracted from cell samples and amplified by long range polymerase chain reaction. The PCT products were resolved and quantitated by an Agilent 4200 tape station system.

The universal SIN vector system utilized the following plasmids:
CTX-506 (SaCas9+L64 and R32)
CTX-1074 (gRNA vector)
CTX-769 (control)

The target specific SIN system utilized the following plasmids:
CTX-1047 (SaCas9 with SIN sites and L64BS and R32BS)
CTX-1070 (gRNA vector)
CTX-525 (control)

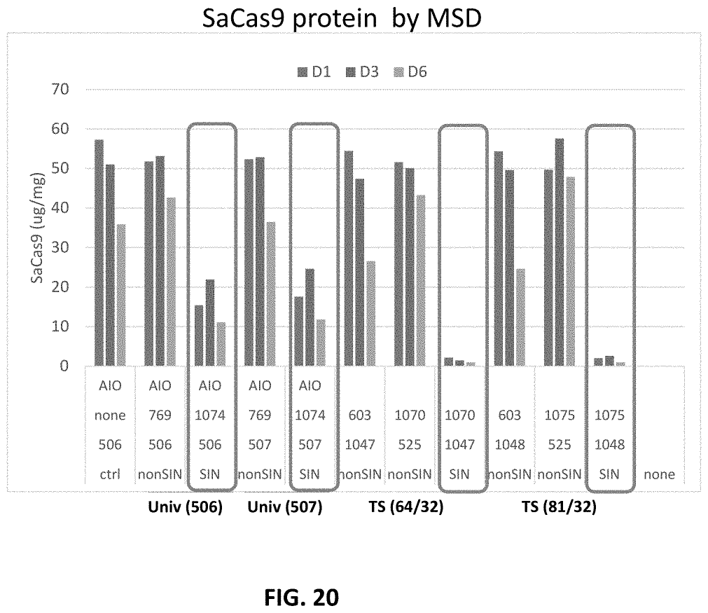

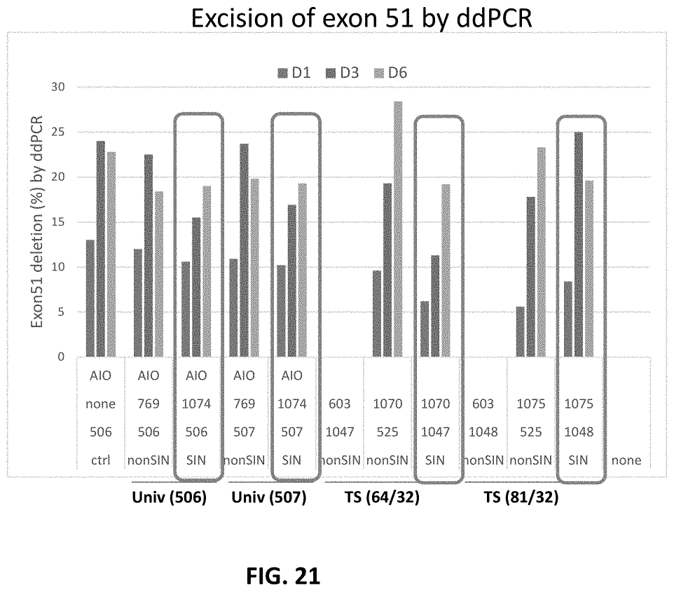

The resulting AAV constructs were transfected into HEK293T to examine kinetics of protein expression at days 1 (D1), 3 (D3) and 6 (D6) post-transfection. As shown in FIG. 20 and FIG. 21, SaCas9 expression was reduced in cells transfected with target specific SIN vectors compared to non-SIN vector systems without impacting editing efficiencies. The most efficient reduction in SaCas9 protein expression was observed in vectors containing gRNA pairs L64/R32 and L81/R32.

### Example 5: AAV Studies with all-in-Two SIN Vector Systems for Excision of Exon 51 of Human DMD

All-in-two AAV vectors were generated based on the plasmids containing the L64 and R32 gRNA from the previous example. HEK293 T cells were transduced with the AAV all-in-two target specific vector system and readouts were taken at days 1 (D1), 3 (D3) and 5 (D5) post-transduction.

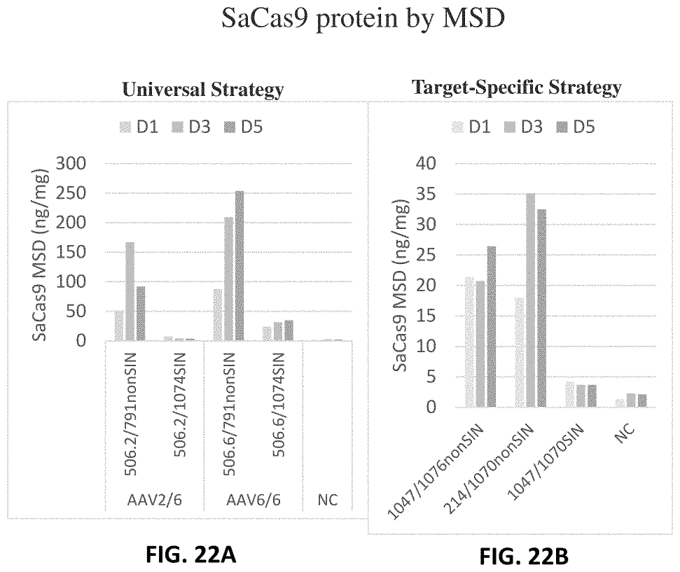

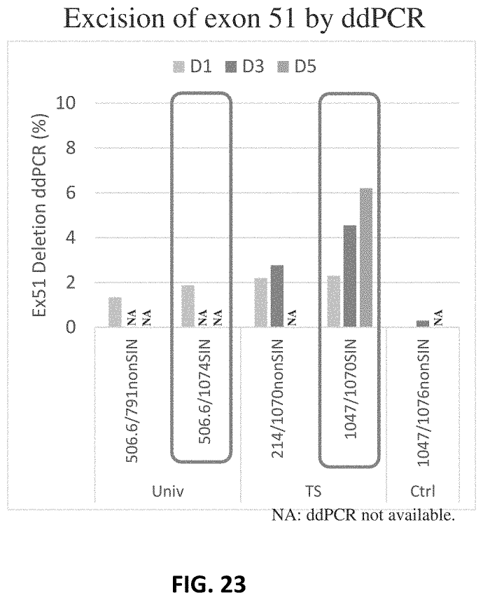

As shown in FIGS. 22A-22B AND FIG. 23, SaCas9 expression was reduced in cells transduced with the target specific SIN vector system compared to cells transduced with the non-SIN vector system without impacting editing efficiencies.

The results of these studies indicate that the all-in-two CRISPR/Cas9 vector systems containing target specific self-inactivating elements have a number of advantages. First, the vectors are more efficiently produced as there in so self-inactivation during production compared to vectors containing universal self-inactivating sites. The all-in-two CRISPR/Cas9 vector systems containing target specific self-inactivating elements also permit the use of different ratios of the two vectors for fine tuning of on-target activity and self-activation. In addition, the all-in-two CRISPR/Cas9 vector systems containing target specific self-inactivating elements permit injection of the two vectors simultaneously or at different time points in order to allow fine tuning the balance between on-target activity and self-inactivation.

### Note Regarding Illustrative Examples and Documents Cited

While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative aspects provided herein.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

TABLE 3
Summary of Spacer Sequences

| Left | Spacer Sequence | Right | Spacer Sequence |
|---|---|---|---|
| L01 | CTGAGTAGGAGCTAAAATATT | R6 | AACTGGTGGGAAATGGTCTAG |
| | (SEQ ID NO: 1) | | (SEQ ID NO: 18) |
| L02 | ACAATAAGTCAAATTTAATTG | R7 | ATTATACTTAGGCTGAATAGT |
| | (SEQ ID NO: 2) | | (SEQ ID NO: 19) |
| L03 | AAGATATATAATGTCATGAAT | R11 | TTTAAATGTAAATAGCTCAG |
| | (SEQ ID NO: 3) | | (SEQ ID NO: 20) |
| L16 | AATGGTTAAGATGCATAGTAC | R14 | TGGCACAGACAACTTAGAAGA |
| | (SEQ ID NO: 4) | | (SEQ ID NO: 21) |
| L18 | TATGTGGCTTTACCAAGGTCC | R15 | AAATTGGCACAGACAACTTAG |
| | (SEQ ID NO: 5) | | (SEQ ID NO: 22) |
| L22 | GTGTATTGCTTGTACTACTCA | R22 | AAAAACAAGAAGTGAGGCAGA |
| | (SEQ ID NO: 6) | | (SEQ ID NO: 23) |
| L34 | TCTCCTCATTAGAGAAGAAG | R26 | CTGCATTTAAAGGCCTTGAGC |
| | (SEQ ID NO: 7) | | (SEQ ID NO: 24) |
| L37 | CTCAAGCTTCTCAGGGACACC | R32 | CTATTCTGAGTACAGAGCATA |
| | (SEQ ID NO: 8) | | (SEQ ID NO: 25) |
| L45 | ATCCTCACACATGCATCCTCT | R41 | AGCAAGTAATAACACAAGCTT |
| | (SEQ ID NO: 9) | | (SEQ ID NO: 26) |
| L52 | AAAGTGAAGGATGAGGAACTA | R42 | GTGTTATTACTTGCTACTGCA |
| | (SEQ ID NO: 10) | | (SEQ ID NO: 27) |
| L57 | AAATTAGCTGAAGCATATTCA | R52 | ACACTTCCTTGTGACGGGTTT |
| | (SEQ ID NO: 11) | | (SEQ ID NO: 28) |
| L61 | TCTTGCATCTTGCACATGTCC | R53 | ATTGATGTGCTCAGTAGTCTC |
| | (SEQ ID NO: 12) | | (SEQ ID NO: 29) |
| L64 | CTTAGAGGTCTTCTACATACA | R91 | TTACACACAGGATGGAGAAAA |
| | (SEQ ID NO: 13) | | (SEQ ID NO: 30) |
| L81 | TTCTGACTGTAAGTACACTAT | R99 | GCAATTCTCCTGAATAGAAA |
| | (SEQ ID NO: 14) | | (SEQ ID NO: 31) |
| L84 | TCTGGAGGGTCAAATCTGGT | | |
| | (SEQ ID NO: 15) | | |
| L85 | AATGGAGAGAGGTAAGTCTG | | |
| | (SEQ ID NO: 16) | | |
| L88 | TGAAATGGCCTGTGCTCATGA | | |
| | (SEQ ID NO: 17) | | |

TABLE 4
Summary of gRNA Sequences

| gRNA | Sequence | SEQ ID NO: |
|---|---|---|
| L64 gRNA | CTTAGAGGTCTTCTACATACAGTTTAAGTACTCTGTGCTGGAA | 32 |
| | ACAGCACAGAATCTACTTAAACAAGGCAAAATGCCGTGTTTA | |
| | TCTCGTCAACTTGTTGGCGAGATTTTTTT | |
| L81 gRNA | TTCTGACTGTAAGTACACTATGTTTAAGTACTCTGTGCTGGAA | 33 |
| | ACAGCACAGAATCTACTTAAACAAGGCAAAATGCCGTGTTTA | |
| | TCTCGTCAACTTGTTGGCGAGATTTTTTT | |
| R32 gRNA | CTATTCTGAGTACAGAGCATAGTTTAAGTACTCTGTGCTGGA | 34 |
| | AACAGCACAGAATCTACTTAAACAAGGCAAAATGCCGTGTTT | |
| | ATCTCGTCAACTTGTTGGCGAGATTTTTTT | |
| LT2 gRNA | ACTATGATTAAATGCTTGATAGTTTAAGTACTCTGTGCTGGA | 35 |
| | AACAGCACAGAATCTACTTAAACAAGGCAAAATGCCGTGTTT | |
| | ATCTCGTCAACTTGTTGGCGAGATTTTTTT | |
| RT2 gRNA | CTTAAAGGCTTCATATAAGGGGTTTAAGTACTCTGTGCTGGA | 36 |
| | AACAGCACAGAATCTACTTAAACAAGGCAAAATGCCGTGTTT | |
| | ATCTCGTCAACTTGTTGGCGAGATTTTTTT | |
| V25 gRNA | CGTTGGAGCGGGGAGAAGGCCGTTTAAGTACTCTGTGCTGGA | 37 |
| | AACAGCACAGAATCTACTTAAACAAGGCAAAATGCCGTGTTT | |
| | ATCTCGTCAACTTGTTGGCGAGATTTTTTT | |

TABLE 5
Summary of gRNA Target Sequences

| Left | gRNA Target Sequence | Right | gRNA Target Sequence |
|---|---|---|---|
| L64BS | ACTCATTGTATGTAGAAGACCTCTAAG | R32BS | CTATTCTGAGTACAGAGCATACAGAGT |
| | (SEQ ID NO: 38) | | (SEQ ID NO: 40) |
| L81BS | ATCCACATAGTGTACTTACAGTCAGAA | | |
| | (SEQ ID NO: 39) | | |

TABLE 6
Summary of Component Sequences for gRNA Vectors

| Component | Sequence | SEQ ID NO: |
|---|---|---|
| 5′AAV ITR | CCTGCAGGCAGCTGCGCGCTCGCTCGCTCACTGAGGCCGCCC | 41 |
| | GGGCGTCGGGCGACCTTTGGTCGCCCGGCCTCAGTGAGCGAG | |
| | CGAGCGCGCAGAGAGGGAGTGGCCAACTCCATCACTAGGGG | |
| | TTCCT | |
| U6 Promoter | GAGGGCCTATTTCCCATGATTCCTTCATATTTGCATATACGAT | 42 |
| | ACAAGGCTGTTAGAGAGATAATTGGAATTAATTTGACTGTAA | |
| | ACACAAAGATATTAGTACAAAATACGTGACGTAGAAAGTAA | |
| | TAATTTCTTGGGTAGTTTGCAGTTTTAAAATTATGTTTTAAAA | |
| | TGGACTATCATATGCTTACCGTAACTTGAAAGTATTTCGATTT | |
| | CTTGGCTTTATATATCTTGTGGAAAGGACGAAACACC | |
| 3′ITR | AGGAACCCCTAGTGATGGAGTTGGCCACTCCCTCTCTGCGCG | 43 |
| | CTCGCTCGCTCACTGAGGCCGGGCGACCAAAGGTCGCCCGAC | |
| | GCCCGGGCTTTGCCCGGGCGGCCTCAGTGAGCGAGCGAGCGC | |
| | GCAGCTGCCTGCAGG | |
| Albumin | AGAAAACGCCAGTAAGTGACAGAGTCACAAATGACTGCACA | 44 |
| | GAGTCCTTGGTGAACAGGCGACCATGCTTTTCAGCTCTGGAA | |
| | GTCGTGAAAACATACGTTCCCAAAGAGTTTTGAACTGAAAAC | |
| | TTCACCTTCCATGCAGATATATGCACACTTTCTGAGAAGGAG | |
| | AGACAAATCAAGAAACAAACTGCACTTGTTGAGCTTGTGAAA | |
| | CACAAGCCCAAGGCAACAAAAGAGCAACTGAAAGCTGTTTG | |
| | AGATGATTTCGCAGCTTTTGTAGAGAAGTGCTGCAAGGCTGA | |
| | CGATAAGGAGACCTGCTTTGCCGAGGAGGGTAAAAAACTTGT | |
| | TGCTGCAAGTCAAGCTGCCTTAGGCTTATAACATCTACATTTA | |
| | AAAGACTCTCAGCCTACCTGAAGAATAAGAGAAAGAAATGA | |
| | AAGATCAAAAGCTTATTCATCTGTTTTCTTTTTCGTTGGTGTA | |
| | AAGCCAACACCCTGTCTAAAAAACATAAATTTCTTTAATCAT | |
| | TTTGCCTCTTTTCTCTGTGCTTCAATTAATAAAAAATGGAAAG | |
| | AATCTAATAGAGTGGTACAGCACTGTTATTTTTCAAAGATGT | |
| | GTTGCTATCCTGAAAATTCTGTAGGTTCTGTGGAAGTTCCAGT | |
| | GTTCTCTCTTATTCCACTTCGGTAGAGGATTTCTAGTTTCTGT | |
| | GGGCTAATTAAATAAATCACTAATACTCTTCTAAGTTAAGTTT | |
| | GCAGAAGTTTCCAAGTTAGTGACAGATCTTACCAAAGTCCAC | |
| | ACGGAATGCTGCCTGAGAGATCTGCTTGAATGTGCTGATGAC | |
| | AGGGCGGACCTTGCCAAGTATATCTGTGAAAATCAGGATTCG | |
| | ATCTCCAGTAAACTGAAGGAATGCTGTGAAAAACCTCTGTTG | |
| | GAAAAATCCCACTGCATTGCCGAAGTGGAAAATGATGAGTG | |
| | ACCTGCTGACTTGCCTTACTTAGCTGCTGATTTTGTTGAAAGT | |
| | AAGGTGATTTGCAAAAACTTGACTGAGGCAAAGGATGTCTTC | |
| | CTGGGCTGATTTTTGTATGAATATGCAAGAAGGACTCCTGAT | |
| | TACTCTGTCGTGCTGCTGCTGAGACTTGCCAAGAACTATGAA | |
| | ACCACAGATCTGAAGTGCTGTGCCGCTGCAGATCCTACTGAA | |
| | TGCTATGCCAAAGTGTTCGATGAATTTAAACCTCTTGTGGAA | |
| | GAGCCTCAGAATTTAATCAAACAAAACTGTGAGCTTTTTGAG | |
| | CAGCTTGGAGAGTACAAATTCCAGAATGCGCTATTAGTTCGT | |
| | TACACCAAGAAAGTACCCCAAGTGTCAACTCCAACTCTTGTA | |
| | 46GAGGTCTCAAGAAACCTCGGAAAAGTGGGCAGCAAATGTT | |
| | GTAAACATCCTGAAGCAAAAAGATGACCCTGTGCAGAAGAC | |
| | TATCTATCCGTGGTCCTGAACCAGTTATGTGTGTTGCATGAGG | |
| | ATGTCTTCTGGCAATTTCATATAAGTATTTTTTCAAAATGATC | |
| | TCTTCTGTCAACCCCACGCCTTTGGCACATGAAAGTGGGTAA | |
| | CCTTTATTTCCCTTCTTTTTCTCTTTAGCTCGGCTTATTCCAGG | |
| | GGTGTGTTTCGTCGAGATGCACACAAGAGTGAGGTTGCTACT | |
| | CGGTTTAAAGATTTGGGAGAAGAAAATTTCAAAGCCTTGGTG | |
| | TTGATTGCCTTTGCTCAGTATCTTCAGCAGTGTCCATTTGAAG | |
| | ATACTGTAAAATTAGTGAATGAAGTAACTGAATTTGCAAAAA | |
| | ACTGTGTAGCTGTGAAGTCAGCTGAAAATTGTGACAAATCAC | |
| | TTCATACCCTTTTTGGAGACAAATTATGCACAGTTGCAACTCT | |
| | TCGTGAAACCTTGAGTGAATGAGCTGACTGCTGTGCAAAACA | |
| | AGAACCTGAGAGATGAAAATGCTTCTTGCAACACAAAGTGA | |
| | ACAACCCAAACCTCCCCCGATTGGTCAGACCAGAGGTTGATG | |
| | TGTGATGCACTGCTTTTACTGACAATGAAGAGACATTTTTGA | |
| | AAAAATACTTATTGAAAATTGCCAGAAGAACTCCTTACTTTT | |
| | TGACCCCGGAACTCCTTTTCTTTGCTAAAAGGTATAAAGCTG | |
| | CTTTTACAGAATGTTGCCAAGCTGCTGATAAAGCTGCCTGCC | |
| | TGTTGCCAAAGCTCGTGAAACTTCGGGTGAAAGGGAAGGCTT | |
| | CGTCTGCCAAACAGAGACTCTGAAATGCCAGTCTCCAAAAAT | |
| | TTGGAGAAAGAGCTTTCAAAGCATGGGCAGTGGCTCGCCTGA | |
| | GCCAGAGATTTCCCAAAGCTGA | |
| HPRT | CCTGCAGGCAGCTGCGCGCTCGCTCGCTCACTGAGGCCGCCC | 45 |
| | GGGCGTCGGGCGACCTTTGGTCGCCCGGCCTCAGTGAGCGAG | |
| | CGAGCGCGCAGAGAGGGAGTGGCCAACTCCATCACTAGGGG | |
| | TTCCTGCGGCCGCACGCGTGAGGGCCTATTTCCCATGATTCCT | |
| | TCATATTTGCATATACGATACAAGGCTGTTAGAGAGATAATT | |
| | GGAATTAATTTGACTGTAAACACAAAGATATTAGTACAAAAT | |
| | ACGTGACGTAGAAAGTAATAATTTCTTGGGTAGTTTGCAGTT | |
| | TTAAAATTATGTTTTAAAATGGACTATCATATGCTTACCGTAA | |
| | CTTGAAAGTATTTCGATTTCTTGGCTTTATATATCTTGTGGAA | |
| | AGGACGAAACACCGTGGAGCAGTACGGCGACGAAGTTTAAG | |
| | TACTCTGTGCTGGAAACAGCACAGAATCTACTTAAACAAGGC | |
| | AAAATGCCGTGTTTATCTCGTCAACTTGTTGGCGAGATTTTTT | |
| | TCACCGGTGGCACACACTGTAGTTCATCTTTACATGGCCTCAT | |
| | TGAAGACTACAGCTCTGGTATGCGTATAAGGAACTAGCATTA | |
| | GGTCATTTCAAGCCGATGCTAGAATCCAGATTCCATGCTGAC | |
| | CGATGAGGATATAGTGAGAATCTTTCAAGAACATTCTTAACC | |
| | GTTGGTATCTTAGCTCCACCCTCACTGGTTCTTCCGGCCAAGC | |
| | TGCTGGCCTCCCTCCTCAACCGTTCTGATCATGCTTGCTTAGT | |
| | CGGCCAGTTAAGCCTGATTATGACCTGGTTACCTGTTGTCTAA | |
| | GGGCAGGAATCACCGCCGTAACTCTAGCACTTAGCACAGTAC | |
| | TTGGCTTGTAAGAGGTCCTCGATGATGTGAATACATTAAATA | |
| | ATTAACCTAAGAAAGATTTCATATTAGGCATTGTAATGACTT | |
| | AAGGTAAAGAGCAGTGCTATTAACAATCCAGCTTGTTTGGGC | |
| | TATTGTGGCTGTGGGCACCTCTCTGGGTGTATATCTGAGGTGC | |
| | TGGCTACCTCTTGGAGGATTATAAGACAATCAGCAACCCTTG | |
| | CATGGTGGCAACAGTAATAATAGCCATCCTTACATAGTCCTA | |
| | CAGCCCTGTAGCAATGGTCCAACAGATGAGGAACCTTTGAAG | |
| | CCTCAGAGAGGCTAACAGACAGACCCTAGGTCATACAGTTAT | |
| | TAAGAGAAGGCGAACCTCTCTCGAGTAATACCAGTTAATAGG | |
| | CTACACAAATGGTAGTGGCTGTTGTATTCAGTTGCTGAGGAA | |
| | TGCTAAACATAATTCTGCCAATTTCCGCACCCGACTTCCCGG | |
| | GCTCGGGTGATTCTAGGGCTGTGTCATTTGTATACGCTCTTGT | |
| | TGCCCGGGCTGGAGTACAGTGGCCTCAGTGCTCCCGGGTTCC | |
| | CTACCTCATGCGCCTGTATAATAGAGACGAGGTTTCACAGGC | |
| | TACCTGATCCAGTGAATATTTGTATTGTAGAGATGGTGGCCA | |
| | TGTTCCTGAGCTCAAGCGATCTGCCCGCCTCTGGCCACCGTG | |
| | CCTGGCCTAGGTAGACGCAGCGTGATGCCTGAGTATATAGTG | |
| | ATGCTAGAGCTGGCTGTTTGTTAGCTTTGAACATAAGATACT | |
| | CATTGTAGTTTGCAAATCCCTCTTCCTAATTTCTTTCCCTTAA | |
| | ATTGTTTGCATGTTAGCGCTTAAATGGTGCTATGTGCTAGAA | |
| | GCCTTAAATTACACAAATCAGAGAGGTGCCCAACTTTGAACC | |
| | TAAGCTGCTCTTAATCTCTAAACAAGTTAGTAGTGACAATAG | |
| | TAGGATACTTAACTATGAGGCATAGCAGGCATTATCACCCTA | |
| | AAGTGTACCCTTTAGGTAAGTATATACTTGCCCAATATCACTT | |
| | ATCAAATGTGTCTGATACAACCCAAACTATCGAAACTGCCAG | |
| | GGTAAACTTGGACACACTTGAGCTAAGAATTAAGTCCTAGAA | |
| | ATGTAATCCTGCCCTAGCCGAGCTTACCCTGCAGAATTGGTC | |
| | GGAGCACCGTCCTTGGCCACACTGTTATCAACAGGGTGTCAA | |
| | TCTGTAGGAATTACTCTTTGTGACCACCAGGAAATAGAGCAG | |
| | TTCAGTTCATTTCTTTCTCACTGTGACCTGCATACTACAAGTC | |
| | TACTTTGCTATCCATTGTTTGTATCTGGGTATTACCAGATCAG | |
| | CAGAGAAGAGTTGCCTTGGAGCAGCTGCAGTTCATTAGATAG | |
| | TAACTAGGCCATGTCAACTCCCTTGTAGTGAAGATTGTACTG | |
| | GTACCTTTCTGTAAATATTGTGTAGATCAATCACCACCTCAAC | |
| | CCAGTGGCTGCCAAATTACAATAATTCACTACTACTAAGATA | |
| | ATCTACTAGTTCGATCACATACTTCCTACTGTCTTCAGCATTG | |
| | TGCTTCTGATTATAATTGTCCAGAGTGAACATGTCTATTCTTC | |
| | CACTGTACACACTAATGGATTGTAATATTGGGTAAATTCATG | |
| | TCCTTACACATGTAGTAGTTATGAGCCCATGTCCCTAGAATG | |
| | AGTAATAACCTTGGTTGAATAGTCAAGAATGCTGAAATTCTT | |
| | CTAACAGCAGAAGGGAAGGCAAGCAAGTGTTACTGATAAGA | |
| | TGAATCTACTATTAGCTTTAATTATACATTTAGGAATATTGCA | |
| | TCAGTAACTCATAAGGCTGTTATCCTGAGTTAACACAAATTA | |
| | TCCAAGGAGATCTGCTTTGAGGTGTGAGTGTATCTGATGCCA | |
| | ACTAGCAATTCCAGAAGTTTGGAATTAAATTATGGTTTATCT | |
| | ATTGTTATACCTCAATTATATCATGTTTGCTGTGCTCTCGGCT | |
| | CACTCTAGCCACCGACTCCCTCTGAGCCTTGCAGGGTAGAGA | |
| | CAGGATTGGCCAGGATGGTCTCCATCATGATCGGCCTCGTGG | |
| | GAGCCACTACGCCTGGCCATAGACTCACTTCCATTAAGTCTT | |
| | GTTTGGACCCACGAACATTGTCTTTAAGATGGAGTTTCACGTT | |
| | GCCCAGACTGTAGTGCAATGGTGCAATCTCAGCTCACTGCAA | |
| | CCAATTCTCCTCCCGAGTAGCTGGAATTACAGGCGCCCGCCA | |
| | CCACGGTGTTTCACCGGCCATGATCCGCCCACCTCAGCCTCG | |
| | TGTGAGCCACCGCATCTGGCCAACATGTCTTCCTAGACTTAA | |
| | GCACAGATGATGAATTGATGTGTCTTAGCTTGGATTAACTTG | |
| | CTTACTGTAAAGATAATATAGCTTGACATGAAGGCCATTATT | |
| | ACAGATGTGACGTGCATAATTATTAGTATTACATGGGTCAGT | |
| | CTGGCAATTATGAAGAATAATGCCAGACATTTCAGTAATCGA | |
| | TTATAGCGTATTGACAGTCCAGACGTCAGAATTTCTCAATAC | |
| | TCTTTCAGATTAATGTACCTGTAGCGATATCATTCACAAGTAT | |
| | ATCACAAGTAAGTTAGAATTTGAGAACTGTGTTCTAGAGATG | |
| | CAGTCAGATTTCTGAACTGTCTCAGCAAATGGAGAGCTAGTA | |
| | ATTAATAACCTGTCCTTTGATTTCTGATTCAGCCAAGAATGGC | |
| | CATATTTGGGAAGGAGAGTAACCACGCATTCATTTACCACAG | |
| | AGCTCTCAGCTTAAAGCCATACAGGACCGTGATCTGTTCTAG | |
| | CCATATGTAGCATTTATGTCCTAGTGTGATGGTATTTGGAGAC | |
| | AGGGCCTTTGGAAGGTAATTGAAGTGGGCCCAGGTCTGATTG | |
| | GATTAGTGCGGGCGCACAAGGCCAATCACGAGGTCAGCCAG | |
| | CCTGGCCAATGTAGTGAAACACCAACATTAGCTGGGTGTGGT | |
| | AGCGGGCTCCTGTCATCCAAGCTACGAGGCATGAGAATCGGG | |
| | ACAGATTGTGCCACTGTGGGTGACTCAAGAGACACCAGAGA | |
| | GCTTGTTAGAAGAGGTCATGTGAGCACGACCTTCAAGCCAAA | |
| | GAAGAGGCCTGAGATTGAAACCTACCTTGCAGGTATTCCGTG | |
| | AGAAATAAGTTTCTGTTAAGTCACTCAGTCTGTGGTAGTTAT | |
| | GGCAGCCTGAGCAGGTAGTTGTTCTTTCAGAAGGTGTTGATA | |
| | ATCAGA | |

TABLE 7
Summary of SaCas9 Amino Acid Sequences

| Polypeptide | Sequence | SEQ ID NO: |
|---|---|---|
| Wild-type | MKRNYILGLDIGITSVGYGIIDYETRDVIDAGVRLFKEANVENNE | 46 |
| S. aureus Cas9 | GRRSKRGARRLKRRRRHRIQRVKKLLFDYNLLTDHSELSGINPY | |
| | EARVKGLSQKLSEEEFSAALLHLAKRRGVHNVNEVEEDTGNEL | |
| | STKEQISRNSKALEEKYVAELQLERLKKDGEVRGSINRFKTSDY | |
| | VKEAKQLLKVQKAYHQLDQSFIDTYIDLLETRRTYYEGPGEGSP | |
| | FGWKDIKEWYEMLMGHCTYFPEELRSVKYAYNADLYNALNDL | |
| | NNLVITRDENEKLEYYEKFQIIENVFKQKKKPTLKQIAKEILVNE | |
| | EDIKGYRVTSTGKPEFTNLKVYHDIKDITARKEIIENAELLDQIAK | |
| | ILTIYQSSEDIQEELTNLNSELTQEEIEQISNLKGYTGTHNLSLKAI | |
| | NLILDELWHTNDNQIAIFNRLKLVPKKVDLSQQKEIPTTLVDDFI | |
| | LSPVVKRSFIQSIKVINAIIKKYGLPNDIIIELAREKNSKDAQKNIIN | |
| | EMQKRNRQTNERIEEIIRTTGKENAKYLIEKIKLHDMQEGKCLY | |
| | SLEAIPLEDLLNNPFNYEVDHIIPRSVSFDNSFNNKVLVKQEENS | |
| | KKGNRTPFQYLSSSDSKISYETFKKHILNLAKGKGRISKTKKEYL | |
| | LEERDINRFSVQKDFINRNLVDTRYATRGLMNLLRSYFRVNNLD | |
| | VKVKSINGGFTSFLRRKWKFKKERNKGYKHHAEDALIIANADFI | |
| | FKEWKKLDKAKKVMENQMFEEKQAESMPEIETEQEYKEIFITPH | |
| | QIKHIKDFKDYKYSHRVDKKPNRELINDTLYSTRKDDKGNTLIV | |
| | NNLNGLYDKDNDKLKKLINKSPEKLLMYHHDPQTYQKLKLIME | |
| | QYGDEKNPLYKYYEETGNYLTKYSKKDNGPVIKKIKYYGNKLN | |
| | AHLDITDDYPNSRNKVVKLSLKPYRFDVYLDNGVYKFVTVKNL | |
| | DVIKKENYYEVNSKCYEEAKKLKKISNQAEFIASFYNNDLIKING | |
| | ELYRVIGVNNDLLNRIEVNMIDITYREYLENMNDKRPPRIIKTIAS | |
| | KTQSIKKYSTDILGNLYEVKSKKHPQIIKKG | |
| S. aureus Cas9 | MKRNYILGLAIGITSVGYGIIDYETRDVIDAGVRLFKEANVENNE | 47 |
| D10 variant | GRRSKRGARRLKRRRRHRIQRVKKLLFDYNLLTDHSELSGINPY | |
| | EARVKGLSQKLSEEEFSAALLHLAKRRGVHNVNEVEEDTGNEL | |
| | STKEQISRNSKALEEKYVAELQLERLKKDGEVRGSINRFKTSDY | |
| | VKEAKQLLKVQKAYHQLDQSFIDTYIDLLETRRTYYEGPGEGSP | |
| | FGWKDIKEWYEMLMGHCTYFPEELRSVKYAYNADLYNALNDL | |
| | NNLVITRDENEKLEYYEKFQIIENVFKQKKKPTLKQIAKEILVNE | |
| | EDIKGYRVTSTGKPEFTNLKVYHDIKDITARKEIIENAELLDQIAK | |
| | ILTIYQSSEDIQEELTNLNSELTQEEIEQISNLKGYTGTHNLSLKAI | |
| | NLILDELWHTNDNQIAIFNRLKLVPKKVDLSQQKEIPTTLVDDFI | |
| | LSPVVKRSFIQSIKVINAIIKKYGLPNDIIIELAREKNSKDAQKNIIN | |
| | EMQKRNRQTNERIEEIIRTTGKENAKYLIEKIKLHDMQEGKCLY | |
| | SLEAIPLEDLLNNPFNYEVDHIIPRSVSFDNSFNNKVLVKQEENS | |
| | KKGNRTPFQYLSSSDSKISYETFKKHILNLAKGKGRISKTKKEYL | |
| | LEERDINRFSVQKDFINRNLVDTRYATRGLMNLLRSYFRVNNLD | |
| | VKVKSINGGFTSFLRRKWKFKKERNKGYKHHAEDALIIANADFI | |
| | FKEWKKLDKAKKVMENQMFEEKQAESMPEIETEQEYKEIFITPH | |
| | QIKHIKDFKDYKYSHRVDKKPNRELINDTLYSTRKDDKGNTLIV | |
| | NNLNGLYDKDNDKLKKLINKSPEKLLMYHHDPQTYQKLKLIME | |
| | QYGDEKNPLYKYYEETGNYLTKYSKKDNGPVIKKIKYYGNKLN | |
| | AHLDITDDYPNSRNKVVKLSLKPYRFDVYLDNGVYKFVTVKNL | |
| | DVIKKENYYEVNSKCYEEAKKLKKISNQAEFIASFYNNDLIKING | |
| | ELYRVIGVNNDLLNRIEVNMIDITYREYLENMNDKRPPRIIKTIAS | |
| | KTQSIKKYSTDILGNLYEVKSKKHPQIIKKG | |
| S. aureus N580A | MKRNYILGLDIGITSVGYGIIDYETRDVIDAGVRLFKEANVENNE | 48 |
| variant | GRRSKRGARRLKRRRRHRIQRVKKLLFDYNLLTDHSELSGINPY | |
| | EARVKGLSQKLSEEEFSAALLHLAKRRGVHNVNEVEEDTGNEL | |
| | STKEQISRNSKALEEKYVAELQLERLKKDGEVRGSINRFKTSDY | |
| | VKEAKQLLKVQKAYHQLDQSFIDTYIDLLETRRTYYEGPGEGSP | |
| | FGWKDIKEWYEMLMGHCTYFPEELRSVKYAYNADLYNALNDL | |
| | NNLVITRDENEKLEYYEKFQIIENVFKQKKKPTLKQIAKEILVNE | |
| | EDIKGYRVTSTGKPEFTNLKVYHDIKDITARKEIIENAELLDQIAK | |
| | ILTIYQSSEDIQEELTNLNSELTQEEIEQISNLKGYTGTHNLSLKAI | |
| | NLILDELWHTNDNQIAIFNRLKLVPKKVDLSQQKEIPTTLVDDFI | |
| | LSPVVKRSFIQSIKVINAIIKKYGLPNDIIIELAREKNSKDAQKNIIN | |
| | EMQKRNRQTNERIEEIIRTTGKENAKYLIEKIKLHDMQEGKCLY | |
| | SLEAIPLEDLLNNPFNYEVDHIIPRSVSFDNSFNNKVLVKQEEAS | |
| | KKGNRTPFQYLSSSDSKISYETFKKHILNLAKGKGRISKTKKEYL | |
| | LEERDINRFSVQKDFINRNLVDTRYATRGLMNLLRSYFRVNNLD | |
| | VKVKSINGGFTSFLRRKWKFKKERNKGYKHHAEDALIIANADFI | |
| | FKEWKKLDKAKKVMENQMFEEKQAESMPEIETEQEYKEIFITPH | |
| | QIKHIKDFKDYKYSHRVDKKPNRELINDTLYSTRKDDKGNTLIV | |
| | NNLNGLYDKDNDKLKKLINKSPEKLLMYHHDPQTYQKLKLIME | |
| | QYGDEKNPLYKYYEETGNYLTKYSKKDNGPVIKKIKYYGNKLN | |
| | AHLDITDDYPNSRNKVVKLSLKPYRFDVYLDNGVYKFVTVKNL | |
| | DVIKKENYYEVNSKCYEEAKKLKKISNQAEFIASFYNNDLIKING | |
| | ELYRVIGVNNDLLNRIEVNMIDITYREYLENMNDKRPPRIIKTIAS | |
| | KTQSIKKYSTDILGNLYEVKSKKHPQIIKKG | |
| S. aureus D10 & | MKRNYILGLAIGITSVGYGIIDYETRDVIDAGVRLFKEANVENNE | 49 |
| N580A valiant | GRRSKRGARRLKRRRRHRIQRVKKLLFDYNLLTDHSELSGINPY | |
| | EARVKGLSQKLSEEEFSAALLHLAKRRGVHNVNEVEEDTGNEL | |
| | STKEQISRNSKALEEKYVAELQLERLKKDGEVRGSINRFKTSDY | |
| | VKEAKQLLKVQKAYHQLDQSFIDTYIDLLETRRTYYEGPGEGSP | |
| | FGWKDIKEWYEMLMGHCTYFPEELRSVKYAYNADLYNALNDL | |
| | NNLVITRDENEKLEYYEKFQIIENVFKQKKKPTLKQIAKEILVNE | |
| | EDIKGYRVTSTGKPEFTNLKVYHDIKDITARKEIIENAELLDQIAK | |
| | ILTIYQSSEDIQEELTNLNSELTQEEIEQISNLKGYTGTHNLSLKAI | |
| | NLILDELWHTNDNQIAIFNRLKLVPKKVDLSQQKEIPTTLVDDFI | |
| | LSPVVKRSFIQSIKVINAIIKKYGLPNDIIIELAREKNSKDAQKNIIN | |
| | EMQKRNRQTNERIEEIIRTTGKENAKYLIEKIKLHDMQEGKCLY | |
| | SLEAIPLEDLLNNPFNYEVDHIIPRSVSFDNSFNNKVLVKQEEAS | |
| | KKGNRTPFQYLSSSDSKISYETFKKHILNLAKGKGRISKTKKEYL | |
| | LEERDINRFSVQKDFINRNLVDTRYATRGLMNLLRSYFRVNNLD | |
| | VKVKSINGGFTSFLRRKWKFKKERNKGYKHHAEDALIIANADFI | |
| | FKEWKKLDKAKKVMENQMFEEKQAESMPEIETEQEYKEIFITPH | |
| | QIKHIKDFKDYKYSHRVDKKPNRELINDTLYSTRKDDKGNTLIV | |
| | NNLNGLYDKDNDKLKKLINKSPEKLLMYHHDPQTYQKLKLIME | |
| | QYGDEKNPLYKYYEETGNYLTKYSKKDNGPVIKKIKYYGNKLN | |
| | AHLDITDDYPNSRNKVVKLSLKPYRFDVYLDNGVYKFVTVKNL | |
| | DVIKKENYYEVNSKCYEEAKKLKKISNQAEFIASFYNNDLIKING | |
| | ELYRVIGVNNDLLNRIEVNMIDITYREYLENMNDKRPPRIIKTIAS | |
| | KTQSIKKYSTDILGNLYEVKSKKHPQIIKKG | |

TABLE 8
Summary of Component Sequences for SaCas9 vectors

| Component | Sequence | SEQ ID NO: |
|---|---|---|
| 5′ITR | CCTGCAGGCAGCTGCGCGCTCGCTCGCTCACTGAGGCCGCCC | 41 |
| | GGGCGTCGGGCGACCTTTGGTCGCCCGGCCTCAGTGAGCGAG | |
| | CGAGCGCGCAGAGAGGGAGTGGCCAACTCCATCACTAGGGG | |
| | TTCCT | |
| CMV Promoter | GGTGTGGAAAGTCCCCAGGCTCCCCAGCAGGCAGAAGTATG | 51 |
| | CAAAGCATGCATCTCAATTAGTCAGCAACCACGTTACATAAC | |
| | TTACGGTAAATGGCCCGCCTGGCTGACCGCCCAACGACCCCC | |
| | GCCCATTGACGTCAATAATGACGTATGTTCCCATAGTAACGC | |
| | CAATAGGGACTTTCCATTGACGTCAATGGGTGGAGTATTTAC | |
| | GGTAAACTGCCCACTTGGCAGTACATCAAGTGTATCATATGC | |
| | CAAGTACGCCCCCTATTGACGTCAATGACGGTAAATGGCCCG | |
| | CCTGGCATTATGCCCAGTACATGACCTTATGGGACTTTCCTAC | |
| | TTGGCAGTACATCTACGTATTAGTCATCGCTATTACCATGGTG | |
| | ATGCGGTTTTGGCAGTACATCAATGGGCGTGGATAGCGGTTT | |
| | GACTCACGGGGATTTCCAAGTCTCCACCCCATTGACGTCAAT | |
| | GGGAGTTTGTTTTGGCACCAAAATCAACGGGACTTTCCAAAA | |
| | TGTCGTAACAACTCCGCCCCATTGACGCAAATGGGCGGTAGG | |
| | CGTGTACGGTGGGAGGTCTATATAAGCAGAGCTCGTTTAGTG | |
| | AACCGT | |
| SaCas9 | ATGGCCCCAAAGAAGAAGCGGAAGGTCGGATCCGGAAAGCG | 52 |
| | GAACTATATCCTGGGACTGGACATCGGAATTACCTCCGTGGG | |
| | ATACGGCATCATCGATTACGAGACTAGGGACGTGATTGACGC | |
| | CGGCGTGAGACTCTTTAAGGAGGCCAACGTGGAAAACAACG | |
| | AAGGTCGCAGATCCAAGCGGGGTGCAAGACGCCTGAAGCGC | |
| | CGGAGGAGACATCGGATACAGCGCGTGAAGAAGCTCCTTTTC | |
| | GACTACAACCTCCTCACTGACCACTCGGAATTGTCCGGTATC | |
| | AACCCCTACGAAGCCCGCGTGAAAGGCCTGAGCCAGAAGCT | |
| | GTCCGAAGAGGAGTTTAGCGCAGCCCTGCTGCACCTGGCTAA | |
| | GCGAAGGGGGGTGCACAACGTGAACGAGGTGGAGGAGGACA | |
| | CTGGCAACGAACTGTCCACCAAGGAGCAGATTTCACGGAACT | |
| | CGAAGGCGCTGGAAGAGAAATATGTGGCCGAGCTGCAGCTG | |
| | GAGAGGCTCAAGAAGGATGGCGAAGTCCGGGGGAGCATCAA | |
| | TCGCTTCAAGACCTCGGACTACGTGAAGGAAGCCAAACAGCT | |
| | GTTGAAGGTGCAGAAGGCCTACCACCAACTGGACCAATCATT | |
| | CATTGACACTTACATCGATCTGCTTGAAACCAGGCGCACCTA | |
| | CTACGAGGGTCCTGGAGAAGGCAGCCCTTTCGGATGGAAGG | |
| | ACATCAAGGAGTGGTATGAGATGCTGATGGGTCATTGCACCT | |
| | ACTTTCCGGAAGAACTGCGCTCAGTGAAGTACGCGTACAACG | |
| | CTGACCTCTACAACGCTCTCAACGATCTGAACAACCTCGTGA | |
| | TCACCCGGGACGAGAACGAAAAGCTGGAGTACTACGAAAAG | |
| | TTCCAGATTATCGAAAACGTGTTCAAGCAGAAGAAGAAGCCC | |
| | ACCCTGAAGCAGATTGCAAAGGAGATCCTTGTGAACGAGGA | |
| | GGATATTAAGGGCTACCGGGTCACCTCCACCGGGAAACCAG | |
| | AGTTCACTAATCTCAAGGTGTACCATGACATTAAGGACATTA | |
| | CTGCCCGCAAGGAGATCATTGAAAACGCGGAACTGCTGGAC | |
| | CAAATCGCGAAGATCCTGACCATCTATCAGAGCTCCGAGGAT | |
| | ATCCAGGAGGAACTTACTAACCTCAATTCCGAGCTGACGCAG | |
| | GAAGAAATCGAGCAAATTAGCAACCTGAAGGGTTACACTGG | |
| | AACCCACAACCTCAGCTTGAAAGCGATTAACCTTATTTTGGA | |
| | TGAACTTTGGCACACTAATGACAATCAGATCGCCATTTTCAA | |
| | CCGGCTGAAACTGGTGCCGAAGAAGGTGGACCTGAGCCAAC | |
| | AGAAGGAAATCCCGACCACCCTTGTGGACGATTTCATCCTGT | |
| | CACCTGTGGTGAAGAGGAGCTTCATCCAGTCGATCAAGGTCA | |
| | TCAACGCCATCATAAAGAAGTACGGCCTTCCCAACGACATCA | |
| | TCATCGAACTGGCCCGCGAGAAGAACTCCAAAGATGCCCAG | |
| | AAGATGATCAACGAGATGCAGAAGCGAAACCGGCAGACGAA | |
| | CGAACGGATCGAGGAGATCATCCGGACCACCGGGAAGGAAA | |
| | ACGCGAAGTACCTGATCGAGAAAATCAAGCTGCATGATATGC | |
| | AGGAAGGGAAGTGTCTCTACTCCCTGGAGGCCATTCCGCTGG | |
| | AGGATTTGCTGAACAACCCTTTCAACTACGAAGTCGATCATA | |
| | TCATTCCTCGCTCCGTGTCCTTCGATAACTCCTTCAACAATAA | |
| | GGTCCTCGTGAAGCAGGAGGAGAACTCGAAGAAGGGCAACA | |
| | GAACCCCGTTCCAGTACCTCTCGTCGTCCGACTCCAAGATCA | |
| | GCTACGAAACTTTCAAGAAGCACATTCTGAACCTGGCCAAGG | |
| | GCAAAGGGAGAATTAGCAAGACCAAGAAGGAATACCTCCTG | |
| | GAAGAGAGAGACATCAACCGCTTCTCGGTGCAAAAGGATTTC | |
| | TCAACCGCAACCTGGTCGATACCAGATACGCCACCAGGGG | |
| | ACTGATGAACCTCCTGCGGTCCTACTTCCGGGTCAACAATCT | |
| | GGACGTGAAGGTCAAATCCATCAACGGGGGCTTTACTTCTTT | |
| | CCTGCGCCGGAAGTGGAAGTTCAAGAAGGAACGGAACAAGG | |
| | GATACAAGCACCACGCTGAAGATGCCCTGATTATTGCCAACG | |
| | CCGACTTCATCTTTAAGGAATGGAAAAAGCTGGACAAGGCTA | |
| | AGAAGGTCATGGAGAACCAGATGTTCGAAGAAAAGCAGGCC | |
| | GAGTCCATGCCCGAAATCGAAACCGAGCAGGAATACAAGGA | |
| | GATCTTCATCACACCGCACCAAATCAAGCACATCAAGGACTT | |
| | CAAGGATTACAAGTACAGCCACCGGGTGGACAAGAAGCCTA | |
| | ACAGAGAGCTTATCAACGACACCCTGTACTCCACGCGCAAGG | |
| | ACGACAAGGGAAACACATTGATCGTGAACAACCTGAACGGA | |
| | CTGTATGACAAGGACAATGACAAACTGAAGAAGCTGATCAA | |
| | CAAATCGCCGGAAAAGCTCCTGATGTACCATCACGACCCTCA | |
| | AACCTACCAGAAACTGAAGCTCATCATGGAGCAGTACGGCG | |
| | ACGAAAAGAATCCCCTGTACAAATACTACGAGGAGACTGGA | |
| | AATTACCTGACTAAGTACTCCAAGAAGGATAACGGCCCCGTG | |
| | ATCAAGAAGATTAAGTACTACGGAAACAAACTGAACGCACA | |
| | TCTCGACATCACCGATGATTATCCAAACTCCCGCAACAAAGT | |
| | CGTGAAGCTCTCCCTCAAACCGTACCGCTTCGACGTGTACCT | |
| | GGATAATGGGGTGTACAAGTTCGTGACCGTGAAGAACCTGG | |
| | ACGTCATTAAGAAGGAAAACTACTACGAAGTGAACTCAAAG | |
| | TGCTACGAGGAAGCCAAGAAGCTCAAGAAGATCAGCAACCA | |
| | GGCCGAGTTCATCGCATCGTTTTACAACAATGACCTCATTAA | |
| | GATTAATGGAGAACTGTACAGAGTGATCGGCGTGAACAACG | |
| | ACCTCCTGAACCGGATTGAAGTGAACATGATCGATATTACCT | |
| | ACCGGGAGTATCTGGAGAACATGAACGACAAGCGCCCACCG | |
| | AGAATCATCAAAACTATTGCCTCCAAGACCCAATCCATTAAG | |
| | AAATACTCCACCGACATCCTGGGCAACCTGTACGAGGTCAAG | |
| | TCGAAGAAGCACCCCCAGATTATCAAGAAGGGAAAGCTTGC | |
| | CCCAAAGAAGAAGCGGAAGGTCTAA | |
| Intron | GTAAGTATCAAGGTTACAAGACAGCTTGTCGAGACAGAGAA | 53 |
| | GACTCTTGCGTTTCTGATAGGCACCTATTGGTCTTACTGACAT | |
| | CCACTTTGCCTTTCTCTCCACAG | |
| Intron | GTAAGTATCAAGGTTACAAGACAGGTTTAAGGAGACCAATA | 54 |
| | GAAACTGGGCTTGTCGAGACAGAGAAGACTCTTGCGTTTCTG | |
| | ATAGGCACCTATTGGTCTTACTGACATCCACTTTGCCTTTCTC | |
| | TCCACAG | |
| BCL11A intron 2 | GTATGTCTACATTTCTCTTAGGTAAACATCTAAGGCATTTCGA | 55 |
| genbank ID | GAACACAGAAAAGGTTTTGAGTTTGAG | |
| LC187302.1 | | |
| Retinoblastoma | GTTAATATTTCATAAATAGTTACTTTTTTTTTCATTTTTAGGAA | 56 |
| intron 16 genbank | G | |
| ID AY260473.1 | | |
| SaCas9 with | ATGGCCCCAAAGAAGAAGCGGAAGGTCGGATCCGGAAAGCG | 57 |
| intron containing | GAACTATATCCTGGGACTGGACATCGGAATTACCTCCGTGGG | |
| R32BS | ATACGGCATCATCGATTACGAGACTAGGGACGTGATTGACGC | |
| | CGGCGTGAGACTCTTTAAGGAGGCCAACGTGGAAAACAACG | |
| | AAGGTCGCAGATCCAAGCGGGGTGCAAGACGCCTGAAGCGC | |
| | CGGAGGAGACATCGGATACAGCGCGTGAAGAAGCTCCTTTTC | |
| | GACTACAACCTCCTCACTGACCACTCGGAATTGTCCGGTATC | |
| | AACCCCTACGAAGCCCGCGTGAAAGGCCTGAGCCAGAAGCT | |
| | GTCCGAAGAGGAGTTTAGCGCAGCCCTGCTGCACCTGGCTAA | |
| | GCGAAGGGGGGTGCACAACGTGAACGAGGTGGAGGAGGACA | |
| | CTGGCAACGAACTGTCCACCAAGGAGCAGATTTCACGGAACT | |
| | CGAAGGCGCTGGAAGAGAAATATGTGGCCGAGCTGCAGCTG | |
| | GAGAGGCTCAAGAAGGATGGCGAAGTCCGGGGGAGCATCAA | |
| | TCGCTTCAAGACCTCGGACTACGTGAAGGAAGCCAAACAGCT | |
| | GTTGAAGGTGCAGAAGGCCTACCACCAACTGGACCAATCATT | |
| | CATTGACACTTACATCGATCTGCTTGAAACCAGGCGCACCTA | |
| | CTACGAGGGTCCTGGAGAAGGCAGCCCTTTCGGATGGAAGG | |
| | ACATCAAGGAGTGGTATGAGATGCTGATGGGTCATTGCACCT | |
| | ACTTTCCGGAAGAACTGCGCTCAGTGAAGTACGCGTACAACG | |
| | CTGACCTCTACAACGCTCTCAACGATCTGAACAACCTCGTGA | |
| | TCACCCGGGACGAGAACGAAAAGCTGGAGTACTACGAAAAG | |
| | TTCCAGATTATCGAAAACGTGTTCAAGCAGAAGAAGAAGCCC | |
| | ACCCTGAAGCAGATTGCAAAGGAGATCCTTGTGAACGAGGA | |
| | GGATATTAAGGGCTACCGGGTCACCTCCACCGGGAAACCAG | |
| | AGTTCACTAATCTCAAGGTGTACCATGACATTAAGGACATTA | |
| | CTGCCCGCAAGGAGATCATTGAAAACGCGGAACTGCTGGAC | |
| | CAAATCGCGAAGATCCTGACCATCTATCAGAGCTCCGAGGAT | |
| | ATCCAGGAGGAACTTACTAACCTCAATTCCGAGCTGACGCAG | |
| | GAAGAAATCGAGCAAATTAGCAACCTGAAGGGTTACACTGG | |
| | AACCCACAACCTCAGCTTGAAAGCGATTAACCTTATTTTGGA | |
| | TGAACTTTGGCACACTAATGACAATCAGATCGCCATTTTCAA | |
| | CCGGCTGAAACTGGTGCCGAAGAAGGTGGACCTGAGCCAAC | |
| | AGAAGGAAATCCCGACCACCCTTGTGGACGATTTCATCCTGT | |
| | CACCTGTGGTGAAGAGGAGCTTCATCCAGTCGATCAAGGTCA | |
| | TCAACGCCATCATAAAGAAGTACGGCCTTCCCAACGACATCA | |
| | TCATCGAACTGGCCCGCGAGAAGAACTCCAAAGATGCCCAG | |
| | AAGATGATCAACGAGATGCAGAAGCGAAACCGGCAGACGAA | |
| | CGAACGGATCGAGGAGATCATCCGGACCACCGGGAAGGAAA | |
| | ACGCGAAGTACCTGATCGAGAAAATCAAGCTGCATGATATGC | |
| | AGGAAGGGAAGTGTCTCTACTCCCTGGAGGCCATTCCGCTGG | |
| | AGGATTTGCTGAACAACCCTTTCAACTACGAAGTCGATCATA | |
| | TCATTCCTCGCTCCGTGTCCTTCGATAACTCCTTCAACAATAA | |
| | GGTCCTCGTGAAGCAGGAGGAGAAGTAAGTATCAAGGTTAC | |
| | AAGACAGCTATTCTGAGTACAGAGCATACAGAGTCTTGTCGA | |
| | GACAGAGAAGACTCTTGCGTTTCTGATAGGCACCTATTGGTC | |
| | TTACTGACATCCACTTTGCCTTTCTCTCCACAGCTCGAAGAAG | |
| | GGCAACAGAACCCCGTTCCAGTACCTCTCGTCGTCCGACTCC | |
| | AAGATCAGCTACGAAACTTTCAAGAAGCACATTCTGAACCTG | |
| | GCCAAGGGCAAAGGGAGAATTAGCAAGACCAAGAAGGAATA | |
| | CCTCCTGGAAGAGAGAGACATCAACCGCTTCTCGGTGCAAAA | |
| | GGATTTCATCAACCGCAACCTGGTCGATACCAGATACGCCAC | |
| | CAGGGGACTGATGAACCTCCTGCGGTCCTACTTCCGGGTCAA | |
| | CAATCTGGACGTGAAGGTCAAATCCATCAACGGGGGCTTTAC | |
| | TTCTTTCCTGCGCCGGAAGTGGAAGTTCAAGAAGGAACGGAA | |
| | CAAGGGATACAAGCACCACGCTGAAGATGCCCTGATTATTGC | |
| | CAACGCCGACTTCATCTTTAAGGAATGGAAAAAGCTGGACAA | |
| | GGCTAAGAAGGTCATGGAGAACCAGATGTTCGAAGAAAAGC | |
| | AGGCCGAGTCCATGCCCGAAATCGAAACCGAGCAGGAATAC | |
| | AAGGAGATCTTCATCACACCGCACCAAATCAAGCACATCAAG | |
| | GACTTCAAGGATTACAAGTACAGCCACCGGGTGGACAAGAA | |
| | GCCTAACAGAGAGCTTATCAACGACACCCTGTACTCCACGCG | |
| | CAAGGACGACAAGGGAAACACATTGATCGTGAACAACCTGA | |
| | ACGGACTGTATGACAAGGACAATGACAAACTGAAGAAGCTG | |
| | ATCAACAAATCGCCGGAAAAGCTCCTGATGTACCATCACGAC | |
| | CCTCAAACCTACCAGAAACTGAAGCTCATCATGGAGCAGTAC | |
| | GGCGACGAAAAGAATCCCCTGTACAAATACTACGAGGAGAC | |
| | TGGAAATTACCTGACTAAGTACTCCAAGAAGGATAACGGCCC | |
| | CGTGATCAAGAAGATTAAGTACTACGGAAACAAACTGAACG | |
| | CACATCTCGACATCACCGATGATTATCCAAACTCCCGCAACA | |
| | AAGTCGTGAAGCTCTCCCTCAAACCGTACCGCTTCGACGTGT | |
| | ACCTGGATAATGGGGTGTACAAGTTCGTGACCGTGAAGAACC | |
| | TGGACGTCATTAAGAAGGAAAACTACTACGAAGTGAACTCA | |
| | AAGTGCTACGAGGAAGCCAAGAAGCTCAAGAAGATCAGCAA | |
| | CCAGGCCGAGTTCATCGCATCGTTTTACAACAATGACCTCAT | |
| | TAAGATTAATGGAGAACTGTACAGAGTGATCGGCGTGAACA | |
| | ACGACCTCCTGAACCGGATTGAAGTGAACATGATCGATATTA | |
| | CCTACCGGGAGTATCTGGAGAACATGAACGACAAGCGCCCA | |
| | CCGAGAATCATCAAAACTATTGCCTCCAAGACCCAATCCATT | |
| | AAGAAATACTCCACCGACATCCTGGGCAACCTGTACGAGGTC | |
| | AAGTCGAAGAAGCACCCCCAGATTATCAAGAAGGGAAAGCT | |
| | TGCCCCAAAGAAGAAGCGGAAGGTCTAA | |
| Poly A | AATAAAATATCTTTATTTTCATTACATCTGTGTGTTGGTTTTTT | 58 |
| | GTGTG | |
| 3′ITR | AGGAACCCCTAGTGATGGAGTTGGCCACTCCCTCTCTGCGCG | 43 |
| | CTCGCTCGCTCACTGAGGCCGGGCGACCAAAGGTCGCCCGAC | |
| | GCCCGGGCTTTGCCCGGGCGGCCTCAGTGAGCGAGCGAGCGC | |
| | GCAGCTGCCTGCAGG | |

TABLE 9
Summary of Vector Sequences

| Vector | Sequence | SEQ ID NO: |
|---|---|---|
| CTX-212 | CCTGCAGGCAGCTGCGCGCTCGCTCGCTCACTGAGGCCGCCCGGGCG | 59 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGGTGTGGAAAGTCCCCAGGCTCCCCAGCAGGCAGAAGTATG | |
| | CAAAGCATGCATCTCAATTAGTCAGCAACCACGTTACATAACTTACG | |
| | GTAAATGGCCCGCCTGGCTGACCGCCCAACGACCCCCGCCCATTGAC | |
| | GTCAATAATGACGTATGTTCCCATAGTAACGCCAATAGGGACTTTCC | |
| | ATTGACGTCAATGGGTGGAGTATTTACGGTAAACTGCCCACTTGGCA | |
| | GTACATCAAGTGTATCATATGCCAAGTACGCCCCCTATTGACGTCAA | |
| | TGACGGTAAATGGCCCGCCTGGCATTATGCCCAGTACATGACCTTAT | |
| | GGGACTTTCCTACTTGGCAGTACATCTACGTATTAGTCATCGCTATTA | |
| | CCATGGTGATGCGGTTTTGGCAGTACATCAATGGGCGTGGATAGCGG | |
| | TTTGACTCACGGGGATTTCCAAGTCTCCACCCCATTGACGTCAATGG | |
| | GAGTTTGTTTTGGCACCAAAATCAACGGGACTTTCCAAAATGTCGTA | |
| | ACAACTCCGCCCCATTGACGCAAATGGGCGGTAGGCGTGTACGGTG | |
| | GGAGGTCTATATAAGCAGAGCTCGTTTAGTGAACCGTCACCGGTGTA | |
| | TTGCTTGTACTACTCACTGAATGCCACCATGGCCCCAAAGAAGAAGC | |
| | GGAAGGTCGGATCCGGAAAGCGGAACTATATCCTGGGACTGGACAT | |
| | CGGAATTACCTCCGTGGGATACGGCATCATCGATTACGAGACTAGG | |
| | GACGTGATTGACGCCGGCGTGAGACTCTTTAAGGAGGCCAACGTGG | |
| | AAAACAACGAAGGTCGCAGATCCAAGCGGGGTGCAAGACGCCTGAA | |
| | GCGCCGGAGGAGACATCGGATACAGCGCGTGAAGAAGCTCCTTTTC | |
| | GACTACAACCTCCTCACTGACCACTCGGAATTGTCCGGTATCAACCC | |
| | CTACGAAGCCCGCGTGAAAGGCCTGAGCCAGAAGCTGTCCGAAGAG | |
| | GAGTTTAGCGCAGCCCTGCTGCACCTGGCTAAGCGAAGGGGGGTGC | |
| | ACAACGTGAACGAGGTGGAGGAGGACACTGGCAACGAACTGTCCAC | |
| | CAAGGAGCAGATTTCACGGAACTCGAAGGCGCTGGAAGAGAAATAT | |
| | GTGGCCGAGCTGCAGCTGGAGAGGCTCAAGAAGGATGGCGAAGTCC | |
| | GGGGGAGCATCAATCGCTTCAAGACCTCGGACTACGTGAAGGAAGC | |
| | CAAACAGCTGTTGAAGGTGCAGAAGGCCTACCACCAACTGGACCAA | |
| | TCATTCATTGACACTTACATCGATCTGCTTGAAACCAGGCGCACCTA | |
| | CTACGAGGGTCCTGGAGAAGGCAGCCCTTTCGGATGGAAGGACATC | |
| | AAGGAGTGGTATGAGATGCTGATGGGTCATTGCACCTACTTTCCGGA | |
| | AGAACTGCGCTCAGTGAAGTACGCGTACAACGCTGACCTCTACAAC | |
| | GCTCTCAACGATCTGAACAACCTCGTGATCACCCGGGACGAGAACG | |
| | AAAAGCTGGAGTACTACGAAAAGTTCCAGATTATCGAAAACGTGTT | |
| | CAAGCAGAAGAAGAAGCCCACCCTGAAGCAGATTGCAAAGGAGATC | |
| | CTTGTGAACGAGGAGGATATTAAGGGCTACCGGGTCACCTCCACCG | |
| | GGAAACCAGAGTTCACTAATCTCAAGGTGTACCATGACATTAAGGA | |
| | CATTACTGCCCGCAAGGAGATCATTGAAAACGCGGAACTGCTGGAC | |
| | CAAATCGCGAAGATCCTGACCATCTATCAGAGCTCCGAGGATATCCA | |
| | GGAGGAACTTACTAACCTCAATTCCGAGCTGACGCAGGAAGAAATC | |
| | GAGCAAATTAGCAACCTGAAGGGTTACACTGGAACCCACAACCTCA | |
| | GCTTGAAAGCGATTAACCTTATTTTGGATGAACTTTGGCACACTAAT | |
| | GACAATCAGATCGCCATTTTCAACCGGCTGAAACTGGTGCCGAAGA | |
| | AGGTGGACCTGAGCCAACAGAAGGAAATCCCGACCACCCTTGTGGA | |
| | CGATTTCATCCTGTCACCTGTGGTGAAGAGGAGCTTCATCCAGTCGA | |
| | TCAAGGTCATCAACGCCATCATAAAGAAGTACGGCCTTCCCAACGA | |
| | CATCATCATCGAACTGGCCCGCGAGAAGAACTCCAAAGATGCCCAG | |
| | AAGATGATCAACGAGATGCAGAAGCGAAACCGGCAGACGAACGAA | |
| | CGGATCGAGGAGATCATCCGGACCACCGGGAAGGAAAACGCGAAGT | |
| | ACCTGATCGAGAAAATCAAGCTGCATGATATGCAGGAAGGGAAGTG | |
| | TCTCTACTCCCTGGAGGCCATTCCGCTGGAGGATTTGCTGAACAACC | |
| | CTTTCAACTACGAAGTCGATCATATCATTCCTCGCTCCGTGTCCTTCG | |
| | ATAACTCCTTCAACAATAAGGTCCTCGTGAAGCAGGAGGAGAAGTA | |
| | AGTATCAAGGTTACAAGACAGGTGTATTGCTTGTACTACTCACTGAA | |
| | TCTTGTCGAGACAGAGAAGACTCTTGCGTTTCTGATAGGCACCTATT | |
| | GGTCTTACTGACATCCACTTTGCCTTTCTCTCCACAGCTCGAAGAAG | |
| | GGCAACAGAACCCCGTTCCAGTACCTCTCGTCGTCCGACTCCAAGAT | |
| | CAGCTACGAAACTTTCAAGAAGCACATTCTGAACCTGGCCAAGGGC | |
| | AAAGGGAGAATTAGCAAGACCAAGAAGGAATACCTCCTGGAAGAG | |
| | AGAGACATCAACCGCTTCTCGGTGCAAAAGGATTTCATCAACCGCA | |
| | ACCTGGTCGATACCAGATACGCCACCAGGGGACTGATGAACCTCCT | |
| | GCGGTCCTACTTCCGGGTCAACAATCTGGACGTGAAGGTCAAATCCA | |
| | TCAACGGGGGCTTTACTTCTTTCCTGCGCCGGAAGTGGAAGTTCAAG | |
| | AAGGAACGGAACAAGGGATACAAGCACCACGCTGAAGATGCCCTGA | |
| | TTATTGCCAACGCCGACTTCATCTTTAAGGAATGGAAAAAGCTGGAC | |
| | AAGGCTAAGAAGGTCATGGAGAACCAGATGTTCGAAGAAAAGCAG | |
| | GCCGAGTCCATGCCCGAAATCGAAACCGAGCAGGAATACAAGGAGA | |
| | TCTTCATCACACCGCACCAAATCAAGCACATCAAGGACTTCAAGGAT | |
| | TACAAGTACAGCCACCGGGTGGACAAGAAGCCTAACAGAGAGCTTA | |
| | TCAACGACACCCTGTACTCCACGCGCAAGGACGACAAGGGAAACAC | |
| | ATTGATCGTGAACAACCTGAACGGACTGTATGACAAGGACAATGAC | |
| | AAACTGAAGAAGCTGATCAACAAATCGCCGGAAAAGCTCCTGATGT | |
| | ACCATCACGACCCTCAAACCTACCAGAAACTGAAGCTCATCATGGA | |
| | GCAGTACGGCGACGAAAAGAATCCCCTGTACAAATACTACGAGGAG | |
| | ACTGGAAATTACCTGACTAAGTACTCCAAGAAGGATAACGGCCCCG | |
| | TGATCAAGAAGATTAAGTACTACGGAAACAAACTGAACGCACATCT | |
| | CGACATCACCGATGATTATCCAAACTCCCGCAACAAAGTCGTGAAG | |
| | CTCTCCCTCAAACCGTACCGCTTCGACGTGTACCTGGATAATGGGGT | |
| | GTACAAGTTCGTGACCGTGAAGAACCTGGACGTCATTAAGAAGGAA | |
| | AACTACTACGAAGTGAACTCAAAGTGCTACGAGGAAGCCAAGAAGC | |
| | TCAAGAAGATCAGCAACCAGGCCGAGTTCATCGCATCGTTTTACAAC | |
| | AATGACCTCATTAAGATTAATGGAGAACTGTACAGAGTGATCGGCGT | |
| | GAACAACGACCTCCTGAACCGGATTGAAGTGAACATGATCGATATT | |
| | ACCTACCGGGAGTATCTGGAGAACATGAACGACAAGCGCCCACCGA | |
| | GAATCATCAAAACTATTGCCTCCAAGACCCAATCCATTAAGAAATAC | |
| | TCCACCGACATCCTGGGCAACCTGTACGAGGTCAAGTCGAAGAAGC | |
| | ACCCCCAGATTATCAAGAAGGGAAAGCTTGCCCCAAAGAAGAAGCG | |
| | GAAGGTCGGTACTAGTGAGGGCAGGGGAAGTCTGCTAACATGCGGG | |
| | GACGTGGAGGAAAATCCCGGCCCCATGGCTAAGACTTCCGAACAGA | |
| | GGGTGAACATTGCTACACTGCTGACAGAAAATAAGAAGAAAATCGT | |
| | GGATAAGGCTTCCCAGGATCTGTGGCGGAGACACCCAGACCTGATC | |
| | GCACCAGGAGGAATTGCTTTCTCTCAGAGGGACCGCGCTCTGTGCCT | |
| | GCGAGATTACGGCTGGTTCCTGCATCTGATCACCTTTTGTCTGCTGGC | |
| | CGGAGATAAGGGCCCCATCGAGTCTATTGGGCTGATCAGTATTCGAG | |
| | AAATGTATAACTCACTGGGAGTGCCCGTCCCTGCAATGATGGAGAG | |
| | CATTAGATGCCTGAAAGAAGCCAGCCTGTCCCTGCTGGACGAAGAG | |
| | GACGCCAACGAGACCGCACCCTACTTTGATTACATTATTAAGGCTAT | |
| | GAGCTAAGCGCTGTGTTATTACTTGCTACTGCAGAGAGTAATAAAAT | |
| | ATCTTTATTTTCATTACATCTGTGTGTTGGTTTTTTGTGTGGTAACCAC | |
| | GTGCGGACCGAGGCTGCAGCGTCGTCCTCCCTAGGAACCCCTAGTGA | |
| | TGGAGTTGGCCACTCCCTCTCTGCGCGCTCGCTCGCTCACTGAGGCC | |
| | GGGCGACCAAAGGTCGCCCGACGCCCGGGCTTTGCCCGGGCGGCCT | |
| | CAGTGAGCGAGCGAGCGCGCAGCTGCCTGCAGG | |
| CTX-214 | CCTGCAGGCAGCTGCGCGCTCGCTCGCTCACTGAGGCCGCCCGGGCG | 60 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGGTGTGGAAAGTCCCCAGGCTCCCCAGCAGGCAGAAGTATG | |
| | CAAAGCATGCATCTCAATTAGTCAGCAACCACGTTACATAACTTACG | |
| | GTAAATGGCCCGCCTGGCTGACCGCCCAACGACCCCCGCCCATTGAC | |
| | GTCAATAATGACGTATGTTCCCATAGTAACGCCAATAGGGACTTTCC | |
| | ATTGACGTCAATGGGTGGAGTATTTACGGTAAACTGCCCACTTGGCA | |
| | GTACATCAAGTGTATCATATGCCAAGTACGCCCCCTATTGACGTCAA | |
| | TGACGGTAAATGGCCCGCCTGGCATTATGCCCAGTACATGACCTTAT | |
| | GGGACTTTCCTACTTGGCAGTACATCTACGTATTAGTCATCGCTATTA | |
| | CCATGGTGATGCGGTTTTGGCAGTACATCAATGGGCGTGGATAGCGG | |
| | TTTGACTCACGGGGATTTCCAAGTCTCCACCCCATTGACGTCAATGG | |
| | GAGTTTGTTTTGGCACCAAAATCAACGGGACTTTCCAAAATGTCGTA | |
| | ACAACTCCGCCCCATTGACGCAAATGGGCGGTAGGCGTGTACGGTG | |
| | GGAGGTCTATATAAGCAGAGCTCGTTTAGTGAACCGTCACCGGTGTA | |
| | TTGCTTGTACTACTCACTGAATGCCACCATGGCCCCAAAGAAGAAGC | |
| | GGAAGGTCGGATCCGGAAAGCGGAACTATATCCTGGGACTGGACAT | |
| | CGGAATTACCTCCGTGGGATACGGCATCATCGATTACGAGACTAGG | |
| | GACGTGATTGACGCCGGCGTGAGACTCTTTAAGGAGGCCAACGTGG | |
| | AAAACAACGAAGGTCGCAGATCCAAGCGGGGTGCAAGACGCCTGAA | |
| | GCGCCGGAGGAGACATCGGATACAGCGCGTGAAGAAGCTCCTTTTC | |
| | GACTACAACCTCCTCACTGACCACTCGGAATTGTCCGGTATCAACCC | |
| | CTACGAAGCCCGCGTGAAAGGCCTGAGCCAGAAGCTGTCCGAAGAG | |
| | GAGTTTAGCGCAGCCCTGCTGCACCTGGCTAAGCGAAGGGGGGTGC | |
| | ACAACGTGAACGAGGTGGAGGAGGACACTGGCAACGAACTGTCCAC | |
| | CAAGGAGCAGATTTCACGGAACTCGAAGGCGCTGGAAGAGAAATAT | |
| | GTGGCCGAGCTGCAGCTGGAGAGGCTCAAGAAGGATGGCGAAGTCC | |
| | GGGGGAGCATCAATCGCTTCAAGACCTCGGACTACGTGAAGGAAGC | |
| | CAAACAGCTGTTGAAGGTGCAGAAGGCCTACCACCAACTGGACCAA | |
| | TCATTCATTGACACTTACATCGATCTGCTTGAAACCAGGCGCACCTA | |
| | CTACGAGGGTCCTGGAGAAGGCAGCCCTTTCGGATGGAAGGACATC | |
| | AAGGAGTGGTATGAGATGCTGATGGGTCATTGCACCTACTTTCCGGA | |
| | AGAACTGCGCTCAGTGAAGTACGCGTACAACGCTGACCTCTACAAC | |
| | GCTCTCAACGATCTGAACAACCTCGTGATCACCCGGGACGAGAACG | |
| | AAAAGCTGGAGTACTACGAAAAGTTCCAGATTATCGAAAACGTGTT | |
| | CAAGCAGAAGAAGAAGCCCACCCTGAAGCAGATTGCAAAGGAGATC | |
| | CTTGTGAACGAGGAGGATATTAAGGGCTACCGGGTCACCTCCACCG | |
| | GGAAACCAGAGTTCACTAATCTCAAGGTGTACCATGACATTAAGGA | |
| | CATTACTGCCCGCAAGGAGATCATTGAAAACGCGGAACTGCTGGAC | |
| | CAAATCGCGAAGATCCTGACCATCTATCAGAGCTCCGAGGATATCCA | |
| | GGAGGAACTTACTAACCTCAATTCCGAGCTGACGCAGGAAGAAATC | |
| | GAGCAAATTAGCAACCTGAAGGGTTACACTGGAACCCACAACCTCA | |
| | GCTTGAAAGCGATTAACCTTATTTTGGATGAACTTTGGCACACTAAT | |
| | GACAATCAGATCGCCATTTTCAACCGGCTGAAACTGGTGCCGAAGA | |
| | AGGTGGACCTGAGCCAACAGAAGGAAATCCCGACCACCCTTGTGGA | |
| | CGATTTCATCCTGTCACCTGTGGTGAAGAGGAGCTTCATCCAGTCGA | |
| | TCAAGGTCATCAACGCCATCATAAAGAAGTACGGCCTTCCCAACGA | |
| | CATCATCATCGAACTGGCCCGCGAGAAGAACTCCAAAGATGCCCAG | |
| | AAGATGATCAACGAGATGCAGAAGCGAAACCGGCAGACGAACGAA | |
| | CGGATCGAGGAGATCATCCGGACCACCGGGAAGGAAAACGCGAAGT | |
| | ACCTGATCGAGAAAATCAAGCTGCATGATATGCAGGAAGGGAAGTG | |
| | TCTCTACTCCCTGGAGGCCATTCCGCTGGAGGATTTGCTGAACAACC | |
| | CTTTCAACTACGAAGTCGATCATATCATTCCTCGCTCCGTGTCCTTCG | |
| | ATAACTCCTTCAACAATAAGGTCCTCGTGAAGCAGGAGGAGAAGTA | |
| | AGTATCAAGGTTACAAGACAGGTGTTATTACTTGCTACTGCAGAGAG | |
| | TCTTGTCGAGACAGAGAAGACTCTTGCGTTTCTGATAGGCACCTATT | |
| | GGTCTTACTGACATCCACTTTGCCTTTCTCTCCACAGCTCGAAGAAG | |
| | GGCAACAGAACCCCGTTCCAGTACCTCTCGTCGTCCGACTCCAAGAT | |
| | CAGCTACGAAACTTTCAAGAAGCACATTCTGAACCTGGCCAAGGGC | |
| | AAAGGGAGAATTAGCAAGACCAAGAAGGAATACCTCCTGGAAGAG | |
| | AGAGACATCAACCGCTTCTCGGTGCAAAAGGATTTCATCAACCGCA | |
| | ACCTGGTCGATACCAGATACGCCACCAGGGGACTGATGAACCTCCT | |
| | GCGGTCCTACTTCCGGGTCAACAATCTGGACGTGAAGGTCAAATCCA | |
| | TCAACGGGGGCTTTACTTCTTTCCTGCGCCGGAAGTGGAAGTTCAAG | |
| | AAGGAACGGAACAAGGGATACAAGCACCACGCTGAAGATGCCCTGA | |
| | TTATTGCCAACGCCGACTTCATCTTTAAGGAATGGAAAAAGCTGGAC | |
| | AAGGCTAAGAAGGTCATGGAGAACCAGATGTTCGAAGAAAAGCAG | |
| | GCCGAGTCCATGCCCGAAATCGAAACCGAGCAGGAATACAAGGAGA | |
| | TCTTCATCACACCGCACCAAATCAAGCACATCAAGGACTTCAAGGAT | |
| | TACAAGTACAGCCACCGGGTGGACAAGAAGCCTAACAGAGAGCTTA | |
| | TCAACGACACCCTGTACTCCACGCGCAAGGACGACAAGGGAAACAC | |
| | ATTGATCGTGAACAACCTGAACGGACTGTATGACAAGGACAATGAC | |
| | AAACTGAAGAAGCTGATCAACAAATCGCCGGAAAAGCTCCTGATGT | |
| | ACCATCACGACCCTCAAACCTACCAGAAACTGAAGCTCATCATGGA | |
| | GCAGTACGGCGACGAAAAGAATCCCCTGTACAAATACTACGAGGAG | |
| | ACTGGAAATTACCTGACTAAGTACTCCAAGAAGGATAACGGCCCCG | |
| | TGATCAAGAAGATTAAGTACTACGGAAACAAACTGAACGCACATCT | |
| | CGACATCACCGATGATTATCCAAACTCCCGCAACAAAGTCGTGAAG | |
| | CTCTCCCTCAAACCGTACCGCTTCGACGTGTACCTGGATAATGGGGT | |
| | GTACAAGTTCGTGACCGTGAAGAACCTGGACGTCATTAAGAAGGAA | |
| | AACTACTACGAAGTGAACTCAAAGTGCTACGAGGAAGCCAAGAAGC | |
| | TCAAGAAGATCAGCAACCAGGCCGAGTTCATCGCATCGTTTTACAAC | |
| | AATGACCTCATTAAGATTAATGGAGAACTGTACAGAGTGATCGGCGT | |
| | GAACAACGACCTCCTGAACCGGATTGAAGTGAACATGATCGATATT | |
| | ACCTACCGGGAGTATCTGGAGAACATGAACGACAAGCGCCCACCGA | |
| | GAATCATCAAAACTATTGCCTCCAAGACCCAATCCATTAAGAAATAC | |
| | TCCACCGACATCCTGGGCAACCTGTACGAGGTCAAGTCGAAGAAGC | |
| | ACCCCCAGATTATCAAGAAGGGAAAGCTTGCCCCAAAGAAGAAGCG | |
| | GAAGGTCGGTACTAGTGAGGGCAGGGGAAGTCTGCTAACATGCGGG | |
| | GACGTGGAGGAAAATCCCGGCCCCATGGCTAAGACTTCCGAACAGA | |
| | GGGTGAACATTGCTACACTGCTGACAGAAAATAAGAAGAAAATCGT | |
| | GGATAAGGCTTCCCAGGATCTGTGGCGGAGACACCCAGACCTGATC | |
| | GCACCAGGAGGAATTGCTTTCTCTCAGAGGGACCGCGCTCTGTGCCT | |
| | GCGAGATTACGGCTGGTTCCTGCATCTGATCACCTTTTGTCTGCTGGC | |
| | CGGAGATAAGGGCCCCATCGAGTCTATTGGGCTGATCAGTATTCGAG | |
| | AAATGTATAACTCACTGGGAGTGCCCGTCCCTGCAATGATGGAGAG | |
| | CATTAGATGCCTGAAAGAAGCCAGCCTGTCCCTGCTGGACGAAGAG | |
| | GACGCCAACGAGACCGCACCCTACTTTGATTACATTATTAAGGCTAT | |
| | GAGCTAAGCGCTAATAAAATATCTTTATTTTCATTACATCTGTGTGTT | |
| | GGTTTTTTGTGTGGTAACCACGTGCGGACCGAGGCTGCAGCGTCGTC | |
| | CTCCCTAGGAACCCCTAGTGATGGAGTTGGCCACTCCCTCTCTGCGC | |
| | GCTCGCTCGCTCACTGAGGCCGGGCGACCAAAGGTCGCCCGACGCC | |
| | CGGGCTTTGCCCGGGCGGCCTCAGTGAGCGAGCGAGCGCGCAGCTG | |
| | CCTGCAGG | |
| CTX-217 | CCTGCAGGCAGCTGCGCGCTCGCTCGCTCACTGAGGCCGCCCGGGCG | 61 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGGTGTGGAAAGTCCCCAGGCTCCCCAGCAGGCAGAAGTATG | |
| | CAAAGCATGCATCTCAATTAGTCAGCAACCACGTTACATAACTTACG | |
| | GTAAATGGCCCGCCTGGCTGACCGCCCAACGACCCCCGCCCATTGAC | |
| | GTCAATAATGACGTATGTTCCCATAGTAACGCCAATAGGGACTTTCC | |
| | ATTGACGTCAATGGGTGGAGTATTTACGGTAAACTGCCCACTTGGCA | |
| | GTACATCAAGTGTATCATATGCCAAGTACGCCCCCTATTGACGTCAA | |
| | TGACGGTAAATGGCCCGCCTGGCATTATGCCCAGTACATGACCTTAT | |
| | GGGACTTTCCTACTTGGCAGTACATCTACGTATTAGTCATCGCTATTA | |
| | CCATGGTGATGCGGTTTTGGCAGTACATCAATGGGCGTGGATAGCGG | |
| | TTTGACTCACGGGGATTTCCAAGTCTCCACCCCATTGACGTCAATGG | |
| | GAGTTTGTTTTGGCACCAAAATCAACGGGACTTTCCAAAATGTCGTA | |
| | ACAACTCCGCCCCATTGACGCAAATGGGCGGTAGGCGTGTACGGTG | |
| | GGAGGTCTATATAAGCAGAGCTCGTTTAGTGAACCGTCACCGGTGTA | |
| | TTGCTTGTACTACTCACTGAATGCCACCATGGCCCCAAAGAAGAAGC | |
| | GGAAGGTCGGATCCGGAAAGCGGAACTATATCCTGGGACTGGGTAA | |
| | GTGTATTGCTTGTACTACTCACTGAATCACCATCGGGCGCGAAGGGG | |
| | GAGACCTGTAGTCAGAGCCCCCGGGCAGCACACACTGACATCCACT | |
| | CCCTTCCTATTGTTTCAGACATCGGAATTACCTCCGTGGGATACGGC | |
| | ATCATCGATTACGAGACTAGGGACGTGATTGACGCCGGCGTGAGAC | |
| | TCTTTAAGGAGGCCAACGTGGAAAACAACGAAGGTCGCAGATCCAA | |
| | GCGGGGTGCAAGACGCCTGAAGCGCCGGAGGAGACATCGGATACAG | |
| | CGCGTGAAGAAGCTCCTTTTCGACTACAACCTCCTCACTGACCACTC | |
| | GGAATTGTCCGGTATCAACCCCTACGAAGCCCGCGTGAAAGGCCTG | |
| | AGCCAGAAGCTGTCCGAAGAGGAGTTTAGCGCAGCCCTGCTGCACC | |
| | TGGCTAAGCGAAGGGGGGTGCACAACGTGAACGAGGTGGAGGAGG | |
| | ACACTGGCAACGAACTGTCCACCAAGGAGCAGATTTCACGGAACTC | |
| | GAAGGCGCTGGAAGAGAAATATGTGGCCGAGCTGCAGCTGGAGAGG | |
| | CTCAAGAAGGATGGCGAAGTCCGGGGGAGCATCAATCGCTTCAAGA | |
| | CCTCGGACTACGTGAAGGAAGCCAAACAGCTGTTGAAGGTGCAGAA | |
| | GGCCTACCACCAACTGGACCAATCATTCATTGACACTTACATCGATC | |
| | TGCTTGAAACCAGGCGCACCTACTACGAGGGTCCTGGAGAAGGCAG | |
| | CCCTTTCGGATGGAAGGACATCAAGGAGTGGTATGAGATGCTGATG | |
| | GGTCATTGCACCTACTTTCCGGAAGAACTGCGCTCAGTGAAGTACGC | |
| | GTACAACGCTGACCTCTACAACGCTCTCAACGATCTGAACAACCTCG | |
| | TGATCACCCGGGACGAGAACGAAAAGCTGGAGTACTACGAAAAGTT | |
| | CCAGATTATCGAAAACGTGTTCAAGCAGAAGAAGAAGCCCACCCTG | |
| | AAGCAGATTGCAAAGGAGATCCTTGTGAACGAGGAGGATATTAAGG | |
| | GCTACCGGGTCACCTCCACCGGGAAACCAGAGTTCACTAATCTCAAG | |
| | GTGTACCATGACATTAAGGACATTACTGCCCGCAAGGAGATCATTGA | |
| | AAACGCGGAACTGCTGGACCAAATCGCGAAGATCCTGACCATCTAT | |
| | CAGAGCTCCGAGGATATCCAGGAGGAACTTACTAACCTCAATTCCG | |
| | AGCTGACGCAGGAAGAAATCGAGCAAATTAGCAACCTGAAGGGTTA | |
| | CACTGGAACCCACAACCTCAGCTTGAAAGCGATTAACCTTATTTTGG | |
| | ATGAACTTTGGCACACTAATGACAATCAGATCGCCATTTTCAACCGG | |
| | CTGAAACTGGTGCCGAAGAAGGTGGACCTGAGCCAACAGAAGGAAA | |
| | TCCCGACCACCCTTGTGGACGATTTCATCCTGTCACCTGTGGTGAAG | |
| | AGGAGCTTCATCCAGTCGATCAAGGTCATCAACGCCATCATAAAGA | |
| | AGTACGGCCTTCCCAACGACATCATCATCGAACTGGCCCGCGAGAA | |
| | GAACTCCAAAGATGCCCAGAAGATGATCAACGAGATGCAGAAGCGA | |
| | AACCGGCAGACGAACGAACGGATCGAGGAGATCATCCGGACCACCG | |
| | GGAAGGAAAACGCGAAGTACCTGATCGAGAAAATCAAGCTGCATGA | |
| | TATGCAGGAAGGGAAGTGTCTCTACTCCCTGGAGGCCATTCCGCTGG | |
| | AGGATTTGCTGAACAACCCTTTCAACTACGAAGTCGATCATATCATT | |
| | CCTCGCTCCGTGTCCTTCGATAACTCCTTCAACAATAAGGTCCTCGTG | |
| | AAGCAGGAGGAGAAGTAAGTATCAAGGTTACAAGACAGGTGTTATT | |
| | ACTTGCTACTGCAGAGAGTCTTGTCGAGACAGAGAAGACTCTTGCGT | |
| | TTCTGATAGGCACCTATTGGTCTTACTGACATCCACTTTGCCTTTCTC | |
| | TCCACAGCTCGAAGAAGGGCAACAGAACCCCGTTCCAGTACCTCTC | |
| | GTCGTCCGACTCCAAGATCAGCTACGAAACTTTCAAGAAGCACATTC | |
| | TGAACCTGGCCAAGGGCAAAGGGAGAATTAGCAAGACCAAGAAGG | |
| | AATACCTCCTGGAAGAGAGAGACATCAACCGCTTCTCGGTGCAAAA | |
| | GGATTTCATCAACCGCAACCTGGTCGATACCAGATACGCCACCAGG | |
| | GGACTGATGAACCTCCTGCGGTCCTACTTCCGGGTCAACAATCTGGA | |
| | CGTGAAGGTCAAATCCATCAACGGGGGCTTTACTTCTTTCCTGCGCC | |
| | GGAAGTGGAAGTTCAAGAAGGAACGGAACAAGGGATACAAGCACC | |
| | ACGCTGAAGATGCCCTGATTATTGCCAACGCCGACTTCATCTTTAAG | |
| | GAATGGAAAAAGCTGGACAAGGCTAAGAAGGTCATGGAGAACCAG | |
| | ATGTTCGAAGAAAAGCAGGCCGAGTCCATGCCCGAAATCGAAACCG | |
| | AGCAGGAATACAAGGAGATCTTCATCACACCGCACCAAATCAAGCA | |
| | CATCAAGGACTTCAAGGATTACAAGTACAGCCACCGGGTGGACAAG | |
| | AAGCCTAACAGAGAGCTTATCAACGACACCCTGTACTCCACGCGCA | |
| | AGGACGACAAGGGAAACACATTGATCGTGAACAACCTGAACGGACT | |
| | GTATGACAAGGACAATGACAAACTGAAGAAGCTGATCAACAAATCG | |
| | CCGGAAAAGCTCCTGATGTACCATCACGACCCTCAAACCTACCAGA | |
| | AACTGAAGCTCATCATGGAGCAGTACGGCGACGAAAAGAATCCCCT | |
| | GTACAAATACTACGAGGAGACTGGAAATTACCTGACTAAGTACTCC | |
| | AAGAAGGATAACGGCCCCGTGATCAAGAAGATTAAGTACTACGGAA | |
| | ACAAACTGAACGCACATCTCGACATCACCGATGATTATCCAAACTCC | |
| | CGCAACAAAGTCGTGAAGCTCTCCCTCAAACCGTACCGCTTCGACGT | |
| | GTACCTGGATAATGGGGTGTACAAGTTCGTGACCGTGAAGAACCTG | |
| | GACGTCATTAAGAAGGAAAACTACTACGAAGTGAACTCAAAGTGCT | |
| | ACGAGGAAGCCAAGAAGCTCAAGAAGATCAGCAACCAGGCCGAGTT | |
| | CATCGCATCGTTTTACAACAATGACCTCATTAAGATTAATGGAGAAC | |
| | TGTACAGAGTGATCGGCGTGAACAACGACCTCCTGAACCGGATTGA | |
| | AGTGAACATGATCGATATTACCTACCGGGAGTATCTGGAGAACATG | |
| | AACGACAAGCGCCCACCGAGAATCATCAAAACTATTGCCTCCAAGA | |
| | CCCAATCCATTAAGAAATACTCCACCGACATCCTGGGCAACCTGTAC | |
| | GAGGTCAAGTCGAAGAAGCACCCCCAGATTATCAAGAAGGGAAAGC | |
| | TTGCCCCAAAGAAGAAGCGGAAGGTCGGTACTAGTGAGGGCAGGGG | |
| | AAGTCTGCTAACATGCGGGGACGTGGAGGAAAATCCCGGCCCCGCT | |
| | AAGACTTCCGAACAGAGGGTGAACATTGCTACACTGCTGACAGAAA | |
| | ATAAGAAGAAAATCGTGGATAAGGCTTCCCAGGATCTGTGGCGGAG | |
| | ACACCCAGACCTGATCGCACCAGGAGGAATTGCTTTCTCTCAGAGGG | |
| | ACCGCGCTCTGTGCCTGCGAGATTACGGCTGGTTCCTGCATCTGATC | |
| | ACCTTTTGTCTGCTGGCCGGAGATAAGGGCCCCATCGAGTCTATTGG | |
| | GCTGATCAGTATTCGAGAAATGTATAACTCACTGGGAGTGCCCGTCC | |
| | CTGCAATGATGGAGAGCATTAGATGCCTGAAAGAAGCCAGCCTGTC | |
| | CCTGCTGGACGAAGAGGACGCCAACGAGACCGCACCCTACTTTGAT | |
| | TACATTATTAAGGCTATGAGCTAAGCGCTGTGTTATTACTTGCTACTG | |
| | CAGAGAGTAATAAAATATCTTTATTTTCATTACATCTGTGTGTTGGTT | |
| | TTTTGTGTGGTAACCACGTGCGGACCGAGGCTGCAGCGTCGTCCTCC | |
| | CTAGGAACCCCTAGTGATGGAGTTGGCCACTCCCTCTCTGCGCGCTC | |
| | GCTCGCTCACTGAGGCCGGGCGACCAAAGGTCGCCCGACGCCCGGG | |
| | CTTTGCCCGGGCGGCCTCAGTGAGCGAGCGAGCGCGCAGCTGCCTG | |
| | CAGG | |
| CTX-506 | CCTGCAGGCAGCTGCGCGCTCGCTCGCTCACTGAGGCCGCCCGGGCG | 62 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGCTTAGAGGTCTTCTACATACAGTTTAAGT | |
| | ACTCTGTGCTGGAAACAGCACAGAATCTACTTAAACAAGGCAAAAT | |
| | GCCGTGTTTATCTCGTCAACTTGTTGGCGAGATTTTTTTCACCGGTGG | |
| | TGTGGAAAGTCCCCAGGCTCCCCAGCAGGCAGAAGTATGCAAAGCA | |
| | TGCATCTCAATTAGTCAGCAACCACGTTACATAACTTACGGTAAATG | |
| | GCCCGCCTGGCTGACCGCCCAACGACCCCCGCCCATTGACGTCAATA | |
| | ATGACGTATGTTCCCATAGTAACGCCAATAGGGACTTTCCATTGACG | |
| | TCAATGGGTGGAGTATTTACGGTAAACTGCCCACTTGGCAGTACATC | |
| | AAGTGTATCATATGCCAAGTACGCCCCCTATTGACGTCAATGACGGT | |
| | AAATGGCCCGCCTGGCATTATGCCCAGTACATGACCTTATGGGACTT | |
| | TCCTACTTGGCAGTACATCTACGTATTAGTCATCGCTATTACCATGGT | |
| | GATGCGGTTTTGGCAGTACATCAATGGGCGTGGATAGCGGTTTGACT | |
| | CACGGGGATTTCCAAGTCTCCACCCCATTGACGTCAATGGGAGTTTG | |
| | TTTTGGCACCAAAATCAACGGGACTTTCCAAAATGTCGTAACAACTC | |
| | CGCCCCATTGACGCAAATGGGCGGTAGGCGTGTACGGTGGGAGGTC | |
| | TATATAAGCAGAGCTCGTTTAGTGAACCGTCACCGGTGCCACCATGG | |
| | CCCCAAAGAAGAAGCGGAAGGTCGGATCCGGAAAGCGGAACTATAT | |
| | CCTGGGACTGGACATCGGAATTACCTCCGTGGGATACGGCATCATCG | |
| | ATTACGAGACTAGGGACGTGATTGACGCCGGCGTGAGACTCTTTAA | |
| | GGAGGCCAACGTGGAAAACAACGAAGGTCGCAGATCCAAGCGGGG | |
| | TGCAAGACGCCTGAAGCGCCGGAGGAGACATCGGATACAGCGCGTG | |
| | AAGAAGCTCCTTTTCGACTACAACCTCCTCACTGACCACTCGGAATT | |
| | GTCCGGTATCAACCCCTACGAAGCCCGCGTGAAAGGCCTGAGCCAG | |
| | AAGCTGTCCGAAGAGGAGTTTAGCGCAGCCCTGCTGCACCTGGCTA | |
| | AGCGAAGGGGGGTGCACAACGTGAACGAGGTGGAGGAGGACACTG | |
| | GCAACGAACTGTCCACCAAGGAGCAGATTTCACGGAACTCGAAGGC | |
| | GCTGGAAGAGAAATATGTGGCCGAGCTGCAGCTGGAGAGGCTCAAG | |
| | AAGGATGGCGAAGTCCGGGGGAGCATCAATCGCTTCAAGACCTCGG | |
| | ACTACGTGAAGGAAGCCAAACAGCTGTTGAAGGTGCAGAAGGCCTA | |
| | CCACCAACTGGACCAATCATTCATTGACACTTACATCGATCTGCTTG | |
| | AAACCAGGCGCACCTACTACGAGGGTCCTGGAGAAGGCAGCCCTTT | |
| | CGGATGGAAGGACATCAAGGAGTGGTATGAGATGCTGATGGGTCAT | |
| | TGCACCTACTTTCCGGAAGAACTGCGCTCAGTGAAGTACGCGTACAA | |
| | CGCTGACCTCTACAACGCTCTCAACGATCTGAACAACCTCGTGATCA | |
| | CCCGGGACGAGAACGAAAAGCTGGAGTACTACGAAAAGTTCCAGAT | |
| | TATCGAAAACGTGTTCAAGCAGAAGAAGAAGCCCACCCTGAAGCAG | |
| | ATTGCAAAGGAGATCCTTGTGAACGAGGAGGATATTAAGGGCTACC | |
| | GGGTCACCTCCACCGGGAAACCAGAGTTCACTAATCTCAAGGTGTAC | |
| | CATGACATTAAGGACATTACTGCCCGCAAGGAGATCATTGAAAACG | |
| | CGGAACTGCTGGACCAAATCGCGAAGATCCTGACCATCTATCAGAG | |
| | CTCCGAGGATATCCAGGAGGAACTTACTAACCTCAATTCCGAGCTGA | |
| | CGCAGGAAGAAATCGAGCAAATTAGCAACCTGAAGGGTTACACTGG | |
| | AACCCACAACCTCAGCTTGAAAGCGATTAACCTTATTTTGGATGAAC | |
| | TTTGGCACACTAATGACAATCAGATCGCCATTTTCAACCGGCTGAAA | |
| | CTGGTGCCGAAGAAGGTGGACCTGAGCCAACAGAAGGAAATCCCGA | |
| | CCACCCTTGTGGACGATTTCATCCTGTCACCTGTGGTGAAGAGGAGC | |
| | TTCATCCAGTCGATCAAGGTCATCAACGCCATCATAAAGAAGTACGG | |
| | CCTTCCCAACGACATCATCATCGAACTGGCCCGCGAGAAGAACTCCA | |
| | AAGATGCCCAGAAGATGATCAACGAGATGCAGAAGCGAAACCGGC | |
| | AGACGAACGAACGGATCGAGGAGATCATCCGGACCACCGGGAAGG | |
| | AAAACGCGAAGTACCTGATCGAGAAAATCAAGCTGCATGATATGCA | |
| | GGAAGGGAAGTGTCTCTACTCCCTGGAGGCCATTCCGCTGGAGGATT | |
| | TGCTGAACAACCCTTTCAACTACGAAGTCGATCATATCATTCCTCGC | |
| | TCCGTGTCCTTCGATAACTCCTTCAACAATAAGGTCCTCGTGAAGCA | |
| | GGAGGAGAACTCGAAGAAGGGCAACAGAACCCCGTTCCAGTACCTC | |
| | TCGTCGTCCGACTCCAAGATCAGCTACGAAACTTTCAAGAAGCACAT | |
| | TCTGAACCTGGCCAAGGGCAAAGGGAGAATTAGCAAGACCAAGAAG | |
| | GAATACCTCCTGGAAGAGAGAGACATCAACCGCTTCTCGGTGCAAA | |
| | AGGATTTCATCAACCGCAACCTGGTCGATACCAGATACGCCACCAG | |
| | GGGACTGATGAACCTCCTGCGGTCCTACTTCCGGGTCAACAATCTGG | |
| | ACGTGAAGGTCAAATCCATCAACGGGGGCTTTACTTCTTTCCTGCGC | |
| | CGGAAGTGGAAGTTCAAGAAGGAACGGAACAAGGGATACAAGCAC | |
| | CACGCTGAAGATGCCCTGATTATTGCCAACGCCGACTTCATCTTTAA | |
| | GGAATGGAAAAAGCTGGACAAGGCTAAGAAGGTCATGGAGAACCA | |
| | GATGTTCGAAGAAAAGCAGGCCGAGTCCATGCCCGAAATCGAAACC | |
| | GAGCAGGAATACAAGGAGATCTTCATCACACCGCACCAAATCAAGC | |
| | ACATCAAGGACTTCAAGGATTACAAGTACAGCCACCGGGTGGACAA | |
| | GAAGCCTAACAGAGAGCTTATCAACGACACCCTGTACTCCACGCGC | |
| | AAGGACGACAAGGGAAACACATTGATCGTGAACAACCTGAACGGAC | |
| | TGTATGACAAGGACAATGACAAACTGAAGAAGCTGATCAACAAATC | |
| | GCCGGAAAAGCTCCTGATGTACCATCACGACCCTCAAACCTACCAG | |
| | AAACTGAAGCTCATCATGGAGCAGTACGGCGACGAAAAGAATCCCC | |
| | TGTACAAATACTACGAGGAGACTGGAAATTACCTGACTAAGTACTCC | |
| | AAGAAGGATAACGGCCCCGTGATCAAGAAGATTAAGTACTACGGAA | |
| | ACAAACTGAACGCACATCTCGACATCACCGATGATTATCCAAACTCC | |
| | CGCAACAAAGTCGTGAAGCTCTCCCTCAAACCGTACCGCTTCGACGT | |
| | GTACCTGGATAATGGGGTGTACAAGTTCGTGACCGTGAAGAACCTG | |
| | GACGTCATTAAGAAGGAAAACTACTACGAAGTGAACTCAAAGTGCT | |
| | ACGAGGAAGCCAAGAAGCTCAAGAAGATCAGCAACCAGGCCGAGTT | |
| | CATCGCATCGTTTTACAACAATGACCTCATTAAGATTAATGGAGAAC | |
| | TGTACAGAGTGATCGGCGTGAACAACGACCTCCTGAACCGGATTGA | |
| | AGTGAACATGATCGATATTACCTACCGGGAGTATCTGGAGAACATG | |
| | AACGACAAGCGCCCACCGAGAATCATCAAAACTATTGCCTCCAAGA | |
| | CCCAATCCATTAAGAAATACTCCACCGACATCCTGGGCAACCTGTAC | |
| | GAGGTCAAGTCGAAGAAGCACCCCCAGATTATCAAGAAGGGAAAGC | |
| | TTGCCCCAAAGAAGAAGCGGAAGGTCTAAGGTACTAGTAATAAAAT | |
| | ATCTTTATTTTCATTACATCTGTGTGTTGGTTTTTTGTGTGAGCGCTG | |
| | AGGGCCTATTTCCCATGATTCCTTCATATTTGCATATACGATACAAG | |
| | GCTGTTAGAGAGATAATTGGAATTAATTTGACTGTAAACACAAAGAT | |
| | ATTAGTACAAAATACGTGACGTAGAAAGTAATAATTTCTTGGGTAGT | |
| | TTGCAGTTTTAAAATTATGTTTTAAAATGGACTATCATATGCTTACCG | |
| | TAACTTGAAAGTATTTCGATTTCTTGGCTTTATATATCTTGTGGAAAG | |
| | GACGAAACACCGCTATTCTGAGTACAGAGCATAGTTTAAGTACTCTG | |
| | TGCTGGAAACAGCACAGAATCTACTTAAACAAGGCAAAATGCCGTG | |
| | TTTATCTCGTCAACTTGTTGGCGAGATTTTTTTGGTAACCGGACCGAG | |
| | GCTGCAGCGTCGTCCTCCCTAGGAACCCCTAGTGATGGAGTTGGCCA | |
| | CTCCCTCTCTGCGCGCTCGCTCGCTCACTGAGGCCGGGCGACCAAAG | |
| | GTCGCCCGACGCCCGGGCTTTGCCCGGGCGGCCTCAGTGAGCGAGC | |
| | GAGCGCGCAGCTGCCTGCAGG | |
| CTX-507 | CCTGCAGGCAGCTGCGCGCTCGCTCGCTCACTGAGGCCGCCCGGGCG | 63 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGTTCTGACTGTAAGTACACTATGTTTAAGTA | |
| | CTCTGTGCTGGAAACAGCACAGAATCTACTTAAACAAGGCAAAATG | |
| | CCGTGTTTATCTCGTCAACTTGTTGGCGAGATTTTTTTCACCGGTGGT | |
| | GTGGAAAGTCCCCAGGCTCCCCAGCAGGCAGAAGTATGCAAAGCAT | |
| | GCATCTCAATTAGTCAGCAACCACGTTACATAACTTACGGTAAATGG | |
| | CCCGCCTGGCTGACCGCCCAACGACCCCCGCCCATTGACGTCAATAA | |
| | TGACGTATGTTCCCATAGTAACGCCAATAGGGACTTTCCATTGACGT | |
| | CAATGGGTGGAGTATTTACGGTAAACTGCCCACTTGGCAGTACATCA | |
| | AGTGTATCATATGCCAAGTACGCCCCCTATTGACGTCAATGACGGTA | |
| | AATGGCCCGCCTGGCATTATGCCCAGTACATGACCTTATGGGACTTT | |
| | CCTACTTGGCAGTACATCTACGTATTAGTCATCGCTATTACCATGGT | |
| | GATGCGGTTTTGGCAGTACATCAATGGGCGTGGATAGCGGTTTGACT | |
| | CACGGGGATTTCCAAGTCTCCACCCCATTGACGTCAATGGGAGTTTG | |
| | TTTTGGCACCAAAATCAACGGGACTTTCCAAAATGTCGTAACAACTC | |
| | CGCCCCATTGACGCAAATGGGCGGTAGGCGTGTACGGTGGGAGGTC | |
| | TATATAAGCAGAGCTCGTTTAGTGAACCGTCACCGGTGCCACCATGG | |
| | CCCCAAAGAAGAAGCGGAAGGTCGGATCCGGAAAGCGGAACTATAT | |
| | CCTGGGACTGGACATCGGAATTACCTCCGTGGGATACGGCATCATCG | |
| | ATTACGAGACTAGGGACGTGATTGACGCCGGCGTGAGACTCTTTAA | |
| | GGAGGCCAACGTGGAAAACAACGAAGGTCGCAGATCCAAGCGGGG | |
| | TGCAAGACGCCTGAAGCGCCGGAGGAGACATCGGATACAGCGCGTG | |
| | AAGAAGCTCCTTTTCGACTACAACCTCCTCACTGACCACTCGGAATT | |
| | GTCCGGTATCAACCCCTACGAAGCCCGCGTGAAAGGCCTGAGCCAG | |
| | AAGCTGTCCGAAGAGGAGTTTAGCGCAGCCCTGCTGCACCTGGCTA | |
| | AGCGAAGGGGGGTGCACAACGTGAACGAGGTGGAGGAGGACACTG | |
| | GCAACGAACTGTCCACCAAGGAGCAGATTTCACGGAACTCGAAGGC | |
| | GCTGGAAGAGAAATATGTGGCCGAGCTGCAGCTGGAGAGGCTCAAG | |
| | AAGGATGGCGAAGTCCGGGGGAGCATCAATCGCTTCAAGACCTCGG | |
| | ACTACGTGAAGGAAGCCAAACAGCTGTTGAAGGTGCAGAAGGCCTA | |
| | CCACCAACTGGACCAATCATTCATTGACACTTACATCGATCTGCTTG | |
| | AAACCAGGCGCACCTACTACGAGGGTCCTGGAGAAGGCAGCCCTTT | |
| | CGGATGGAAGGACATCAAGGAGTGGTATGAGATGCTGATGGGTCAT | |
| | TGCACCTACTTTCCGGAAGAACTGCGCTCAGTGAAGTACGCGTACAA | |
| | CGCTGACCTCTACAACGCTCTCAACGATCTGAACAACCTCGTGATCA | |
| | CCCGGGACGAGAACGAAAAGCTGGAGTACTACGAAAAGTTCCAGAT | |
| | TATCGAAAACGTGTTCAAGCAGAAGAAGAAGCCCACCCTGAAGCAG | |
| | ATTGCAAAGGAGATCCTTGTGAACGAGGAGGATATTAAGGGCTACC | |
| | GGGTCACCTCCACCGGGAAACCAGAGTTCACTAATCTCAAGGTGTAC | |
| | CATGACATTAAGGACATTACTGCCCGCAAGGAGATCATTGAAAACG | |
| | CGGAACTGCTGGACCAAATCGCGAAGATCCTGACCATCTATCAGAG | |
| | CTCCGAGGATATCCAGGAGGAACTTACTAACCTCAATTCCGAGCTGA | |
| | CGCAGGAAGAAATCGAGCAAATTAGCAACCTGAAGGGTTACACTGG | |
| | AACCCACAACCTCAGCTTGAAAGCGATTAACCTTATTTTGGATGAAC | |
| | TTTGGCACACTAATGACAATCAGATCGCCATTTTCAACCGGCTGAAA | |
| | CTGGTGCCGAAGAAGGTGGACCTGAGCCAACAGAAGGAAATCCCGA | |
| | CCACCCTTGTGGACGATTTCATCCTGTCACCTGTGGTGAAGAGGAGC | |
| | TTCATCCAGTCGATCAAGGTCATCAACGCCATCATAAAGAAGTACGG | |
| | CCTTCCCAACGACATCATCATCGAACTGGCCCGCGAGAAGAACTCCA | |
| | AAGATGCCCAGAAGATGATCAACGAGATGCAGAAGCGAAACCGGC | |
| | AGACGAACGAACGGATCGAGGAGATCATCCGGACCACCGGGAAGG | |
| | AAAACGCGAAGTACCTGATCGAGAAAATCAAGCTGCATGATATGCA | |
| | GGAAGGGAAGTGTCTCTACTCCCTGGAGGCCATTCCGCTGGAGGATT | |
| | TGCTGAACAACCCTTTCAACTACGAAGTCGATCATATCATTCCTCGC | |
| | TCCGTGTCCTTCGATAACTCCTTCAACAATAAGGTCCTCGTGAAGCA | |
| | GGAGGAGAACTCGAAGAAGGGCAACAGAACCCCGTTCCAGTACCTC | |
| | TCGTCGTCCGACTCCAAGATCAGCTACGAAACTTTCAAGAAGCACAT | |
| | TCTGAACCTGGCCAAGGGCAAAGGGAGAATTAGCAAGACCAAGAAG | |
| | GAATACCTCCTGGAAGAGAGAGACATCAACCGCTTCTCGGTGCAAA | |
| | AGGATTTCATCAACCGCAACCTGGTCGATACCAGATACGCCACCAG | |
| | GGGACTGATGAACCTCCTGCGGTCCTACTTCCGGGTCAACAATCTGG | |
| | ACGTGAAGGTCAAATCCATCAACGGGGGCTTTACTTCTTTCCTGCGC | |
| | CGGAAGTGGAAGTTCAAGAAGGAACGGAACAAGGGATACAAGCAC | |
| | CACGCTGAAGATGCCCTGATTATTGCCAACGCCGACTTCATCTTTAA | |
| | GGAATGGAAAAAGCTGGACAAGGCTAAGAAGGTCATGGAGAACCA | |
| | GATGTTCGAAGAAAAGCAGGCCGAGTCCATGCCCGAAATCGAAACC | |
| | GAGCAGGAATACAAGGAGATCTTCATCACACCGCACCAAATCAAGC | |
| | ACATCAAGGACTTCAAGGATTACAAGTACAGCCACCGGGTGGACAA | |
| | GAAGCCTAACAGAGAGCTTATCAACGACACCCTGTACTCCACGCGC | |
| | AAGGACGACAAGGGAAACACATTGATCGTGAACAACCTGAACGGAC | |
| | TGTATGACAAGGACAATGACAAACTGAAGAAGCTGATCAACAAATC | |
| | GCCGGAAAAGCTCCTGATGTACCATCACGACCCTCAAACCTACCAG | |
| | AAACTGAAGCTCATCATGGAGCAGTACGGCGACGAAAAGAATCCCC | |
| | TGTACAAATACTACGAGGAGACTGGAAATTACCTGACTAAGTACTCC | |
| | AAGAAGGATAACGGCCCCGTGATCAAGAAGATTAAGTACTACGGAA | |
| | ACAAACTGAACGCACATCTCGACATCACCGATGATTATCCAAACTCC | |
| | CGCAACAAAGTCGTGAAGCTCTCCCTCAAACCGTACCGCTTCGACGT | |
| | GTACCTGGATAATGGGGTGTACAAGTTCGTGACCGTGAAGAACCTG | |
| | GACGTCATTAAGAAGGAAAACTACTACGAAGTGAACTCAAAGTGCT | |
| | ACGAGGAAGCCAAGAAGCTCAAGAAGATCAGCAACCAGGCCGAGTT | |
| | CATCGCATCGTTTTACAACAATGACCTCATTAAGATTAATGGAGAAC | |
| | TGTACAGAGTGATCGGCGTGAACAACGACCTCCTGAACCGGATTGA | |
| | AGTGAACATGATCGATATTACCTACCGGGAGTATCTGGAGAACATG | |
| | AACGACAAGCGCCCACCGAGAATCATCAAAACTATTGCCTCCAAGA | |
| | CCCAATCCATTAAGAAATACTCCACCGACATCCTGGGCAACCTGTAC | |
| | GAGGTCAAGTCGAAGAAGCACCCCCAGATTATCAAGAAGGGAAAGC | |
| | TTGCCCCAAAGAAGAAGCGGAAGGTCTAAGGTACTAGTAATAAAAT | |
| | ATCTTTATTTTCATTACATCTGTGTGTTGGTTTTTTGTGTGAGCGCTG | |
| | AGGGCCTATTTCCCATGATTCCTTCATATTTGCATATACGATACAAG | |
| | GCTGTTAGAGAGATAATTGGAATTAATTTGACTGTAAACACAAAGAT | |
| | ATTAGTACAAAATACGTGACGTAGAAAGTAATAATTTCTTGGGTAGT | |
| | TTGCAGTTTTAAAATTATGTTTTAAAATGGACTATCATATGCTTACCG | |
| | TAACTTGAAAGTATTTCGATTTCTTGGCTTTATATATCTTGTGGAAAG | |
| | GACGAAACACCGCTATTCTGAGTACAGAGCATAGTTTAAGTACTCTG | |
| | TGCTGGAAACAGCACAGAATCTACTTAAACAAGGCAAAATGCCGTG | |
| | TTTATCTCGTCAACTTGTTGGCGAGATTTTTTTGGTAACCGGACCGAG | |
| | GCTGCAGCGTCGTCCTCCCTAGGAACCCCTAGTGATGGAGTTGGCCA | |
| | CTCCCTCTCTGCGCGCTCGCTCGCTCACTGAGGCCGGGCGACCAAAG | |
| | GTCGCCCGACGCCCGGGCTTTGCCCGGGCGGCCTCAGTGAGCGAGC | |
| | GAGCGCGCAGAGAGGGAGTGGCCAA | |
| CTX-603 | CCTGCAGGCAGCTGCGCGCTCGCTCGCTCACTGAGGCCGCCCGGGCG | 64 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGACTATGATTAAATGCTTGATAGTTTAAGT | |
| | ACTCTGTGCTGGAAACAGCACAGAATCTACTTAAACAAGGCAAAAT | |
| | GCCGTGTTTATCTCGTCAACTTGTTGGCGAGATTTTTTTCACCGGTGG | |
| | TGTGGAAAGTCCCCAGGCTCCCCAGCAGGCAGAAGTATGCAAAGCA | |
| | TGCATCTCAATTAGTCAGCAACCACGTTACATAACTTACGGTAAATG | |
| | GCCCGCCTGGCTGACCGCCCAACGACCCCCGCCCATTGACGTCAATA | |
| | ATGACGTATGTTCCCATAGTAACGCCAATAGGGACTTTCCATTGACG | |
| | TCAATGGGTGGAGTATTTACGGTAAACTGCCCACTTGGCAGTACATC | |
| | AAGTGTATCATATGCCAAGTACGCCCCCTATTGACGTCAATGACGGT | |
| | AAATGGCCCGCCTGGCATTATGCCCAGTACATGACCTTATGGGACTT | |
| | TCCTACTTGGCAGTACATCTACGTATTAGTCATCGCTATTACCATGGT | |
| | GATGCGGTTTTGGCAGTACATCAATGGGCGTGGATAGCGGTTTGACT | |
| | CACGGGGATTTCCAAGTCTCCACCCCATTGACGTCAATGGGAGTTTG | |
| | TTTTGGCACCAAAATCAACGGGACTTTCCAAAATGTCGTAACAACTC | |
| | CGCCCCATTGACGCAAATGGGCGGTAGGCGTGTACGGTGGGAGGTC | |
| | TATATAAGCAGAGCTCGTTTAGTGAACCGTCACCGGTGCCACCATGG | |
| | CCCCAAAGAAGAAGCGGAAGGTCGGATCCGAGAGCGACGAGAGCG | |
| | GCCTGCCCGCCATGGAGATCGAGTGCCGCATCACCGGCACCCTGAA | |
| | CGGCGTGGAGTTCGAGCTGGTGGGCGGCGGAGAGGGCACCCCCGAG | |
| | CAGGGCCGCATGACCAACAAGATGAAGAGCACCAAAGGCGCCCTGA | |
| | CCTTCAGCCCCTACCTGCTGAGCCACGTGATGGGCTACGGCTTCTAC | |
| | CACTTCGGCACCTACCCCAGCGGCTACGAGAACCCCTTCCTGCACGC | |
| | CATCAACAACGGCGGCTACACCAACACCCGCATCGAGAAGTACGAG | |
| | GACGGCGGCGTGCTGCACGTGAGCTTCAGCTACCGCTACGAGGCCG | |
| | GCCGCGTGATCGGCGACTTCAAGGTGATGGGCACCGGCTTCCCCGA | |
| | GGACAGCGTGATCTTCACCGACAAGATCATCCGCAGCAACGCCACC | |
| | GTGGAGCACCTGCACCCCATGGGCGATAACGATCTGGATGGCAGCT | |
| | TCACCCGCACCTTCAGCCTGCGCGACGGCGGCTACTACAGCTCCGTG | |
| | GTGGACAGCCACATGCACTTCAAGAGCGCCATCCACCCCAGCATCCT | |
| | GCAGAACGGGGGCCCCATGTTCGCCTTCCGCCGCGTGGAGGAGGAT | |
| | CACAGCAACACCGAGCTGGGCATCGTGGAGTACCAGCACGCCTTCA | |
| | AGACCCCGGATGCAGATGCCGGTGAAGAATAAGCGCTAATAAAATA | |
| | TCTTTATTTTCATTACATCTGTGTGTTGGTTTTTTGTGTGAGAAAACG | |
| | CCAGTAAGTGACAGAGTCACAAATGACTGCACAGAGTCCTTGGTGA | |
| | ACAGGCGACCATGCTTTTCAGCTCTGGAAGTCGTGAAAACATACGTT | |
| | CCCAAAGAGTTTTGAACTGAAAACTTCACCTTCCATGCAGATATATG | |
| | CACACTTTCTGAGAAGGAGAGACAAATCAAGAAACAAACTGCACTT | |
| | GTTGAGCTTGTGAAACACAAGCCCAAGGCAACAAAAGAGCAACTGA | |
| | AAGCTGTTTGAGATGATTTCGCAGCTTTTGTAGAGAAGTGCTGCAAG | |
| | GCTGACGATAAGGAGACCTGCTTTGCCGAGGAGGGTAAAAAACTTG | |
| | TTGCTGCAAGTCAAGCTGCCTTAGGCTTATAACATCTACATTTAAAA | |
| | GACTCTCAGCCTACCTGAAGAATAAGAGAAAGAAATGAAAGATCAA | |
| | AAGCTTATTCATCTGTTTTCTTTTTCGTTGGTGTAAAGCCAACACCCT | |
| | GTCTAAAAAACATAAATTTCTTTAATCATTTTGCCTCTTTTCTCTGTG | |
| | CTTCAATTAATAAAAAATGGAAAGAATCTAATAGAGTGGTACAGCA | |
| | CTGTTATTTTTCAAAGATGTGTTGCTATCCTGAAAATTCTGTAGGTTC | |
| | TGTGGAAGTTCCAGTGTTCTCTCTTATTCCACTTCGGTAGAGGATTTC | |
| | TAGTTTCTGTGGGCTAATTAAATAAATCACTAATACTCTTCTAAGTTA | |
| | AGTTTGCAGAAGTTTCCAAGTTAGTGACAGATCTTACCAAAGTCCAC | |
| | ACGGAATGCTGCCTGAGAGATCTGCTTGAATGTGCTGATGACAGGG | |
| | CGGACCTTGCCAAGTATATCTGTGAAAATCAGGATTCGATCTCCAGT | |
| | AAACTGAAGGAATGCTGTGAAAAACCTCTGTTGGAAAAATCCCACT | |
| | GCATTGCCGAAGTGGAAAATGATGAGTGACCTGCTGACTTGCCTTAC | |
| | TTAGCTGCTGATTTTGTTGAAAGTAAGGTGATTTGCAAAAACTTGAC | |
| | TGAGGCAAAGGATGTCTTCCTGGGCTGATTTTTGTATGAATATGCAA | |
| | GAAGGACTCCTGATTACTCTGTCGTGCTGCTGCTGAGACTTGCCAAG | |
| | AACTATGAAACCACAGATCTGAAGTGCTGTGCCGCTGCAGATCCTAC | |
| | TGAATGCTATGCCAAAGTGTTCGATGAATTTAAACCTCTTGTGGAAG | |
| | AGCCTCAGAATTTAATCAAACAAAACTGTGAGCTTTTTGAGCAGCTT | |
| | GGAGAGTACAAATTCCAGAATGCGCTATTAGTTCGTTACACCAAGA | |
| | AAGTACCCCAAGTGTCAACTCCAACTCTTGTAGAGGTCTCAAGAAAC | |
| | CTCGGAAAAGTGGGCAGCAAATGTTGTAAACATCCTGAAGCAAAAA | |
| | GATGACCCTGTGCAGAAGACTATCTATCCGTGGTCCTGAACCAGTTA | |
| | TGTGTGTTGCATGAGGATGTCTTCTGGCAATTTCATATAAGTATTTTT | |
| | TCAAAATGATCTCTTCTGTCAACCCCACGCCTTTGGCACATGAAAGT | |
| | GGGTAACCTTTATTTCCCTTCTTTTTCTCTTTAGCTCGGCTTATTCCAG | |
| | GGGTGTGTTTCGTCGAGATGCACACAAGAGTGAGGTTGCTACTCGGT | |
| | TTAAAGATTTGGGAGAAGAAAATTTCAAAGCCTTGGTGTTGATTGCC | |
| | TTTGCTCAGTATCTTCAGCAGTGTCCATTTGAAGATACTGTAAAATTA | |
| | GTGAATGAAGTAACTGAATTTGCAAAAAACTGTGTAGCTGTGAAGTC | |
| | AGCTGAAAATTGTGACAAATCACTTCATACCCTTTTTGGAGACAAAT | |
| | TATGCACAGTTGCAACTCTTCGTGAAACCTTGAGTGAATGAGCTGAC | |
| | TGCTGTGCAAAACAAGAACCTGAGAGATGAAAATGCTTCTTGCAAC | |
| | ACAAAGTGAACAACCCAAACCTCCCCCGATTGGTCAGACCAGAGGT | |
| | TGATGTGTGATGCACTGCTTTTACTGACAATGAAGAGACATTTTTGA | |
| | AAAAATACTTATTGAAAATTGCCAGAAGAACTCCTTACTTTTTGACC | |
| | CCGGAACTCCTTTTCTTTGCTAAAAGGTATAAAGCTGCTTTTACAGA | |
| | ATGTTGCCAAGCTGCTGATAAAGCTGCCTGCCTGTTGCCAAAGCTCG | |
| | TGAAACTTCGGGTGAAAGGGAAGGCTTCGTCTGCCAAACAGAGACT | |
| | CTGAAATGCCAGTCTCCAAAAATTTGGAGAAAGAGCTTTCAAAGCA | |
| | TGGGCAGTGGCTCGCCTGAGCCAGAGATTTCCCAAAGCTGAGCTAG | |
| | CGAGGGCCTATTTCCCATGATTCCTTCATATTTGCATATACGATACA | |
| | AGGCTGTTAGAGAGATAATTGGAATTAATTTGACTGTAAACACAAA | |
| | GATATTAGTACAAAATACGTGACGTAGAAAGTAATAATTTCTTGGGT | |
| | AGTTTGCAGTTTTAAAATTATGTTTTAAAATGGACTATCATATGCTTA | |
| | CCGTAACTTGAAAGTATTTCGATTTCTTGGCTTTATATATCTTGTGGA | |
| | AAGGACGAAACACCGCTTAAAGGCTTCATATAAGGGGTTTAAGTAC | |
| | TCTGTGCTGGAAACAGCACAGAATCTACTTAAACAAGGCAAAATGC | |
| | CGTGTTTATCTCGTCAACTTGTTGGCGAGATTTTTTTGGTAACCGGAC | |
| | CGAGGCTGCAGCGTCGTCCTCCCTAGGAACCCCTAGTGATGGAGTTG | |
| | GCCACTCCCTCTCTGCGCGCTCGCTCGCTCACTGAGGCCGGGCGACC | |
| | AAAGGTCGCCCGACGCCCGGGCTTTGCCCGGGCGGCCTCAGTGAGC | |
| | GAGCGAGCGCGCAGCTGCCTGCAGG | |
| CTX-1074 | CCTGCAGGCAGCTGCGCGCTCGCTCGCTCACTGAGGCCGCCCGGGCG | 65 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGTGGAGCAGTACGGCGACGAAGTTTAAGTA | |
| | CTCTGTGCTGGAAACAGCACAGAATCTACTTAAACAAGGCAAAATG | |
| | CCGTGTTTATCTCGTCAACTTGTTGGCGAGATTTTTTTCACCGGTGGC | |
| | ACACACTGTAGTTCATCTTTACATGGCCTCATTGAAGACTACAGCTC | |
| | TGGTATGCGTATAAGGAACTAGCATTAGGTCATTTCAAGCCGATGCT | |
| | AGAATCCAGATTCCATGCTGACCGATGAGGATATAGTGAGAATCTTT | |
| | CAAGAACATTCTTAACCGTTGGTATCTTAGCTCCACCCTCACTGGTTC | |
| | TTCCGGCCAAGCTGCTGGCCTCCCTCCTCAACCGTTCTGATCATGCTT | |
| | GCTTAGTCGGCCAGTTAAGCCTGATTATGACCTGGTTACCTGTTGTCT | |
| | AAGGGCAGGAATCACCGCCGTAACTCTAGCACTTAGCACAGTACTT | |
| | GGCTTGTAAGAGGTCCTCGATGATGTGAATACATTAAATAATTAACC | |
| | TAAGAAAGATTTCATATTAGGCATTGTAATGACTTAAGGTAAAGAGC | |
| | AGTGCTATTAACAATCCAGCTTGTTTGGGCTATTGTGGCTGTGGGCA | |
| | CCTCTCTGGGTGTATATCTGAGGTGCTGGCTACCTCTTGGAGGATTAT | |
| | AAGACAATCAGCAACCCTTGCATGGTGGCAACAGTAATAATAGCCA | |
| | TCCTTACATAGTCCTACAGCCCTGTAGCAATGGTCCAACAGATGAGG | |
| | AACCTTTGAAGCCTCAGAGAGGCTAACAGACAGACCCTAGGTCATA | |
| | CAGTTATTAAGAGAAGGCGAACCTCTCTCGAGTAATACCAGTTAATA | |
| | GGCTACACAAATGGTAGTGGCTGTTGTATTCAGTTGCTGAGGAATGC | |
| | TAAACATAATTCTGCCAATTTCCGCACCCGACTTCCCGGGCTCGGGT | |
| | GATTCTAGGGCTGTGTCATTTGTATACGCTCTTGTTGCCCGGGCTGGA | |
| | GTACAGTGGCCTCAGTGCTCCCGGGTTCCCTACCTCATGCGCCTGTA | |
| | TAATAGAGACGAGGTTTCACAGGCTACCTGATCCAGTGAATATTTGT | |
| | ATTGTAGAGATGGTGGCCATGTTCCTGAGCTCAAGCGATCTGCCCGC | |
| | CTCTGGCCACCGTGCCTGGCCTAGGTAGACGCAGCGTGATGCCTGAG | |
| | TATATAGTGATGCTAGAGCTGGCTGTTTGTTAGCTTTGAACATAAGA | |
| | TACTCATTGTAGTTTGCAAATCCCTCTTCCTAATTTCTTTCCCTTAAAT | |
| | TGTTTGCATGTTAGCGCTTAAATGGTGCTATGTGCTAGAAGCCTTAA | |
| | ATTACACAAATCAGAGAGGTGCCCAACTTTGAACCTAAGCTGCTCTT | |
| | AATCTCTAAACAAGTTAGTAGTGACAATAGTAGGATACTTAACTATG | |
| | AGGCATAGCAGGCATTATCACCCTAAAGTGTACCCTTTAGGTAAGTA | |
| | TATACTTGCCCAATATCACTTATCAAATGTGTCTGATACAACCCAAA | |
| | CTATCGAAACTGCCAGGGTAAACTTGGACACACTTGAGCTAAGAATT | |
| | AAGTCCTAGAAATGTAATCCTGCCCTAGCCGAGCTTACCCTGCAGAA | |
| | TTGGTCGGAGCACCGTCCTTGGCCACACTGTTATCAACAGGGTGTCA | |
| | ATCTGTAGGAATTACTCTTTGTGACCACCAGGAAATAGAGCAGTTCA | |
| | GTTCATTTCTTTCTCACTGTGACCTGCATACTACAAGTCTACTTTGCT | |
| | ATCCATTGTTTGTATCTGGGTATTACCAGATCAGCAGAGAAGAGTTG | |
| | CCTTGGAGCAGCTGCAGTTCATTAGATAGTAACTAGGCCATGTCAAC | |
| | TCCCTTGTAGTGAAGATTGTACTGGTACCTTTCTGTAAATATTGTGTA | |
| | GATCAATCACCACCTCAACCCAGTGGCTGCCAAATTACAATAATTCA | |
| | CTACTACTAAGATAATCTACTAGTTCGATCACATACTTCCTACTGTCT | |
| | TCAGCATTGTGCTTCTGATTATAATTGTCCAGAGTGAACATGTCTATT | |
| | CTTCCACTGTACACACTAATGGATTGTAATATTGGGTAAATTCATGT | |
| | CCTTACACATGTAGTAGTTATGAGCCCATGTCCCTAGAATGAGTAAT | |
| | AACCTTGGTTGAATAGTCAAGAATGCTGAAATTCTTCTAACAGCAGA | |
| | AGGGAAGGCAAGCAAGTGTTACTGATAAGATGAATCTACTATTAGC | |
| | TTTAATTATACATTTAGGAATATTGCATCAGTAACTCATAAGGCTGTT | |
| | ATCCTGAGTTAACACAAATTATCCAAGGAGATCTGCTTTGAGGTGTG | |
| | AGTGTATCTGATGCCAACTAGCAATTCCAGAAGTTTGGAATTAAATT | |
| | ATGGTTTATCTATTGTTATACCTCAATTATATCATGTTTGCTGTGCTC | |
| | TCGGCTCACTCTAGCCACCGACTCCCTCTGAGCCTTGCAGGGTAGAG | |
| | ACAGGATTGGCCAGGATGGTCTCCATCATGATCGGCCTCGTGGGAGC | |
| | CACTACGCCTGGCCATAGACTCACTTCCATTAAGTCTTGTTTGGACC | |
| | CACGAACATTGTCTTTAAGATGGAGTTTCACGTTGCCCAGACTGTAG | |
| | TGCAATGGTGCAATCTCAGCTCACTGCAACCAATTCTCCTCCCGAGT | |
| | AGCTGGAATTACAGGCGCCCGCCACCACGGTGTTTCACCGGCCATGA | |
| | TCCGCCCACCTCAGCCTCGTGTGAGCCACCGCATCTGGCCAACATGT | |
| | CTTCCTAGACTTAAGCACAGATGATGAATTGATGTGTCTTAGCTTGG | |
| | ATTAACTTGCTTACTGTAAAGATAATATAGCTTGACATGAAGGCCAT | |
| | TATTACAGATGTGACGTGCATAATTATTAGTATTACATGGGTCAGTC | |
| | TGGCAATTATGAAGAATAATGCCAGACATTTCAGTAATCGATTATAG | |
| | CGTATTGACAGTCCAGACGTCAGAATTTCTCAATACTCTTTCAGATT | |
| | AATGTACCTGTAGCGATATCATTCACAAGTATATCACAAGTAAGTTA | |
| | GAATTTGAGAACTGTGTTCTAGAGATGCAGTCAGATTTCTGAACTGT | |
| | CTCAGCAAATGGAGAGCTAGTAATTAATAACCTGTCCTTTGATTTCT | |
| | GATTCAGCCAAGAATGGCCATATTTGGGAAGGAGAGTAACCACGCA | |
| | TTCATTTACCACAGAGCTCTCAGCTTAAAGCCATACAGGACCGTGAT | |
| | CTGTTCTAGCCATATGTAGCATTTATGTCCTAGTGTGATGGTATTTGG | |
| | AGACAGGGCCTTTGGAAGGTAATTGAAGTGGGCCCAGGTCTGATTG | |
| | GATTAGTGCGGGCGCACAAGGCCAATCACGAGGTCAGCCAGCCTGG | |
| | CCAATGTAGTGAAACACCAACATTAGCTGGGTGTGGTAGCGGGCTC | |
| | CTGTCATCCAAGCTACGAGGCATGAGAATCGGGACAGATTGTGCCA | |
| | CTGTGGGTGACTCAAGAGACACCAGAGAGCTTGTTAGAAGAGGTCA | |
| | TGTGAGCACGACCTTCAAGCCAAAGAAGAGGCCTGAGATTGAAACC | |
| | TACCTTGCAGGTATTCCGTGAGAAATAAGTTTCTGTTAAGTCACTCA | |
| | GTCTGTGGTAGTTATGGCAGCCTGAGCAGGTAGTTGTTCTTTCAGAA | |
| | GGTGTTGATAATCAGATGCTAGCGGTAACCGGACCGAGGCTGCAGC | |
| | GTCGTCCTCCCTAGGAACCCCTAGTGATGGAGTTGGCCACTCCCTCT | |
| | CTGCGCGCTCGCTCGCTCACTGAGGCCGGGCGACCAAAGGTCGCCC | |
| | GACGCCCGGGCTTTGCCCGGGCGGCCTCAGTGAGCGAGCGAGCGCG | |
| | CAGCTGCCTGCAGG | |
| CTX-769 | CCTGCAGGCAGCTGCGCGCTCGCTCGCTCACTGAGGCCGCCCGGGCG | 66 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGCGTTGGAGCGGGGAGAAGGCCGTTTAAGT | |
| | ACTCTGTGCTGGAAACAGCACAGAATCTACTTAAACAAGGCAAAAT | |
| | GCCGTGTTTATCTCGTCAACTTGTTGGCGAGATTTTTTTCACCGGTGG | |
| | TGTGGAAAGTCCCCAGGCTCCCCAGCAGGCAGAAGTATGCAAAGCA | |
| | TGCATCTCAATTAGTCAGCAACCACGTTACATAACTTACGGTAAATG | |
| | GCCCGCCTGGCTGACCGCCCAACGACCCCCGCCCATTGACGTCAATA | |
| | ATGACGTATGTTCCCATAGTAACGCCAATAGGGACTTTCCATTGACG | |
| | TCAATGGGTGGAGTATTTACGGTAAACTGCCCACTTGGCAGTACATC | |
| | AAGTGTATCATATGCCAAGTACGCCCCCTATTGACGTCAATGACGGT | |
| | AAATGGCCCGCCTGGCATTATGCCCAGTACATGACCTTATGGGACTT | |
| | TCCTACTTGGCAGTACATCTACGCTAATAAAATATCTTTATTTTCATT | |
| | ACATCTGTGTGTTGGTTTTTTGTGTGAGAAAACGCCAGTAAGTGACA | |
| | GAGTCACAAATGACTGCACAGAGTCCTTGGTGAACAGGCGACCATG | |
| | CTTTTCAGCTCTGGAAGTCGTGAAAACATACGTTCCCAAAGAGTTTT | |
| | GAACTGAAAACTTCACCTTCCATGCAGATATATGCACACTTTCTGAG | |
| | AAGGAGAGACAAATCAAGAAACAAACTGCACTTGTTGAGCTTGTGA | |
| | AACACAAGCCCAAGGCAACAAAAGAGCAACTGAAAGCTGTTTGAGA | |
| | TGATTTCGCAGCTTTTGTAGAGAAGTGCTGCAAGGCTGACGATAAGG | |
| | AGACCTGCTTTGCCGAGGAGGGTAAAAAACTTGTTGCTGCAAGTCA | |
| | AGCTGCCTTAGGCTTATAACATCTACATTTAAAAGACTCTCAGCCTA | |
| | CCTGAAGAATAAGAGAAAGAAATGAAAGATCAAAAGCTTATTCATC | |
| | TGTTTTCTTTTTCGTTGGTGTAAAGCCAACACCCTGTCTAAAAAACAT | |
| | AAATTTCTTTAATCATTTTGCCTCTTTTCTCTGTGCTTCAATTAATAAA | |
| | AAATGGAAAGAATCTAATAGAGTGGTACAGCACTGTTATTTTTCAAA | |
| | GATGTGTTGCTATCCTGAAAATTCTGTAGGTTCTGTGGAAGTTCCAG | |
| | TGTTCTCTCTTATTCCACTTCGGTAGAGGATTTCTAGTTTCTGTGGGC | |
| | TAATTAAATAAATCACTAATACTCTTCTAAGTTAAGTTTGCAGAAGT | |
| | TTCCAAGTTAGTGACAGATCTTACCAAAGTCCACACGGAATGCTGCC | |
| | TGAGAGATCTGCTTGAATGTGCTGATGACAGGGCGGACCTTGCCAA | |
| | GTATATCTGTGAAAATCAGGATTCGATCTCCAGTAAACTGAAGGAAT | |
| | GCTGTGAAAAACCTCTGTTGGAAAAATCCCACTGCATTGCCGAAGTG | |
| | GAAAATGATGAGTGACCTGCTGACTTGCCTTACTTAGCTGCTGATTT | |
| | TGTTGAAAGTAAGGTGATTTGCAAAAACTTGACTGAGGCAAAGGAT | |
| | GTCTTCCTGGGCTGATTTTTGTATGAATATGCAAGAAGGACTCCTGA | |
| | TTACTCTGTCGTGCTGCTGCTGAGACTTGCCAAGAACTATGAAACCA | |
| | CAGATCTGAAGTGCTGTGCCGCTGCAGATCCTACTGAATGCTATGCC | |
| | AAAGTGTTCGATGAATTTAAACCTCTTGTGGAAGAGCCTCAGAATTT | |
| | AATCAAACAAAACTGTGAGCTTTTTGAGCAGCTTGGAGAGTACAAAT | |
| | TCCAGAATGCGCTATTAGTTCGTTACACCAAGAAAGTACCCCAAGTG | |
| | TCAACTCCAACTCTTGTAGAGGTCTCAAGAAACCTCGGAAAAGTGG | |
| | GCAGCAAATGTTGTAAACATCCTGAAGCAAAAAGATGACCCTGTGC | |
| | AGAAGACTATCTATCCGTGGTCCTGAACCAGTTATGTGTGTTGCATG | |
| | AGGATGTCTTCTGGCAATTTCATATAAGTATTTTTTCAAAATGATCTC | |
| | TTCTGTCAACCCCACGCCTTTGGCACATGAAAGTGGGTAACCTTTAT | |
| | TTCCCTTCTTTTTCTCTTTAGCTCGGCTTATTCCAGGGGTGTGTTTCGT | |
| | CGAGATGCACACAAGAGTGAGGTTGCTACTCGGTTTAAAGATTTGGG | |
| | AGAAGAAAATTTCAAAGCCTTGGTGTTGATTGCCTTTGCTCAGTATC | |
| | TTCAGCAGTGTCCATTTGAAGATACTGTAAAATTAGTGAATGAAGTA | |
| | ACTGAATTTGCAAAAAACTGTGTAGCTGTGAAGTCAGCTGAAAATTG | |
| | TGACAAATCACTTCATACCCTTTTTGGAGACAAATTATGCACAGTTG | |
| | CAACTCTTCGTGAAACCTTGAGTGAATGAGCTGACTGCTGTGCAAAA | |
| | CAAGAACCTGAGAGATGAAAATGCTTCTTGCAACACAAAGTGAACA | |
| | ACCCAAACCTCCCCCGATTGGTCAGACCAGAGGTTGATGTGTGATGC | |
| | ACTGCTTTTACTGACAATGAAGAGACATTTTTGAAAAAATACTTATT | |
| | GAAAATTGCCAGAAGAACTCCTTACTTTTTGACCCCGGAACTCCTTT | |
| | TCTTTGCTAAAAGGTATAAAGCTGCTTTTACAGAATGTTGCCAAGCT | |
| | GCTGATAAAGCTGCCTGCCTGTTGCCAAAGCTCGTGAAACTTCGGGT | |
| | GAAAGGGAAGGCTTCGTCTGCCAAACAGAGACTCTGAAATGCCAGT | |
| | CTCCAAAAATTTGGAGAAAGAGCTTTCAAAGCATGGGCAGTGGCTC | |
| | GCCTGAGCCAGAGATTTCCCAAAGCTGAGCTAGCGGTACCCGGACC | |
| | GAGGCTGCAGCGTCGTCCTCCCTAGGAACCCCTAGTGATGGAGTTGG | |
| | CCACTCCCTCTCTGCGCGCTCGCTCGCTCACTGAGGCCGGGCGACCA | |
| | AAGGTCGCCCGACGCCCGGGCTTTGCCCGGGCGGCCTCAGTGAGCG | |
| | AGCGAGCGCGCAGCTGCCTGCAGG | |
| CTX-1047 | CCTGCAGGCAGCTGCGCGCTCGCTCGCTCACTGAGGCCGCCCGGGCG | 67 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAATTCCGTTACATAACTTACGGTAAATGGCCCGCCTGGCTG | |
| | ACCGCCCAACGACCCCCGCCCATTGACGTCAATAATGACGTATGTTC | |
| | CCATAGTAACGCCAATAGGGACTTTCCATTGACGTCAATGGGTGGAG | |
| | TATTTACGGTAAACTGCCCACTTGGCAGTACATCAAGTGTATCATAT | |
| | GCCAAGTACGCCCCCTATTGACGTCAATGACGGTAAATGGCCCGCCT | |
| | GGCATTATGCCCAGTACATGACCTTATGGGACTTTCCTACTTGGCAG | |
| | TACATCTACGTATTAGTCATCGCTATTACCATGGTGATGCGGTTTTGG | |
| | CAGTACATCAATGGGCGTGGATAGCGGTTTGACTCACGGGGATTTCC | |
| | AAGTCTCCACCCCATTGACGTCAATGGGAGTTTGTTTTGGCACCAAA | |
| | ATCAACGGGACTTTCCAAAATGTCGTAACAACTCCGCCCCATTGACG | |
| | CAAATGGGCGGTAGGCGTGTACGGTGGGAGGTCTATATAAGCAGAG | |
| | CTCGTTTAGTGAACCGTGCTAGCACTCATTGTATGTAGAAGACCTCT | |
| | AAGGCCACCATGGCCCCAAAGAAGAAGCGGAAGGTCGGATCCGGA | |
| | AAGCGGAACTATATCCTGGGACTGGACATCGGAATTACCTCCGTGG | |
| | GATACGGCATCATCGATTACGAGACTAGGGACGTGATTGACGCCGG | |
| | CGTGAGACTCTTTAAGGAGGCCAACGTGGAAAACAACGAAGGTCGC | |
| | AGATCCAAGCGGGGTGCAAGACGCCTGAAGCGCCGGAGGAGACATC | |
| | GGATACAGCGCGTGAAGAAGCTCCTTTTCGACTACAACCTCCTCACT | |
| | GACCACTCGGAATTGTCCGGTATCAACCCCTACGAAGCCCGCGTGAA | |
| | AGGCCTGAGCCAGAAGCTGTCCGAAGAGGAGTTTAGCGCAGCCCTG | |
| | CTGCACCTGGCTAAGCGAAGGGGGGTGCACAACGTGAACGAGGTGG | |
| | AGGAGGACACTGGCAACGAACTGTCCACCAAGGAGCAGATTTCACG | |
| | GAACTCGAAGGCGCTGGAAGAGAAATATGTGGCCGAGCTGCAGCTG | |
| | GAGAGGCTCAAGAAGGATGGCGAAGTCCGGGGGAGCATCAATCGCT | |
| | TCAAGACCTCGGACTACGTGAAGGAAGCCAAACAGCTGTTGAAGGT | |
| | GCAGAAGGCCTACCACCAACTGGACCAATCATTCATTGACACTTACA | |
| | TCGATCTGCTTGAAACCAGGCGCACCTACTACGAGGGTCCTGGAGA | |
| | AGGCAGCCCTTTCGGATGGAAGGACATCAAGGAGTGGTATGAGATG | |
| | CTGATGGGTCATTGCACCTACTTTCCGGAAGAACTGCGCTCAGTGAA | |
| | GTACGCGTACAACGCTGACCTCTACAACGCTCTCAACGATCTGAACA | |
| | ACCTCGTGATCACCCGGGACGAGAACGAAAAGCTGGAGTACTACGA | |
| | AAAGTTCCAGATTATCGAAAACGTGTTCAAGCAGAAGAAGAAGCCC | |
| | ACCCTGAAGCAGATTGCAAAGGAGATCCTTGTGAACGAGGAGGATA | |
| | TTAAGGGCTACCGGGTCACCTCCACCGGGAAACCAGAGTTCACTAAT | |
| | CTCAAGGTGTACCATGACATTAAGGACATTACTGCCCGCAAGGAGA | |
| | TCATTGAAAACGCGGAACTGCTGGACCAAATCGCGAAGATCCTGAC | |
| | CATCTATCAGAGCTCCGAGGATATCCAGGAGGAACTTACTAACCTCA | |
| | ATTCCGAGCTGACGCAGGAAGAAATCGAGCAAATTAGCAACCTGAA | |
| | GGGTTACACTGGAACCCACAACCTCAGCTTGAAAGCGATTAACCTTA | |
| | TTTTGGATGAACTTTGGCACACTAATGACAATCAGATCGCCATTTTC | |
| | AACCGGCTGAAACTGGTGCCGAAGAAGGTGGACCTGAGCCAACAGA | |
| | AGGAAATCCCGACCACCCTTGTGGACGATTTCATCCTGTCACCTGTG | |
| | GTGAAGAGGAGCTTCATCCAGTCGATCAAGGTCATCAACGCCATCAT | |
| | AAAGAAGTACGGCCTTCCCAACGACATCATCATCGAACTGGCCCGC | |
| | GAGAAGAACTCCAAAGATGCCCAGAAGATGATCAACGAGATGCAGA | |
| | AGCGAAACCGGCAGACGAACGAACGGATCGAGGAGATCATCCGGA | |
| | CCACCGGGAAGGAAAACGCGAAGTACCTGATCGAGAAAATCAAGCT | |
| | GCATGATATGCAGGAAGGGAAGTGTCTCTACTCCCTGGAGGCCATTC | |
| | CGCTGGAGGATTTGCTGAACAACCCTTTCAACTACGAAGTCGATCAT | |
| | ATCATTCCTCGCTCCGTGTCCTTCGATAACTCCTTCAACAATAAGGTC | |
| | CTCGTGAAGCAGGAGGAGAAGTAAGTATCAAGGTTACAAGACAGCT | |
| | ATTCTGAGTACAGAGCATACAGAGTCTTGTCGAGACAGAGAAGACT | |
| | CTTGCGTTTCTGATAGGCACCTATTGGTCTTACTGACATCCACTTTGC | |
| | CTTTCTCTCCACAGCTCGAAGAAGGGCAACAGAACCCCGTTCCAGTA | |
| | CCTCTCGTCGTCCGACTCCAAGATCAGCTACGAAACTTTCAAGAAGC | |
| | ACATTCTGAACCTGGCCAAGGGCAAAGGGAGAATTAGCAAGACCAA | |
| | GAAGGAATACCTCCTGGAAGAGAGAGACATCAACCGCTTCTCGGTG | |
| | CAAAAGGATTTCATCAACCGCAACCTGGTCGATACCAGATACGCCA | |
| | CCAGGGGACTGATGAACCTCCTGCGGTCCTACTTCCGGGTCAACAAT | |
| | CTGGACGTGAAGGTCAAATCCATCAACGGGGGCTTTACTTCTTTCCT | |
| | GCGCCGGAAGTGGAAGTTCAAGAAGGAACGGAACAAGGGATACAA | |
| | GCACCACGCTGAAGATGCCCTGATTATTGCCAACGCCGACTTCATCT | |
| | TTAAGGAATGGAAAAAGCTGGACAAGGCTAAGAAGGTCATGGAGAA | |
| | CCAGATGTTCGAAGAAAAGCAGGCCGAGTCCATGCCCGAAATCGAA | |
| | ACCGAGCAGGAATACAAGGAGATCTTCATCACACCGCACCAAATCA | |
| | AGCACATCAAGGACTTCAAGGATTACAAGTACAGCCACCGGGTGGA | |
| | CAAGAAGCCTAACAGAGAGCTTATCAACGACACCCTGTACTCCACG | |
| | CGCAAGGACGACAAGGGAAACACATTGATCGTGAACAACCTGAACG | |
| | GACTGTATGACAAGGACAATGACAAACTGAAGAAGCTGATCAACAA | |
| | ATCGCCGGAAAAGCTCCTGATGTACCATCACGACCCTCAAACCTACC | |
| | AGAAACTGAAGCTCATCATGGAGCAGTACGGCGACGAAAAGAATCC | |
| | CCTGTACAAATACTACGAGGAGACTGGAAATTACCTGACTAAGTACT | |
| | CCAAGAAGGATAACGGCCCCGTGATCAAGAAGATTAAGTACTACGG | |
| | AAACAAACTGAACGCACATCTCGACATCACCGATGATTATCCAAACT | |
| | CCCGCAACAAAGTCGTGAAGCTCTCCCTCAAACCGTACCGCTTCGAC | |
| | GTGTACCTGGATAATGGGGTGTACAAGTTCGTGACCGTGAAGAACCT | |
| | GGACGTCATTAAGAAGGAAAACTACTACGAAGTGAACTCAAAGTGC | |
| | TACGAGGAAGCCAAGAAGCTCAAGAAGATCAGCAACCAGGCCGAGT | |
| | TCATCGCATCGTTTTACAACAATGACCTCATTAAGATTAATGGAGAA | |
| | CTGTACAGAGTGATCGGCGTGAACAACGACCTCCTGAACCGGATTG | |
| | AAGTGAACATGATCGATATTACCTACCGGGAGTATCTGGAGAACAT | |
| | GAACGACAAGCGCCCACCGAGAATCATCAAAACTATTGCCTCCAAG | |
| | ACCCAATCCATTAAGAAATACTCCACCGACATCCTGGGCAACCTGTA | |
| | CGAGGTCAAGTCGAAGAAGCACCCCCAGATTATCAAGAAGGGAAAG | |
| | CTTGCCCCAAAGAAGAAGCGGAAGGTCTAAGGTACTAGTAATAAAA | |
| | TATCTTTATTTTCATTACATCTGTGTGTTGGTTTTTTGTGTGAGCGCTG | |
| | GTAACCGGACCGAGGCTGCAGCGTCGTCCTCCCTAGGAACCCCTAGT | |
| | GATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGC | |
| | CTCAGTGAGCGAGCGAGCGCGCAGCTGCCTGCAGG | |
| CTX-1070 | CCTGCAGGCAGCTGCGCGCTCGCTCGCTCACTGAGGCCGCCCGGGCG | 68 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGCTTAGAGGTCTTCTACATACAGTTTAAGT | |
| | ACTCTGTGCTGGAAACAGCACAGAATCTACTTAAACAAGGCAAAAT | |
| | GCCGTGTTTATCTCGTCAACTTGTTGGCGAGATTTTTTTCACCGGTGG | |
| | CACACACTGTAGTTCATCTTTACATGGCCTCATTGAAGACTACAGCT | |
| | CTGGTATGCGTATAAGGAACTAGCATTAGGTCATTTCAAGCCGATGC | |
| | TAGAATCCAGATTCCATGCTGACCGATGAGGATATAGTGAGAATCTT | |
| | TCAAGAACATTCTTAACCGTTGGTATCTTAGCTCCACCCTCACTGGTT | |
| | CTTCCGGCCAAGCTGCTGGCCTCCCTCCTCAACCGTTCTGATCATGCT | |
| | TGCTTAGTCGGCCAGTTAAGCCTGATTATGACCTGGTTACCTGTTGTC | |
| | TAAGGGCAGGAATCACCGCCGTAACTCTAGCACTTAGCACAGTACTT | |
| | GGCTTGTAAGAGGTCCTCGATGATGTGAATACATTAAATAATTAACC | |
| | TAAGAAAGATTTCATATTAGGCATTGTAATGACTTAAGGTAAAGAGC | |
| | AGTGCTATTAACAATCCAGCTTGTTTGGGCTATTGTGGCTGTGGGCA | |
| | CCTCTCTGGGTGTATATCTGAGGTGCTGGCTACCTCTTGGAGGATTAT | |
| | AAGACAATCAGCAACCCTTGCATGGTGGCAACAGTAATAATAGCCA | |
| | TCCTTACATAGTCCTACAGCCCTGTAGCAATGGTCCAACAGATGAGG | |
| | AACCTTTGAAGCCTCAGAGAGGCTAACAGACAGACCCTAGGTCATA | |
| | CAGTTATTAAGAGAAGGCGAACCTCTCTCGAGTAATACCAGTTAATA | |
| | GGCTACACAAATGGTAGTGGCTGTTGTATTCAGTTGCTGAGGAATGC | |
| | TAAACATAATTCTGCCAATTTCCGCACCCGACTTCCCGGGCTCGGGT | |
| | GATTCTAGGGCTGTGTCATTTGTATACGCTCTTGTTGCCCGGGCTGGA | |
| | GTACAGTGGCCTCAGTGCTCCCGGGTTCCCTACCTCATGCGCCTGTA | |
| | TAATAGAGACGAGGTTTCACAGGCTACCTGATCCAGTGAATATTTGT | |
| | ATTGTAGAGATGGTGGCCATGTTCCTGAGCTCAAGCGATCTGCCCGC | |
| | CTCTGGCCACCGTGCCTGGCCTAGGTAGACGCAGCGTGATGCCTGAG | |
| | TATATAGTGATGCTAGAGCTGGCTGTTTGTTAGCTTTGAACATAAGA | |
| | TACTCATTGTAGTTTGCAAATCCCTCTTCCTAATTTCTTTCCCTTAAAT | |
| | TGTTTGCATGTTAGCGCTTAAATGGTGCTATGTGCTAGAAGCCTTAA | |
| | ATTACACAAATCAGAGAGGTGCCCAACTTTGAACCTAAGCTGCTCTT | |
| | AATCTCTAAACAAGTTAGTAGTGACAATAGTAGGATACTTAACTATG | |
| | AGGCATAGCAGGCATTATCACCCTAAAGTGTACCCTTTAGGTAAGTA | |
| | TATACTTGCCCAATATCACTTATCAAATGTGTCTGATACAACCCAAA | |
| | CTATCGAAACTGCCAGGGTAAACTTGGACACACTTGAGCTAAGAATT | |
| | AAGTCCTAGAAATGTAATCCTGCCCTAGCCGAGCTTACCCTGCAGAA | |
| | TTGGTCGGAGCACCGTCCTTGGCCACACTGTTATCAACAGGGTGTCA | |
| | ATCTGTAGGAATTACTCTTTGTGACCACCAGGAAATAGAGCAGTTCA | |
| | GTTCATTTCTTTCTCACTGTGACCTGCATACTACAAGTCTACTTTGCT | |
| | ATCCATTGTTTGTATCTGGGTATTACCAGATCAGCAGAGAAGAGTTG | |
| | CCTTGGAGCAGCTGCAGTTCATTAGATAGTAACTAGGCCATGTCAAC | |
| | TCCCTTGTAGTGAAGATTGTACTGGTACCTTTCTGTAAATATTGTGTA | |
| | GATCAATCACCACCTCAACCCAGTGGCTGCCAAATTACAATAATTCA | |
| | CTACTACTAAGATAATCTACTAGTTCGATCACATACTTCCTACTGTCT | |
| | TCAGCATTGTGCTTCTGATTATAATTGTCCAGAGTGAACATGTCTATT | |
| | CTTCCACTGTACACACTAATGGATTGTAATATTGGGTAAATTCATGT | |
| | CCTTACACATGTAGTAGTTATGAGCCCATGTCCCTAGAATGAGTAAT | |
| | AACCTTGGTTGAATAGTCAAGAATGCTGAAATTCTTCTAACAGCAGA | |
| | AGGGAAGGCAAGCAAGTGTTACTGATAAGATGAATCTACTATTAGC | |
| | TTTAATTATACATTTAGGAATATTGCATCAGTAACTCATAAGGCTGTT | |
| | ATCCTGAGTTAACACAAATTATCCAAGGAGATCTGCTTTGAGGTGTG | |
| | AGTGTATCTGATGCCAACTAGCAATTCCAGAAGTTTGGAATTAAATT | |
| | ATGGTTTATCTATTGTTATACCTCAATTATATCATGTTTGCTGTGCTC | |
| | TCGGCTCACTCTAGCCACCGACTCCCTCTGAGCCTTGCAGGGTAGAG | |
| | ACAGGATTGGCCAGGATGGTCTCCATCATGATCGGCCTCGTGGGAGC | |
| | CACTACGCCTGGCCATAGACTCACTTCCATTAAGTCTTGTTTGGACC | |
| | CACGAACATTGTCTTTAAGATGGAGTTTCACGTTGCCCAGACTGTAG | |
| | TGCAATGGTGCAATCTCAGCTCACTGCAACCAATTCTCCTCCCGAGT | |
| | AGCTGGAATTACAGGCGCCCGCCACCACGGTGTTTCACCGGCCATGA | |
| | TCCGCCCACCTCAGCCTCGTGTGAGCCACCGCATCTGGCCAACATGT | |
| | CTTCCTAGACTTAAGCACAGATGATGAATTGATGTGTCTTAGCTTGG | |
| | ATTAACTTGCTTACTGTAAAGATAATATAGCTTGACATGAAGGCCAT | |
| | TATTACAGATGTGACGTGCATAATTATTAGTATTACATGGGTCAGTC | |
| | TGGCAATTATGAAGAATAATGCCAGACATTTCAGTAATCGATTATAG | |
| | CGTATTGACAGTCCAGACGTCAGAATTTCTCAATACTCTTTCAGATT | |
| | AATGTACCTGTAGCGATATCATTCACAAGTATATCACAAGTAAGTTA | |
| | GAATTTGAGAACTGTGTTCTAGAGATGCAGTCAGATTTCTGAACTGT | |
| | CTCAGCAAATGGAGAGCTAGTAATTAATAACCTGTCCTTTGATTTCT | |
| | GATTCAGCCAAGAATGGCCATATTTGGGAAGGAGAGTAACCACGCA | |
| | TTCATTTACCACAGAGCTCTCAGCTTAAAGCCATACAGGACCGTGAT | |
| | CTGTTCTAGCCATATGTAGCATTTATGTCCTAGTGTGATGGTATTTGG | |
| | AGACAGGGCCTTTGGAAGGTAATTGAAGTGGGCCCAGGTCTGATTG | |
| | GATTAGTGCGGGCGCACAAGGCCAATCACGAGGTCAGCCAGCCTGG | |
| | CCAATGTAGTGAAACACCAACATTAGCTGGGTGTGGTAGCGGGCTC | |
| | CTGTCATCCAAGCTACGAGGCATGAGAATCGGGACAGATTGTGCCA | |
| | CTGTGGGTGACTCAAGAGACACCAGAGAGCTTGTTAGAAGAGGTCA | |
| | TGTGAGCACGACCTTCAAGCCAAAGAAGAGGCCTGAGATTGAAACC | |
| | TACCTTGCAGGTATTCCGTGAGAAATAAGTTTCTGTTAAGTCACTCA | |
| | GTCTGTGGTAGTTATGGCAGCCTGAGCAGGTAGTTGTTCTTTCAGAA | |
| | GGTGTTGATAATCAGATGCTAGCGAGGGCCTATTTCCCATGATTCCT | |
| | TCATATTTGCATATACGATACAAGGCTGTTAGAGAGATAATTGGAAT | |
| | TAATTTGACTGTAAACACAAAGATATTAGTACAAAATACGTGACGTA | |
| | GAAAGTAATAATTTCTTGGGTAGTTTGCAGTTTTAAAATTATGTTTTA | |
| | AAATGGACTATCATATGCTTACCGTAACTTGAAAGTATTTCGATTTCT | |
| | TGGCTTTATATATCTTGTGGAAAGGACGAAACACCGCTATTCTGAGT | |
| | ACAGAGCATAGTTTAAGTACTCTGTGCTGGAAACAGCACAGAATCT | |
| | ACTTAAACAAGGCAAAATGCCGTGTTTATCTCGTCAACTTGTTGGCG | |
| | AGATTTTTTTGGTAACCGGACCGAGGCTGCAGCGTCGTCCTCCCTAG | |
| | GAACCCCTAGTGATGGAGTTGGCCACTCCCTCTCTGCGCGCTCGCTC | |
| | GCTCACTGAGGCCGGGCGACCAAAGGTCGCCCGACGCCCGGGCTTT | |
| | GCCCGGGCGGCCTCAGTGAGCGAGCGAGCGCGCAGCTGCCTGCAGG | |
| CTX-525 | CCTGCAGGCAGCTGCGCGCTCGCTCGCTCACTGAGGCCGCCCGGGCG | 69 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGGTGTGGAAAGTCCCCAGGCTCCCCAGCAGGCAGAAGTATG | |
| | CAAAGCATGCATCTCAATTAGTCAGCAACCACGTTACATAACTTACG | |
| | GTAAATGGCCCGCCTGGCTGACCGCCCAACGACCCCCGCCCATTGAC | |
| | GTCAATAATGACGTATGTTCCCATAGTAACGCCAATAGGGACTTTCC | |
| | ATTGACGTCAATGGGTGGAGTATTTACGGTAAACTGCCCACTTGGCA | |
| | GTACATCAAGTGTATCATATGCCAAGTACGCCCCCTATTGACGTCAA | |
| | TGACGGTAAATGGCCCGCCTGGCATTATGCCCAGTACATGACCTTAT | |
| | GGGACTTTCCTACTTGGCAGTACATCTACGTATTAGTCATCGCTATTA | |
| | CCATGGTGATGCGGTTTTGGCAGTACATCAATGGGCGTGGATAGCGG | |
| | TTTGACTCACGGGGATTTCCAAGTCTCCACCCCATTGACGTCAATGG | |
| | GAGTTTGTTTTGGCACCAAAATCAACGGGACTTTCCAAAATGTCGTA | |
| | ACAACTCCGCCCCATTGACGCAAATGGGCGGTAGGCGTGTACGGTG | |
| | GGAGGTCTATATAAGCAGAGCTCGTTTAGTGAACCGTCACCGACTAT | |
| | GATTAAATGCTTGATATTGAGTGCCACCATGGCCCCAAAGAAGAAG | |
| | CGGAAGGTCGGATCCGGAAAGCGGAACTATATCCTGGGACTGGACA | |
| | TCGGAATTACCTCCGTGGGATACGGCATCATCGATTACGAGACTAGG | |
| | GACGTGATTGACGCCGGCGTGAGACTCTTTAAGGAGGCCAACGTGG | |
| | AAAACAACGAAGGTCGCAGATCCAAGCGGGGTGCAAGACGCCTGAA | |
| | GCGCCGGAGGAGACATCGGATACAGCGCGTGAAGAAGCTCCTTTTC | |
| | GACTACAACCTCCTCACTGACCACTCGGAATTGTCCGGTATCAACCC | |
| | CTACGAAGCCCGCGTGAAAGGCCTGAGCCAGAAGCTGTCCGAAGAG | |
| | GAGTTTAGCGCAGCCCTGCTGCACCTGGCTAAGCGAAGGGGGGTGC | |
| | ACAACGTGAACGAGGTGGAGGAGGACACTGGCAACGAACTGTCCAC | |
| | CAAGGAGCAGATTTCACGGAACTCGAAGGCGCTGGAAGAGAAATAT | |
| | GTGGCCGAGCTGCAGCTGGAGAGGCTCAAGAAGGATGGCGAAGTCC | |
| | GGGGGAGCATCAATCGCTTCAAGACCTCGGACTACGTGAAGGAAGC | |
| | CAAACAGCTGTTGAAGGTGCAGAAGGCCTACCACCAACTGGACCAA | |
| | TCATTCATTGACACTTACATCGATCTGCTTGAAACCAGGCGCACCTA | |
| | CTACGAGGGTCCTGGAGAAGGCAGCCCTTTCGGATGGAAGGACATC | |
| | AAGGAGTGGTATGAGATGCTGATGGGTCATTGCACCTACTTTCCGGA | |
| | AGAACTGCGCTCAGTGAAGTACGCGTACAACGCTGACCTCTACAAC | |
| | GCTCTCAACGATCTGAACAACCTCGTGATCACCCGGGACGAGAACG | |
| | AAAAGCTGGAGTACTACGAAAAGTTCCAGATTATCGAAAACGTGTT | |
| | CAAGCAGAAGAAGAAGCCCACCCTGAAGCAGATTGCAAAGGAGATC | |
| | CTTGTGAACGAGGAGGATATTAAGGGCTACCGGGTCACCTCCACCG | |
| | GGAAACCAGAGTTCACTAATCTCAAGGTGTACCATGACATTAAGGA | |
| | CATTACTGCCCGCAAGGAGATCATTGAAAACGCGGAACTGCTGGAC | |
| | CAAATCGCGAAGATCCTGACCATCTATCAGAGCTCCGAGGATATCCA | |
| | GGAGGAACTTACTAACCTCAATTCCGAGCTGACGCAGGAAGAAATC | |
| | GAGCAAATTAGCAACCTGAAGGGTTACACTGGAACCCACAACCTCA | |
| | GCTTGAAAGCGATTAACCTTATTTTGGATGAACTTTGGCACACTAAT | |
| | GACAATCAGATCGCCATTTTCAACCGGCTGAAACTGGTGCCGAAGA | |
| | AGGTGGACCTGAGCCAACAGAAGGAAATCCCGACCACCCTTGTGGA | |
| | CGATTTCATCCTGTCACCTGTGGTGAAGAGGAGCTTCATCCAGTCGA | |
| | TCAAGGTCATCAACGCCATCATAAAGAAGTACGGCCTTCCCAACGA | |
| | CATCATCATCGAACTGGCCCGCGAGAAGAACTCCAAAGATGCCCAG | |
| | AAGATGATCAACGAGATGCAGAAGCGAAACCGGCAGACGAACGAA | |
| | CGGATCGAGGAGATCATCCGGACCACCGGGAAGGAAAACGCGAAGT | |
| | ACCTGATCGAGAAAATCAAGCTGCATGATATGCAGGAAGGGAAGTG | |
| | TCTCTACTCCCTGGAGGCCATTCCGCTGGAGGATTTGCTGAACAACC | |
| | CTTTCAACTACGAAGTCGATCATATCATTCCTCGCTCCGTGTCCTTCG | |
| | ATAACTCCTTCAACAATAAGGTCCTCGTGAAGCAGGAGGAGAAGTA | |
| | AGTATCAAGGTTACAAGACAGCTTAAAGGCTTCATATAAGGGTGGA | |
| | ATCTTGTCGAGACAGAGAAGACTCTTGCGTTTCTGATAGGCACCTAT | |
| | TGGTCTTACTGACATCCACTTTGCCTTTCTCTCCACAGCTCGAAGAAG | |
| | GGCAACAGAACCCCGTTCCAGTACCTCTCGTCGTCCGACTCCAAGAT | |
| | CAGCTACGAAACTTTCAAGAAGCACATTCTGAACCTGGCCAAGGGC | |
| | AAAGGGAGAATTAGCAAGACCAAGAAGGAATACCTCCTGGAAGAG | |
| | AGAGACATCAACCGCTTCTCGGTGCAAAAGGATTTCATCAACCGCA | |
| | ACCTGGTCGATACCAGATACGCCACCAGGGGACTGATGAACCTCCT | |
| | GCGGTCCTACTTCCGGGTCAACAATCTGGACGTGAAGGTCAAATCCA | |
| | TCAACGGGGGCTTTACTTCTTTCCTGCGCCGGAAGTGGAAGTTCAAG | |
| | AAGGAACGGAACAAGGGATACAAGCACCACGCTGAAGATGCCCTGA | |
| | TTATTGCCAACGCCGACTTCATCTTTAAGGAATGGAAAAAGCTGGAC | |
| | AAGGCTAAGAAGGTCATGGAGAACCAGATGTTCGAAGAAAAGCAG | |
| | GCCGAGTCCATGCCCGAAATCGAAACCGAGCAGGAATACAAGGAGA | |
| | TCTTCATCACACCGCACCAAATCAAGCACATCAAGGACTTCAAGGAT | |
| | TACAAGTACAGCCACCGGGTGGACAAGAAGCCTAACAGAGAGCTTA | |
| | TCAACGACACCCTGTACTCCACGCGCAAGGACGACAAGGGAAACAC | |
| | ATTGATCGTGAACAACCTGAACGGACTGTATGACAAGGACAATGAC | |
| | AAACTGAAGAAGCTGATCAACAAATCGCCGGAAAAGCTCCTGATGT | |
| | ACCATCACGACCCTCAAACCTACCAGAAACTGAAGCTCATCATGGA | |
| | GCAGTACGGCGACGAAAAGAATCCCCTGTACAAATACTACGAGGAG | |
| | ACTGGAAATTACCTGACTAAGTACTCCAAGAAGGATAACGGCCCCG | |
| | TGATCAAGAAGATTAAGTACTACGGAAACAAACTGAACGCACATCT | |
| | CGACATCACCGATGATTATCCAAACTCCCGCAACAAAGTCGTGAAG | |
| | CTCTCCCTCAAACCGTACCGCTTCGACGTGTACCTGGATAATGGGGT | |
| | GTACAAGTTCGTGACCGTGAAGAACCTGGACGTCATTAAGAAGGAA | |
| | AACTACTACGAAGTGAACTCAAAGTGCTACGAGGAAGCCAAGAAGC | |
| | TCAAGAAGATCAGCAACCAGGCCGAGTTCATCGCATCGTTTTACAAC | |
| | AATGACCTCATTAAGATTAATGGAGAACTGTACAGAGTGATCGGCGT | |
| | GAACAACGACCTCCTGAACCGGATTGAAGTGAACATGATCGATATT | |
| | ACCTACCGGGAGTATCTGGAGAACATGAACGACAAGCGCCCACCGA | |
| | GAATCATCAAAACTATTGCCTCCAAGACCCAATCCATTAAGAAATAC | |
| | TCCACCGACATCCTGGGCAACCTGTACGAGGTCAAGTCGAAGAAGC | |
| | ACCCCCAGATTATCAAGAAGGGAAAGCTTGCCCCAAAGAAGAAGCG | |
| | GAAGGTCGGTACTAGTGAGGGCAGGGGAAGTCTGCTAACATGCGGG | |
| | GACGTGGAGGAAAATCCCGGCCCCATGGCTAAGACTTCCGAACAGA | |
| | GGGTGAACATTGCTACACTGCTGACAGAAAATAAGAAGAAAATCGT | |
| | GGATAAGGCTTCCCAGGATCTGTGGCGGAGACACCCAGACCTGATC | |
| | GCACCAGGAGGAATTGCTTTCTCTCAGAGGGACCGCGCTCTGTGCCT | |
| | GCGAGATTACGGCTGGTTCCTGCATCTGATCACCTTTTGTCTGCTGGC | |
| | CGGAGATAAGGGCCCCATCGAGTCTATTGGGCTGATCAGTATTCGAG | |
| | AAATGTATAACTCACTGGGAGTGCCCGTCCCTGCAATGATGGAGAG | |
| | CATTAGATGCCTGAAAGAAGCCAGCCTGTCCCTGCTGGACGAAGAG | |
| | GACGCCAACGAGACCGCACCCTACTTTGATTACATTATTAAGGCTAT | |
| | GAGCTAAGCGCTAATAAAATATCTTTATTTTCATTACATCTGTGTGTT | |
| | GGTTTTTTGTGTGGTAACCACGTGCGGACCGAGGCTGCAGCGTCGTC | |
| | CTCCCTAGGAACCCCTAGTGATGGAGTTGGCCACTCCCTCTCTGCGC | |
| | GCTCGCTCGCTCACTGAGGCCGGGCGACCAAAGGTCGCCCGACGCC | |
| | CGGGCTTTGCCCGGGCGGCCTCAGTGAGCGAGCGAGCGCGCAGCTG | |
| | CCTGCAGG | |
| CTX-1048 | CCTGCAGGCAGCTGCGCGCTCGCTCGCTCACTGAGGCCGCCCGGGCG | 70 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAATTCCGTTACATAACTTACGGTAAATGGCCCGCCTGGCTG | |
| | ACCGCCCAACGACCCCCGCCCATTGACGTCAATAATGACGTATGTTC | |
| | CCATAGTAACGCCAATAGGGACTTTCCATTGACGTCAATGGGTGGAG | |
| | TATTTACGGTAAACTGCCCACTTGGCAGTACATCAAGTGTATCATAT | |
| | GCCAAGTACGCCCCCTATTGACGTCAATGACGGTAAATGGCCCGCCT | |
| | GGCATTATGCCCAGTACATGACCTTATGGGACTTTCCTACTTGGCAG | |
| | TACATCTACGTATTAGTCATCGCTATTACCATGGTGATGCGGTTTTGG | |
| | CAGTACATCAATGGGCGTGGATAGCGGTTTGACTCACGGGGATTTCC | |
| | AAGTCTCCACCCCATTGACGTCAATGGGAGTTTGTTTTGGCACCAAA | |
| | ATCAACGGGACTTTCCAAAATGTCGTAACAACTCCGCCCCATTGACG | |
| | CAAATGGGCGGTAGGCGTGTACGGTGGGAGGTCTATATAAGCAGAG | |
| | CTCGTTTAGTGAACCGTGCTAGCATCCACATAGTGTACTTACAGTCA | |
| | GAAGCCACCATGGCCCCAAAGAAGAAGCGGAAGGTCGGATCCGGA | |
| | AAGCGGAACTATATCCTGGGACTGGACATCGGAATTACCTCCGTGG | |
| | GATACGGCATCATCGATTACGAGACTAGGGACGTGATTGACGCCGG | |
| | CGTGAGACTCTTTAAGGAGGCCAACGTGGAAAACAACGAAGGTCGC | |
| | AGATCCAAGCGGGGTGCAAGACGCCTGAAGCGCCGGAGGAGACATC | |
| | GGATACAGCGCGTGAAGAAGCTCCTTTTCGACTACAACCTCCTCACT | |
| | GACCACTCGGAATTGTCCGGTATCAACCCCTACGAAGCCCGCGTGAA | |
| | AGGCCTGAGCCAGAAGCTGTCCGAAGAGGAGTTTAGCGCAGCCCTG | |
| | CTGCACCTGGCTAAGCGAAGGGGGGTGCACAACGTGAACGAGGTGG | |
| | AGGAGGACACTGGCAACGAACTGTCCACCAAGGAGCAGATTTCACG | |
| | GAACTCGAAGGCGCTGGAAGAGAAATATGTGGCCGAGCTGCAGCTG | |
| | GAGAGGCTCAAGAAGGATGGCGAAGTCCGGGGGAGCATCAATCGCT | |
| | TCAAGACCTCGGACTACGTGAAGGAAGCCAAACAGCTGTTGAAGGT | |
| | GCAGAAGGCCTACCACCAACTGGACCAATCATTCATTGACACTTACA | |
| | TCGATCTGCTTGAAACCAGGCGCACCTACTACGAGGGTCCTGGAGA | |
| | AGGCAGCCCTTTCGGATGGAAGGACATCAAGGAGTGGTATGAGATG | |
| | CTGATGGGTCATTGCACCTACTTTCCGGAAGAACTGCGCTCAGTGAA | |
| | GTACGCGTACAACGCTGACCTCTACAACGCTCTCAACGATCTGAACA | |
| | ACCTCGTGATCACCCGGGACGAGAACGAAAAGCTGGAGTACTACGA | |
| | AAAGTTCCAGATTATCGAAAACGTGTTCAAGCAGAAGAAGAAGCCC | |
| | ACCCTGAAGCAGATTGCAAAGGAGATCCTTGTGAACGAGGAGGATA | |
| | TTAAGGGCTACCGGGTCACCTCCACCGGGAAACCAGAGTTCACTAAT | |
| | CTCAAGGTGTACCATGACATTAAGGACATTACTGCCCGCAAGGAGA | |
| | TCATTGAAAACGCGGAACTGCTGGACCAAATCGCGAAGATCCTGAC | |
| | CATCTATCAGAGCTCCGAGGATATCCAGGAGGAACTTACTAACCTCA | |
| | ATTCCGAGCTGACGCAGGAAGAAATCGAGCAAATTAGCAACCTGAA | |
| | GGGTTACACTGGAACCCACAACCTCAGCTTGAAAGCGATTAACCTTA | |
| | TTTTGGATGAACTTTGGCACACTAATGACAATCAGATCGCCATTTTC | |
| | AACCGGCTGAAACTGGTGCCGAAGAAGGTGGACCTGAGCCAACAGA | |
| | AGGAAATCCCGACCACCCTTGTGGACGATTTCATCCTGTCACCTGTG | |
| | GTGAAGAGGAGCTTCATCCAGTCGATCAAGGTCATCAACGCCATCAT | |
| | AAAGAAGTACGGCCTTCCCAACGACATCATCATCGAACTGGCCCGC | |
| | GAGAAGAACTCCAAAGATGCCCAGAAGATGATCAACGAGATGCAGA | |
| | AGCGAAACCGGCAGACGAACGAACGGATCGAGGAGATCATCCGGA | |
| | CCACCGGGAAGGAAAACGCGAAGTACCTGATCGAGAAAATCAAGCT | |
| | GCATGATATGCAGGAAGGGAAGTGTCTCTACTCCCTGGAGGCCATTC | |
| | CGCTGGAGGATTTGCTGAACAACCCTTTCAACTACGAAGTCGATCAT | |
| | ATCATTCCTCGCTCCGTGTCCTTCGATAACTCCTTCAACAATAAGGTC | |
| | CTCGTGAAGCAGGAGGAGAAGTAAGTATCAAGGTTACAAGACAGCT | |
| | ATTCTGAGTACAGAGCATACAGAGTCTTGTCGAGACAGAGAAGACT | |
| | CTTGCGTTTCTGATAGGCACCTATTGGTCTTACTGACATCCACTTTGC | |
| | CTTTCTCTCCACAGCTCGAAGAAGGGCAACAGAACCCCGTTCCAGTA | |
| | CCTCTCGTCGTCCGACTCCAAGATCAGCTACGAAACTTTCAAGAAGC | |
| | ACATTCTGAACCTGGCCAAGGGCAAAGGGAGAATTAGCAAGACCAA | |
| | GAAGGAATACCTCCTGGAAGAGAGAGACATCAACCGCTTCTCGGTG | |
| | CAAAAGGATTTCATCAACCGCAACCTGGTCGATACCAGATACGCCA | |
| | CCAGGGGACTGATGAACCTCCTGCGGTCCTACTTCCGGGTCAACAAT | |
| | CTGGACGTGAAGGTCAAATCCATCAACGGGGGCTTTACTTCTTTCCT | |
| | GCGCCGGAAGTGGAAGTTCAAGAAGGAACGGAACAAGGGATACAA | |
| | GCACCACGCTGAAGATGCCCTGATTATTGCCAACGCCGACTTCATCT | |
| | TTAAGGAATGGAAAAAGCTGGACAAGGCTAAGAAGGTCATGGAGAA | |
| | CCAGATGTTCGAAGAAAAGCAGGCCGAGTCCATGCCCGAAATCGAA | |
| | ACCGAGCAGGAATACAAGGAGATCTTCATCACACCGCACCAAATCA | |
| | AGCACATCAAGGACTTCAAGGATTACAAGTACAGCCACCGGGTGGA | |
| | CAAGAAGCCTAACAGAGAGCTTATCAACGACACCCTGTACTCCACG | |
| | CGCAAGGACGACAAGGGAAACACATTGATCGTGAACAACCTGAACG | |
| | GACTGTATGACAAGGACAATGACAAACTGAAGAAGCTGATCAACAA | |
| | ATCGCCGGAAAAGCTCCTGATGTACCATCACGACCCTCAAACCTACC | |
| | AGAAACTGAAGCTCATCATGGAGCAGTACGGCGACGAAAAGAATCC | |
| | CCTGTACAAATACTACGAGGAGACTGGAAATTACCTGACTAAGTACT | |
| | CCAAGAAGGATAACGGCCCCGTGATCAAGAAGATTAAGTACTACGG | |
| | AAACAAACTGAACGCACATCTCGACATCACCGATGATTATCCAAACT | |
| | CCCGCAACAAAGTCGTGAAGCTCTCCCTCAAACCGTACCGCTTCGAC | |
| | GTGTACCTGGATAATGGGGTGTACAAGTTCGTGACCGTGAAGAACCT | |
| | GGACGTCATTAAGAAGGAAAACTACTACGAAGTGAACTCAAAGTGC | |
| | TACGAGGAAGCCAAGAAGCTCAAGAAGATCAGCAACCAGGCCGAGT | |
| | TCATCGCATCGTTTTACAACAATGACCTCATTAAGATTAATGGAGAA | |
| | CTGTACAGAGTGATCGGCGTGAACAACGACCTCCTGAACCGGATTG | |
| | AAGTGAACATGATCGATATTACCTACCGGGAGTATCTGGAGAACAT | |
| | GAACGACAAGCGCCCACCGAGAATCATCAAAACTATTGCCTCCAAG | |
| | ACCCAATCCATTAAGAAATACTCCACCGACATCCTGGGCAACCTGTA | |
| | CGAGGTCAAGTCGAAGAAGCACCCCCAGATTATCAAGAAGGGAAAG | |
| | CTTGCCCCAAAGAAGAAGCGGAAGGTCTAAGGTACTAGTAATAAAA | |
| | TATCTTTATTTTCATTACATCTGTGTGTTGGTTTTTTGTGTGAGCGCTG | |
| | GTAACCGGACCGAGGCTGCAGCGTCGTCCTCCCTAGGAACCCCTAGT | |
| | GATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGC | |
| | CTCAGTGAGCGAGCGAGCGCGCAGCTGCCTGCAGG | |
| CTX-1075 | CCTGCAGGCAGCTGCGCGCTCGCTCGCTCACTGAGGCCGCCCGGGCG | 71 |
| | TCGGGCGACCTTTGGTCGCCCGGCCTCAGTGAGCGAGCGAGCGCGC | |
| | AGAGAGGGAGTGGCCAACTCCATCACTAGGGGTTCCTGCGGCCGCA | |
| | CGCGTGAGGGCCTATTTCCCATGATTCCTTCATATTTGCATATACGAT | |
| | ACAAGGCTGTTAGAGAGATAATTGGAATTAATTTGACTGTAAACACA | |
| | AAGATATTAGTACAAAATACGTGACGTAGAAAGTAATAATTTCTTGG | |
| | GTAGTTTGCAGTTTTAAAATTATGTTTTAAAATGGACTATCATATGCT | |
| | TACCGTAACTTGAAAGTATTTCGATTTCTTGGCTTTATATATCTTGTG | |
| | GAAAGGACGAAACACCGTTCTGACTGTAAGTACACTATGTTTAAGTA | |
| | CTCTGTGCTGGAAACAGCACAGAATCTACTTAAACAAGGCAAAATG | |
| | CCGTGTTTATCTCGTCAACTTGTTGGCGAGATTTTTTTCACCGGTGGC | |
| | ACACACTGTAGTTCATCTTTACATGGCCTCATTGAAGACTACAGCTC | |
| | TGGTATGCGTATAAGGAACTAGCATTAGGTCATTTCAAGCCGATGCT | |
| | AGAATCCAGATTCCATGCTGACCGATGAGGATATAGTGAGAATCTTT | |
| | CAAGAACATTCTTAACCGTTGGTATCTTAGCTCCACCCTCACTGGTTC | |
| | TTCCGGCCAAGCTGCTGGCCTCCCTCCTCAACCGTTCTGATCATGCTT | |
| | GCTTAGTCGGCCAGTTAAGCCTGATTATGACCTGGTTACCTGTTGTCT | |
| | AAGGGCAGGAATCACCGCCGTAACTCTAGCACTTAGCACAGTACTT | |
| | GGCTTGTAAGAGGTCCTCGATGATGTGAATACATTAAATAATTAACC | |
| | TAAGAAAGATTTCATATTAGGCATTGTAATGACTTAAGGTAAAGAGC | |
| | AGTGCTATTAACAATCCAGCTTGTTTGGGCTATTGTGGCTGTGGGCA | |
| | CCTCTCTGGGTGTATATCTGAGGTGCTGGCTACCTCTTGGAGGATTAT | |
| | AAGACAATCAGCAACCCTTGCATGGTGGCAACAGTAATAATAGCCA | |
| | TCCTTACATAGTCCTACAGCCCTGTAGCAATGGTCCAACAGATGAGG | |
| | AACCTTTGAAGCCTCAGAGAGGCTAACAGACAGACCCTAGGTCATA | |
| | CAGTTATTAAGAGAAGGCGAACCTCTCTCGAGTAATACCAGTTAATA | |
| | GGCTACACAAATGGTAGTGGCTGTTGTATTCAGTTGCTGAGGAATGC | |
| | TAAACATAATTCTGCCAATTTCCGCACCCGACTTCCCGGGCTCGGGT | |
| | GATTCTAGGGCTGTGTCATTTGTATACGCTCTTGTTGCCCGGGCTGGA | |
| | GTACAGTGGCCTCAGTGCTCCCGGGTTCCCTACCTCATGCGCCTGTA | |
| | TAATAGAGACGAGGTTTCACAGGCTACCTGATCCAGTGAATATTTGT | |
| | ATTGTAGAGATGGTGGCCATGTTCCTGAGCTCAAGCGATCTGCCCGC | |
| | CTCTGGCCACCGTGCCTGGCCTAGGTAGACGCAGCGTGATGCCTGAG | |
| | TATATAGTGATGCTAGAGCTGGCTGTTTGTTAGCTTTGAACATAAGA | |
| | TACTCATTGTAGTTTGCAAATCCCTCTTCCTAATTTCTTTCCCTTAAAT | |
| | TGTTTGCATGTTAGCGCTTAAATGGTGCTATGTGCTAGAAGCCTTAA | |
| | ATTACACAAATCAGAGAGGTGCCCAACTTTGAACCTAAGCTGCTCTT | |
| | AATCTCTAAACAAGTTAGTAGTGACAATAGTAGGATACTTAACTATG | |
| | AGGCATAGCAGGCATTATCACCCTAAAGTGTACCCTTTAGGTAAGTA | |
| | TATACTTGCCCAATATCACTTATCAAATGTGTCTGATACAACCCAAA | |
| | CTATCGAAACTGCCAGGGTAAACTTGGACACACTTGAGCTAAGAATT | |
| | AAGTCCTAGAAATGTAATCCTGCCCTAGCCGAGCTTACCCTGCAGAA | |
| | TTGGTCGGAGCACCGTCCTTGGCCACACTGTTATCAACAGGGTGTCA | |
| | ATCTGTAGGAATTACTCTTTGTGACCACCAGGAAATAGAGCAGTTCA | |
| | GTTCATTTCTTTCTCACTGTGACCTGCATACTACAAGTCTACTTTGCT | |
| | ATCCATTGTTTGTATCTGGGTATTACCAGATCAGCAGAGAAGAGTTG | |
| | CCTTGGAGCAGCTGCAGTTCATTAGATAGTAACTAGGCCATGTCAAC | |
| | TCCCTTGTAGTGAAGATTGTACTGGTACCTTTCTGTAAATATTGTGTA | |
| | GATCAATCACCACCTCAACCCAGTGGCTGCCAAATTACAATAATTCA | |
| | CTACTACTAAGATAATCTACTAGTTCGATCACATACTTCCTACTGTCT | |
| | TCAGCATTGTGCTTCTGATTATAATTGTCCAGAGTGAACATGTCTATT | |
| | CTTCCACTGTACACACTAATGGATTGTAATATTGGGTAAATTCATGT | |
| | CCTTACACATGTAGTAGTTATGAGCCCATGTCCCTAGAATGAGTAAT | |
| | AACCTTGGTTGAATAGTCAAGAATGCTGAAATTCTTCTAACAGCAGA | |
| | AGGGAAGGCAAGCAAGTGTTACTGATAAGATGAATCTACTATTAGC | |
| | TTTAATTATACATTTAGGAATATTGCATCAGTAACTCATAAGGCTGTT | |
| | ATCCTGAGTTAACACAAATTATCCAAGGAGATCTGCTTTGAGGTGTG | |
| | AGTGTATCTGATGCCAACTAGCAATTCCAGAAGTTTGGAATTAAATT | |
| | ATGGTTTATCTATTGTTATACCTCAATTATATCATGTTTGCTGTGCTC | |
| | TCGGCTCACTCTAGCCACCGACTCCCTCTGAGCCTTGCAGGGTAGAG | |
| | ACAGGATTGGCCAGGATGGTCTCCATCATGATCGGCCTCGTGGGAGC | |
| | CACTACGCCTGGCCATAGACTCACTTCCATTAAGTCTTGTTTGGACC | |
| | CACGAACATTGTCTTTAAGATGGAGTTTCACGTTGCCCAGACTGTAG | |
| | TGCAATGGTGCAATCTCAGCTCACTGCAACCAATTCTCCTCCCGAGT | |
| | AGCTGGAATTACAGGCGCCCGCCACCACGGTGTTTCACCGGCCATGA | |
| | TCCGCCCACCTCAGCCTCGTGTGAGCCACCGCATCTGGCCAACATGT | |
| | CTTCCTAGACTTAAGCACAGATGATGAATTGATGTGTCTTAGCTTGG | |
| | ATTAACTTGCTTACTGTAAAGATAATATAGCTTGACATGAAGGCCAT | |
| | TATTACAGATGTGACGTGCATAATTATTAGTATTACATGGGTCAGTC | |
| | TGGCAATTATGAAGAATAATGCCAGACATTTCAGTAATCGATTATAG | |
| | CGTATTGACAGTCCAGACGTCAGAATTTCTCAATACTCTTTCAGATT | |
| | AATGTACCTGTAGCGATATCATTCACAAGTATATCACAAGTAAGTTA | |
| | GAATTTGAGAACTGTGTTCTAGAGATGCAGTCAGATTTCTGAACTGT | |
| | CTCAGCAAATGGAGAGCTAGTAATTAATAACCTGTCCTTTGATTTCT | |
| | GATTCAGCCAAGAATGGCCATATTTGGGAAGGAGAGTAACCACGCA | |
| | TTCATTTACCACAGAGCTCTCAGCTTAAAGCCATACAGGACCGTGAT | |
| | CTGTTCTAGCCATATGTAGCATTTATGTCCTAGTGTGATGGTATTTGG | |
| | AGACAGGGCCTTTGGAAGGTAATTGAAGTGGGCCCAGGTCTGATTG | |
| | GATTAGTGCGGGCGCACAAGGCCAATCACGAGGTCAGCCAGCCTGG | |
| | CCAATGTAGTGAAACACCAACATTAGCTGGGTGTGGTAGCGGGCTC | |
| | CTGTCATCCAAGCTACGAGGCATGAGAATCGGGACAGATTGTGCCA | |
| | CTGTGGGTGACTCAAGAGACACCAGAGAGCTTGTTAGAAGAGGTCA | |
| | TGTGAGCACGACCTTCAAGCCAAAGAAGAGGCCTGAGATTGAAACC | |
| | TACCTTGCAGGTATTCCGTGAGAAATAAGTTTCTGTTAAGTCACTCA | |
| | GTCTGTGGTAGTTATGGCAGCCTGAGCAGGTAGTTGTTCTTTCAGAA | |
| | GGTGTTGATAATCAGATGCTAGCGAGGGCCTATTTCCCATGATTCCT | |
| | TCATATTTGCATATACGATACAAGGCTGTTAGAGAGATAATTGGAAT | |
| | TAATTTGACTGTAAACACAAAGATATTAGTACAAAATACGTGACGTA | |
| | GAAAGTAATAATTTCTTGGGTAGTTTGCAGTTTTAAAATTATGTTTTA | |
| | AAATGGACTATCATATGCTTACCGTAACTTGAAAGTATTTCGATTTCT | |
| | TGGCTTTATATATCTTGTGGAAAGGACGAAACACCGCTATTCTGAGT | |
| | ACAGAGCATAGTTTAAGTACTCTGTGCTGGAAACAGCACAGAATCT | |
| | ACTTAAACAAGGCAAAATGCCGTGTTTATCTCGTCAACTTGTTGGCG | |
| | AGATTTTTTTGGTAACCGGACCGAGGCTGCAGCGTCGTCCTCCCTAG | |
| | GAACCCCTAGTGATGGAGTTGGCCACTCCCTCTCTGCGCGCTCGCTC | |
| | GCTCACTGAGGCCGGGCGACCAAAGGTCGCCCGACGCCCGGGCTTT | |
| | GCCCGGGCGGCCTCAGTGAGCGAGCGAGCGCGCAGCTGCCTGCAGG | |

1. A CRISPR/Cas two vector system comprising:
(a) a first vector comprising a nucleic acid encoding (i) a first guide RNA (gRNA) comprising a DNA targeting sequence that is complementary to a first portion of the human DMD gene, wherein the DNA targeting sequence is 19-24 nucleotides in length and comprises a nucleotide sequence selected from the SEQ ID NOs:1-17; and (ii) a second gRNA comprising a DNA targeting sequence that is complementary to a second portion of the human DMD gene, wherein the DNA targeting sequence is 19-24 nucleotides in length and comprises a nucleotide sequence selected from SEQ ID NOs:18-31; and
(b) a second vector comprising a nucleic acid encoding a site-directed Cas9 polypeptide or variant thereof, wherein the nucleic acid encoding the site-directed Cas9 polypeptide comprises (i) a first gRNA target sequence which binds the first gRNA; and (ii) a second gRNA target sequence which binds the second gRNA,
wherein binding of the first and second gRNAs to the nucleic acid encoding the site-directed Cas9 polypeptide inhibits expression of the Cas9 polypeptide.

2. The CRISPR/Cas two vector system of claim 1, wherein the first vector comprises a nucleic acid encoding from 5′ to 3′
(i) a first inverted terminal repeat (ITR);
(ii) a first promoter;
(iii) the first gRNA;
(iv) a detectable polypeptide;
(v) a second promoter;
(vi) the second gRNA; and
(vii) a second ITR; and/or
the second vector comprises a nucleic acid encoding from 5′ to 3′
(i) a first inverted terminal repeat (ITR);
(ii) a promoter;
(iii) the site directed Cas9 polypeptide or variant thereof comprising the first and second gRNA target sequences.

3. (canceled)

4. The CRISPR/Cas two vector system of claim 1, wherein the first gRNA target sequence is located at the 5′ end of the nucleic acid encoding the site directed Cas9 polypeptide or variant thereof.

5. The CRISPR/Cas two vector system of claim 1, wherein the second gRNA target sequence is located within the open reading frame (ORF) of the site directed Cas9 polypeptide or variant thereof.

6. The CRISPR/Cas two vector system of claim 1, wherein the second target sequence is located in a chimeric intron inserted into the open reading frame of the directed Cas9 polypeptide or variant thereof.

7. The CRISPR/Cas two vector system of claim 1, wherein the DNA targeting sequence of the first gRNA comprises SEQ ID NO: 13 or SEQ ID NO: 14.

8. The CRISPR/Cas two vector system of claim 1, wherein the DNA targeting sequence of the second gRNA comprises SEQ ID NO: 25.

9. The CRISPR/Cas two vector system of claim 1, wherein the DNA targeting sequence of the first gRNA comprises SEQ ID NO: 13, and the DNA targeting sequence of the second gRNA comprises SEQ ID NO: 25.

10. The CRISPR/Cas two vector system of claim 1, wherein the first gRNA that is complementary to a portion of the DMD gene and/or the second gRNA that is complementary to a portion of the DMD gene is a single RNA molecule.

11. (canceled)

12. The CRISPR/Cas two vector system of claim 1, wherein the first gRNA that is complementary to a portion of the DMD gene and/or the second gRNA that is complementary to a portion of the DMD gene is a two-molecule guide RNA.

13. (canceled)

14. (canceled)

15. The CRISPR/Cas two vector system of claim 1, wherein the first gRNA target sequence of the second vector comprises the nucleotide sequence set forth in SEQ ID NO: 32 or SEQ ID NO: 33.

16. The CRISPR/Cas two vector system of claim 1, wherein the second gRNA target sequence of the second vector comprises the nucleotide set forth in SEQ ID NO: 34.

17. The CRISPR/Cas two vector system of claim 1, wherein the first and second gRNA target sequences are in the opposite orientation.

18. The CRISPR/Cas two vector system of claim 1, wherein the first vector is an adeno-associated virus (AAV) vector and/or the second vector is an AAV vector.

19. (canceled)

20. The CRISPR/Cas two vector system of claim 1, wherein the site-directed Cas9 polypeptide is Staphylococcus aureus Cas9 (SaCas9) or a variant thereof, optionally wherein the nucleotide sequence encoding the Cas9 polypeptide or variant thereof is codon optimized.

21. The CRISPR/Cas two vector system of claim 17, wherein the site-directed Cas9 polypeptide comprises the amino acid sequence set forth in SEQ ID NO: 46, SEQ ID NO: 47, SEQ ID NO: 48 or SEQ ID NO: 49.

22. (canceled)

23. The CRISPR/Cas two vector system of claim 1, wherein:
a) the first vector comprises the nucleotide sequence set forth in SEQ ID NO: 68 or SEQ ID NO: 71, and/or
b) the second vector comprises the nucleotide sequence set forth in SEQ ID NO: 67 or SEQ ID NO: 70.

24-27. (canceled)

28. A genetically modified cell comprising the CRISPR/Cas two vector system of claim 1, optionally wherein the genetically modified cell is a somatic cell, a stem cell selected from an embryonic stem (ES) cell or an induced pluripotent stem (iPS) cell, or a muscle cell.

29-31. (canceled)

32. A method of correcting a mutation in the human DMD gene in a cell, the method comprising contacting the cell with the CRISPR/Cas two vector system of claim 1 wherein the correction of the mutant dystrophin gene comprises deletion of exon 51 of the human DMD gene.

33-39. (canceled)

40. A pharmaceutical composition comprising the CRISPR/Cas two vector system of claim 1.


SEQUENCE LISTING (75 sequences)

SEQ ID NO: 1 - DNA, 21 bp, Artificial Sequence
  Synthetic L01
ctgagtaggagctaaaatatt

SEQ ID NO: 2 - DNA, 21 bp, Artificial Sequence
  Synthetic L02
acaataagtcaaatttaattg

SEQ ID NO: 3 - DNA, 21 bp, Artificial Sequence
  Synthetic L03
aagatatataatgtcatgaat

SEQ ID NO: 4 - DNA, 21 bp, Artificial Sequence
  Synthetic L16
aatggttaagatgcatagtac

SEQ ID NO: 5 - DNA, 21 bp, Artificial Sequence
  Synthetic L18
tatgtggctttaccaaggtcc

SEQ ID NO: 6 - DNA, 21 bp, Artificial Sequence
  Synthetic L22
gtgtattgcttgtactactca

SEQ ID NO: 7 - DNA, 20 bp, Artificial Sequence
  Synthetic L34
tctcctcattagagaagaag

SEQ ID NO: 8 - DNA, 21 bp, Artificial Sequence
  Synthetic L37
ctcaagcttctcagggacacc

SEQ ID NO: 9 - DNA, 21 bp, Artificial Sequence
  Synthetic L45
atcctcacacatgcatcctct

SEQ ID NO: 10 - DNA, 21 bp, Artificial Sequence
  Synthetic L52
aaagtgaaggatgaggaacta

SEQ ID NO: 11 - DNA, 21 bp, Artificial Sequence
  Synthetic L57
aaattagctgaagcatattca

SEQ ID NO: 12 - DNA, 21 bp, Artificial Sequence
  Synthetic L61
tcttgcatcttgcacatgtcc

SEQ ID NO: 13 - DNA, 21 bp, Artificial Sequence
  Synthetic L64
cttagaggtcttctacataca

SEQ ID NO: 14 - DNA, 21 bp, Artificial Sequence
  Synthetic L81
ttctgactgtaagtacactat

SEQ ID NO: 15 - DNA, 20 bp, Artificial Sequence
  Synthetic L84
tctggagggtcaaatctggt

SEQ ID NO: 16 - DNA, 20 bp, Artificial Sequence
  Synthetic L85
aatggagagaggtaagtctg

SEQ ID NO: 17 - DNA, 21 bp, Artificial Sequence
  Synthetic L88
tgaaatggcctgtgctcatga

SEQ ID NO: 18 - DNA, 21 bp, Artificial Sequence
  Synthetic R6
aactggtgggaaatggtctag

SEQ ID NO: 19 - DNA, 21 bp, Artificial Sequence
  Synthetic R7
attatacttaggctgaatagt

SEQ ID NO: 20 - DNA, 20 bp, Artificial Sequence
  Synthetic R11
tttaaatgtaaatagctcag

SEQ ID NO: 21 - DNA, 21 bp, Artificial Sequence
  Synthetic R14
tggcacagacaacttagaaga

SEQ ID NO: 22 - DNA, 21 bp, Artificial Sequence
  Synthetic R15
aaattggcacagacaacttag

SEQ ID NO: 23 - DNA, 21 bp, Artificial Sequence
  Synthetic R22
aaaaacaagaagtgaggcaga

SEQ ID NO: 24 - DNA, 21 bp, Artificial Sequence
  Synthetic R26
ctgcatttaaaggccttgagc

SEQ ID NO: 25 - DNA, 21 bp, Artificial Sequence
  Synthetic R32
ctattctgagtacagagcata

SEQ ID NO: 26 - DNA, 21 bp, Artificial Sequence
  Synthetic R41
agcaagtaataacacaagctt

SEQ ID NO: 27 - DNA, 21 bp, Artificial Sequence
  Synthetic R42
gtgttattacttgctactgca

SEQ ID NO: 28 - DNA, 21 bp, Artificial Sequence
  Synthetic R52
acacttccttgtgacgggttt

SEQ ID NO: 29 - DNA, 21 bp, Artificial Sequence
  Synthetic R53
attgatgtgctcagtagtctc

SEQ ID NO: 30 - DNA, 21 bp, Artificial Sequence
  Synthetic R91
ttacacacaggatggagaaaa

SEQ ID NO: 31 - DNA, 20 bp, Artificial Sequence
  Synthetic R99
gcaattctcctgaatagaaa

SEQ ID NO: 32 - DNA, 114 bp, Artificial Sequence
  Synthetic L64 gRNA
cttagaggtcttctacatacagtttaagtactctgtgctggaaacagcacagaatctacttaaacaaggcaaaatgccgtgtttatctcgtcaacttgttggcgagattttttt

SEQ ID NO: 33 - DNA, 114 bp, Artificial Sequence
  Synthetic L81 gRNA
ttctgactgtaagtacactatgtttaagtactctgtgctggaaacagcacagaatctacttaaacaaggcaaaatgccgtgtttatctcgtcaacttgttggcgagattttttt

SEQ ID NO: 34 - DNA, 114 bp, Artificial Sequence
  Synthetic R32 gRNA
ctattctgagtacagagcatagtttaagtactctgtgctggaaacagcacagaatctacttaaacaaggcaaaatgccgtgtttatctcgtcaacttgttggcgagattttttt

SEQ ID NO: 35 - DNA, 114 bp, Artificial Sequence
  Synthetic LT2 gRNA
actatgattaaatgcttgatagtttaagtactctgtgctggaaacagcacagaatctacttaaacaaggcaaaatgccgtgtttatctcgtcaacttgttggcgagattttttt

SEQ ID NO: 36 - DNA, 114 bp, Artificial Sequence
  Synthetic RT2 gRNA
cttaaaggcttcatataaggggtttaagtactctgtgctggaaacagcacagaatctacttaaacaaggcaaaatgccgtgtttatctcgtcaacttgttggcgagattttttt

SEQ ID NO: 37 - DNA, 114 bp, Artificial Sequence
  Synthetic V25 gRNA
cgttggagcggggagaaggccgtttaagtactctgtgctggaaacagcacagaatctacttaaacaaggcaaaatgccgtgtttatctcgtcaacttgttggcgagattttttt

SEQ ID NO: 38 - DNA, 27 bp, Artificial Sequence
  Synthetic L64BS
actcattgtatgtagaagacctctaag

SEQ ID NO: 39 - DNA, 27 bp, Artificial Sequence
  Synthetic L81BS
atccacatagtgtacttacagtcagaa

SEQ ID NO: 40 - DNA, 27 bp, Artificial Sequence
  Synthetic R32BS
ctattctgagtacagagcatacagagt

SEQ ID NO: 41 - DNA, 130 bp, Artificial Sequence
  Synthetic 5 AAV ITR
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 42 - DNA, 249 bp, Artificial Sequence
  Synthetic U6 Promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 43 - DNA, 141 bp, Artificial Sequence
  Synthetic 3ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 2253 bp, Artificial Sequence
  Synthetic Albumin
agaaaacgccagtaagtgacagagtcacaaatgactgcacagagtccttggtgaacaggcgaccatgcttttcagctctggaagtcgtgaaaacatacgttcccaaagagttttgaactgaaaacttcaccttccatgcagatatatgcacactttctgagaaggagagacaaatcaagaaacaaactgcacttgttgagcttgtgaaacacaagcccaaggcaacaaaagagcaactgaaagctgtttgagatgatttcgcagcttttgtagagaagtgctgcaaggctgacgataaggagacctgctttgccgaggagggtaaaaaacttgttgctgcaagtcaagctgccttaggcttataacatctacatttaaaagactctcagcctacctgaagaataagagaaagaaatgaaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagttaagtttgcagaagtttccaagttagtgacagatcttaccaaagtccacacggaatgctgcctgagagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagtgacctgctgacttgccttacttagctgctgattttgttgaaagtaaggtgatttgcaaaaacttgactgaggcaaaggatgtcttcctgggctgatttttgtatgaatatgcaagaaggactcctgattactctgtcgtgctgctgctgagacttgccaagaactatgaaaccacagatctgaagtgctgtgccgctgcagatcctactgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctcggaaaagtgggcagcaaatgttgtaaacatcctgaagcaaaaagatgaccctgtgcagaagactatctatccgtggtcctgaaccagttatgtgtgttgcatgaggatgtcttctggcaatttcatataagtattttttcaaaatgatctcttctgtcaaccccacgcctttggcacatgaaagtgggtaacctttatttcccttctttttctctttagctcggcttattccaggggtgtgtttcgtcgagatgcacacaagagtgaggttgctactcggtttaaagatttgggagaagaaaatttcaaagccttggtgttgattgcctttgctcagtatcttcagcagtgtccatttgaagatactgtaaaattagtgaatgaagtaactgaatttgcaaaaaactgtgtagctgtgaagtcagctgaaaattgtgacaaatcacttcataccctttttggagacaaattatgcacagttgcaactcttcgtgaaaccttgagtgaatgagctgactgctgtgcaaaacaagaacctgagagatgaaaatgcttcttgcaacacaaagtgaacaacccaaacctcccccgattggtcagaccagaggttgatgtgtgatgcactgcttttactgacaatgaagagacatttttgaaaaaatacttattgaaaattgccagaagaactccttactttttgaccccggaactccttttctttgctaaaaggtataaagctgcttttacagaatgttgccaagctgctgataaagctgcctgcctgttgccaaagctcgtgaaacttcgggtgaaagggaaggcttcgtctgccaaacagagactctgaaatgccagtctccaaaaatttggagaaagagctttcaaagcatgggcagtggctcgcctgagccagagatttcccaaagctga

SEQ ID NO: 45 - DNA, 4014 bp, Artificial Sequence
  Synthetic HPRT
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggagcagtacggcgacgaagtttaagtactctgtgctggaaacagcacagaatctacttaaacaaggcaaaatgccgtgtttatctcgtcaacttgttggcgagatttttttcaccggtggcacacactgtagttcatctttacatggcctcattgaagactacagctctggtatgcgtataaggaactagcattaggtcatttcaagccgatgctagaatccagattccatgctgaccgatgaggatatagtgagaatctttcaagaacattcttaaccgttggtatcttagctccaccctcactggttcttccggccaagctgctggcctccctcctcaaccgttctgatcatgcttgcttagtcggccagttaagcctgattatgacctggttacctgttgtctaagggcaggaatcaccgccgtaactctagcacttagcacagtacttggcttgtaagaggtcctcgatgatgtgaatacattaaataattaacctaagaaagatttcatattaggcattgtaatgacttaaggtaaagagcagtgctattaacaatccagcttgtttgggctattgtggctgtgggcacctctctgggtgtatatctgaggtgctggctacctcttggaggattataagacaatcagcaacccttgcatggtggcaacagtaataatagccatccttacatagtcctacagccctgtagcaatggtccaacagatgaggaacctttgaagcctcagagaggctaacagacagaccctaggtcatacagttattaagagaaggcgaacctctctcgagtaataccagttaataggctacacaaatggtagtggctgttgtattcagttgctgaggaatgctaaacataattctgccaatttccgcacccgacttcccgggctcgggtgattctagggctgtgtcatttgtatacgctcttgttgcccgggctggagtacagtggcctcagtgctcccgggttccctacctcatgcgcctgtataatagagacgaggtttcacaggctacctgatccagtgaatatttgtattgtagagatggtggccatgttcctgagctcaagcgatctgcccgcctctggccaccgtgcctggcctaggtagacgcagcgtgatgcctgagtatatagtgatgctagagctggctgtttgttagctttgaacataagatactcattgtagtttgcaaatccctcttcctaatttctttcccttaaattgtttgcatgttagcgcttaaatggtgctatgtgctagaagccttaaattacacaaatcagagaggtgcccaactttgaacctaagctgctcttaatctctaaacaagttagtagtgacaatagtaggatacttaactatgaggcatagcaggcattatcaccctaaagtgtaccctttaggtaagtatatacttgcccaatatcacttatcaaatgtgtctgatacaacccaaactatcgaaactgccagggtaaacttggacacacttgagctaagaattaagtcctagaaatgtaatcctgccctagccgagcttaccctgcagaattggtcggagcaccgtccttggccacactgttatcaacagggtgtcaatctgtaggaattactctttgtgaccaccaggaaatagagcagttcagttcatttctttctcactgtgacctgcatactacaagtctactttgctatccattgtttgtatctgggtattaccagatcagcagagaagagttgccttggagcagctgcagttcattagatagtaactaggccatgtcaactcccttgtagtgaagattgtactggtacctttctgtaaatattgtgtagatcaatcaccacctcaacccagtggctgccaaattacaataattcactactactaagataatctactagttcgatcacatacttcctactgtcttcagcattgtgcttctgattataattgtccagagtgaacatgtctattcttccactgtacacactaatggattgtaatattgggtaaattcatgtccttacacatgtagtagttatgagcccatgtccctagaatgagtaataaccttggttgaatagtcaagaatgctgaaattcttctaacagcagaagggaaggcaagcaagtgttactgataagatgaatctactattagctttaattatacatttaggaatattgcatcagtaactcataaggctgttatcctgagttaacacaaattatccaaggagatctgctttgaggtgtgagtgtatctgatgccaactagcaattccagaagtttggaattaaattatggtttatctattgttatacctcaattatatcatgtttgctgtgctctcggctcactctagccaccgactccctctgagccttgcagggtagagacaggattggccaggatggtctccatcatgatcggcctcgtgggagccactacgcctggccatagactcacttccattaagtcttgtttggacccacgaacattgtctttaagatggagtttcacgttgcccagactgtagtgcaatggtgcaatctcagctcactgcaaccaattctcctcccgagtagctggaattacaggcgcccgccaccacggtgtttcaccggccatgatccgcccacctcagcctcgtgtgagccaccgcatctggccaacatgtcttcctagacttaagcacagatgatgaattgatgtgtcttagcttggattaacttgcttactgtaaagataatatagcttgacatgaaggccattattacagatgtgacgtgcataattattagtattacatgggtcagtctggcaattatgaagaataatgccagacatttcagtaatcgattatagcgtattgacagtccagacgtcagaatttctcaatactctttcagattaatgtacctgtagcgatatcattcacaagtatatcacaagtaagttagaatttgagaactgtgttctagagatgcagtcagatttctgaactgtctcagcaaatggagagctagtaattaataacctgtcctttgatttctgattcagccaagaatggccatatttgggaaggagagtaaccacgcattcatttaccacagagctctcagcttaaagccatacaggaccgtgatctgttctagccatatgtagcatttatgtcctagtgtgatggtatttggagacagggcctttggaaggtaattgaagtgggcccaggtctgattggattagtgcgggcgcacaaggccaatcacgaggtcagccagcctggccaatgtagtgaaacaccaacattagctgggtgtggtagcgggctcctgtcatccaagctacgaggcatgagaatcgggacagattgtgccactgtgggtgactcaagagacaccagagagcttgttagaagaggtcatgtgagcacgaccttcaagccaaagaagaggcctgagattgaaacctaccttgcaggtattccgtgagaaataagtttctgttaagtcactcagtctgtggtagttatggcagcctgagcaggtagttgttctttcagaaggtgttgataatcaga

SEQ ID NO: 46 - protein, 1053 aa, S. aureus
  misc_feature (1)..(1053): Wild-type  S. aureus Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 47 - protein, 1053 aa, S. aureus
  misc_feature (1)..(1053): S. aureus Cas9 D10 variant
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 48 - protein, 1053 aa, S. aureus
  misc_feature (1)..(1053): S. aureus N580A variant
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 49 - protein, 1053 aa, S. aureus
  misc_feature (1)..(1053): S. aureus D10 & N580A variant
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 50 - DNA, 27 bp, Artificial Sequence
  Synthetic R42BS
gtgttattacttgctactgcagagagt

SEQ ID NO: 51 - DNA, 595 bp, Artificial Sequence
  Synthetic CMV Promoter
ggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgt

SEQ ID NO: 52 - DNA, 3225 bp, Artificial Sequence
  Synthetic SaCas9
atggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaa

SEQ ID NO: 53 - DNA, 107 bp, Artificial Sequence
  Synthetic Intron
gtaagtatcaaggttacaagacagcttgtcgagacagagaagactcttgcgtttctgataggcacctattggtcttactgacatccactttgcctttctctccacag

SEQ ID NO: 54 - DNA, 133 bp, Artificial Sequence
  Synthetic Intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 55 - DNA, 70 bp, Homo sapiens
  misc_feature (1)..(70): BCL11A intron 2
gtatgtctacatttctcttaggtaaacatctaaggcatttcgagaacacagaaaaggttttgagtttgag

SEQ ID NO: 56 - DNA, 45 bp, Homo sapiens
  misc_feature (1)..(45): Retinoblastoma intron 16
gttaatatttcataaatagttactttttttttcatttttaggaag

SEQ ID NO: 57 - DNA, 3359 bp, Artificial Sequence
  Synthetic SaCas9 with intron containing R32BS
atggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacagctattctgagtacagagcatacagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaa

SEQ ID NO: 58 - DNA, 49 bp, Artificial Sequence
  Synthetic Poly A
aataaaatatctttattttcattacatctgtgtgttggttttttgtgtg

SEQ ID NO: 59 - DNA, 4860 bp, Artificial Sequence
  Synthetic CTX-212
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgtattgcttgtactactcactgaatcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 60 - DNA, 4833 bp, Artificial Sequence
  Synthetic CTX-214
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgttattacttgctactgcagagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 61 - DNA, 4972 bp, Artificial Sequence
  Synthetic CTX-217
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgtattgcttgtactactcactgaatgccaccatggccccaaagaagaagcggaaggtcggatccggaaagcggaactatatcctgggactgggtaagtgtattgcttgtactactcactgaatcaccatcgggcgcgaagggggagacctgtagtcagagcccccgggcagcacacactgacatccactcccttcctattgtttcagacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtgttattacttgctactgcagagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccgctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctgtgttattacttgctactgcagagagtaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 62 - DNA, 4952 bp, Artificial Sequence
  Synthetic CTX-506
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttagaggtcttctacatacagtttaagtactctgtgctggaaacagcacagaatctacttaaacaaggcaaaatgccgtgtttatctcgtcaacttgttggcgagatttttttcaccggtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaaggtactagtaataaaatatctttattttcattacatctgtgtgttggttttttgtgtgagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctgagtacagagcatagtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 63 - DNA, 4956 bp, Artificial Sequence
  Synthetic CTX-507
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttctgactgtaagtacactatgtttaagtactctgtgctggaaacagcacagaatctacttaaacaaggcaaaatgccgtgtttatctcgtcaacttgttggcgagatttttttcaccggtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaaggtactagtaataaaatatctttattttcattacatctgtgtgttggttttttgtgtgagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctgagtacagagcatagtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 64 - DNA, 4705 bp, Artificial Sequence
  Synthetic CTX-603
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactatgattaaatgcttgatagtttaagtactctgtgctggaaacagcacagaatctacttaaacaaggcaaaatgccgtgtttatctcgtcaacttgttggcgagatttttttcaccggtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccggtgccaccatggccccaaagaagaagcggaaggtcggatccgagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataagcgctaataaaatatctttattttcattacatctgtgtgttggttttttgtgtgagaaaacgccagtaagtgacagagtcacaaatgactgcacagagtccttggtgaacaggcgaccatgcttttcagctctggaagtcgtgaaaacatacgttcccaaagagttttgaactgaaaacttcaccttccatgcagatatatgcacactttctgagaaggagagacaaatcaagaaacaaactgcacttgttgagcttgtgaaacacaagcccaaggcaacaaaagagcaactgaaagctgtttgagatgatttcgcagcttttgtagagaagtgctgcaaggctgacgataaggagacctgctttgccgaggagggtaaaaaacttgttgctgcaagtcaagctgccttaggcttataacatctacatttaaaagactctcagcctacctgaagaataagagaaagaaatgaaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagttaagtttgcagaagtttccaagttagtgacagatcttaccaaagtccacacggaatgctgcctgagagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagtgacctgctgacttgccttacttagctgctgattttgttgaaagtaaggtgatttgcaaaaacttgactgaggcaaaggatgtcttcctgggctgatttttgtatgaatatgcaagaaggactcctgattactctgtcgtgctgctgctgagacttgccaagaactatgaaaccacagatctgaagtgctgtgccgctgcagatcctactgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctcggaaaagtgggcagcaaatgttgtaaacatcctgaagcaaaaagatgaccctgtgcagaagactatctatccgtggtcctgaaccagttatgtgtgttgcatgaggatgtcttctggcaatttcatataagtattttttcaaaatgatctcttctgtcaaccccacgcctttggcacatgaaagtgggtaacctttatttcccttctttttctctttagctcggcttattccaggggtgtgtttcgtcgagatgcacacaagagtgaggttgctactcggtttaaagatttgggagaagaaaatttcaaagccttggtgttgattgcctttgctcagtatcttcagcagtgtccatttgaagatactgtaaaattagtgaatgaagtaactgaatttgcaaaaaactgtgtagctgtgaagtcagctgaaaattgtgacaaatcacttcataccctttttggagacaaattatgcacagttgcaactcttcgtgaaaccttgagtgaatgagctgactgctgtgcaaaacaagaacctgagagatgaaaatgcttcttgcaacacaaagtgaacaacccaaacctcccccgattggtcagaccagaggttgatgtgtgatgcactgcttttactgacaatgaagagacatttttgaaaaaatacttattgaaaattgccagaagaactccttactttttgaccccggaactccttttctttgctaaaaggtataaagctgcttttacagaatgttgccaagctgctgataaagctgcctgcctgttgccaaagctcgtgaaacttcgggtgaaagggaaggcttcgtctgccaaacagagactctgaaatgccagtctccaaaaatttggagaaagagctttcaaagcatgggcagtggctcgcctgagccagagatttcccaaagctgagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttaaaggcttcatataaggggtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 4197 bp, Artificial Sequence
  Synthetic CTX-1074
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggagcagtacggcgacgaagtttaagtactctgtgctggaaacagcacagaatctacttaaacaaggcaaaatgccgtgtttatctcgtcaacttgttggcgagatttttttcaccggtggcacacactgtagttcatctttacatggcctcattgaagactacagctctggtatgcgtataaggaactagcattaggtcatttcaagccgatgctagaatccagattccatgctgaccgatgaggatatagtgagaatctttcaagaacattcttaaccgttggtatcttagctccaccctcactggttcttccggccaagctgctggcctccctcctcaaccgttctgatcatgcttgcttagtcggccagttaagcctgattatgacctggttacctgttgtctaagggcaggaatcaccgccgtaactctagcacttagcacagtacttggcttgtaagaggtcctcgatgatgtgaatacattaaataattaacctaagaaagatttcatattaggcattgtaatgacttaaggtaaagagcagtgctattaacaatccagcttgtttgggctattgtggctgtgggcacctctctgggtgtatatctgaggtgctggctacctcttggaggattataagacaatcagcaacccttgcatggtggcaacagtaataatagccatccttacatagtcctacagccctgtagcaatggtccaacagatgaggaacctttgaagcctcagagaggctaacagacagaccctaggtcatacagttattaagagaaggcgaacctctctcgagtaataccagttaataggctacacaaatggtagtggctgttgtattcagttgctgaggaatgctaaacataattctgccaatttccgcacccgacttcccgggctcgggtgattctagggctgtgtcatttgtatacgctcttgttgcccgggctggagtacagtggcctcagtgctcccgggttccctacctcatgcgcctgtataatagagacgaggtttcacaggctacctgatccagtgaatatttgtattgtagagatggtggccatgttcctgagctcaagcgatctgcccgcctctggccaccgtgcctggcctaggtagacgcagcgtgatgcctgagtatatagtgatgctagagctggctgtttgttagctttgaacataagatactcattgtagtttgcaaatccctcttcctaatttctttcccttaaattgtttgcatgttagcgcttaaatggtgctatgtgctagaagccttaaattacacaaatcagagaggtgcccaactttgaacctaagctgctcttaatctctaaacaagttagtagtgacaatagtaggatacttaactatgaggcatagcaggcattatcaccctaaagtgtaccctttaggtaagtatatacttgcccaatatcacttatcaaatgtgtctgatacaacccaaactatcgaaactgccagggtaaacttggacacacttgagctaagaattaagtcctagaaatgtaatcctgccctagccgagcttaccctgcagaattggtcggagcaccgtccttggccacactgttatcaacagggtgtcaatctgtaggaattactctttgtgaccaccaggaaatagagcagttcagttcatttctttctcactgtgacctgcatactacaagtctactttgctatccattgtttgtatctgggtattaccagatcagcagagaagagttgccttggagcagctgcagttcattagatagtaactaggccatgtcaactcccttgtagtgaagattgtactggtacctttctgtaaatattgtgtagatcaatcaccacctcaacccagtggctgccaaattacaataattcactactactaagataatctactagttcgatcacatacttcctactgtcttcagcattgtgcttctgattataattgtccagagtgaacatgtctattcttccactgtacacactaatggattgtaatattgggtaaattcatgtccttacacatgtagtagttatgagcccatgtccctagaatgagtaataaccttggttgaatagtcaagaatgctgaaattcttctaacagcagaagggaaggcaagcaagtgttactgataagatgaatctactattagctttaattatacatttaggaatattgcatcagtaactcataaggctgttatcctgagttaacacaaattatccaaggagatctgctttgaggtgtgagtgtatctgatgccaactagcaattccagaagtttggaattaaattatggtttatctattgttatacctcaattatatcatgtttgctgtgctctcggctcactctagccaccgactccctctgagccttgcagggtagagacaggattggccaggatggtctccatcatgatcggcctcgtgggagccactacgcctggccatagactcacttccattaagtcttgtttggacccacgaacattgtctttaagatggagtttcacgttgcccagactgtagtgcaatggtgcaatctcagctcactgcaaccaattctcctcccgagtagctggaattacaggcgcccgccaccacggtgtttcaccggccatgatccgcccacctcagcctcgtgtgagccaccgcatctggccaacatgtcttcctagacttaagcacagatgatgaattgatgtgtcttagcttggattaacttgcttactgtaaagataatatagcttgacatgaaggccattattacagatgtgacgtgcataattattagtattacatgggtcagtctggcaattatgaagaataatgccagacatttcagtaatcgattatagcgtattgacagtccagacgtcagaatttctcaatactctttcagattaatgtacctgtagcgatatcattcacaagtatatcacaagtaagttagaatttgagaactgtgttctagagatgcagtcagatttctgaactgtctcagcaaatggagagctagtaattaataacctgtcctttgatttctgattcagccaagaatggccatatttgggaaggagagtaaccacgcattcatttaccacagagctctcagcttaaagccatacaggaccgtgatctgttctagccatatgtagcatttatgtcctagtgtgatggtatttggagacagggcctttggaaggtaattgaagtgggcccaggtctgattggattagtgcgggcgcacaaggccaatcacgaggtcagccagcctggccaatgtagtgaaacaccaacattagctgggtgtggtagcgggctcctgtcatccaagctacgaggcatgagaatcgggacagattgtgccactgtgggtgactcaagagacaccagagagcttgttagaagaggtcatgtgagcacgaccttcaagccaaagaagaggcctgagattgaaacctaccttgcaggtattccgtgagaaataagtttctgttaagtcactcagtctgtggtagttatggcagcctgagcaggtagttgttctttcagaaggtgttgataatcagatgctagcggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 66 - DNA, 3354 bp, Artificial Sequence
  Synthetic CTX-769
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgttggagcggggagaaggccgtttaagtactctgtgctggaaacagcacagaatctacttaaacaaggcaaaatgccgtgtttatctcgtcaacttgttggcgagatttttttcaccggtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgctaataaaatatctttattttcattacatctgtgtgttggttttttgtgtgagaaaacgccagtaagtgacagagtcacaaatgactgcacagagtccttggtgaacaggcgaccatgcttttcagctctggaagtcgtgaaaacatacgttcccaaagagttttgaactgaaaacttcaccttccatgcagatatatgcacactttctgagaaggagagacaaatcaagaaacaaactgcacttgttgagcttgtgaaacacaagcccaaggcaacaaaagagcaactgaaagctgtttgagatgatttcgcagcttttgtagagaagtgctgcaaggctgacgataaggagacctgctttgccgaggagggtaaaaaacttgttgctgcaagtcaagctgccttaggcttataacatctacatttaaaagactctcagcctacctgaagaataagagaaagaaatgaaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagttaagtttgcagaagtttccaagttagtgacagatcttaccaaagtccacacggaatgctgcctgagagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagtgacctgctgacttgccttacttagctgctgattttgttgaaagtaaggtgatttgcaaaaacttgactgaggcaaaggatgtcttcctgggctgatttttgtatgaatatgcaagaaggactcctgattactctgtcgtgctgctgctgagacttgccaagaactatgaaaccacagatctgaagtgctgtgccgctgcagatcctactgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctcggaaaagtgggcagcaaatgttgtaaacatcctgaagcaaaaagatgaccctgtgcagaagactatctatccgtggtcctgaaccagttatgtgtgttgcatgaggatgtcttctggcaatttcatataagtattttttcaaaatgatctcttctgtcaaccccacgcctttggcacatgaaagtgggtaacctttatttcccttctttttctctttagctcggcttattccaggggtgtgtttcgtcgagatgcacacaagagtgaggttgctactcggtttaaagatttgggagaagaaaatttcaaagccttggtgttgattgcctttgctcagtatcttcagcagtgtccatttgaagatactgtaaaattagtgaatgaagtaactgaatttgcaaaaaactgtgtagctgtgaagtcagctgaaaattgtgacaaatcacttcataccctttttggagacaaattatgcacagttgcaactcttcgtgaaaccttgagtgaatgagctgactgctgtgcaaaacaagaacctgagagatgaaaatgcttcttgcaacacaaagtgaacaacccaaacctcccccgattggtcagaccagaggttgatgtgtgatgcactgcttttactgacaatgaagagacatttttgaaaaaatacttattgaaaattgccagaagaactccttactttttgaccccggaactccttttctttgctaaaaggtataaagctgcttttacagaatgttgccaagctgctgataaagctgcctgcctgttgccaaagctcgtgaaacttcgggtgaaagggaaggcttcgtctgccaaacagagactctgaaatgccagtctccaaaaatttggagaaagagctttcaaagcatgggcagtggctcgcctgagccagagatttcccaaagctgagctagcggtacccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 4311 bp, Artificial Sequence
  Synthetic CTX-1047
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgaattccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtgctagcactcattgtatgtagaagacctctaaggccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacagctattctgagtacagagcatacagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaaggtactagtaataaaatatctttattttcattacatctgtgtgttggttttttgtgtgagcgctggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 68 - DNA, 4562 bp, Artificial Sequence
  Synthetic CTX-1070
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttagaggtcttctacatacagtttaagtactctgtgctggaaacagcacagaatctacttaaacaaggcaaaatgccgtgtttatctcgtcaacttgttggcgagatttttttcaccggtggcacacactgtagttcatctttacatggcctcattgaagactacagctctggtatgcgtataaggaactagcattaggtcatttcaagccgatgctagaatccagattccatgctgaccgatgaggatatagtgagaatctttcaagaacattcttaaccgttggtatcttagctccaccctcactggttcttccggccaagctgctggcctccctcctcaaccgttctgatcatgcttgcttagtcggccagttaagcctgattatgacctggttacctgttgtctaagggcaggaatcaccgccgtaactctagcacttagcacagtacttggcttgtaagaggtcctcgatgatgtgaatacattaaataattaacctaagaaagatttcatattaggcattgtaatgacttaaggtaaagagcagtgctattaacaatccagcttgtttgggctattgtggctgtgggcacctctctgggtgtatatctgaggtgctggctacctcttggaggattataagacaatcagcaacccttgcatggtggcaacagtaataatagccatccttacatagtcctacagccctgtagcaatggtccaacagatgaggaacctttgaagcctcagagaggctaacagacagaccctaggtcatacagttattaagagaaggcgaacctctctcgagtaataccagttaataggctacacaaatggtagtggctgttgtattcagttgctgaggaatgctaaacataattctgccaatttccgcacccgacttcccgggctcgggtgattctagggctgtgtcatttgtatacgctcttgttgcccgggctggagtacagtggcctcagtgctcccgggttccctacctcatgcgcctgtataatagagacgaggtttcacaggctacctgatccagtgaatatttgtattgtagagatggtggccatgttcctgagctcaagcgatctgcccgcctctggccaccgtgcctggcctaggtagacgcagcgtgatgcctgagtatatagtgatgctagagctggctgtttgttagctttgaacataagatactcattgtagtttgcaaatccctcttcctaatttctttcccttaaattgtttgcatgttagcgcttaaatggtgctatgtgctagaagccttaaattacacaaatcagagaggtgcccaactttgaacctaagctgctcttaatctctaaacaagttagtagtgacaatagtaggatacttaactatgaggcatagcaggcattatcaccctaaagtgtaccctttaggtaagtatatacttgcccaatatcacttatcaaatgtgtctgatacaacccaaactatcgaaactgccagggtaaacttggacacacttgagctaagaattaagtcctagaaatgtaatcctgccctagccgagcttaccctgcagaattggtcggagcaccgtccttggccacactgttatcaacagggtgtcaatctgtaggaattactctttgtgaccaccaggaaatagagcagttcagttcatttctttctcactgtgacctgcatactacaagtctactttgctatccattgtttgtatctgggtattaccagatcagcagagaagagttgccttggagcagctgcagttcattagatagtaactaggccatgtcaactcccttgtagtgaagattgtactggtacctttctgtaaatattgtgtagatcaatcaccacctcaacccagtggctgccaaattacaataattcactactactaagataatctactagttcgatcacatacttcctactgtcttcagcattgtgcttctgattataattgtccagagtgaacatgtctattcttccactgtacacactaatggattgtaatattgggtaaattcatgtccttacacatgtagtagttatgagcccatgtccctagaatgagtaataaccttggttgaatagtcaagaatgctgaaattcttctaacagcagaagggaaggcaagcaagtgttactgataagatgaatctactattagctttaattatacatttaggaatattgcatcagtaactcataaggctgttatcctgagttaacacaaattatccaaggagatctgctttgaggtgtgagtgtatctgatgccaactagcaattccagaagtttggaattaaattatggtttatctattgttatacctcaattatatcatgtttgctgtgctctcggctcactctagccaccgactccctctgagccttgcagggtagagacaggattggccaggatggtctccatcatgatcggcctcgtgggagccactacgcctggccatagactcacttccattaagtcttgtttggacccacgaacattgtctttaagatggagtttcacgttgcccagactgtagtgcaatggtgcaatctcagctcactgcaaccaattctcctcccgagtagctggaattacaggcgcccgccaccacggtgtttcaccggccatgatccgcccacctcagcctcgtgtgagccaccgcatctggccaacatgtcttcctagacttaagcacagatgatgaattgatgtgtcttagcttggattaacttgcttactgtaaagataatatagcttgacatgaaggccattattacagatgtgacgtgcataattattagtattacatgggtcagtctggcaattatgaagaataatgccagacatttcagtaatcgattatagcgtattgacagtccagacgtcagaatttctcaatactctttcagattaatgtacctgtagcgatatcattcacaagtatatcacaagtaagttagaatttgagaactgtgttctagagatgcagtcagatttctgaactgtctcagcaaatggagagctagtaattaataacctgtcctttgatttctgattcagccaagaatggccatatttgggaaggagagtaaccacgcattcatttaccacagagctctcagcttaaagccatacaggaccgtgatctgttctagccatatgtagcatttatgtcctagtgtgatggtatttggagacagggcctttggaaggtaattgaagtgggcccaggtctgattggattagtgcgggcgcacaaggccaatcacgaggtcagccagcctggccaatgtagtgaaacaccaacattagctgggtgtggtagcgggctcctgtcatccaagctacgaggcatgagaatcgggacagattgtgccactgtgggtgactcaagagacaccagagagcttgttagaagaggtcatgtgagcacgaccttcaagccaaagaagaggcctgagattgaaacctaccttgcaggtattccgtgagaaataagtttctgttaagtcactcagtctgtggtagttatggcagcctgagcaggtagttgttctttcagaaggtgttgataatcagatgctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctgagtacagagcatagtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 69 - DNA, 4833 bp, Artificial Sequence
  Synthetic CTX-525
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgtggaaagtccccaggctccccagcaggcagaagtatgcaaagcatgcatctcaattagtcagcaaccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcaccgactatgattaaatgcttgatattgagtgccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacagcttaaaggcttcatataagggtggaatcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtcggtactagtgagggcaggggaagtctgctaacatgcggggacgtggaggaaaatcccggccccatggctaagacttccgaacagagggtgaacattgctacactgctgacagaaaataagaagaaaatcgtggataaggcttcccaggatctgtggcggagacacccagacctgatcgcaccaggaggaattgctttctctcagagggaccgcgctctgtgcctgcgagattacggctggttcctgcatctgatcaccttttgtctgctggccggagataagggccccatcgagtctattgggctgatcagtattcgagaaatgtataactcactgggagtgcccgtccctgcaatgatggagagcattagatgcctgaaagaagccagcctgtccctgctggacgaagaggacgccaacgagaccgcaccctactttgattacattattaaggctatgagctaagcgctaataaaatatctttattttcattacatctgtgtgttggttttttgtgtggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 70 - DNA, 4311 bp, Artificial Sequence
  Synthetic CTX-1048
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgaattccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtgctagcatccacatagtgtacttacagtcagaagccaccatggccccaaagaagaagcggaaggtcggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacagctattctgagtacagagcatacagagtcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttgccccaaagaagaagcggaaggtctaaggtactagtaataaaatatctttattttcattacatctgtgtgttggttttttgtgtgagcgctggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 71 - DNA, 4562 bp, Artificial Sequence
  Synthetic CTX-1075
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttctgactgtaagtacactatgtttaagtactctgtgctggaaacagcacagaatctacttaaacaaggcaaaatgccgtgtttatctcgtcaacttgttggcgagatttttttcaccggtggcacacactgtagttcatctttacatggcctcattgaagactacagctctggtatgcgtataaggaactagcattaggtcatttcaagccgatgctagaatccagattccatgctgaccgatgaggatatagtgagaatctttcaagaacattcttaaccgttggtatcttagctccaccctcactggttcttccggccaagctgctggcctccctcctcaaccgttctgatcatgcttgcttagtcggccagttaagcctgattatgacctggttacctgttgtctaagggcaggaatcaccgccgtaactctagcacttagcacagtacttggcttgtaagaggtcctcgatgatgtgaatacattaaataattaacctaagaaagatttcatattaggcattgtaatgacttaaggtaaagagcagtgctattaacaatccagcttgtttgggctattgtggctgtgggcacctctctgggtgtatatctgaggtgctggctacctcttggaggattataagacaatcagcaacccttgcatggtggcaacagtaataatagccatccttacatagtcctacagccctgtagcaatggtccaacagatgaggaacctttgaagcctcagagaggctaacagacagaccctaggtcatacagttattaagagaaggcgaacctctctcgagtaataccagttaataggctacacaaatggtagtggctgttgtattcagttgctgaggaatgctaaacataattctgccaatttccgcacccgacttcccgggctcgggtgattctagggctgtgtcatttgtatacgctcttgttgcccgggctggagtacagtggcctcagtgctcccgggttccctacctcatgcgcctgtataatagagacgaggtttcacaggctacctgatccagtgaatatttgtattgtagagatggtggccatgttcctgagctcaagcgatctgcccgcctctggccaccgtgcctggcctaggtagacgcagcgtgatgcctgagtatatagtgatgctagagctggctgtttgttagctttgaacataagatactcattgtagtttgcaaatccctcttcctaatttctttcccttaaattgtttgcatgttagcgcttaaatggtgctatgtgctagaagccttaaattacacaaatcagagaggtgcccaactttgaacctaagctgctcttaatctctaaacaagttagtagtgacaatagtaggatacttaactatgaggcatagcaggcattatcaccctaaagtgtaccctttaggtaagtatatacttgcccaatatcacttatcaaatgtgtctgatacaacccaaactatcgaaactgccagggtaaacttggacacacttgagctaagaattaagtcctagaaatgtaatcctgccctagccgagcttaccctgcagaattggtcggagcaccgtccttggccacactgttatcaacagggtgtcaatctgtaggaattactctttgtgaccaccaggaaatagagcagttcagttcatttctttctcactgtgacctgcatactacaagtctactttgctatccattgtttgtatctgggtattaccagatcagcagagaagagttgccttggagcagctgcagttcattagatagtaactaggccatgtcaactcccttgtagtgaagattgtactggtacctttctgtaaatattgtgtagatcaatcaccacctcaacccagtggctgccaaattacaataattcactactactaagataatctactagttcgatcacatacttcctactgtcttcagcattgtgcttctgattataattgtccagagtgaacatgtctattcttccactgtacacactaatggattgtaatattgggtaaattcatgtccttacacatgtagtagttatgagcccatgtccctagaatgagtaataaccttggttgaatagtcaagaatgctgaaattcttctaacagcagaagggaaggcaagcaagtgttactgataagatgaatctactattagctttaattatacatttaggaatattgcatcagtaactcataaggctgttatcctgagttaacacaaattatccaaggagatctgctttgaggtgtgagtgtatctgatgccaactagcaattccagaagtttggaattaaattatggtttatctattgttatacctcaattatatcatgtttgctgtgctctcggctcactctagccaccgactccctctgagccttgcagggtagagacaggattggccaggatggtctccatcatgatcggcctcgtgggagccactacgcctggccatagactcacttccattaagtcttgtttggacccacgaacattgtctttaagatggagtttcacgttgcccagactgtagtgcaatggtgcaatctcagctcactgcaaccaattctcctcccgagtagctggaattacaggcgcccgccaccacggtgtttcaccggccatgatccgcccacctcagcctcgtgtgagccaccgcatctggccaacatgtcttcctagacttaagcacagatgatgaattgatgtgtcttagcttggattaacttgcttactgtaaagataatatagcttgacatgaaggccattattacagatgtgacgtgcataattattagtattacatgggtcagtctggcaattatgaagaataatgccagacatttcagtaatcgattatagcgtattgacagtccagacgtcagaatttctcaatactctttcagattaatgtacctgtagcgatatcattcacaagtatatcacaagtaagttagaatttgagaactgtgttctagagatgcagtcagatttctgaactgtctcagcaaatggagagctagtaattaataacctgtcctttgatttctgattcagccaagaatggccatatttgggaaggagagtaaccacgcattcatttaccacagagctctcagcttaaagccatacaggaccgtgatctgttctagccatatgtagcatttatgtcctagtgtgatggtatttggagacagggcctttggaaggtaattgaagtgggcccaggtctgattggattagtgcgggcgcacaaggccaatcacgaggtcagccagcctggccaatgtagtgaaacaccaacattagctgggtgtggtagcgggctcctgtcatccaagctacgaggcatgagaatcgggacagattgtgccactgtgggtgactcaagagacaccagagagcttgttagaagaggtcatgtgagcacgaccttcaagccaaagaagaggcctgagattgaaacctaccttgcaggtattccgtgagaaataagtttctgttaagtcactcagtctgtggtagttatggcagcctgagcaggtagttgttctttcagaaggtgttgataatcagatgctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattctgagtacagagcatagtttaagtactctgtgctggaaacagcacagaatctacttaaacaaggcaaaatgccgtgtttatctcgtcaacttgttggcgagatttttttggtaaccggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 72 - protein, 7 aa, Artificial Sequence
  Synthetic SV40 NLS
PKKKRKV

SEQ ID NO: 73 - protein, 7 aa, Artificial Sequence
  Synthetic SV40 NLS
PKKKRRV

SEQ ID NO: 74 - protein, 16 aa, Artificial Sequence
  Synthetic NLS of nucleoplasmin
KRPAATKKAGQAKKKK

SEQ ID NO: 75 - DNA, 27 bp, Artificial Sequence
  Synthetic L22BS
gtgtattgcttgtactactcactgaat